SEQUENCE LISTING (977 sequences)

SEQ ID NO: 1 - DNA, 36 bp, Artificial Sequence
  Synthetic
gcacgtagtataatcaactttgaaaaactgtaataa

SEQ ID NO: 2 - DNA, 36 bp, Artificial Sequence
  Synthetic
gcacgttctattatcaacttcgaaaaactataataa

SEQ ID NO: 3 - DNA, 36 bp, Artificial Sequence
  Synthetic
gcccgcagtattatcaatttcgaaaaattataataa

SEQ ID NO: 4 - DNA, 36 bp, Artificial Sequence
  Synthetic
gcgcgctctataattaacttcgaaaaactttaataa

SEQ ID NO: 5 - DNA, 36 bp, Artificial Sequence
  Synthetic
gcacgctccattattaactttgaaaaactttaataa

SEQ ID NO: 6 - DNA, 36 bp, Artificial Sequence
  Synthetic
gctcgctctatcatcaatttcgaaaaactttaataa

SEQ ID NO: 7 - DNA, 36 bp, Artificial Sequence
  Synthetic
gcacgtagtattattaacttcgaaaagttataataa

SEQ ID NO: 8 - DNA, 36 bp, Artificial Sequence
  Synthetic
gcacgttccatcattaactttgaaaaactataataa

SEQ ID NO: 9 - DNA, 36 bp, Artificial Sequence
  Synthetic
gctcgctcaatcatcaactttgaaaagctataataa

SEQ ID NO: 10 - DNA, 36 bp, Artificial Sequence
  Synthetic
gctcgctctatcatcaacttcgaaaaattgtaataa

SEQ ID NO: 11 - DNA, 36 bp, Artificial Sequence
  Synthetic
gctcgctctattatcaattttgaaaaattataataa

SEQ ID NO: 12 - DNA, 36 bp, Artificial Sequence
  Synthetic
gctcgtagtattattaatttcgaaaaattataataa

SEQ ID NO: 13 - DNA, 36 bp, Artificial Sequence
  Synthetic
gctcgttcgattatcaacttcgaaaaactgtaataa

SEQ ID NO: 14 - DNA, 36 bp, Artificial Sequence
  Synthetic
gcaagaagcatcatcaacttcgaaaaactgtaataa

SEQ ID NO: 15 - DNA, 36 bp, Artificial Sequence
  Synthetic
gcgcgttctattattaattttgaaaaattataataa

SEQ ID NO: 16 - protein, 10 aa, Artificial Sequence
  Synthetic
ARSIINFEKL

SEQ ID NO: 17 - DNA, 66 bp, Artificial Sequence
  Synthetic
gattataaagatcatgacggagactataaagaccatgacattgattacaaagacgacgatgacaaa

SEQ ID NO: 18 - DNA, 66 bp, Artificial Sequence
  Synthetic
gactataaagaccacgatggcgattataaagaccatgatattgactacaaagatgatgatgataag

SEQ ID NO: 19 - DNA, 66 bp, Artificial Sequence
  Synthetic
gattataaagatcatgatggcgactataaagatcatgatatcgattacaaagatgacgatgacaaa

SEQ ID NO: 20 - DNA, 66 bp, Artificial Sequence
  Synthetic
gactacaaagatcacgatggtgactacaaagatcacgacattgattataaagacgatgatgacaaa

SEQ ID NO: 21 - DNA, 66 bp, Artificial Sequence
  Synthetic
gattacaaagatcacgatggtgattataaggatcacgatattgattacaaagacgacgacgataaa

SEQ ID NO: 22 - DNA, 66 bp, Artificial Sequence
  Synthetic
gattacaaagatcacgatggcgattacaaagatcatgacattgactacaaagacgatgatgataaa

SEQ ID NO: 23 - DNA, 66 bp, Artificial Sequence
  Synthetic
gattacaaggatcatgatggtgattacaaagatcacgatatcgactacaaagatgatgacgataaa

SEQ ID NO: 24 - DNA, 66 bp, Artificial Sequence
  Synthetic
gactacaaagatcatgatggtgattacaaagatcatgacattgattataaagatgatgatgacaaa

SEQ ID NO: 25 - DNA, 66 bp, Artificial Sequence
  Synthetic
gattataaagaccatgatggtgattataaggatcatgatatcgattataaggatgacgacgataaa

SEQ ID NO: 26 - DNA, 66 bp, Artificial Sequence
  Synthetic
gattataaagatcacgatggcgattataaagaccacgatattgattataaagacgacgatgacaaa

SEQ ID NO: 27 - DNA, 66 bp, Artificial Sequence
  Synthetic
gactataaagaccacgatggtgattataaagatcacgacatcgactacaaagacgatgatgataaa

SEQ ID NO: 28 - DNA, 66 bp, Artificial Sequence
  Synthetic
gactacaaagatcacgacggcgattataaagatcacgatattgactataaagatgacgatgataaa

SEQ ID NO: 29 - DNA, 66 bp, Artificial Sequence
  Synthetic
gattataaagaccatgatggagattacaaagatcatgatattgactataaagacgacgacgataaa

SEQ ID NO: 30 - DNA, 66 bp, Artificial Sequence
  Synthetic
gattataaagatcacgatggtgactacaaagatcacgatatcgattataaagacgatgacgataaa

SEQ ID NO: 31 - DNA, 66 bp, Artificial Sequence
  Synthetic
gactacaaagatcacgatggtgattataaagaccatgatattgattacaaagatgatgatgacaaa

SEQ ID NO: 32 - protein, 22 aa, Artificial Sequence
  Synthetic
DYKDHDGDYKDHDIDYKDDDDK

SEQ ID NO: 33 - protein, 6 aa, Artificial Sequence
  Synthetic
GASGAS

SEQ ID NO: 34 - protein, 6 aa, Artificial Sequence
  Synthetic
GSAGSA

SEQ ID NO: 35 - protein, 4 aa, Artificial Sequence
  Synthetic
GGGG

SEQ ID NO: 36 - protein, 5 aa, Artificial Sequence
  Synthetic
GGGGS

SEQ ID NO: 37 - protein, 8 aa, Artificial Sequence
  Synthetic
VGKGGSGG

SEQ ID NO: 38 - protein, 5 aa, Artificial Sequence
  Synthetic
PAPAP

SEQ ID NO: 39 - protein, 5 aa, Artificial Sequence
  Synthetic
EAAAK

SEQ ID NO: 40 - protein, 6 aa, Artificial Sequence
  Synthetic
AYLAYL

SEQ ID NO: 41 - protein, 6 aa, Artificial Sequence
  Synthetic
LRALRA

SEQ ID NO: 42 - protein, 4 aa, Artificial Sequence
  Synthetic
RLRA

SEQ ID NO: 43 - protein, 32 aa, Artificial Sequence
  Synthetic
KENSISSMAPPASPPASPKTPIEKKHADEIDK

SEQ ID NO: 44 - protein, 19 aa, Artificial Sequence
  Synthetic
KENSISSMAPPASPPASPK

SEQ ID NO: 45 - protein, 14 aa, Artificial Sequence
  Synthetic
KTEEQPSEVNTGPR

SEQ ID NO: 46 - protein, 28 aa, Artificial Sequence
  Synthetic
KESVVDASESDLDSSMQSADESTPQPLK

SEQ ID NO: 47 - protein, 20 aa, Artificial Sequence
  Synthetic
KSEEVNASDFPPPPTDEELR

SEQ ID NO: 48 - protein, 33 aa, Artificial Sequence
  Synthetic
RGGRPTSEEFSSLNSGDFTDDENSETTEEEIDR

SEQ ID NO: 49 - protein, 17 aa, Artificial Sequence
  Synthetic
KQNTASTETTTTNEQPK

SEQ ID NO: 50 - protein, 17 aa, Artificial Sequence
  Synthetic
KQNTANTETTTTNEQPK

SEQ ID NO: 51 - protein, 19 aa, Artificial Sequence
  Synthetic
RSEVTISPAETPESPPATP

SEQ ID NO: 52 - protein, 28 aa, Artificial Sequence
  Synthetic
KASVTDTSEGDLDSSMQSADESTPQPLK

SEQ ID NO: 53 - protein, 20 aa, Artificial Sequence
  Synthetic
KNEEVNASDFPPPPTDEELR

SEQ ID NO: 54 - protein, 33 aa, Artificial Sequence
  Synthetic
RGGIPTSEEFSSLNSGDFTDDENSETTEEEIDR

SEQ ID NO: 55 - protein, 529 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 56 - protein, 529 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 57 - protein, 441 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 58 - protein, 416 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 59 - protein, 441 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 60 - DNA, 1323 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgat

SEQ ID NO: 61 - protein, 633 aa, Artificial Sequence
  Synthetic
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 62 - protein, 639 aa, Artificial Sequence
  Synthetic
MGLNRFMRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 63 - protein, 93 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASG

SEQ ID NO: 64 - protein, 200 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 65 - protein, 303 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSS

SEQ ID NO: 66 - protein, 370 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSE

SEQ ID NO: 67 - protein, 390 aa, Artificial Sequence
  Synthetic
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 68 - DNA, 1170 bp, Artificial Sequence
  Synthetic
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 69 - protein, 100 aa, Artificial Sequence
  Synthetic
MGLNRFMRAMMVVFITANCITINPDIIFAATDSEDSSLNTDEWEEEKTEEQPSEVNTGPRYETAREVSSRDIKELEKSNKVRNTNKADLIAMLKEKAEKG

SEQ ID NO: 70 - protein, 390 aa, Artificial Sequence
  Synthetic
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRP

SEQ ID NO: 71 - DNA, 1170 bp, Artificial Sequence
  Synthetic
atgcgtgcgatgatggtagttttcattactgccaactgcattacgattaaccccgacataatatttgcagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtccgaataacaataataacaacggtgagcaaacaggaaatgtggctataaatgaagaggcttcaggagtcgaccgaccaactctgcaagtggagcgtcgtcatccaggtctgtcatcggatagcgcagcggaaattaaaaaaagaagaaaagccatagcgtcgtcggatagtgagcttgaaagccttacttatccagataaaccaacaaaagcaaataagagaaaagtggcgaaagagtcagttgtggatgcttctgaaagtgacttagattctagcatgcagtcagcagacgagtctacaccacaacctttaaaagcaaatcaaaaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccgatgcttctcggttttaatgctcctactccatcggaaccgagctcattcgaatttccgccgccacctacggatgaagagttaagacttgctttgccagagacgccaatgcttcttggttttaatgctcctgctacatcggaaccgagctcattcgaatttccaccgcctccaacagaagatgaactagaaattatgcgggaaacagcaccttcgctagattctagttttacaagcggggatttagctagtttgagaagtgctattaatcgccatagcgaaaatttctctgatttcccactaatcccaacagaagaagagttgaacgggagaggcggtagacca

SEQ ID NO: 72 - protein, 226 aa, Artificial Sequence
  Synthetic
MKKIMLVFITLILVSLPIAQQTEASRATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 73 - protein, 5 aa, Artificial Sequence
  Synthetic
QDNKR

SEQ ID NO: 74 - protein, 11 aa, Artificial Sequence
  Synthetic
ECTGLAWEWWR

SEQ ID NO: 75 - protein, 11 aa, Artificial Sequence
  Synthetic
ESLLMWITQCR

SEQ ID NO: 76 - protein, 368 aa, Artificial Sequence
  Synthetic
MVTGWHRPTWIEIDRAAIRENIKNEQNKLPESVDLWAVVKANAYGHGIIEVARTAKEAGAKGFCVAILDEALALREAGFQDDFILVLGATRKEDANLAAKNHISLTVFREDWLENLTLEATLRIHLKVDSGMGRLGIRTTEEARRIEATSTNDHQLQLEGIYTHFATADQLETSYFEQQLAKFQTILTSLKKRPTYVHTANSAASLLQPQIGFDAIRFGISMYGLTPSTEIKTSLPFELKPALALYTEMVHVKELAPGDSVSYGATYTATEREWVATLPIGYADGLIRHYSGFHVLVDGEPAPIIGRVCMDQTIIKLPREFQTGSKVTIIGKDHGNTVTADDAAQYLDTINYEVTCLLNERIPRKYIH

SEQ ID NO: 77 - protein, 289 aa, Artificial Sequence
  Synthetic
MKVLVNNHLVEREDATVDIEDRGYQFGDGVYEVVRLYNGKFFTYNEHIDRLYASAAKIDLVIPYSKEELRELLEKLVAENNINTGNVYLQVTRGVQNPRNHVIPDDFPLEGVLTAAAREVPRNERQFVEGGTAITEEDVRWLRCDIKSLNLLGNILAKNKAHQQNALEAILHRGEQVTECSASNVSIIKDGVLWTHAADNLILNGITRQVIIDVAKKNGIPVKEADFTLTDLREADEVFISSTTIEITPITHIDGVQVADGKRGPITAQLHQYFVEEITRACGELEFAK

SEQ ID NO: 78 - DNA, 1107 bp, Artificial Sequence
  Synthetic
atggtgacaggctggcatcgtccaacatggattgaaatagaccgcgcagcaattcgcgaaaatataaaaaatgaacaaaataaactcccggaaagtgtcgacttatgggcagtagtcaaagctaatgcatatggtcacggaattatcgaagttgctaggacggcgaaagaagctggagcaaaaggtttctgcgtagccattttagatgaggcactggctcttagagaagctggatttcaagatgactttattcttgtgcttggtgcaaccagaaaagaagatgctaatctggcagccaaaaaccacatttcacttactgtttttagagaagattggctagagaatctaacgctagaagcaacacttcgaattcatttaaaagtagatagcggtatggggcgtctcggtattcgtacgactgaagaagcacggcgaattgaagcaaccagtactaatgatcaccaattacaactggaaggtatttacacgcattttgcaacagccgaccagctagaaactagttattttgaacaacaattagctaagttccaaacgattttaacgagtttaaaaaaacgaccaacttatgttcatacagccaattcagctgcttcattgttacagccacaaatcgggtttgatgcgattcgctttggtatttcgatgtatggattaactccctccacagaaatcaaaactagcttgccgtttgagcttaaacctgcacttgcactctataccgagatggttcatgtgaaagaacttgcaccaggcgatagcgttagctacggagcaacttatacagcaacagagcgagaatgggttgcgacattaccaattggctatgcggatggattgattcgtcattacagtggtttccatgttttagtagacggtgaaccagctccaatcattggtcgagtttgtatggatcaaaccatcataaaactaccacgtgaatttcaaactggttcaaaagtaacgataattggcaaagatcatggtaacacggtaacagcagatgatgccgctcaatatttagatacaattaattatgaggtaacttgtttgttaaatgagcgcatacctagaaaatacatccattag

SEQ ID NO: 79 - DNA, 870 bp, Artificial Sequence
  Synthetic
atgaaagtattagtaaataaccatttagttgaaagagaagatgccacagttgacattgaagaccgcggatatcagtttggtgatggtgtatatgaagtagttcgtctatataatggaaaattctttacttataatgaacacattgatcgcttatatgctagtgcagcaaaaattgacttagttattccttattccaaagaagagctacgtgaattacttgaaaaattagttgccgaaaataatatcaatacagggaatgtctatttacaagtgactcgtggtgttcaaaacccacgtaatcatgtaatccctgatgatttccctctagaaggcgttttaacagcagcagctcgtgaagtacctagaaacgagcgtcaattcgttgaaggtggaacggcgattacagaagaagatgtgcgctggttacgctgtgatattaagagcttaaaccttttaggaaatattctagcaaaaaataaagcacatcaacaaaatgctttggaagctattttacatcgcggggaacaagtaacagaatgttctgcttcaaacgtttctattattaaagatggtgtattatggacgcatgcggcagataacttaatcttaaatggtatcactcgtcaagttatcattgatgttgcgaaaaagaatggcattcctgttaaagaagcggatttcactttaacagaccttcgtgaagcggatgaagtgttcatttcaagtacaactattgaaattacacctattacgcatattgacggagttcaagtagctgacggaaaacgtggaccaattacagcgcaacttcatcaatattttgtagaagaaatcactcgtgcatgtggcgaattagagtttgcaaaataa

SEQ ID NO: 80 - protein, 237 aa, Artificial Sequence
  Synthetic
MNAQAEEFKKYLETNGIKPKQFHKKELIFNQWDPQEYCIFLYDGITKLTSISENGTIMNLQYYKGAFVIMSGFIDTETSVGYYNLEVISEQATAYVIKINELKELLSKNLTHFFYVFQTLQKQVSYSLAKFNDFSINGKLGSICGQLLILTYVYGKETPDGIKITLDNLTMQELGYSSGIAHSSAVSRIISKLKQEKVIVYKNSCFYVQNLDYLKRYAPKLDEWFYLACPATWGKLN

SEQ ID NO: 81 - DNA, 714 bp, Artificial Sequence
  Synthetic
atgaacgctcaagcagaagaattcaaaaaatatttagaaactaacgggataaaaccaaaacaatttcataaaaaagaacttatttttaaccaatgggatccacaagaatattgtatttttctatatgatggtatcacaaagctcacgagtattagcgagaacgggaccatcatgaatttacaatactacaaaggggctttcgttataatgtctggctttattgatacagaaacatcggttggctattataatttagaagtcattagcgagcaggctaccgcatacgttatcaaaataaacgaactaaaagaactactgagcaaaaatcttacgcactttttctatgttttccaaaccctacaaaaacaagtttcatacagcctagctaaatttaatgatttttcgattaacgggaagcttggctctatttgcggtcaacttttaatcctgacctatgtgtatggtaaagaaactcctgatggcatcaagattacactggataatttaacaatgcaggagttaggatattcaagtggcatcgcacatagctcagctgttagcagaattatttccaaattaaagcaagagaaagttatcgtgtataaaaattcatgcttttatgtacaaaatcttgattatctcaaaagatatgcccctaaattagatgaatggttttatttagcatgtcctgctacttggggaaaattaaattaa

SEQ ID NO: 82 - protein, 237 aa, Artificial Sequence
  Synthetic
MNAQAEEFKKYLETNGIKPKQFHKKELIFNQWDPQEYCIFLYDGITKLTSISENGTIMNLQYYKGAFVIMSGFIDTETSVGYYNLEVISEQATAYVIKINELKELLSKNLTHFFYVFQTLQKQVSYSLAKFNVFSINGKLGSICGQLLILTYVYGKETPDGIKITLDNLTMQELGYSSGIAHSSAVSRIISKLKQEKVIVYKNSCFYVQNRDYLKRYAPKLDEWFYLACPATWGKLN

SEQ ID NO: 83 - DNA, 713 bp, Artificial Sequence
  Synthetic
atgaacgctcaagcagaagaattcaaaaaatatttagaaactaacgggataaaaccaaaacaatttcataaaaaagaacttatttttaaccaatgggatccacaagaatattgtatttttctatatgatggtatcacaaagctcacgagtattagcgagaacgggaccatcatgaatttacaatactacaaaggggctttcgttataatgtctggctttattgatacagaaacatcggttggctattataatttagaagtcattagcgagcaggctaccgcatacgttatcaaaataaacgaactaaaagaactactgagcaaaaatcttacgcactttttctatgttttccaaaccctacaaaaacaagtttcatacagcctagctaaatttaatgttttttcgattaacgggaagcttggctctatttgcggtcaacttttaatcctgacctatgtgtatggtaaagaaactcctgatggcatcaagattacactggataatttaacaatgcaggagttaggatattcaagtggcatcgcacatagctcagctgttagcagaattatttccaaattaaagcaagagaaagttatcgtgtataaaaattcatgcttttatgtacaaaatctgattatctcaaaagatatgcccctaaattagatgaatggttttatttagcatgtcctgctacttggggaaaattaaattaa

SEQ ID NO: 84 - DNA, 12 bp, Artificial Sequence
  Synthetic
ggtggtggagga

SEQ ID NO: 85 - DNA, 12 bp, Artificial Sequence
  Synthetic
ggtggaggtgga

SEQ ID NO: 86 - DNA, 12 bp, Artificial Sequence
  Synthetic
ggtggaggaggt

SEQ ID NO: 87 - DNA, 12 bp, Artificial Sequence
  Synthetic
ggaggtggtgga

SEQ ID NO: 88 - DNA, 12 bp, Artificial Sequence
  Synthetic
ggaggaggtggt

SEQ ID NO: 89 - DNA, 12 bp, Artificial Sequence
  Synthetic
ggaggtggaggt

SEQ ID NO: 90 - DNA, 12 bp, Artificial Sequence
  Synthetic
ggaggaggaggt

SEQ ID NO: 91 - DNA, 12 bp, Artificial Sequence
  Synthetic
ggaggaggtgga

SEQ ID NO: 92 - DNA, 12 bp, Artificial Sequence
  Synthetic
ggaggtggagga

SEQ ID NO: 93 - DNA, 12 bp, Artificial Sequence
  Synthetic
ggtggaggagga

SEQ ID NO: 94 - DNA, 12 bp, Artificial Sequence
  Synthetic
ggaggaggagga

SEQ ID NO: 95 - protein, 529 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKEATGLAWEAARTVIDDRNLPLVKNRNISIWGTTLYPKYSNKVDNPIE

SEQ ID NO: 96 - protein, 11 aa, Artificial Sequence
  Synthetic
EATGLAWEAAR

SEQ ID NO: 97 - protein, 25 aa, Artificial Sequence
  Synthetic
MKKIMLVFITLILVSLPIAQQTEAK

SEQ ID NO: 98 - protein, 29 aa, Artificial Sequence
  Synthetic
MGLNRFMRAMMVVFITANCITINPDIIFA

SEQ ID NO: 99 - protein, 21 aa, Artificial Sequence
  Synthetic
DYKDHDGDYKDHDIDYKDDDK

SEQ ID NO: 100 - protein, 9 aa, Artificial Sequence
  Synthetic
ILIGVLVGV

SEQ ID NO: 101 - protein, 9 aa, Artificial Sequence
  Synthetic
IMIGVLVGV

SEQ ID NO: 102 - protein, 9 aa, Artificial Sequence
  Synthetic
ILMGVLVGV

SEQ ID NO: 103 - protein, 9 aa, Artificial Sequence
  Synthetic
IMIGVLVGV

SEQ ID NO: 104 - protein, 9 aa, Artificial Sequence
  Synthetic
HVFGYSWYK

SEQ ID NO: 105 - protein, 9 aa, Artificial Sequence
  Synthetic
HLFGYSWYK

SEQ ID NO: 106 - protein, 9 aa, Artificial Sequence
  Synthetic
IYPNASLLF

SEQ ID NO: 107 - protein, 9 aa, Artificial Sequence
  Synthetic
IYPNASLLI

SEQ ID NO: 108 - protein, 9 aa, Artificial Sequence
  Synthetic
IPQVHTQVL

SEQ ID NO: 109 - protein, 9 aa, Artificial Sequence
  Synthetic
IPQQHTQVL

SEQ ID NO: 110 - protein, 9 aa, Artificial Sequence
  Synthetic
SLYYWPRPR

SEQ ID NO: 111 - protein, 9 aa, Artificial Sequence
  Synthetic
STYYWPRPR

SEQ ID NO: 112 - protein, 9 aa, Artificial Sequence
  Synthetic
WPRPRRYVM

SEQ ID NO: 113 - protein, 9 aa, Artificial Sequence
  Synthetic
WPRPRRYVQ

SEQ ID NO: 114 - protein, 9 aa, Artificial Sequence
  Synthetic
IMAKFLHWL

SEQ ID NO: 115 - protein, 9 aa, Artificial Sequence
  Synthetic
ILAKFLHWL

SEQ ID NO: 116 - protein, 9 aa, Artificial Sequence
  Synthetic
VYILGGSQF

SEQ ID NO: 117 - protein, 9 aa, Artificial Sequence
  Synthetic
VYILGGSQL

SEQ ID NO: 118 - protein, 9 aa, Artificial Sequence
  Synthetic
KVPEIVHFL

SEQ ID NO: 119 - protein, 9 aa, Artificial Sequence
  Synthetic
KVAELVHFL

SEQ ID NO: 120 - protein, 9 aa, Artificial Sequence
  Synthetic
YMFPVIFSK

SEQ ID NO: 121 - protein, 9 aa, Artificial Sequence
  Synthetic
YFFPVIFSK

SEQ ID NO: 122 - protein, 9 aa, Artificial Sequence
  Synthetic
IMPKAGLLF

SEQ ID NO: 123 - protein, 9 aa, Artificial Sequence
  Synthetic
IMPKAGLLI

SEQ ID NO: 124 - protein, 9 aa, Artificial Sequence
  Synthetic
LPWTMNYPL

SEQ ID NO: 125 - protein, 9 aa, Artificial Sequence
  Synthetic
LPTTMNYPL

SEQ ID NO: 126 - protein, 9 aa, Artificial Sequence
  Synthetic
MPSLREAAL

SEQ ID NO: 127 - protein, 9 aa, Artificial Sequence
  Synthetic
YPSLREAAL

SEQ ID NO: 128 - protein, 9 aa, Artificial Sequence
  Synthetic
YLFPVIFSK

SEQ ID NO: 129 - protein, 9 aa, Artificial Sequence
  Synthetic
YFFPVIFSK

SEQ ID NO: 130 - protein, 9 aa, Artificial Sequence
  Synthetic
YLMPVNSEV

SEQ ID NO: 131 - protein, 9 aa, Artificial Sequence
  Synthetic
YMMPVNSEV

SEQ ID NO: 132 - protein, 9 aa, Artificial Sequence
  Synthetic
VWGIRLEHF

SEQ ID NO: 133 - protein, 9 aa, Artificial Sequence
  Synthetic
VYGIRLEHF

SEQ ID NO: 134 - protein, 9 aa, Artificial Sequence
  Synthetic
RLLEFYLAV

SEQ ID NO: 135 - protein, 9 aa, Artificial Sequence
  Synthetic
RLLEFYLAM

SEQ ID NO: 136 - protein, 9 aa, Artificial Sequence
  Synthetic
APRGPHGGM

SEQ ID NO: 137 - protein, 9 aa, Artificial Sequence
  Synthetic
APRGPHGGA

SEQ ID NO: 138 - protein, 9 aa, Artificial Sequence
  Synthetic
MAPDVVAFV

SEQ ID NO: 139 - protein, 9 aa, Artificial Sequence
  Synthetic
EAPDVVAFV

SEQ ID NO: 140 - protein, 9 aa, Artificial Sequence
  Synthetic
NMTHVLYPL

SEQ ID NO: 141 - protein, 9 aa, Artificial Sequence
  Synthetic
NLTHVLYPV

SEQ ID NO: 142 - protein, 9 aa, Artificial Sequence
  Synthetic
GMAPLILSR

SEQ ID NO: 143 - protein, 9 aa, Artificial Sequence
  Synthetic
GAAPLILSR

SEQ ID NO: 144 - protein, 9 aa, Artificial Sequence
  Synthetic
TYSVSFFSW

SEQ ID NO: 145 - protein, 9 aa, Artificial Sequence
  Synthetic
TYSVSFDSL

SEQ ID NO: 146 - protein, 9 aa, Artificial Sequence
  Synthetic
NPQPVWLCL

SEQ ID NO: 147 - protein, 9 aa, Artificial Sequence
  Synthetic
NSQPVWLCL

SEQ ID NO: 148 - protein, 9 aa, Artificial Sequence
  Synthetic
LMQAEAPRL

SEQ ID NO: 149 - protein, 9 aa, Artificial Sequence
  Synthetic
LLQAEAPRL

SEQ ID NO: 150 - protein, 9 aa, Artificial Sequence
  Synthetic
RLQGISPKV

SEQ ID NO: 151 - protein, 9 aa, Artificial Sequence
  Synthetic
RLQGISPKI

SEQ ID NO: 152 - protein, 9 aa, Artificial Sequence
  Synthetic
LLLGTIHAV

SEQ ID NO: 153 - protein, 9 aa, Artificial Sequence
  Synthetic
LLLGTIHAL

SEQ ID NO: 154 - protein, 9 aa, Artificial Sequence
  Synthetic
KYKKFPWWL

SEQ ID NO: 155 - protein, 9 aa, Artificial Sequence
  Synthetic
KYKKFPHWL

SEQ ID NO: 156 - protein, 9 aa, Artificial Sequence
  Synthetic
KMSSGCAFL

SEQ ID NO: 157 - protein, 9 aa, Artificial Sequence
  Synthetic
KHSSGCAFL

SEQ ID NO: 158 - protein, 9 aa, Artificial Sequence
  Synthetic
SWFKNWPFF

SEQ ID NO: 159 - protein, 9 aa, Artificial Sequence
  Synthetic
STFKNWPFL

SEQ ID NO: 160 - protein, 9 aa, Artificial Sequence
  Synthetic
FMFPNAPYL

SEQ ID NO: 161 - protein, 9 aa, Artificial Sequence
  Synthetic
YLGEQQYSV

SEQ ID NO: 162 - protein, 9 aa, Artificial Sequence
  Synthetic
YLLPAVPSL

SEQ ID NO: 163 - protein, 9 aa, Artificial Sequence
  Synthetic
YLNALLPAV

SEQ ID NO: 164 - protein, 9 aa, Artificial Sequence
  Synthetic
ALLLRTPYV

SEQ ID NO: 165 - protein, 9 aa, Artificial Sequence
  Synthetic
YLGATLKGV

SEQ ID NO: 166 - protein, 9 aa, Artificial Sequence
  Synthetic
KLYFKLSHL

SEQ ID NO: 167 - protein, 9 aa, Artificial Sequence
  Synthetic
YMTWNQMNL

SEQ ID NO: 168 - protein, 9 aa, Artificial Sequence
  Synthetic
GLRRGIQDV

SEQ ID NO: 169 - protein, 9 aa, Artificial Sequence
  Synthetic
YMFPNAPYL

SEQ ID NO: 170 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 171 - protein, 139 aa, Homo sapiens
MSWRGRSTYYWPRPRRYVQPPEMIGPMRPEQFSDEVEPATPEEGEPATQRQDPAAAQEGEDEGASAGQGPKPEADSQEQGHPQTGCECEDGPDGQEMDPPNPEEVKTPEEEMRSHYVAQTGILWLLMNNCFLNLSPRKP

SEQ ID NO: 172 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 173 - protein, 586 aa, Homo sapiens
MAASGVEKSSKKKTEKKLAAREEAKLLAGFMGVMNNMRKQKTLCDVILMVQERKIPAHRVVLAAASHFFNLMFTTNMLESKSFEVELKDAEPDIIEQLVEFAYTARISVNSNNVQSLLDAANQYQIEPVKKMCVDFLKEQVDASNCLGISVLAECLDCPELKATADDFIHQHFTEVYKTDEFLQLDVKRVTHLLNQDTLTVRAEDQVYDAAVRWLKYDEPNRQPFMVDILAKVRFPLISKNFLSKTVQAEPLIQDNPECLKMVISGMRYHLLSPEDREELVDGTRPRRKKHDYRIALFGGSQPQSCRYFNPKDYSWTDIRCPFEKRRDAACVFWDNVVYILGGSQLFPIKRMDCYNVVKDSWYSKLGPPTPRDSLAACAAEGKIYTSGGSEVGNSALYLFECYDTRTESWHTKPSMLTQRCSHGMVEANGLIYVCGGSLGNNVSGRVLNSCEVYDPATETWTELCPMIEARKNHGLVFVKDKIFAVGGQNGLGGLDNVEYYDIKLNEWKMVSPMPWKGVTVKCAAVGSIVYVLAGFQGVGRLGHILEYNTETDKWVANSKVRAFPVTSCLICVVDTCGANEETLET

SEQ ID NO: 174 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 175 - protein, 317 aa, Homo sapiens
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 176 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAKLVHFLLLKYRAREPVTKAEMLGSVVGNWQYFFPVIFSKASDSLQLVFGIELMEVDPIGHVYIFATCLGLSYDGLLGDNQIMPKTGFLIIILAIIAKEGDCAPEEKIWEELSVLEVFEGREDSIFGDPKKLLTQYFVQENYLEYRQVPGSDPACYEFLWGPRALIETSYVKVLHHMVKISGGPRISYPLLHEWALREGEE

SEQ ID NO: 177 - protein, 464 aa, Homo sapiens
METLSFPRYNVAEIVIHIRNKILTGADGKNLTKNDLYPNPKPEVLHMIYMRALQIVYGIRLEHFYMMPVNSEVMYPHLMEGFLPFSNLVTHLDSFLPICRVNDFETADILCPKAKRTSRFLSGIINFIHFREACRETYMEFLWQYKSSADKMQQLNAAHQEALMKLERLDSVPVEEQEEFKQLSDGIQELQQSLNQDFHQKTIVLQEGNSQKKSNISEKTKRLNELKLSVVSLKEIQESLKTKIVDSPEKLKNYKEKMKDTVQKLKNARQEVVEKYEIYGDSVDCLPSCQLEVQLYQKKIQDLSDNREKLASILKESLNLEDQIESDESELKKLKTEENSFKRLMIVKKEKLATAQFKINKKHEDVKQYKRTVIEDCNKVQEKRGAVYERVTTINQEIQKIKLGIQQLKDAAEREKLKSQEIFLNLKTALEKYHDGIEKAAEDSYAKIDEKTAELKRKMFKMST

SEQ ID NO: 178 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 179 - protein, 102 aa, Homo sapiens
MSARVRSRSRGRGDGQEAPDVVAFVAPGESQQEEPPTDNQDIEPGQEREGTPPIEERKVEGDCQEMDLEKTRSERGDGSDVKEKTPPNPKHAKTKEAGDGQP

SEQ ID NO: 180 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 181 - protein, 261 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 182 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 183 - protein, 783 aa, Homo sapiens
MSGGHQLQLAALWPWLLMATLQAGFGRTGLVLAAAVESERSAEQKAIIRVIPLKMDPTGKLNLTLEGVFAGVAEITPAEGKLMQSHPLYLCNASDDDNLEPGFISIVKLESPRRAPRPCLSLASKARMAGERGASAVLFDITEDRAAAEQLQQPLGLTWPVVLIWGNDAEKLMEFVYKNQKAHVRIELKEPPAWPDYDVWILMTVVGTIFVIILASVLRIRCRPRHSRPDPLQQRTAWAISQLATRRYQASCRQARGEWPDSGSSCSSAPVCAICLEEFSEGQELRVISCLHEFHRNCVDPWLHQHRTCPLCMFNITEGDSFSQSLGPSRSYQEPGRRLHLIRQHPGHAHYHLPAAYLLGPSRSAVARPPRPGPFLPSQEPGMGPRHHRFPRAAHPRAPGEQQRLAGAQHPYAQGWGLSHLQSTSQHPAACPVPLRRARPPDSSGSGESYCTERSGYLADGPASDSSSGPCHGSSSDSVVNCTDISLQGVHGSSSTFCSSLSSDFDPLVYCSPKGDPQRVDMQPSVTSRPRSLDSVVPTGETQVSSHVHYHRHRHHHYKKRFQWHGRKPGPETGVPQSRPPIPRTQPQPEPPSPDQQVTRSNSAAPSGRLSNPQCPRALPEPAPGPVDASSICPSTSSLFNLQKSSLSARHPQRKRRGGPSEPTPGSRPQDATVHPACQIFPHYTPSVAYPWSPEAHPLICGPPGLDKRLLPETPGPCYSNSQPVWLCLTPRQPLEPHPPGEGPSEWSSDTAEGRPCPYPHCQVLSAQPGSEEELEELCEQAV

SEQ ID NO: 184 - protein, 963 aa, Homo sapiens
MATAAETSASEPEAESKAGPKADGEEDEVKAARTRRKVLSRAVAAATYKTMGPAWDQQEEGVSESDGDEYAMASSAESSPGEYEWEYDEEEEKNQLEIERLEEQLSINVYDYNCHVDLIRLLRLEGELTKVRMARQKMSEIFPLTEELWLEWLHDEISMAQDGLDREHVYDLFEKAVKDYICPNIWLEYGQYSVGGIGQKGGLEKVRSVFERALSSVGLHMTKGLALWEAYREFESAIVEAARLEKVHSLFRRQLAIPLYDMEATFAEYEEWSEDPIPESVIQNYNKALQQLEKYKPYEEALLQAEAPRLAEYQAYIDFEMKIGDPARIQLIFERALVENCLVPDLWIRYSQYLDRQLKVKDLVLSVHNRAIRNCPWTVALWSRYLLAMERHGVDHQVISVTFEKALNAGFIQATDYVEIWQAYLDYLRRRVDFKQDSSKELEELRAAFTRALEYLKQEVEERFNESGDPSCVIMQNWARIEARLCNNMQKARELWDSIMTRGNAKYANMWLEYYNLERAHGDTQHCRKALHRAVQCTSDYPEHVCEVLLTMERTEGSLEDWDIAVQKTETRLARVNEQRMKAAEKEAALVQQEEEKAEQRKRARAEKKALKKKKKIRGPEKRGADEDDEKEWGDDEEEQPSKRRRVENSIPAAGETQNVEVAAGPAGKCAAVDVEPPSKQKEKAASLKRDMPKVLHDSSKDSITVFVSNLPYSMQEPDTKLRPLFEACGEVVQIRPIFSNRGDFRGYCYVEFKEEKSALQALEMDRKSVEGRPMFVSPCVDKSKNPDFKVFRYSTSLEKHKLFISGLPFSCTKEELEEICKAHGTVKDLRLVTNRAGKPKGLAYVEYENESQASQAVMKMDGMTIKENIIKVAISNPPQRKVPEKPETRKAPGGPMLLPQTYGARGKGRTQLSLLPRALQRPSAAAPQAENGPAAAPAVAAPAATEAPKMSNADFAKLFLRK

SEQ ID NO: 185 - protein, 188 aa, Homo sapiens
MNGDDAFARRPTVGAQIPEKIQKAFDDIAKYFSKEEWEKMKASEKIFYVYMKRKYEAMTKLGFKATLPPFMCNKRAEDFQGNDLDNDPNRGNQVERPQMTFGRLQGISPKIMPKKPAEEGNDSEEVPEASGPQNDGKELCPPGKPTTSEKIHERSGPKRGEHAWTHRLRERKQLVIYEEISDPEEDDE

SEQ ID NO: 186 - protein, 339 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 187 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 188 - protein, 75 aa, Artificial Sequence [sequence omitted]

SEQ ID NO: 189 - protein, 546 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDDYKDHDGDYKDHDIDYKDDDKQIFVKTLTGKTITLEVEPSDTIENVKAKIQDKEGIPPDQQRLIFAGKQLEDGRTLSDYNIQKESTLHLVLRLRGGFMFPNAPYL

SEQ ID NO: 190 - protein, 19 aa, Artificial Sequence
  Synthetic
RSDELVRHHNMHQRNMTKL

SEQ ID NO: 191 - protein, 22 aa, Artificial Sequence
  Synthetic
PGCNKRYFKLSHLQMHSRKHTG

SEQ ID NO: 192 - protein, 19 aa, Artificial Sequence
  Synthetic
SGQAYMFPNAPYLPSCLES

SEQ ID NO: 193 - protein, 19 aa, Artificial Sequence
  Synthetic
SGQARMFPNAPYLPSCLES

SEQ ID NO: 194 - protein, 616 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDRSDELVRHHNMHQRNMTKLGGGGGPGCNKRYFKLSHLQMHSRKHTGGGGGGSGQAYMFPNAPYLPSCLESDYKDHDGDYKDHDIDYKDDDKQIFVKTLTGKTITLEVEPSDTIENVKAKIQDKEGIPPDQQRLIFAGKQLEDGRTLSDYNIQKESTLHLVLRLRGGYMFPNAPYL

SEQ ID NO: 195 - DNA, 16 bp, Artificial Sequence
  Synthetic
catcgatcactctgga

SEQ ID NO: 196 - DNA, 19 bp, Artificial Sequence
  Synthetic
ctaactccaatgttacttg

SEQ ID NO: 197 - protein, 9 aa, Artificial Sequence
  Synthetic
RMFPNAPYL

SEQ ID NO: 198 - protein, 9 aa, Artificial Sequence
  Synthetic
SLGEQQYSV

SEQ ID NO: 199 - protein, 9 aa, Artificial Sequence
  Synthetic
ALLPAVPSL

SEQ ID NO: 200 - protein, 9 aa, Artificial Sequence
  Synthetic
DLNALLPAV

SEQ ID NO: 201 - protein, 9 aa, Artificial Sequence
  Synthetic
ALLLRTPYS

SEQ ID NO: 202 - protein, 9 aa, Artificial Sequence
  Synthetic
NLGATLKGV

SEQ ID NO: 203 - protein, 9 aa, Artificial Sequence
  Synthetic
KRYFKLSHL

SEQ ID NO: 204 - protein, 9 aa, Artificial Sequence
  Synthetic
CMTWNQMNL

SEQ ID NO: 205 - protein, 9 aa, Artificial Sequence
  Synthetic
GVFRGIQDV

SEQ ID NO: 206 - protein, 105 aa, Artificial Sequence
  Synthetic
DYKDHDGDYKDHDIDYKDDDKQIFVKTLTGKTITLEVEPSDTIENVKAKIQDKEGIPPDQQRLIFAGKQLEDGRTLSDYNIQKESTLHLVLRLRGGMPKYAYHML

SEQ ID NO: 207 - protein, 9 aa, Artificial Sequence
  Synthetic
SPSYVYHQF

SEQ ID NO: 208 - protein, 9 aa, Artificial Sequence
  Synthetic
MPKYAYHML

SEQ ID NO: 209 - protein, 6 aa, Artificial Sequence
  Synthetic
ADLVVG

SEQ ID NO: 210 - protein, 12 aa, Artificial Sequence
  Synthetic
ADLIEATAEEVL

SEQ ID NO: 211 - protein, 12 aa, Artificial Sequence
  Synthetic
GDGSIVSLAKTA

SEQ ID NO: 212 - protein, 12 aa, Artificial Sequence
  Synthetic
RDGSVADLAKVA

SEQ ID NO: 213 - protein, 12 aa, Artificial Sequence
  Synthetic
ADGSVKTLSKVL

SEQ ID NO: 214 - protein, 12 aa, Artificial Sequence
  Synthetic
GDGSIVDGSKEL

SEQ ID NO: 215 - protein, 12 aa, Artificial Sequence
  Synthetic
GDGSIKTAVKSL

SEQ ID NO: 216 - protein, 12 aa, Artificial Sequence
  Synthetic
ADLSVATLAKSL

SEQ ID NO: 217 - protein, 12 aa, Artificial Sequence
  Synthetic
ADLAVKTLAKVL

SEQ ID NO: 218 - protein, 369 aa, Artificial Sequence
  Synthetic
VGKGGSGGADLIEATAEEVLHVFGYSWYKGDGSIVSLAKTAYLFPVIFSKRDGSVADLAKVAIPQVHTQVLADGSVKTLSKVLMPSLREAALGDGSIVSLAKTAWPRPRRYVMGDGSIVDGSKELIYPNASLLFADLIEATAEEVLRLLEFYLAVGDGSIKTAVKSLLLLGTIHAVADGSVKTLSKVLKYKKFPWWLADLSVATLAKSLNPQPVWLCLADLAVKTLAKVLVGKGGSGGDYKDHDGDYKDHDIDYKDDDKADGSVKTLSKVLSIINFEKLADLVVGQIFVKTLTGKTITLEVEPSDTIENVKAKIQDKEGIPPDQQRLIFAGKQLEDGRTLSDYNIQKESTLHLVLRLRGGILIGVLVGV

SEQ ID NO: 219 - protein, 294 aa, Artificial Sequence
  Synthetic
VGKGGSGGADLIEATAEEVLHVFGYSWYKGDGSIVSLAKTAYLFPVIFSKRDGSVADLAKVAIPQVHTQVLADGSVKTLSKVLMPSLREAALGDGSIVSLAKTAWPRPRRYVMGDGSIVDGSKELIYPNASLLFADLIEATAEEVLRLLEFYLAVGDGSIKTAVKSLLLLGTIHAVADGSVKTLSKVLKYKKFPWWLADLSVATLAKSLNPQPVWLCLADLAVKTLAKVLVGKGGSGGDYKDHDGDYKDHDIDYKDDDKADGSVKTLSKVLSIINFEKLADLVVGILIGVLVGV

SEQ ID NO: 220 - DNA, 1113 bp, Artificial Sequence
  Synthetic
gttggtaaaggtggatctggaggagcagaccttatcgaagcaacagcagaagaagtattacatgtttttggttatagttggtacaaaggagatggtagtattgtaagtttagctaaaacagcttatttatttcccgttatttttagtaaacgtgacggtagtgttgcagatttagcaaaagtagcaattccacaagttcatacacaagttttagctgacggaagtgttaaaacattatctaaagtattaatgccaagtttaagagaagcagcattaggagacggaagtattgtaagtttagctaagacagcttggccacgtcctcgtcgttatgttatgggtgacggtagtatcgtagacggttctaaagaattaatttatccaaatgctagtttattatttgcagatttaattgaagctacagctgaggaagttttacgtttacttgaattttacttagcagttggtgatggaagtattaagacagctgtaaaaagtttattattattaggtacaattcacgcagttgctgacggttctgtaaaaacattaagtaaagttttaaaatacaaaaaatttccatggtggttagctgatttatctgttgcaacattagcaaaaagtttaaatccacaaccagtatggttatgtcttgctgatttagctgttaaaacacttgcaaaagttttagttggaaaaggtggtagtggtggtgactataaggatcatgatggtgactacaaagatcacgatattgattacaaagacgatgataaagcagatggtagtgttaaaactctttctaaagttttaagtattattaatttcgaaaaattagcagatttagttgttggacaaatttttgttaagacattaactggtaaaacaattacattagaggtagaaccatctgatacaatcgaaaatgtaaaagcaaaaatccaagataaagaaggtatcccaccagaccagcagcgtcttatcttcgctggtaaacaattagaagatggtcgtacattatctgattataacattcaaaaagaaagtacattacatttagttcttcgtttacgtggaggtattttaattggagttttagtaggtgtataataa

SEQ ID NO: 221 - DNA, 888 bp, Artificial Sequence
  Synthetic
gtgggtaaaggcggtagcggtggtgctgatttaattgaagctacagctgaagaagtattacatgtttttggatatagttggtataaaggtgatggatctattgtaagtttagcaaaaacagcttatttatttccagttatttttagtaaacgtgatggaagtgtagcagatttagctaaagtagcaattccacaagtacatacacaagtattagctgatggtagtgttaaaacattaagtaaagttttaatgccaagtttacgtgaagcagctttaggagatggttctattgtttctttagctaaaacagcatggccacgtccacgtcgttatgttatgggtgatggtagtattgtagatggaagtaaagaattaatttatccaaatgcttctttattatttgcagatttaattgaagcaacagcagaagaagtattacgtttattagaattttatttagcagtaggtgatggaagtattaaaacagcagttaaaagtcttcttcttcttggtacaattcatgcagtggcggatggaagtgtaaaaacactttctaaagttcttaaatataaaaaatttccatggtggttagcagatttaagtgttgcaacacttgctaaatctttaaatccacaaccagtatggctttgtcttgcagatcttgctgttaaaacattagctaaagtattagtaggaaaaggtggaagtggaggagattataaagatcatgatggtgattataaagatcatgatattgattataaagatgatgataaagcagatggtagtgtaaaaacattatctaaagtattaagtattattaattttgaaaaattagcagatttagtagttggaattcttattggtgtgcttgttggtgtttgataa

SEQ ID NO: 222 - DNA, 888 bp, Artificial Sequence
  Synthetic
gttggaaagggaggtagtggtggtgctgatttaattgaagctacagcagaggaagttttacacgtttttggttatagttggtataaaggagacggaagtatcgtatctttagcaaaaacagcttatttatttccagttattttttctaaaagagatggtagtgtagctgatttagcaaaagttgctatcccacaagtacatacacaagttttagctgacggttctgttaaaactctttctaaagtattaatgccaagtttacgtgaggctgcacttggtgatggttctatcgtttctcttgcaaaaactgcttggccacgtccacgtcgttatgttatgggagacggtagtatcgttgacggatctaaagaattaatttatccaaacgcaagtttattatttgcagatcttattgaagcaactgctgaagaagttttacgtcttttagaattttatcttgcagttggagatggaagtatcaaaacagctgttaaaagtcttttacttttaggtacaattcatgcagtagctgatggaagtgtaaaaacattaagtaaagttttaaaatataaaaaatttccatggtggttagctgatttaagtgtagcaactttagcaaaatctttaaatccacagccagtatggttatgccttgcagatttagctgtaaaaacacttgctaaagttttagttggaaaaggaggttctggtggtgactacaaagaccatgacggagattacaaagatcatgatattgattataaagatgatgataaagctgacggtagtgtaaagacacttagtaaagttcttagtattattaattttgaaaaattagctgacttagttgttggtattttaattggtgttttagttggagtttaataa

SEQ ID NO: 223 - DNA, 27 bp, Artificial Sequence
  Synthetic
cacgtatttggttatagttggtataaa

SEQ ID NO: 224 - DNA, 27 bp, Artificial Sequence
  Synthetic
cacgtatttggttatagttggtataaa

SEQ ID NO: 225 - DNA, 27 bp, Artificial Sequence
  Synthetic
catgtttttggttatagttggtataaa

SEQ ID NO: 226 - DNA, 27 bp, Artificial Sequence
  Synthetic
catgtattcggttatagctggtacaaa

SEQ ID NO: 227 - DNA, 27 bp, Artificial Sequence
  Synthetic
cacgttttcggttatagctggtacaaa

SEQ ID NO: 228 - DNA, 27 bp, Artificial Sequence
  Synthetic
catgtgtttggttatagctggtataaa

SEQ ID NO: 229 - DNA, 27 bp, Artificial Sequence
  Synthetic
cacgtgttcgggtatagttggtataag

SEQ ID NO: 230 - DNA, 27 bp, Artificial Sequence
  Synthetic
catgtttttggatattcttggtataaa

SEQ ID NO: 231 - DNA, 27 bp, Artificial Sequence
  Synthetic
catgtatttggttatagttggtataaa

SEQ ID NO: 232 - DNA, 27 bp, Artificial Sequence
  Synthetic
catgtttttggttatagttggtataaa

SEQ ID NO: 233 - DNA, 27 bp, Artificial Sequence
  Synthetic
catgtttttggatatagttggtataaa

SEQ ID NO: 234 - DNA, 27 bp, Artificial Sequence
  Synthetic
catgtttttggttattcttggtataaa

SEQ ID NO: 235 - DNA, 27 bp, Artificial Sequence
  Synthetic
catgtttttggttattcttggtacaaa

SEQ ID NO: 236 - DNA, 27 bp, Artificial Sequence
  Synthetic
catgtatttggttatagttggtacaaa

SEQ ID NO: 237 - DNA, 27 bp, Artificial Sequence
  Synthetic
catgtttttggatattcttggtataaa

SEQ ID NO: 238 - DNA, 27 bp, Artificial Sequence
  Synthetic
catgtattcggttatagttggtataaa

SEQ ID NO: 239 - DNA, 27 bp, Artificial Sequence
  Synthetic
catgtatttggttatagttggtataaa

SEQ ID NO: 240 - DNA, 27 bp, Artificial Sequence
  Synthetic
catgtttttggatatagttggtataaa

SEQ ID NO: 241 - DNA, 27 bp, Artificial Sequence
  Synthetic
catgtttttggatatagttggtataaa

SEQ ID NO: 242 - DNA, 27 bp, Artificial Sequence
  Synthetic
tatctatttccagtgatcttcagcaag

SEQ ID NO: 243 - DNA, 27 bp, Artificial Sequence
  Synthetic
tatttattcccagtgatcttcagtaaa

SEQ ID NO: 244 - DNA, 27 bp, Artificial Sequence
  Synthetic
tatttatttccagtgatcttctctaaa

SEQ ID NO: 245 - DNA, 27 bp, Artificial Sequence
  Synthetic
tatctttttccagtgattttcagcaaa

SEQ ID NO: 246 - DNA, 27 bp, Artificial Sequence
  Synthetic
tatttatttccagtgattttcagcaaa

SEQ ID NO: 247 - DNA, 27 bp, Artificial Sequence
  Synthetic
tatttatttccagtgatttttagtaaa

SEQ ID NO: 248 - DNA, 27 bp, Artificial Sequence
  Synthetic
tatttgtttccagtgattttttctaaa

SEQ ID NO: 249 - DNA, 27 bp, Artificial Sequence
  Synthetic
tatttatttccagttatttttagtaaa

SEQ ID NO: 250 - DNA, 27 bp, Artificial Sequence
  Synthetic
tatttatttccagtaatttttagtaaa

SEQ ID NO: 251 - DNA, 27 bp, Artificial Sequence
  Synthetic
tatttatttccagtaatttttagtaaa

SEQ ID NO: 252 - DNA, 27 bp, Artificial Sequence
  Synthetic
tatttatttccagttatttttagtaaa

SEQ ID NO: 253 - DNA, 27 bp, Artificial Sequence
  Synthetic
tatttatttccagttatttttagtaaa

SEQ ID NO: 254 - DNA, 27 bp, Artificial Sequence
  Synthetic
tatctttttccagttatttttagtaaa

SEQ ID NO: 255 - DNA, 27 bp, Artificial Sequence
  Synthetic
tatctttttccagttatttttagtaaa

SEQ ID NO: 256 - DNA, 27 bp, Artificial Sequence
  Synthetic
tatttatttccagttatttttagtaaa

SEQ ID NO: 257 - DNA, 27 bp, Artificial Sequence
  Synthetic
tatctttttccagttatttttagtaaa

SEQ ID NO: 258 - DNA, 27 bp, Artificial Sequence
  Synthetic
tacttatttccagtaattttctctaaa

SEQ ID NO: 259 - DNA, 27 bp, Artificial Sequence
  Synthetic
tatttatttccagttatttttagtaaa

SEQ ID NO: 260 - DNA, 27 bp, Artificial Sequence
  Synthetic
tatttatttccagttatttttagtaaa

SEQ ID NO: 261 - DNA, 27 bp, Artificial Sequence
  Synthetic
attccacaagttcatacacaagttttg

SEQ ID NO: 262 - DNA, 27 bp, Artificial Sequence
  Synthetic
attccgcaagttcatacacaagtgcta

SEQ ID NO: 263 - DNA, 27 bp, Artificial Sequence
  Synthetic
attccacaagttcatacacaagtgctt

SEQ ID NO: 264 - DNA, 27 bp, Artificial Sequence
  Synthetic
atcccacaagttcatacacaagtttta

SEQ ID NO: 265 - DNA, 27 bp, Artificial Sequence
  Synthetic
atcccacaagttcatacacaagtttta

SEQ ID NO: 266 - DNA, 27 bp, Artificial Sequence
  Synthetic
attccgcaagttcatacacaagtgctg

SEQ ID NO: 267 - DNA, 27 bp, Artificial Sequence
  Synthetic
atccctcaagttcatacacaagtactc

SEQ ID NO: 268 - DNA, 27 bp, Artificial Sequence
  Synthetic
attccacaagtacatacacaagtttta

SEQ ID NO: 269 - DNA, 27 bp, Artificial Sequence
  Synthetic
attccacaagtacatacacaagtatta

SEQ ID NO: 270 - DNA, 27 bp, Artificial Sequence
  Synthetic
attccacaagttcatacacaagtatta

SEQ ID NO: 271 - DNA, 27 bp, Artificial Sequence
  Synthetic
attccacaagtacatacacaagtatta

SEQ ID NO: 272 - DNA, 27 bp, Artificial Sequence
  Synthetic
attccacaagttcatacacaagtttta

SEQ ID NO: 273 - DNA, 27 bp, Artificial Sequence
  Synthetic
atcccacaagttcatacacaagtatta

SEQ ID NO: 274 - DNA, 27 bp, Artificial Sequence
  Synthetic
ataccacaagttcatacacaagtatta

SEQ ID NO: 275 - DNA, 27 bp, Artificial Sequence
  Synthetic
attccacaagtacatacacaagtttta

SEQ ID NO: 276 - DNA, 27 bp, Artificial Sequence
  Synthetic
atcccacaagtacacacacaagtactt

SEQ ID NO: 277 - DNA, 27 bp, Artificial Sequence
  Synthetic
attccacaagttcacactcaagtactt

SEQ ID NO: 278 - DNA, 27 bp, Artificial Sequence
  Synthetic
attccacaagtacatacacaagtatta

SEQ ID NO: 279 - DNA, 27 bp, Artificial Sequence
  Synthetic
attccacaagtacatacacaagtatta

SEQ ID NO: 280 - DNA, 27 bp, Artificial Sequence
  Synthetic
atgccgagtctacgtgaggcggcatta

SEQ ID NO: 281 - DNA, 27 bp, Artificial Sequence
  Synthetic
atgccatctctgcgcgaagcagccttg

SEQ ID NO: 282 - DNA, 27 bp, Artificial Sequence
  Synthetic
atgcctagtcttcgtgaagcagcacta

SEQ ID NO: 283 - DNA, 27 bp, Artificial Sequence
  Synthetic
atgccaagccttagagaagcagcatta

SEQ ID NO: 284 - DNA, 27 bp, Artificial Sequence
  Synthetic
atgccaagccttagagaagcagcatta

SEQ ID NO: 285 - DNA, 27 bp, Artificial Sequence
  Synthetic
atgccaagtttacgtgaagcagcattg

SEQ ID NO: 286 - DNA, 27 bp, Artificial Sequence
  Synthetic
atgccgagcttaagagaagcagcactt

SEQ ID NO: 287 - DNA, 27 bp, Artificial Sequence
  Synthetic
atgccatctttacgtgaagctgcatta

SEQ ID NO: 288 - DNA, 27 bp, Artificial Sequence
  Synthetic
atgccaagtttacgtgaagcagcatta

SEQ ID NO: 289 - DNA, 27 bp, Artificial Sequence
  Synthetic
atgccatctttacgtgaagcagcttta

SEQ ID NO: 290 - DNA, 27 bp, Artificial Sequence
  Synthetic
atgccaagtttacgtgaagcagcttta

SEQ ID NO: 291 - DNA, 27 bp, Artificial Sequence
  Synthetic
atgccatctttacgtgaagcagcttta

SEQ ID NO: 292 - DNA, 27 bp, Artificial Sequence
  Synthetic
atgccatctttacgtgaagcagcttta

SEQ ID NO: 293 - DNA, 27 bp, Artificial Sequence
  Synthetic
atgccatctttacgtgaagcagcttta

SEQ ID NO: 294 - DNA, 27 bp, Artificial Sequence
  Synthetic
atgccatctttacgtgaagctgcatta

SEQ ID NO: 295 - DNA, 27 bp, Artificial Sequence
  Synthetic
atgcctagtcttcgtgaagctgcactt

SEQ ID NO: 296 - DNA, 27 bp, Artificial Sequence
  Synthetic
atgccaagtcttcgtgaggcagcatta

SEQ ID NO: 297 - DNA, 27 bp, Artificial Sequence
  Synthetic
atgccaagtttacgtgaagcagcttta

SEQ ID NO: 298 - DNA, 27 bp, Artificial Sequence
  Synthetic
atgccaagtttacgtgaagcagcttta

SEQ ID NO: 299 - DNA, 27 bp, Artificial Sequence
  Synthetic
tggccaagacctcgaagatatgttatg

SEQ ID NO: 300 - DNA, 27 bp, Artificial Sequence
  Synthetic
tggccaagaccaagacgctatgtaatg

SEQ ID NO: 301 - DNA, 27 bp, Artificial Sequence
  Synthetic
tggcctcgtccacgcagatatgttatg

SEQ ID NO: 302 - DNA, 27 bp, Artificial Sequence
  Synthetic
tggccaagaccaagaagatatgttatg

SEQ ID NO: 303 - DNA, 27 bp, Artificial Sequence
  Synthetic
tggccaagaccaagaagatatgttatg

SEQ ID NO: 304 - DNA, 27 bp, Artificial Sequence
  Synthetic
tggccgagaccaagaagatatgtcatg

SEQ ID NO: 305 - DNA, 27 bp, Artificial Sequence
  Synthetic
tggccgcgcccccgtcgctatgttatg

SEQ ID NO: 306 - DNA, 27 bp, Artificial Sequence
  Synthetic
tggccacgtccacgtcgttatgttatg

SEQ ID NO: 307 - DNA, 27 bp, Artificial Sequence
  Synthetic
tggccacgtccacgtcgttatgtaatg

SEQ ID NO: 308 - DNA, 27 bp, Artificial Sequence
  Synthetic
tggccacgtccacgtcgttatgttatg

SEQ ID NO: 309 - DNA, 27 bp, Artificial Sequence
  Synthetic
tggccacgtccacgtcgttatgttatg

SEQ ID NO: 310 - DNA, 27 bp, Artificial Sequence
  Synthetic
tggccacgtccacgtcgttatgttatg

SEQ ID NO: 311 - DNA, 27 bp, Artificial Sequence
  Synthetic
tggccaagaccaagaagatatgtaatg

SEQ ID NO: 312 - DNA, 27 bp, Artificial Sequence
  Synthetic
tggccaagaccaagaagatatgttatg

SEQ ID NO: 313 - DNA, 27 bp, Artificial Sequence
  Synthetic
tggccacgtccacgtcgttatgttatg

SEQ ID NO: 314 - DNA, 27 bp, Artificial Sequence
  Synthetic
tggcctcgtcctcgtcgttatgttatg

SEQ ID NO: 315 - DNA, 27 bp, Artificial Sequence
  Synthetic
tggccacgtcctcgtcgttatgttatg

SEQ ID NO: 316 - DNA, 27 bp, Artificial Sequence
  Synthetic
tggccacgtccacgtcgttatgttatg

SEQ ID NO: 317 - DNA, 27 bp, Artificial Sequence
  Synthetic
tggccacgtccacgtcgttatgttatg

SEQ ID NO: 318 - DNA, 27 bp, Artificial Sequence
  Synthetic
atttatccaaatgcaagtcttttattt

SEQ ID NO: 319 - DNA, 27 bp, Artificial Sequence
  Synthetic
atctatccaaatgcaagtttgttattt

SEQ ID NO: 320 - DNA, 27 bp, Artificial Sequence
  Synthetic
atatatccaaatgcaagtcttttattt

SEQ ID NO: 321 - DNA, 27 bp, Artificial Sequence
  Synthetic
atctacccaaatgcgagcctcttattt

SEQ ID NO: 322 - DNA, 27 bp, Artificial Sequence
  Synthetic
atttacccaaatgcgagcctcttattt

SEQ ID NO: 323 - DNA, 27 bp, Artificial Sequence
  Synthetic
atttatccaaatgcaagcttattattt

SEQ ID NO: 324 - DNA, 27 bp, Artificial Sequence
  Synthetic
atttatccaaatgcgagccttttattc

SEQ ID NO: 325 - DNA, 27 bp, Artificial Sequence
  Synthetic
atttatccaaatgcttctttattattt

SEQ ID NO: 326 - DNA, 27 bp, Artificial Sequence
  Synthetic
atttatccaaatgcttctttattattt

SEQ ID NO: 327 - DNA, 27 bp, Artificial Sequence
  Synthetic
atttatccaaatgctagtttattattt

SEQ ID NO: 328 - DNA, 27 bp, Artificial Sequence
  Synthetic
atttatccaaatgcttctttattattt

SEQ ID NO: 329 - DNA, 27 bp, Artificial Sequence
  Synthetic
atttatccaaatgcaagtttattattt

SEQ ID NO: 330 - DNA, 27 bp, Artificial Sequence
  Synthetic
atatatccaaatgcatctcttcttttt

SEQ ID NO: 331 - DNA, 27 bp, Artificial Sequence
  Synthetic
atctatccaaatgcaagtcttcttttc

SEQ ID NO: 332 - DNA, 27 bp, Artificial Sequence
  Synthetic
atttatccaaatgcttctttattattt

SEQ ID NO: 333 - DNA, 27 bp, Artificial Sequence
  Synthetic
atttatccaaatgcatctttattattt

SEQ ID NO: 334 - DNA, 27 bp, Artificial Sequence
  Synthetic
atctatcctaatgcttctttacttttc

SEQ ID NO: 335 - DNA, 27 bp, Artificial Sequence
  Synthetic
atttatccaaatgcttctttattattt

SEQ ID NO: 336 - DNA, 27 bp, Artificial Sequence
  Synthetic
atttatccaaatgcttctttattattt

SEQ ID NO: 337 - DNA, 27 bp, Artificial Sequence
  Synthetic
cgcttattagaattctaccttgcggta

SEQ ID NO: 338 - DNA, 27 bp, Artificial Sequence
  Synthetic
cgtctattggaattctaccttgcggtg

SEQ ID NO: 339 - DNA, 27 bp, Artificial Sequence
  Synthetic
cgtttattggaattctaccttgcggtg

SEQ ID NO: 340 - DNA, 27 bp, Artificial Sequence
  Synthetic
cgattactagaattctatcttgcggtt

SEQ ID NO: 341 - DNA, 27 bp, Artificial Sequence
  Synthetic
agattacttgaattctatcttgcggtt

SEQ ID NO: 342 - DNA, 27 bp, Artificial Sequence
  Synthetic
cgtttattggaattttatttagcggtt

SEQ ID NO: 343 - DNA, 27 bp, Artificial Sequence
  Synthetic
agacttttagaattttatttagcggta

SEQ ID NO: 344 - DNA, 27 bp, Artificial Sequence
  Synthetic
cgtttattagaattttatttagctgta

SEQ ID NO: 345 - DNA, 27 bp, Artificial Sequence
  Synthetic
cgtttattagaattttatttagctgtt

SEQ ID NO: 346 - DNA, 27 bp, Artificial Sequence
  Synthetic
cgtttattagaattttatttagctgta

SEQ ID NO: 347 - DNA, 27 bp, Artificial Sequence
  Synthetic
cgtttattagaattttatttagcagta

SEQ ID NO: 348 - DNA, 27 bp, Artificial Sequence
  Synthetic
cgtttattagaattttatttagctgtt

SEQ ID NO: 349 - DNA, 27 bp, Artificial Sequence
  Synthetic
agacttttagaattttatttagcagtt

SEQ ID NO: 350 - DNA, 27 bp, Artificial Sequence
  Synthetic
agacttttagaattttatttagcagtt

SEQ ID NO: 351 - DNA, 27 bp, Artificial Sequence
  Synthetic
cgtttattagaattttatttagctgta

SEQ ID NO: 352 - DNA, 27 bp, Artificial Sequence
  Synthetic
agattattagaattttatttagctgtt

SEQ ID NO: 353 - DNA, 27 bp, Artificial Sequence
  Synthetic
cgtttacttgaattttaccttgcagtt

SEQ ID NO: 354 - DNA, 27 bp, Artificial Sequence
  Synthetic
cgtttattagaattttatttagcagta

SEQ ID NO: 355 - DNA, 27 bp, Artificial Sequence
  Synthetic
cgtttattagaattttatttagcagta

SEQ ID NO: 356 - DNA, 27 bp, Artificial Sequence
  Synthetic
atcttaattggcgttttagttggtgtt

SEQ ID NO: 357 - DNA, 27 bp, Artificial Sequence
  Synthetic
atcttaattggcgttttagttggtgtt

SEQ ID NO: 358 - DNA, 27 bp, Artificial Sequence
  Synthetic
atcttaattggcgttttagttggtgtt

SEQ ID NO: 359 - DNA, 27 bp, Artificial Sequence
  Synthetic
atcttaattggcgttttagttggtgtt

SEQ ID NO: 360 - DNA, 27 bp, Artificial Sequence
  Synthetic
atcttaattggcgttttagttggtgtt

SEQ ID NO: 361 - DNA, 27 bp, Artificial Sequence
  Synthetic
atcttaattggcgttttagttggtgtt

SEQ ID NO: 362 - DNA, 27 bp, Artificial Sequence
  Synthetic
atcttaattggcgttttagttggtgtt

SEQ ID NO: 363 - DNA, 27 bp, Artificial Sequence
  Synthetic
attcttattggtgtgcttgttggtgtg

SEQ ID NO: 364 - DNA, 27 bp, Artificial Sequence
  Synthetic
attcttattggtgtgttagtaggcgtt

SEQ ID NO: 365 - DNA, 27 bp, Artificial Sequence
  Synthetic
attcttattggcgttttagttggcgtt

SEQ ID NO: 366 - DNA, 27 bp, Artificial Sequence
  Synthetic
attcttattggtgtgcttgttggtgtt

SEQ ID NO: 367 - DNA, 27 bp, Artificial Sequence
  Synthetic
attcttattggcgtgttagtgggagtt

SEQ ID NO: 368 - DNA, 27 bp, Artificial Sequence
  Synthetic
attcttattggagtgttagtaggtgtt

SEQ ID NO: 369 - DNA, 27 bp, Artificial Sequence
  Synthetic
attcttattggcgtgcttgtgggcgtt

SEQ ID NO: 370 - DNA, 27 bp, Artificial Sequence
  Synthetic
attcttattggtgtgcttgttggtgtg

SEQ ID NO: 371 - DNA, 27 bp, Artificial Sequence
  Synthetic
attttaattggtgttttagttggagtt

SEQ ID NO: 372 - DNA, 27 bp, Artificial Sequence
  Synthetic
attttaattggtgttttagtaggagtt

SEQ ID NO: 373 - DNA, 27 bp, Artificial Sequence
  Synthetic
attcttattggtgtgcttgttggtgtt

SEQ ID NO: 374 - DNA, 27 bp, Artificial Sequence
  Synthetic
attcttattggtgtgcttgttggtgtt

SEQ ID NO: 375 - DNA, 27 bp, Artificial Sequence
  Synthetic
atgcctagtttaagagaagcagcatta

SEQ ID NO: 376 - DNA, 27 bp, Artificial Sequence
  Synthetic
atgccaagtttaagagaagcagcatta

SEQ ID NO: 377 - DNA, 27 bp, Artificial Sequence
  Synthetic
atgccgagtttaagagaagcagcactt

SEQ ID NO: 378 - DNA, 27 bp, Artificial Sequence
  Synthetic
atgccaagtcttcgtgaagcagcatta

SEQ ID NO: 379 - DNA, 27 bp, Artificial Sequence
  Synthetic
atgccaagtcttcgtgaagcagcatta

SEQ ID NO: 380 - DNA, 27 bp, Artificial Sequence
  Synthetic
atgcctagtttaagagaagcagcacta

SEQ ID NO: 381 - DNA, 27 bp, Artificial Sequence
  Synthetic
atgccaagtttaagagaagcggcacta

SEQ ID NO: 382 - DNA, 27 bp, Artificial Sequence
  Synthetic
atgccatctttacgtgaagcagcatta

SEQ ID NO: 383 - DNA, 27 bp, Artificial Sequence
  Synthetic
atgccatctttacgtgaagctgcttta

SEQ ID NO: 384 - DNA, 27 bp, Artificial Sequence
  Synthetic
atgccatctttacgtgaagctgcatta

SEQ ID NO: 385 - DNA, 27 bp, Artificial Sequence
  Synthetic
atgccaagtttacgtgaagcagcttta

SEQ ID NO: 386 - DNA, 27 bp, Artificial Sequence
  Synthetic
atgccaagtttacgtgaagcagcttta

SEQ ID NO: 387 - DNA, 27 bp, Artificial Sequence
  Synthetic
atgccatctttacgtgaagcagcttta

SEQ ID NO: 388 - DNA, 27 bp, Artificial Sequence
  Synthetic
atgccaagtttacgtgaagctgcatta

SEQ ID NO: 389 - DNA, 27 bp, Artificial Sequence
  Synthetic
atgccaagtttacgtgaggcagcttta

SEQ ID NO: 390 - DNA, 27 bp, Artificial Sequence
  Synthetic
atgcctagtcttcgtgaggctgctctt

SEQ ID NO: 391 - DNA, 27 bp, Artificial Sequence
  Synthetic
atgccatctttacgtgaagcagcatta

SEQ ID NO: 392 - DNA, 27 bp, Artificial Sequence
  Synthetic
atgccaagtttacgtgaagcagcttta

SEQ ID NO: 393 - DNA, 27 bp, Artificial Sequence
  Synthetic
cttttattaggcacaattcatgcagtt

SEQ ID NO: 394 - DNA, 27 bp, Artificial Sequence
  Synthetic
cttttattaggcacaattcatgcagtt

SEQ ID NO: 395 - DNA, 27 bp, Artificial Sequence
  Synthetic
cttttattaggcacaattcatgcagtt

SEQ ID NO: 396 - DNA, 27 bp, Artificial Sequence
  Synthetic
cttttattaggcacaattcatgcagtt

SEQ ID NO: 397 - DNA, 27 bp, Artificial Sequence
  Synthetic
cttttattaggcacaattcatgcagtt

SEQ ID NO: 398 - DNA, 27 bp, Artificial Sequence
  Synthetic
cttttattaggcacaattcatgcagtt

SEQ ID NO: 399 - DNA, 27 bp, Artificial Sequence
  Synthetic
cttttattaggcacaattcatgcagtt

SEQ ID NO: 400 - DNA, 27 bp, Artificial Sequence
  Synthetic
ttacttcttggaactattcatgctgtt

SEQ ID NO: 401 - DNA, 27 bp, Artificial Sequence
  Synthetic
cttcttttaggcactattcatgctgtg

SEQ ID NO: 402 - DNA, 27 bp, Artificial Sequence
  Synthetic
cttttacttggcactattcatgctgtt

SEQ ID NO: 403 - DNA, 27 bp, Artificial Sequence
  Synthetic
cttcttcttggcactattcatgctgtg

SEQ ID NO: 404 - DNA, 27 bp, Artificial Sequence
  Synthetic
ttacttttaggcactattcatgctgtg

SEQ ID NO: 405 - DNA, 27 bp, Artificial Sequence
  Synthetic
cttcttttaggcactattcatgcggtt

SEQ ID NO: 406 - DNA, 27 bp, Artificial Sequence
  Synthetic
cttcttcttggcactattcatgcggtg

SEQ ID NO: 407 - DNA, 27 bp, Artificial Sequence
  Synthetic
ttacttttaggaacaattcacgcagtt

SEQ ID NO: 408 - DNA, 27 bp, Artificial Sequence
  Synthetic
ttattattaggaacaattcacgcagta

SEQ ID NO: 409 - DNA, 27 bp, Artificial Sequence
  Synthetic
ttacttcttggaactattcatgctgtt

SEQ ID NO: 410 - DNA, 27 bp, Artificial Sequence
  Synthetic
cttcttcttggcactattcatgctgtg

SEQ ID NO: 411 - DNA, 27 bp, Artificial Sequence
  Synthetic
aaatacaaaaaatttccatggtggctt

SEQ ID NO: 412 - DNA, 27 bp, Artificial Sequence
  Synthetic
aaatataaaaaatttccatggtggtta

SEQ ID NO: 413 - DNA, 27 bp, Artificial Sequence
  Synthetic
aaatacaaaaaatttccatggtggtta

SEQ ID NO: 414 - DNA, 27 bp, Artificial Sequence
  Synthetic
aagtacaagaagttcccatggtggtta

SEQ ID NO: 415 - DNA, 27 bp, Artificial Sequence
  Synthetic
aagtacaagaagttcccatggtggtta

SEQ ID NO: 416 - DNA, 27 bp, Artificial Sequence
  Synthetic
aaatataaaaaatttccatggtggtta

SEQ ID NO: 417 - DNA, 27 bp, Artificial Sequence
  Synthetic
aaatataaaaaatttccatggtggtta

SEQ ID NO: 418 - DNA, 27 bp, Artificial Sequence
  Synthetic
aaatataaaaaatttccatggtggtta

SEQ ID NO: 419 - DNA, 27 bp, Artificial Sequence
  Synthetic
aaatataaaaaatttccatggtggtta

SEQ ID NO: 420 - DNA, 27 bp, Artificial Sequence
  Synthetic
aaatataaaaaatttccatggtggtta

SEQ ID NO: 421 - DNA, 27 bp, Artificial Sequence
  Synthetic
aaatataaaaaatttccatggtggtta

SEQ ID NO: 422 - DNA, 27 bp, Artificial Sequence
  Synthetic
aaatataaaaaatttccatggtggtta

SEQ ID NO: 423 - DNA, 27 bp, Artificial Sequence
  Synthetic
aaatataaaaaatttccatggtggctt

SEQ ID NO: 424 - DNA, 27 bp, Artificial Sequence
  Synthetic
aaatataaaaaatttccatggtggctt

SEQ ID NO: 425 - DNA, 27 bp, Artificial Sequence
  Synthetic
aaatataagaaattcccatggtggtta

SEQ ID NO: 426 - DNA, 27 bp, Artificial Sequence
  Synthetic
aaatataaaaaatttccttggtggctt

SEQ ID NO: 427 - DNA, 27 bp, Artificial Sequence
  Synthetic
aaatataaaaaatttccatggtggtta

SEQ ID NO: 428 - DNA, 27 bp, Artificial Sequence
  Synthetic
aaatataaaaaatttccatggtggtta

SEQ ID NO: 429 - DNA, 27 bp, Artificial Sequence
  Synthetic
cgcctacaaggcatctctccaaaagtc

SEQ ID NO: 430 - DNA, 27 bp, Artificial Sequence
  Synthetic
cgcttgcaaggtatctcaccaaaagtg

SEQ ID NO: 431 - DNA, 27 bp, Artificial Sequence
  Synthetic
cgtttacaaggaatttccccaaaggtt

SEQ ID NO: 432 - DNA, 27 bp, Artificial Sequence
  Synthetic
agattacaaggcattagcccaaaagtt

SEQ ID NO: 433 - DNA, 27 bp, Artificial Sequence
  Synthetic
agattacaaggtattagcccaaaagtt

SEQ ID NO: 434 - DNA, 27 bp, Artificial Sequence
  Synthetic
cgcttacaaggtattagtcctaaggtt

SEQ ID NO: 435 - DNA, 27 bp, Artificial Sequence
  Synthetic
agacttcaaggtattagtccaaaagtt

SEQ ID NO: 436 - DNA, 27 bp, Artificial Sequence
  Synthetic
cgtttacaaggtatttctccaaaagtt

SEQ ID NO: 437 - DNA, 27 bp, Artificial Sequence
  Synthetic
cgtttacaaggtatttctccaaaagtt

SEQ ID NO: 438 - DNA, 27 bp, Artificial Sequence
  Synthetic
cgtttacaaggtattagtccaaaagta

SEQ ID NO: 439 - DNA, 27 bp, Artificial Sequence
  Synthetic
cgtttacaaggaattagtccaaaagta

SEQ ID NO: 440 - DNA, 27 bp, Artificial Sequence
  Synthetic
cgtttacaaggtattagtccaaaagtt

SEQ ID NO: 441 - DNA, 27 bp, Artificial Sequence
  Synthetic
cgtttacaaggtattagtccaaaagtt

SEQ ID NO: 442 - DNA, 27 bp, Artificial Sequence
  Synthetic
cgtttacaaggaattagtccaaaagtt

SEQ ID NO: 443 - DNA, 27 bp, Artificial Sequence
  Synthetic
cgtttacaaggtatctctccaaaagta

SEQ ID NO: 444 - DNA, 27 bp, Artificial Sequence
  Synthetic
agattacaaggtatttctcctaaggtt

SEQ ID NO: 445 - DNA, 27 bp, Artificial Sequence
  Synthetic
cgtttacaaggtatttctccaaaagtt

SEQ ID NO: 446 - DNA, 27 bp, Artificial Sequence
  Synthetic
cgtttacaaggaattagtccaaaagta

SEQ ID NO: 447 - DNA, 27 bp, Artificial Sequence
  Synthetic
ttaatgcaagcagaagcaccccggctt

SEQ ID NO: 448 - DNA, 27 bp, Artificial Sequence
  Synthetic
ctaatgcaagcagaagcaccacgcctc

SEQ ID NO: 449 - DNA, 27 bp, Artificial Sequence
  Synthetic
ttgatgcaagcagaagcaccacgttta

SEQ ID NO: 450 - DNA, 27 bp, Artificial Sequence
  Synthetic
ttaatgcaagcagaagcaccaagatta

SEQ ID NO: 451 - DNA, 27 bp, Artificial Sequence
  Synthetic
ttaatgcaagcagaagcaccaagatta

SEQ ID NO: 452 - DNA, 27 bp, Artificial Sequence
  Synthetic
ttaatgcaagcggaagcaccaagactt

SEQ ID NO: 453 - DNA, 27 bp, Artificial Sequence
  Synthetic
ctcatgcaggcagaagcaccccgttta

SEQ ID NO: 454 - DNA, 27 bp, Artificial Sequence
  Synthetic
ttaatgcaagcagaagctccacgttta

SEQ ID NO: 455 - DNA, 27 bp, Artificial Sequence
  Synthetic
ttaatgcaagcagaagcaccacgttta

SEQ ID NO: 456 - DNA, 27 bp, Artificial Sequence
  Synthetic
ttaatgcaagctgaagctccacgttta

SEQ ID NO: 457 - DNA, 27 bp, Artificial Sequence
  Synthetic
ttaatgcaagcagaagcaccacgttta

SEQ ID NO: 458 - DNA, 27 bp, Artificial Sequence
  Synthetic
ttaatgcaagctgaagcaccacgttta

SEQ ID NO: 459 - DNA, 27 bp, Artificial Sequence
  Synthetic
ttaatgcaagctgaagctccacgttta

SEQ ID NO: 460 - DNA, 27 bp, Artificial Sequence
  Synthetic
ttaatgcaagctgaagcaccacgttta

SEQ ID NO: 461 - DNA, 27 bp, Artificial Sequence
  Synthetic
cttatgcaagcagaggctccacgtctt

SEQ ID NO: 462 - DNA, 27 bp, Artificial Sequence
  Synthetic
ttaatgcaagctgaagcaccacgttta

SEQ ID NO: 463 - DNA, 27 bp, Artificial Sequence
  Synthetic
ttaatgcaagcagaagctccacgttta

SEQ ID NO: 464 - DNA, 27 bp, Artificial Sequence
  Synthetic
ttaatgcaagcagaagcaccacgttta

SEQ ID NO: 465 - DNA, 27 bp, Artificial Sequence
  Synthetic
atggctcctgatgttgtagcatttgtg

SEQ ID NO: 466 - DNA, 27 bp, Artificial Sequence
  Synthetic
atggctcctgatgtcgtagcattcgtt

SEQ ID NO: 467 - DNA, 27 bp, Artificial Sequence
  Synthetic
atggctccagatgttgtagcatttgta

SEQ ID NO: 468 - DNA, 27 bp, Artificial Sequence
  Synthetic
atggcaccagatgttgttgcatttgtt

SEQ ID NO: 469 - DNA, 27 bp, Artificial Sequence
  Synthetic
atggcaccagatgttgttgcatttgtt

SEQ ID NO: 470 - DNA, 27 bp, Artificial Sequence
  Synthetic
atggcaccagatgttgttgcgtttgta

SEQ ID NO: 471 - DNA, 27 bp, Artificial Sequence
  Synthetic
atggcaccagatgttgttgcgtttgta

SEQ ID NO: 472 - DNA, 27 bp, Artificial Sequence
  Synthetic
atggcaccagatgttgtagcttttgtt

SEQ ID NO: 473 - DNA, 27 bp, Artificial Sequence
  Synthetic
atggctccagatgtagttgcttttgta

SEQ ID NO: 474 - DNA, 27 bp, Artificial Sequence
  Synthetic
atggcaccagatgttgttgcatttgta

SEQ ID NO: 475 - DNA, 27 bp, Artificial Sequence
  Synthetic
atggcaccagatgtagttgcatttgta

SEQ ID NO: 476 - DNA, 27 bp, Artificial Sequence
  Synthetic
atggctccagatgttgtagcttttgtt

SEQ ID NO: 477 - DNA, 27 bp, Artificial Sequence
  Synthetic
atggcgccagatgttgtagcatttgtt

SEQ ID NO: 478 - DNA, 27 bp, Artificial Sequence
  Synthetic
atggcgccagatgtagttgcatttgta

SEQ ID NO: 479 - DNA, 27 bp, Artificial Sequence
  Synthetic
atggcaccagacgtagtagctttcgta

SEQ ID NO: 480 - DNA, 27 bp, Artificial Sequence
  Synthetic
atggcacccgatgttgttgctttcgta

SEQ ID NO: 481 - DNA, 27 bp, Artificial Sequence
  Synthetic
atggcaccagatgttgtagcttttgtt

SEQ ID NO: 482 - DNA, 27 bp, Artificial Sequence
  Synthetic
atggcaccagatgtagttgcatttgta

SEQ ID NO: 483 - DNA, 27 bp, Artificial Sequence
  Synthetic
acatatagtgtgagcttcttctcttgg

SEQ ID NO: 484 - DNA, 27 bp, Artificial Sequence
  Synthetic
acttatagtgtgagcttcttttcttgg

SEQ ID NO: 485 - DNA, 27 bp, Artificial Sequence
  Synthetic
acgtacagtgtgagcttcttcagctgg

SEQ ID NO: 486 - DNA, 27 bp, Artificial Sequence
  Synthetic
acatatagtgttagcttttttagctgg

SEQ ID NO: 487 - DNA, 27 bp, Artificial Sequence
  Synthetic
acatatagtgttagcttttttagctgg

SEQ ID NO: 488 - DNA, 27 bp, Artificial Sequence
  Synthetic
acatatagtgttagcttcttttcatgg

SEQ ID NO: 489 - DNA, 27 bp, Artificial Sequence
  Synthetic
acatatagtgttagctttttttcctgg

SEQ ID NO: 490 - DNA, 27 bp, Artificial Sequence
  Synthetic
acgtatagtgttagtttctttagttgg

SEQ ID NO: 491 - DNA, 27 bp, Artificial Sequence
  Synthetic
acatattctgtatctttctttagttgg

SEQ ID NO: 492 - DNA, 27 bp, Artificial Sequence
  Synthetic
acatatagtgtaagtttctttagttgg

SEQ ID NO: 493 - DNA, 27 bp, Artificial Sequence
  Synthetic
acatatagtgtttctttctttagttgg

SEQ ID NO: 494 - DNA, 27 bp, Artificial Sequence
  Synthetic
acatatagtgtttctttctttagttgg

SEQ ID NO: 495 - DNA, 27 bp, Artificial Sequence
  Synthetic
acatatagtgtttctttcttttcttgg

SEQ ID NO: 496 - DNA, 27 bp, Artificial Sequence
  Synthetic
acatattctgttagtttctttagttgg

SEQ ID NO: 497 - DNA, 27 bp, Artificial Sequence
  Synthetic
acatatagtgttagtttcttcagttgg

SEQ ID NO: 498 - DNA, 27 bp, Artificial Sequence
  Synthetic
acatattctgtaagttttttttcttgg

SEQ ID NO: 499 - DNA, 27 bp, Artificial Sequence
  Synthetic
acgtatagtgttagtttctttagttgg

SEQ ID NO: 500 - DNA, 27 bp, Artificial Sequence
  Synthetic
acatatagtgtttctttctttagttgg

SEQ ID NO: 501 - DNA, 27 bp, Artificial Sequence
  Synthetic
ggtatggcaccattaattttatctaga

SEQ ID NO: 502 - DNA, 27 bp, Artificial Sequence
  Synthetic
ggtatggcaccattaattcttagtcgg

SEQ ID NO: 503 - DNA, 27 bp, Artificial Sequence
  Synthetic
ggcatggcaccattaattttgtcacgc

SEQ ID NO: 504 - DNA, 27 bp, Artificial Sequence
  Synthetic
ggtatggcaccacttattttaagtaga

SEQ ID NO: 505 - DNA, 27 bp, Artificial Sequence
  Synthetic
ggtatggcaccacttattttaagtaga

SEQ ID NO: 506 - DNA, 27 bp, Artificial Sequence
  Synthetic
ggcatggcaccattaatcttatcaaga

SEQ ID NO: 507 - DNA, 27 bp, Artificial Sequence
  Synthetic
gggatggcaccattaattttaagcaga

SEQ ID NO: 508 - DNA, 27 bp, Artificial Sequence
  Synthetic
ggtatggcaccattaattttaagtcgt

SEQ ID NO: 509 - DNA, 27 bp, Artificial Sequence
  Synthetic
ggtatggctccattaattttatctcgt

SEQ ID NO: 510 - DNA, 27 bp, Artificial Sequence
  Synthetic
ggtatggctccattaattttatctcgt

SEQ ID NO: 511 - DNA, 27 bp, Artificial Sequence
  Synthetic
ggaatggctccattaattttaagtcgt

SEQ ID NO: 512 - DNA, 27 bp, Artificial Sequence
  Synthetic
ggaatggcaccattaattttatctcgt

SEQ ID NO: 513 - DNA, 27 bp, Artificial Sequence
  Synthetic
ggtatggctccattaattttaagtcgt

SEQ ID NO: 514 - DNA, 27 bp, Artificial Sequence
  Synthetic
ggaatggcaccattaattttaagtcgt

SEQ ID NO: 515 - DNA, 27 bp, Artificial Sequence
  Synthetic
ggtatggctccacttatcctttctcgt

SEQ ID NO: 516 - DNA, 27 bp, Artificial Sequence
  Synthetic
ggtatggcaccattaattcttagtcgt

SEQ ID NO: 517 - DNA, 27 bp, Artificial Sequence
  Synthetic
ggtatggcaccattaattttaagtcgt

SEQ ID NO: 518 - DNA, 27 bp, Artificial Sequence
  Synthetic
ggaatggctccattaattttaagtcgt

SEQ ID NO: 519 - DNA, 27 bp, Artificial Sequence
  Synthetic
tggccacggccgcgtcgttatgttatg

SEQ ID NO: 520 - DNA, 27 bp, Artificial Sequence
  Synthetic
tggccacgtccacgtcgttatgttatg

SEQ ID NO: 521 - DNA, 27 bp, Artificial Sequence
  Synthetic
tggcctcgtccaagacgttacgttatg

SEQ ID NO: 522 - DNA, 27 bp, Artificial Sequence
  Synthetic
tggccacgtccacgtcgttacgtaatg

SEQ ID NO: 523 - DNA, 27 bp, Artificial Sequence
  Synthetic
tggccacgtccacgtcgttatgttatg

SEQ ID NO: 524 - DNA, 27 bp, Artificial Sequence
  Synthetic
tggcctcgtccacgtcgttatgtaatg

SEQ ID NO: 525 - DNA, 27 bp, Artificial Sequence
  Synthetic
tggccacgtccacgtcgttatgttatg

SEQ ID NO: 526 - DNA, 27 bp, Artificial Sequence
  Synthetic
tggccacgtccacgtcgttatgttatg

SEQ ID NO: 527 - DNA, 27 bp, Artificial Sequence
  Synthetic
tggccacgtccacgtcgttatgtaatg

SEQ ID NO: 528 - DNA, 27 bp, Artificial Sequence
  Synthetic
tggccacgtccacgtcgttatgttatg

SEQ ID NO: 529 - DNA, 27 bp, Artificial Sequence
  Synthetic
tggccacgtccacgtcgttatgtaatg

SEQ ID NO: 530 - DNA, 27 bp, Artificial Sequence
  Synthetic
tggccacgtccacgtcgttatgtaatg

SEQ ID NO: 531 - DNA, 27 bp, Artificial Sequence
  Synthetic
tggcctcgtccacgtcgttatgtaatg

SEQ ID NO: 532 - DNA, 27 bp, Artificial Sequence
  Synthetic
tggcctcgtccaagacgttacgttatg

SEQ ID NO: 533 - DNA, 27 bp, Artificial Sequence
  Synthetic
tggccacgtccaagacgttacgtaatg

SEQ ID NO: 534 - DNA, 27 bp, Artificial Sequence
  Synthetic
tggcctcgtccacgtcgttacgttatg

SEQ ID NO: 535 - DNA, 27 bp, Artificial Sequence
  Synthetic
tggccacgtccacgtcgttatgttatg

SEQ ID NO: 536 - DNA, 27 bp, Artificial Sequence
  Synthetic
tggccaagaccacgtcgttatgttatg

SEQ ID NO: 537 - DNA, 27 bp, Artificial Sequence
  Synthetic
cgtttacttgaattctatcttgcagtt

SEQ ID NO: 538 - DNA, 27 bp, Artificial Sequence
  Synthetic
cgtcttttagaattttatttagcggtg

SEQ ID NO: 539 - DNA, 27 bp, Artificial Sequence
  Synthetic
cgtttattagaattttacttagcagtt

SEQ ID NO: 540 - DNA, 27 bp, Artificial Sequence
  Synthetic
cgtttattagaattttaccttgctgta

SEQ ID NO: 541 - DNA, 27 bp, Artificial Sequence
  Synthetic
cgtttattagagttttacttagcagta

SEQ ID NO: 542 - DNA, 27 bp, Artificial Sequence
  Synthetic
cgtttacttgaattttacttagctgtt

SEQ ID NO: 543 - DNA, 27 bp, Artificial Sequence
  Synthetic
cgtttacttgaattctacttagctgtt

SEQ ID NO: 544 - DNA, 27 bp, Artificial Sequence
  Synthetic
cgtcttttagaattttatcttgcggta

SEQ ID NO: 545 - DNA, 27 bp, Artificial Sequence
  Synthetic
cgtttacttgaattttatcttgctgtt

SEQ ID NO: 546 - DNA, 27 bp, Artificial Sequence
  Synthetic
cgtttacttgaattttatcttgcggta

SEQ ID NO: 547 - DNA, 27 bp, Artificial Sequence
  Synthetic
cgtttacttgaattttatcttgcggta

SEQ ID NO: 548 - DNA, 27 bp, Artificial Sequence
  Synthetic
cgtttacttgaattttatcttgctgtt

SEQ ID NO: 549 - DNA, 27 bp, Artificial Sequence
  Synthetic
cgtttacttgaattttacttagctgtt

SEQ ID NO: 550 - DNA, 27 bp, Artificial Sequence
  Synthetic
cgtttattagaattttacttagcagtt

SEQ ID NO: 551 - DNA, 27 bp, Artificial Sequence
  Synthetic
cgtttattagaattctaccttgcagtt

SEQ ID NO: 552 - DNA, 27 bp, Artificial Sequence
  Synthetic
cgtcttttagagttttacttagctgtt

SEQ ID NO: 553 - DNA, 27 bp, Artificial Sequence
  Synthetic
cgtcttttagaattttatcttgcagtt

SEQ ID NO: 554 - DNA, 27 bp, Artificial Sequence
  Synthetic
cgtcttttagaattttatttagcagtt

SEQ ID NO: 555 - DNA, 27 bp, Artificial Sequence
  Synthetic
tacttaatgccagtcaactcagaagtc

SEQ ID NO: 556 - DNA, 27 bp, Artificial Sequence
  Synthetic
tatttaatgccagttaatagtgaagtt

SEQ ID NO: 557 - DNA, 27 bp, Artificial Sequence
  Synthetic
taccttatgccagttaacagtgaggtt

SEQ ID NO: 558 - DNA, 27 bp, Artificial Sequence
  Synthetic
tacttaatgccagttaacagtgaggta

SEQ ID NO: 559 - DNA, 27 bp, Artificial Sequence
  Synthetic
taccttatgcccgttaacagtgaggta

SEQ ID NO: 560 - DNA, 27 bp, Artificial Sequence
  Synthetic
tatttaatgccagtaaattctgaagtt

SEQ ID NO: 561 - DNA, 27 bp, Artificial Sequence
  Synthetic
tatttaatgccagtaaattctgaagtt

SEQ ID NO: 562 - DNA, 27 bp, Artificial Sequence
  Synthetic
tatcttatgccagtaaatagtgaagtt

SEQ ID NO: 563 - DNA, 27 bp, Artificial Sequence
  Synthetic
tatcttatgccagtaaatagtgaagtt

SEQ ID NO: 564 - DNA, 27 bp, Artificial Sequence
  Synthetic
tatcttatgccagtaaatagtgaagtt

SEQ ID NO: 565 - DNA, 27 bp, Artificial Sequence
  Synthetic
tatcttatgccagtaaatagtgaagtt

SEQ ID NO: 566 - DNA, 27 bp, Artificial Sequence
  Synthetic
tatcttatgccagtaaatagtgaagtt

SEQ ID NO: 567 - DNA, 27 bp, Artificial Sequence
  Synthetic
tatttaatgccagtaaattctgaagtt

SEQ ID NO: 568 - DNA, 27 bp, Artificial Sequence
  Synthetic
taccttatgccagttaacagtgaggtt

SEQ ID NO: 569 - DNA, 27 bp, Artificial Sequence
  Synthetic
tacttaatgccagttaattctgaagtt

SEQ ID NO: 570 - DNA, 27 bp, Artificial Sequence
  Synthetic
tatttaatgccagtaaattctgaagtt

SEQ ID NO: 571 - DNA, 27 bp, Artificial Sequence
  Synthetic
tatttaatgccagttaatagtgaagta

SEQ ID NO: 572 - DNA, 27 bp, Artificial Sequence
  Synthetic
tatttaatgccagttaatagtgaagta

SEQ ID NO: 573 - DNA, 27 bp, Artificial Sequence
  Synthetic
gtttggggtattagacttgaacatttt

SEQ ID NO: 574 - DNA, 27 bp, Artificial Sequence
  Synthetic
gtttggggaattcgtttagaacatttt

SEQ ID NO: 575 - DNA, 27 bp, Artificial Sequence
  Synthetic
gtttggggtatccgtcttgaacacttc

SEQ ID NO: 576 - DNA, 27 bp, Artificial Sequence
  Synthetic
gtttggggtatccgtttagagcatttc

SEQ ID NO: 577 - DNA, 27 bp, Artificial Sequence
  Synthetic
gtttggggtattcgtcttgagcacttc

SEQ ID NO: 578 - DNA, 27 bp, Artificial Sequence
  Synthetic
gtatggggtattcgtttagaacacttc

SEQ ID NO: 579 - DNA, 27 bp, Artificial Sequence
  Synthetic
gtttggggaatccgtcttgaacatttt

SEQ ID NO: 580 - DNA, 27 bp, Artificial Sequence
  Synthetic
gtttggggaattcgtttagaacatttc

SEQ ID NO: 581 - DNA, 27 bp, Artificial Sequence
  Synthetic
gtttggggtattcgtttagaacatttc

SEQ ID NO: 582 - DNA, 27 bp, Artificial Sequence
  Synthetic
gtttggggaattcgtttagaacatttc

SEQ ID NO: 583 - DNA, 27 bp, Artificial Sequence
  Synthetic
gtatggggtattcgtttagaacatttt

SEQ ID NO: 584 - DNA, 27 bp, Artificial Sequence
  Synthetic
gtatggggaattcgtttagaacatttt

SEQ ID NO: 585 - DNA, 27 bp, Artificial Sequence
  Synthetic
gtatggggtattcgtttagaacacttc

SEQ ID NO: 586 - DNA, 27 bp, Artificial Sequence
  Synthetic
gtttggggtatccgtcttgaacacttc

SEQ ID NO: 587 - DNA, 27 bp, Artificial Sequence
  Synthetic
gtatggggtatccgtcttgagcatttt

SEQ ID NO: 588 - DNA, 27 bp, Artificial Sequence
  Synthetic
gtatggggtattcgtttagaacacttt

SEQ ID NO: 589 - DNA, 27 bp, Artificial Sequence
  Synthetic
gtttggggaattcgtttagaacatttc

SEQ ID NO: 590 - DNA, 27 bp, Artificial Sequence
  Synthetic
gtatggggaattcgtttagaacatttt

SEQ ID NO: 591 - DNA, 27 bp, Artificial Sequence
  Synthetic
gtctatattcttggtggaagtcaattc

SEQ ID NO: 592 - DNA, 27 bp, Artificial Sequence
  Synthetic
gtttatattttaggtggaagtcaattt

SEQ ID NO: 593 - DNA, 27 bp, Artificial Sequence
  Synthetic
gtttacatccttggtggtagtcaattc

SEQ ID NO: 594 - DNA, 27 bp, Artificial Sequence
  Synthetic
gtatatattttaggaggtagtcaattc

SEQ ID NO: 595 - DNA, 27 bp, Artificial Sequence
  Synthetic
gtatacattttaggtggtagtcagttc

SEQ ID NO: 596 - DNA, 27 bp, Artificial Sequence
  Synthetic
gtttatattttaggtggttctcaattt

SEQ ID NO: 597 - DNA, 27 bp, Artificial Sequence
  Synthetic
gtttatattcttggtggttctcaattt

SEQ ID NO: 598 - DNA, 27 bp, Artificial Sequence
  Synthetic
gtttacattttaggtggaagtcaattt

SEQ ID NO: 599 - DNA, 27 bp, Artificial Sequence
  Synthetic
gtttacattttaggtggtagtcaattc

SEQ ID NO: 600 - DNA, 27 bp, Artificial Sequence
  Synthetic
gtttacattttaggtggaagtcaattt

SEQ ID NO: 601 - DNA, 27 bp, Artificial Sequence
  Synthetic
gtttatattttaggtggatctcaattt

SEQ ID NO: 602 - DNA, 27 bp, Artificial Sequence
  Synthetic
gtttatattttaggtggtagtcaattt

SEQ ID NO: 603 - DNA, 27 bp, Artificial Sequence
  Synthetic
gtttatattttaggtggttctcaattt

SEQ ID NO: 604 - DNA, 27 bp, Artificial Sequence
  Synthetic
gtttacatccttggtggtagtcaattc

SEQ ID NO: 605 - DNA, 27 bp, Artificial Sequence
  Synthetic
gtttacatcttaggaggttctcagttc

SEQ ID NO: 606 - DNA, 27 bp, Artificial Sequence
  Synthetic
gtttacattcttggaggaagtcaattc

SEQ ID NO: 607 - DNA, 27 bp, Artificial Sequence
  Synthetic
gtttacattttaggtggatctcaattt

SEQ ID NO: 608 - DNA, 27 bp, Artificial Sequence
  Synthetic
gtatatattttaggtggatctcaattt

SEQ ID NO: 609 - DNA, 27 bp, Artificial Sequence
  Synthetic
ataatgccaaaagcaggccttcttttt

SEQ ID NO: 610 - DNA, 27 bp, Artificial Sequence
  Synthetic
attatgccaaaagctggattattattt

SEQ ID NO: 611 - DNA, 27 bp, Artificial Sequence
  Synthetic
atcatgccaaaagctggtttattattt

SEQ ID NO: 612 - DNA, 27 bp, Artificial Sequence
  Synthetic
atcatgccaaaggctggtcttcttttc

SEQ ID NO: 613 - DNA, 27 bp, Artificial Sequence
  Synthetic
atcatgccaaaggctggacttttattc

SEQ ID NO: 614 - DNA, 27 bp, Artificial Sequence
  Synthetic
attatgccaaaagctggtttacttttt

SEQ ID NO: 615 - DNA, 27 bp, Artificial Sequence
  Synthetic
attatgcctaaagctggtttattattc

SEQ ID NO: 616 - DNA, 27 bp, Artificial Sequence
  Synthetic
attatgccaaaagcaggtttacttttt

SEQ ID NO: 617 - DNA, 27 bp, Artificial Sequence
  Synthetic
attatgccaaaagcaggtttacttttt

SEQ ID NO: 618 - DNA, 27 bp, Artificial Sequence
  Synthetic
attatgccaaaagcaggtttacttttt

SEQ ID NO: 619 - DNA, 27 bp, Artificial Sequence
  Synthetic
attatgccaaaagctggattacttttt

SEQ ID NO: 620 - DNA, 27 bp, Artificial Sequence
  Synthetic
attatgccaaaagctggtttacttttt

SEQ ID NO: 621 - DNA, 27 bp, Artificial Sequence
  Synthetic
attatgccaaaagctggtttacttttt

SEQ ID NO: 622 - DNA, 27 bp, Artificial Sequence
  Synthetic
atcatgccaaaagctggtttattattt

SEQ ID NO: 623 - DNA, 27 bp, Artificial Sequence
  Synthetic
atcatgccaaaagctggtttattattc

SEQ ID NO: 624 - DNA, 27 bp, Artificial Sequence
  Synthetic
atcatgccaaaggctggtttacttttc

SEQ ID NO: 625 - DNA, 27 bp, Artificial Sequence
  Synthetic
attatgccaaaagctggtcttcttttt

SEQ ID NO: 626 - DNA, 27 bp, Artificial Sequence
  Synthetic
attatgccaaaagcaggtcttcttttt

SEQ ID NO: 627 - DNA, 27 bp, Artificial Sequence
  Synthetic
tcgctatattattggcctagaccacgt

SEQ ID NO: 628 - DNA, 27 bp, Artificial Sequence
  Synthetic
agtttatattattggccacgtccacgt

SEQ ID NO: 629 - DNA, 27 bp, Artificial Sequence
  Synthetic
agtctttactactggccacgtccacgt

SEQ ID NO: 630 - DNA, 27 bp, Artificial Sequence
  Synthetic
agtctttactactggccacgtccacgt

SEQ ID NO: 631 - DNA, 27 bp, Artificial Sequence
  Synthetic
agtctttactactggccacgtcctcgt

SEQ ID NO: 632 - DNA, 27 bp, Artificial Sequence
  Synthetic
agtctttattactggccacgtcctcgt

SEQ ID NO: 633 - DNA, 27 bp, Artificial Sequence
  Synthetic
agtttatattattggccaagaccacgt

SEQ ID NO: 634 - DNA, 27 bp, Artificial Sequence
  Synthetic
agtttatattattggccacgtccacgt

SEQ ID NO: 635 - DNA, 27 bp, Artificial Sequence
  Synthetic
agtttatattattggccacgtccacgt

SEQ ID NO: 636 - DNA, 27 bp, Artificial Sequence
  Synthetic
agtttatattattggccacgtccacgt

SEQ ID NO: 637 - DNA, 27 bp, Artificial Sequence
  Synthetic
tctctttattattggccacgtccacgt

SEQ ID NO: 638 - DNA, 27 bp, Artificial Sequence
  Synthetic
tctctttattattggccacgtccacgt

SEQ ID NO: 639 - DNA, 27 bp, Artificial Sequence
  Synthetic
agtctttattactggccacgtcctcgt

SEQ ID NO: 640 - DNA, 27 bp, Artificial Sequence
  Synthetic
agtctttactactggccacgtccacgt

SEQ ID NO: 641 - DNA, 27 bp, Artificial Sequence
  Synthetic
agtctttactactggccacgtccaaga

SEQ ID NO: 642 - DNA, 27 bp, Artificial Sequence
  Synthetic
agtctttattactggccacgtccacgt

SEQ ID NO: 643 - DNA, 27 bp, Artificial Sequence
  Synthetic
agtttatattattggccacgtccacgt

SEQ ID NO: 644 - DNA, 27 bp, Artificial Sequence
  Synthetic
agtctttattattggccacgtccacgt

SEQ ID NO: 645 - DNA, 27 bp, Artificial Sequence
  Synthetic
tacatgttcccggtgattttcagcaaa

SEQ ID NO: 646 - DNA, 27 bp, Artificial Sequence
  Synthetic
tatatgtttccagttatttttagtaaa

SEQ ID NO: 647 - DNA, 27 bp, Artificial Sequence
  Synthetic
tatatgtttccagtaattttttctaaa

SEQ ID NO: 648 - DNA, 27 bp, Artificial Sequence
  Synthetic
tatatgtttccagttatttttagtaaa

SEQ ID NO: 649 - DNA, 27 bp, Artificial Sequence
  Synthetic
tacatgttccccgttattttttctaaa

SEQ ID NO: 650 - DNA, 27 bp, Artificial Sequence
  Synthetic
tatatgtttccagttattttcagtaaa

SEQ ID NO: 651 - DNA, 27 bp, Artificial Sequence
  Synthetic
tacatgtttccagtaatttttagtaag

SEQ ID NO: 652 - DNA, 27 bp, Artificial Sequence
  Synthetic
tacatgtttccagtaatttttagtaaa

SEQ ID NO: 653 - DNA, 27 bp, Artificial Sequence
  Synthetic
tacatgtttccagtaatttttagtaaa

SEQ ID NO: 654 - DNA, 27 bp, Artificial Sequence
  Synthetic
tacatgtttccagtaatttttagtaaa

SEQ ID NO: 655 - DNA, 27 bp, Artificial Sequence
  Synthetic
tatatgtttccagtaatttttagtaaa

SEQ ID NO: 656 - DNA, 27 bp, Artificial Sequence
  Synthetic
tatatgtttccagtaatttttagtaaa

SEQ ID NO: 657 - DNA, 27 bp, Artificial Sequence
  Synthetic
tatatgtttccagttattttcagtaaa

SEQ ID NO: 658 - DNA, 27 bp, Artificial Sequence
  Synthetic
tatatgtttccagtaattttttctaaa

SEQ ID NO: 659 - DNA, 27 bp, Artificial Sequence
  Synthetic
tatatgtttccagttatttttagtaag

SEQ ID NO: 660 - DNA, 27 bp, Artificial Sequence
  Synthetic
tatatgtttccagtaatctttagtaaa

SEQ ID NO: 661 - DNA, 27 bp, Artificial Sequence
  Synthetic
tatatgtttccagtaatttttagtaaa

SEQ ID NO: 662 - DNA, 27 bp, Artificial Sequence
  Synthetic
tatatgtttccagttatttttagtaaa

SEQ ID NO: 663 - DNA, 27 bp, Artificial Sequence
  Synthetic
gctccacgtggtccgcatggtggtatg

SEQ ID NO: 664 - DNA, 27 bp, Artificial Sequence
  Synthetic
gctccacgtggaccacatggaggaatg

SEQ ID NO: 665 - DNA, 27 bp, Artificial Sequence
  Synthetic
gctccacgtggtccacatggaggaatg

SEQ ID NO: 666 - DNA, 27 bp, Artificial Sequence
  Synthetic
gcaccacgtggtccacatggtggaatg

SEQ ID NO: 667 - DNA, 27 bp, Artificial Sequence
  Synthetic
gctccacgtggtccacatggtggaatg

SEQ ID NO: 668 - DNA, 27 bp, Artificial Sequence
  Synthetic
gcaccacgtggaccacacggtggtatg

SEQ ID NO: 669 - DNA, 27 bp, Artificial Sequence
  Synthetic
gcaccacgtggaccacacggaggtatg

SEQ ID NO: 670 - DNA, 27 bp, Artificial Sequence
  Synthetic
gctccacgtggtccacatggtggaatg

SEQ ID NO: 671 - DNA, 27 bp, Artificial Sequence
  Synthetic
gctccacgtggtccacatggtggtatg

SEQ ID NO: 672 - DNA, 27 bp, Artificial Sequence
  Synthetic
gctccacgtggtccacatggtggaatg

SEQ ID NO: 673 - DNA, 27 bp, Artificial Sequence
  Synthetic
gctccacgtggtccacatggtggaatg

SEQ ID NO: 674 - DNA, 27 bp, Artificial Sequence
  Synthetic
gctccacgtggaccacatggtggtatg

SEQ ID NO: 675 - DNA, 27 bp, Artificial Sequence
  Synthetic
gcaccacgtggaccacacggtggtatg

SEQ ID NO: 676 - DNA, 27 bp, Artificial Sequence
  Synthetic
gctccacgtggtccacatggaggaatg

SEQ ID NO: 677 - DNA, 27 bp, Artificial Sequence
  Synthetic
gctccacgtggtccacatggtggaatg

SEQ ID NO: 678 - DNA, 27 bp, Artificial Sequence
  Synthetic
gctcctagaggtccacatggaggtatg

SEQ ID NO: 679 - DNA, 27 bp, Artificial Sequence
  Synthetic
gcaccacgtggaccacatggtggaatg

SEQ ID NO: 680 - DNA, 27 bp, Artificial Sequence
  Synthetic
gcaccacgtggaccacatggtggaatg

SEQ ID NO: 681 - DNA, 27 bp, Artificial Sequence
  Synthetic
ttaccatggacaatgaactatccacta

SEQ ID NO: 682 - DNA, 27 bp, Artificial Sequence
  Synthetic
ttaccatggacaatgaattatccatta

SEQ ID NO: 683 - DNA, 27 bp, Artificial Sequence
  Synthetic
ttaccatggactatgaactacccactt

SEQ ID NO: 684 - DNA, 27 bp, Artificial Sequence
  Synthetic
ttaccatggactatgaattatccatta

SEQ ID NO: 685 - DNA, 27 bp, Artificial Sequence
  Synthetic
cttccatggacaatgaactacccactt

SEQ ID NO: 686 - DNA, 27 bp, Artificial Sequence
  Synthetic
cttccatggacaatgaattatccttta

SEQ ID NO: 687 - DNA, 27 bp, Artificial Sequence
  Synthetic
ttaccatggactatgaactatccatta

SEQ ID NO: 688 - DNA, 27 bp, Artificial Sequence
  Synthetic
ttaccatggacaatgaattatccatta

SEQ ID NO: 689 - DNA, 27 bp, Artificial Sequence
  Synthetic
ttaccatggacaatgaattatccatta

SEQ ID NO: 690 - DNA, 27 bp, Artificial Sequence
  Synthetic
ttaccatggacaatgaattatccatta

SEQ ID NO: 691 - DNA, 27 bp, Artificial Sequence
  Synthetic
ttaccatggacaatgaattatccatta

SEQ ID NO: 692 - DNA, 27 bp, Artificial Sequence
  Synthetic
ttaccatggacaatgaattatccatta

SEQ ID NO: 693 - DNA, 27 bp, Artificial Sequence
  Synthetic
cttccatggacaatgaattatccttta

SEQ ID NO: 694 - DNA, 27 bp, Artificial Sequence
  Synthetic
ttaccatggactatgaactacccactt

SEQ ID NO: 695 - DNA, 27 bp, Artificial Sequence
  Synthetic
ttaccatggacaatgaactatccatta

SEQ ID NO: 696 - DNA, 27 bp, Artificial Sequence
  Synthetic
ttaccatggactatgaattacccatta

SEQ ID NO: 697 - DNA, 27 bp, Artificial Sequence
  Synthetic
ttaccatggacaatgaattatccatta

SEQ ID NO: 698 - DNA, 27 bp, Artificial Sequence
  Synthetic
ttaccatggacaatgaattatccatta

SEQ ID NO: 699 - DNA, 27 bp, Artificial Sequence
  Synthetic
cacgtatttggttatagttggtacaag

SEQ ID NO: 700 - DNA, 27 bp, Artificial Sequence
  Synthetic
cacgttttcggatacagttggtataag

SEQ ID NO: 701 - DNA, 27 bp, Artificial Sequence
  Synthetic
cacgtttttggatactcttggtataaa

SEQ ID NO: 702 - DNA, 27 bp, Artificial Sequence
  Synthetic
catgttttcggatatagttggtacaaa

SEQ ID NO: 703 - DNA, 27 bp, Artificial Sequence
  Synthetic
cacgtatttggatattcttggtacaaa

SEQ ID NO: 704 - DNA, 27 bp, Artificial Sequence
  Synthetic
catgtatttggttatagttggtataaa

SEQ ID NO: 705 - DNA, 27 bp, Artificial Sequence
  Synthetic
catgtatttggttatagttggtataaa

SEQ ID NO: 706 - DNA, 27 bp, Artificial Sequence
  Synthetic
catgtatttggttatagttggtataaa

SEQ ID NO: 707 - DNA, 27 bp, Artificial Sequence
  Synthetic
catgtatttggatatagttggtataaa

SEQ ID NO: 708 - DNA, 27 bp, Artificial Sequence
  Synthetic
catgtatttggatatagttggtataaa

SEQ ID NO: 709 - DNA, 27 bp, Artificial Sequence
  Synthetic
atccctcaagttcacacacaagttctt

SEQ ID NO: 710 - DNA, 27 bp, Artificial Sequence
  Synthetic
attccacaagttcacacacaagtatta

SEQ ID NO: 711 - DNA, 27 bp, Artificial Sequence
  Synthetic
atccctcaagttcatacacaagttctt

SEQ ID NO: 712 - DNA, 27 bp, Artificial Sequence
  Synthetic
atcccacaagttcatacacaagtttta

SEQ ID NO: 713 - DNA, 27 bp, Artificial Sequence
  Synthetic
atcccacaagttcatacacaagttctt

SEQ ID NO: 714 - DNA, 27 bp, Artificial Sequence
  Synthetic
ataccacaagtacatacacaagtttta

SEQ ID NO: 715 - DNA, 27 bp, Artificial Sequence
  Synthetic
ataccgcaagtacatacacaagtttta

SEQ ID NO: 716 - DNA, 27 bp, Artificial Sequence
  Synthetic
ataccgcaagtacatacacaagtttta

SEQ ID NO: 717 - DNA, 27 bp, Artificial Sequence
  Synthetic
attccacaagtacatacacaagttctt

SEQ ID NO: 718 - DNA, 27 bp, Artificial Sequence
  Synthetic
attccacaagtacatacacaagttctt

SEQ ID NO: 719 - DNA, 27 bp, Artificial Sequence
  Synthetic
atttatccaaacgcatctttattattt

SEQ ID NO: 720 - DNA, 27 bp, Artificial Sequence
  Synthetic
atttatccaaatgctagtcttttattt

SEQ ID NO: 721 - DNA, 27 bp, Artificial Sequence
  Synthetic
atctaccctaatgcatctttattattt

SEQ ID NO: 722 - DNA, 27 bp, Artificial Sequence
  Synthetic
atttatccaaatgctagtttattattc

SEQ ID NO: 723 - DNA, 27 bp, Artificial Sequence
  Synthetic
atttacccaaatgcaagtcttcttttt

SEQ ID NO: 724 - DNA, 27 bp, Artificial Sequence
  Synthetic
atatatccaaatgctagtcttcttttc

SEQ ID NO: 725 - DNA, 27 bp, Artificial Sequence
  Synthetic
atctatccaaatgcaagtcttttattc

SEQ ID NO: 726 - DNA, 27 bp, Artificial Sequence
  Synthetic
atctatccaaatgcaagtcttttattc

SEQ ID NO: 727 - DNA, 27 bp, Artificial Sequence
  Synthetic
atttatccaaatgcaagtcttcttttt

SEQ ID NO: 728 - DNA, 27 bp, Artificial Sequence
  Synthetic
atttatccaaatgctagtcttcttttt

SEQ ID NO: 729 - DNA, 27 bp, Artificial Sequence
  Synthetic
atccttatcggtgttcttgttggagta

SEQ ID NO: 730 - DNA, 27 bp, Artificial Sequence
  Synthetic
attttaattggtgtacttgttggtgtt

SEQ ID NO: 731 - DNA, 27 bp, Artificial Sequence
  Synthetic
atcttaattggtgttttagttggtgtt

SEQ ID NO: 732 - DNA, 27 bp, Artificial Sequence
  Synthetic
attcttattggagttttagtaggtgtt

SEQ ID NO: 733 - DNA, 27 bp, Artificial Sequence
  Synthetic
attttaatcggagttttagtaggtgtt

SEQ ID NO: 734 - DNA, 27 bp, Artificial Sequence
  Synthetic
attcttattggagttttagttggtgtt

SEQ ID NO: 735 - DNA, 27 bp, Artificial Sequence
  Synthetic
atacttattggagttttagttggtgtt

SEQ ID NO: 736 - DNA, 27 bp, Artificial Sequence
  Synthetic
atacttattggagttttagttggtgtt

SEQ ID NO: 737 - DNA, 27 bp, Artificial Sequence
  Synthetic
attcttattggagttttagtaggtgtt

SEQ ID NO: 738 - DNA, 27 bp, Artificial Sequence
  Synthetic
attcttattggagttttagtaggtgtt

SEQ ID NO: 739 - DNA, 27 bp, Artificial Sequence
  Synthetic
ttattacttggtacaattcatgctgta

SEQ ID NO: 740 - DNA, 27 bp, Artificial Sequence
  Synthetic
ttattacttggtacaatccacgctgta

SEQ ID NO: 741 - DNA, 27 bp, Artificial Sequence
  Synthetic
ttacttttaggaacaattcatgctgtt

SEQ ID NO: 742 - DNA, 27 bp, Artificial Sequence
  Synthetic
ttattattaggtactattcacgcagtt

SEQ ID NO: 743 - DNA, 27 bp, Artificial Sequence
  Synthetic
ttattattaggtacaattcatgctgtt

SEQ ID NO: 744 - DNA, 27 bp, Artificial Sequence
  Synthetic
ttacttttaggcactattcatgcggtt

SEQ ID NO: 745 - DNA, 27 bp, Artificial Sequence
  Synthetic
ttacttttaggcactattcatgctgtt

SEQ ID NO: 746 - DNA, 27 bp, Artificial Sequence
  Synthetic
cttcttcttggaactattcatgctgtg

SEQ ID NO: 747 - DNA, 27 bp, Artificial Sequence
  Synthetic
ttacttcttggaactattcatgctgtt

SEQ ID NO: 748 - DNA, 27 bp, Artificial Sequence
  Synthetic
cttcttcttggaactattcatgctgtt

SEQ ID NO: 749 - DNA, 27 bp, Artificial Sequence
  Synthetic
aaatataaaaaattcccatggtggtta

SEQ ID NO: 750 - DNA, 27 bp, Artificial Sequence
  Synthetic
aaatataaaaagttcccatggtggtta

SEQ ID NO: 751 - DNA, 27 bp, Artificial Sequence
  Synthetic
aaatataagaaatttccatggtggtta

SEQ ID NO: 752 - DNA, 27 bp, Artificial Sequence
  Synthetic
aagtataaaaaatttccatggtggctt

SEQ ID NO: 753 - DNA, 27 bp, Artificial Sequence
  Synthetic
aaatataaaaaatttccatggtggctt

SEQ ID NO: 754 - DNA, 27 bp, Artificial Sequence
  Synthetic
aaatataaaaaatttccatggtggtta

SEQ ID NO: 755 - DNA, 27 bp, Artificial Sequence
  Synthetic
aagtataaaaaatttccatggtggtta

SEQ ID NO: 756 - DNA, 27 bp, Artificial Sequence
  Synthetic
aagtataaaaaatttccatggtggtta

SEQ ID NO: 757 - DNA, 27 bp, Artificial Sequence
  Synthetic
aaatataaaaaatttccatggtggctt

SEQ ID NO: 758 - DNA, 27 bp, Artificial Sequence
  Synthetic
aaatataaaaaatttccatggtggctt

SEQ ID NO: 759 - DNA, 27 bp, Artificial Sequence
  Synthetic
aaccctcaaccagtatggttatgcctt

SEQ ID NO: 760 - DNA, 27 bp, Artificial Sequence
  Synthetic
aacccacaaccagtttggctttgctta

SEQ ID NO: 761 - DNA, 27 bp, Artificial Sequence
  Synthetic
aatccacaaccagtttggttatgcctt

SEQ ID NO: 762 - DNA, 27 bp, Artificial Sequence
  Synthetic
aacccacaaccagtttggttatgctta

SEQ ID NO: 763 - DNA, 27 bp, Artificial Sequence
  Synthetic
aatcctcaaccagtttggctttgctta

SEQ ID NO: 764 - DNA, 27 bp, Artificial Sequence
  Synthetic
aatccacaaccagtatggttatgctta

SEQ ID NO: 765 - DNA, 27 bp, Artificial Sequence
  Synthetic
aatccacaaccagtatggttatgctta

SEQ ID NO: 766 - DNA, 27 bp, Artificial Sequence
  Synthetic
aatccacaaccagtatggttatgctta

SEQ ID NO: 767 - DNA, 27 bp, Artificial Sequence
  Synthetic
aatccacaaccagtatggctttgtctt

SEQ ID NO: 768 - DNA, 27 bp, Artificial Sequence
  Synthetic
aatccacaaccagtatggctttgtctt

SEQ ID NO: 769 - DNA, 27 bp, Artificial Sequence
  Synthetic
atcatgccaaaggctggtcttttattc

SEQ ID NO: 770 - DNA, 27 bp, Artificial Sequence
  Synthetic
attatgccaaaggctggtcttttattc

SEQ ID NO: 771 - DNA, 27 bp, Artificial Sequence
  Synthetic
atcatgccaaaagctggattattattc

SEQ ID NO: 772 - DNA, 27 bp, Artificial Sequence
  Synthetic
attatgccaaaggctggtttattattc

SEQ ID NO: 773 - DNA, 27 bp, Artificial Sequence
  Synthetic
attatgccaaaggctggtcttcttttc

SEQ ID NO: 774 - DNA, 27 bp, Artificial Sequence
  Synthetic
atcatgccaaaagcaggtttacttttt

SEQ ID NO: 775 - DNA, 27 bp, Artificial Sequence
  Synthetic
ataatgccaaaagctggtttattattt

SEQ ID NO: 776 - DNA, 27 bp, Artificial Sequence
  Synthetic
ataatgccaaaagctggtttattattt

SEQ ID NO: 777 - DNA, 27 bp, Artificial Sequence
  Synthetic
attatgccaaaagctggtttacttttt

SEQ ID NO: 778 - DNA, 27 bp, Artificial Sequence
  Synthetic
attatgccaaaagcaggtttacttttt

SEQ ID NO: 779 - DNA, 27 bp, Artificial Sequence
  Synthetic
tacatgttcccagtaatctttagtaag

SEQ ID NO: 780 - DNA, 27 bp, Artificial Sequence
  Synthetic
tatatgtttccagtaatttttagtaaa

SEQ ID NO: 781 - DNA, 27 bp, Artificial Sequence
  Synthetic
tatatgttcccagttatttttagtaaa

SEQ ID NO: 782 - DNA, 27 bp, Artificial Sequence
  Synthetic
tatatgtttccagttattttttctaaa

SEQ ID NO: 783 - DNA, 27 bp, Artificial Sequence
  Synthetic
tacatgtttccagttatttttagtaag

SEQ ID NO: 784 - DNA, 27 bp, Artificial Sequence
  Synthetic
tatatgtttccagttatttttagtaaa

SEQ ID NO: 785 - DNA, 27 bp, Artificial Sequence
  Synthetic
tatatgtttccagttatttttagtaaa

SEQ ID NO: 786 - DNA, 27 bp, Artificial Sequence
  Synthetic
tatatgtttccagttatttttagtaaa

SEQ ID NO: 787 - DNA, 27 bp, Artificial Sequence
  Synthetic
tatatgtttccagtaatttttagtaaa

SEQ ID NO: 788 - DNA, 27 bp, Artificial Sequence
  Synthetic
tatatgtttccagtaatttttagtaaa

SEQ ID NO: 789 - DNA, 27 bp, Artificial Sequence
  Synthetic
aatatgactcacgttttatacccactt

SEQ ID NO: 790 - DNA, 27 bp, Artificial Sequence
  Synthetic
aacatgacacatgttctttacccatta

SEQ ID NO: 791 - DNA, 27 bp, Artificial Sequence
  Synthetic
aatatgacacatgtattatatcctctt

SEQ ID NO: 792 - DNA, 27 bp, Artificial Sequence
  Synthetic
aatatgactcatgttttatatccatta

SEQ ID NO: 793 - DNA, 27 bp, Artificial Sequence
  Synthetic
aatatgactcatgttctttacccactt

SEQ ID NO: 794 - DNA, 27 bp, Artificial Sequence
  Synthetic
aatatgacacatgttctttatccatta

SEQ ID NO: 795 - DNA, 27 bp, Artificial Sequence
  Synthetic
aacatgacacatgttctttatccatta

SEQ ID NO: 796 - DNA, 27 bp, Artificial Sequence
  Synthetic
aacatgacacatgttctttatccatta

SEQ ID NO: 797 - DNA, 27 bp, Artificial Sequence
  Synthetic
aatatgacacatgtactttatccatta

SEQ ID NO: 798 - DNA, 27 bp, Artificial Sequence
  Synthetic
aatatgacacatgtactttatccatta

SEQ ID NO: 799 - DNA, 27 bp, Artificial Sequence
  Synthetic
attatggcaaaatttttacattggtta

SEQ ID NO: 800 - DNA, 27 bp, Artificial Sequence
  Synthetic
attatggctaaatttttacattggtta

SEQ ID NO: 801 - DNA, 27 bp, Artificial Sequence
  Synthetic
attatggctaaattccttcattggctt

SEQ ID NO: 802 - DNA, 27 bp, Artificial Sequence
  Synthetic
attatggcaaaattccttcattggtta

SEQ ID NO: 803 - DNA, 27 bp, Artificial Sequence
  Synthetic
atcatggctaagttcttacactggtta

SEQ ID NO: 804 - DNA, 27 bp, Artificial Sequence
  Synthetic
atcatggctaaatttcttcattggtta

SEQ ID NO: 805 - DNA, 27 bp, Artificial Sequence
  Synthetic
attatggcaaaatttcttcattggtta

SEQ ID NO: 806 - DNA, 27 bp, Artificial Sequence
  Synthetic
attatggcaaaatttcttcattggtta

SEQ ID NO: 807 - DNA, 27 bp, Artificial Sequence
  Synthetic
attatggcaaaatttcttcattggtta

SEQ ID NO: 808 - DNA, 27 bp, Artificial Sequence
  Synthetic
attatggctaaatttcttcattggtta

SEQ ID NO: 809 - DNA, 27 bp, Artificial Sequence
  Synthetic
catgtatttggttattcttggtataaa

SEQ ID NO: 810 - DNA, 27 bp, Artificial Sequence
  Synthetic
catgtatttggttattcttggtataaa

SEQ ID NO: 811 - DNA, 27 bp, Artificial Sequence
  Synthetic
catgtatttggttattcttggtataaa

SEQ ID NO: 812 - DNA, 27 bp, Artificial Sequence
  Synthetic
catgtttttggatatagttggtataaa

SEQ ID NO: 813 - DNA, 27 bp, Artificial Sequence
  Synthetic
catgtttttggatattcttggtataaa

SEQ ID NO: 814 - DNA, 27 bp, Artificial Sequence
  Synthetic
cacgtattcggttactcttggtacaag

SEQ ID NO: 815 - DNA, 27 bp, Artificial Sequence
  Synthetic
cacgttttcggatacagttggtacaag

SEQ ID NO: 816 - DNA, 27 bp, Artificial Sequence
  Synthetic
cacgtattcggttacagttggtacaag

SEQ ID NO: 817 - DNA, 27 bp, Artificial Sequence
  Synthetic
catgtattcggttactcttggtacaag

SEQ ID NO: 818 - DNA, 27 bp, Artificial Sequence
  Synthetic
catgttttcggatacagttggtataaa

SEQ ID NO: 819 - DNA, 27 bp, Artificial Sequence
  Synthetic
atcccacaagtacatacacaagtttta

SEQ ID NO: 820 - DNA, 27 bp, Artificial Sequence
  Synthetic
atcccacaagtacatacacaagtttta

SEQ ID NO: 821 - DNA, 27 bp, Artificial Sequence
  Synthetic
atcccacaagtacatacacaagtttta

SEQ ID NO: 822 - DNA, 27 bp, Artificial Sequence
  Synthetic
attccacaagtacatacacaagttctt

SEQ ID NO: 823 - DNA, 27 bp, Artificial Sequence
  Synthetic
attccacaagtacatacacaagttctt

SEQ ID NO: 824 - DNA, 27 bp, Artificial Sequence
  Synthetic
atcccacaagttcacacacaagttctt

SEQ ID NO: 825 - DNA, 27 bp, Artificial Sequence
  Synthetic
atcccacaagttcacacacaagttctt

SEQ ID NO: 826 - DNA, 27 bp, Artificial Sequence
  Synthetic
attccacaagttcatacacaagttctt

SEQ ID NO: 827 - DNA, 27 bp, Artificial Sequence
  Synthetic
attccacaagttcatactcaagtttta

SEQ ID NO: 828 - DNA, 27 bp, Artificial Sequence
  Synthetic
attccacaagtacatacacaagtttta

SEQ ID NO: 829 - DNA, 27 bp, Artificial Sequence
  Synthetic
atatatccaaatgcttctcttcttttc

SEQ ID NO: 830 - DNA, 27 bp, Artificial Sequence
  Synthetic
atatatccaaatgcatctcttcttttc

SEQ ID NO: 831 - DNA, 27 bp, Artificial Sequence
  Synthetic
atatatccaaatgcatctcttcttttc

SEQ ID NO: 832 - DNA, 27 bp, Artificial Sequence
  Synthetic
atttatccaaatgcatctcttcttttt

SEQ ID NO: 833 - DNA, 27 bp, Artificial Sequence
  Synthetic
atttatccaaatgcttctcttcttttt

SEQ ID NO: 834 - DNA, 27 bp, Artificial Sequence
  Synthetic
atctaccctaacgctagtttattattt

SEQ ID NO: 835 - DNA, 27 bp, Artificial Sequence
  Synthetic
atctacccaaatgctagtttattattt

SEQ ID NO: 836 - DNA, 27 bp, Artificial Sequence
  Synthetic
atttacccaaacgcaagtcttcttttc

SEQ ID NO: 837 - DNA, 27 bp, Artificial Sequence
  Synthetic
atttacccaaatgctagtttattattc

SEQ ID NO: 838 - DNA, 27 bp, Artificial Sequence
  Synthetic
atttatccaaatgctagtcttttattc

SEQ ID NO: 839 - DNA, 27 bp, Artificial Sequence
  Synthetic
attcttattggtgttttagttggtgtt

SEQ ID NO: 840 - DNA, 27 bp, Artificial Sequence
  Synthetic
attcttattggtgttttagttggcgta

SEQ ID NO: 841 - DNA, 27 bp, Artificial Sequence
  Synthetic
attcttattggcgttttagtgggcgta

SEQ ID NO: 842 - DNA, 27 bp, Artificial Sequence
  Synthetic
attcttattggtgttcttgttggtgtt

SEQ ID NO: 843 - DNA, 27 bp, Artificial Sequence
  Synthetic
attcttattggcgttttagtgggcgta

SEQ ID NO: 844 - DNA, 27 bp, Artificial Sequence
  Synthetic
atcttaattggagttttagtaggtgtt

SEQ ID NO: 845 - DNA, 27 bp, Artificial Sequence
  Synthetic
attttaatcggagttttagttggtgtt

SEQ ID NO: 846 - DNA, 27 bp, Artificial Sequence
  Synthetic
attttaattggtgttttagtaggtgtt

SEQ ID NO: 847 - DNA, 27 bp, Artificial Sequence
  Synthetic
attttaattggtgttttagttggagta

SEQ ID NO: 848 - DNA, 27 bp, Artificial Sequence
  Synthetic
attttaattggagtattagttggtgtt

SEQ ID NO: 849 - DNA, 27 bp, Artificial Sequence
  Synthetic
ttacttcttggaacaattcatgctgtt

SEQ ID NO: 850 - DNA, 27 bp, Artificial Sequence
  Synthetic
ttacttcttggtacaattcatgcagtt

SEQ ID NO: 851 - DNA, 27 bp, Artificial Sequence
  Synthetic
ttacttcttggaacaattcatgcagtt

SEQ ID NO: 852 - DNA, 27 bp, Artificial Sequence
  Synthetic
cttcttcttggaacaattcatgctgta

SEQ ID NO: 853 - DNA, 27 bp, Artificial Sequence
  Synthetic
cttcttcttggaacaattcatgcagta

SEQ ID NO: 854 - DNA, 27 bp, Artificial Sequence
  Synthetic
cttttattaggaacaatccatgcagta

SEQ ID NO: 855 - DNA, 27 bp, Artificial Sequence
  Synthetic
ttattattaggtacaatccatgcagta

SEQ ID NO: 856 - DNA, 27 bp, Artificial Sequence
  Synthetic
ttacttttaggaactattcatgctgtt

SEQ ID NO: 857 - DNA, 27 bp, Artificial Sequence
  Synthetic
ttattattaggtactattcacgcagta

SEQ ID NO: 858 - DNA, 27 bp, Artificial Sequence
  Synthetic
ttattattaggaacaattcacgctgta

SEQ ID NO: 859 - DNA, 27 bp, Artificial Sequence
  Synthetic
aaatataaaaaatttccatggtggtta

SEQ ID NO: 860 - DNA, 27 bp, Artificial Sequence
  Synthetic
aagtataaaaaatttccatggtggtta

SEQ ID NO: 861 - DNA, 27 bp, Artificial Sequence
  Synthetic
aagtataaaaaatttccatggtggtta

SEQ ID NO: 862 - DNA, 27 bp, Artificial Sequence
  Synthetic
aaatataaaaaatttccatggtggtta

SEQ ID NO: 863 - DNA, 27 bp, Artificial Sequence
  Synthetic
aaatataaaaaatttccatggtggtta

SEQ ID NO: 864 - DNA, 27 bp, Artificial Sequence
  Synthetic
aaatataagaagtttccatggtggctt

SEQ ID NO: 865 - DNA, 27 bp, Artificial Sequence
  Synthetic
aaatataaaaaatttccatggtggtta

SEQ ID NO: 866 - DNA, 27 bp, Artificial Sequence
  Synthetic
aaatataaaaaatttccatggtggtta

SEQ ID NO: 867 - DNA, 27 bp, Artificial Sequence
  Synthetic
aaatacaagaaattcccatggtggctt

SEQ ID NO: 868 - DNA, 27 bp, Artificial Sequence
  Synthetic
aaatataaaaagtttccttggtggtta

SEQ ID NO: 869 - DNA, 27 bp, Artificial Sequence
  Synthetic
tacatgtttccagttatttttagtaaa

SEQ ID NO: 870 - DNA, 27 bp, Artificial Sequence
  Synthetic
tacatgtttccagttatttttagtaaa

SEQ ID NO: 871 - DNA, 27 bp, Artificial Sequence
  Synthetic
tacatgtttccagttatttttagtaaa

SEQ ID NO: 872 - DNA, 27 bp, Artificial Sequence
  Synthetic
tatatgtttccagtaatttttagtaaa

SEQ ID NO: 873 - DNA, 27 bp, Artificial Sequence
  Synthetic
tatatgtttccagtaatttttagtaaa

SEQ ID NO: 874 - DNA, 27 bp, Artificial Sequence
  Synthetic
tatatgtttccagtaattttcagtaag

SEQ ID NO: 875 - DNA, 27 bp, Artificial Sequence
  Synthetic
tacatgtttccagtaatctttagtaaa

SEQ ID NO: 876 - DNA, 27 bp, Artificial Sequence
  Synthetic
tacatgtttccagtaatttttagtaaa

SEQ ID NO: 877 - DNA, 27 bp, Artificial Sequence
  Synthetic
tacatgttcccagttattttttctaaa

SEQ ID NO: 878 - DNA, 27 bp, Artificial Sequence
  Synthetic
tacatgtttcccgttatttttagtaag

SEQ ID NO: 879 - DNA, 27 bp, Artificial Sequence
  Synthetic
aatatgacacatgttctttatccatta

SEQ ID NO: 880 - DNA, 27 bp, Artificial Sequence
  Synthetic
aacatgacacatgttctttatccatta

SEQ ID NO: 881 - DNA, 27 bp, Artificial Sequence
  Synthetic
aacatgacacatgttctttatccatta

SEQ ID NO: 882 - DNA, 27 bp, Artificial Sequence
  Synthetic
aatatgacacatgttctttatccatta

SEQ ID NO: 883 - DNA, 27 bp, Artificial Sequence
  Synthetic
aatatgacacatgttctttatccatta

SEQ ID NO: 884 - DNA, 27 bp, Artificial Sequence
  Synthetic
aacatgacacacgttttatatccactt

SEQ ID NO: 885 - DNA, 27 bp, Artificial Sequence
  Synthetic
aacatgactcatgtactttatccactt

SEQ ID NO: 886 - DNA, 27 bp, Artificial Sequence
  Synthetic
aacatgactcacgtactttatccactt

SEQ ID NO: 887 - DNA, 27 bp, Artificial Sequence
  Synthetic
aatatgacacacgtactttacccatta

SEQ ID NO: 888 - DNA, 27 bp, Artificial Sequence
  Synthetic
aatatgacacatgtattatatccatta

SEQ ID NO: 889 - DNA, 27 bp, Artificial Sequence
  Synthetic
attatggcaaaatttcttcattggtta

SEQ ID NO: 890 - DNA, 27 bp, Artificial Sequence
  Synthetic
atcatggctaaatttcttcattggtta

SEQ ID NO: 891 - DNA, 27 bp, Artificial Sequence
  Synthetic
atcatggctaaatttcttcattggtta

SEQ ID NO: 892 - DNA, 27 bp, Artificial Sequence
  Synthetic
attatggcaaaatttcttcattggtta

SEQ ID NO: 893 - DNA, 27 bp, Artificial Sequence
  Synthetic
attatggctaaatttcttcattggtta

SEQ ID NO: 894 - DNA, 27 bp, Artificial Sequence
  Synthetic
attatggcaaaatttttacactggctt

SEQ ID NO: 895 - DNA, 27 bp, Artificial Sequence
  Synthetic
attatggctaaattccttcactggctt

SEQ ID NO: 896 - DNA, 27 bp, Artificial Sequence
  Synthetic
attatggctaaatttttacattggtta

SEQ ID NO: 897 - DNA, 27 bp, Artificial Sequence
  Synthetic
attatggctaaatttttacattggctt

SEQ ID NO: 898 - DNA, 27 bp, Artificial Sequence
  Synthetic
attatggcaaaattccttcattggctt

SEQ ID NO: 899 - DNA, 27 bp, Artificial Sequence
  Synthetic
aaggtaccagaaattgttcattttctt

SEQ ID NO: 900 - DNA, 27 bp, Artificial Sequence
  Synthetic
aaagtaccagaaattgttcattttctt

SEQ ID NO: 901 - DNA, 27 bp, Artificial Sequence
  Synthetic
aaagtaccagaaattgttcattttctt

SEQ ID NO: 902 - DNA, 27 bp, Artificial Sequence
  Synthetic
aaagttccagaaattgtacattttctt

SEQ ID NO: 903 - DNA, 27 bp, Artificial Sequence
  Synthetic
aaagttccagaaattgtacattttctt

SEQ ID NO: 904 - DNA, 27 bp, Artificial Sequence
  Synthetic
aaggttccagagatcgtacatttcctt

SEQ ID NO: 905 - DNA, 27 bp, Artificial Sequence
  Synthetic
aaggttccagaaattgttcatttcctt

SEQ ID NO: 906 - DNA, 27 bp, Artificial Sequence
  Synthetic
aaggttccagaaattgttcacttttta

SEQ ID NO: 907 - DNA, 27 bp, Artificial Sequence
  Synthetic
aaagttccagaaattgttcatttttta

SEQ ID NO: 908 - DNA, 27 bp, Artificial Sequence
  Synthetic
aaagtaccagaaattgtacatttcctt

SEQ ID NO: 909 - DNA, 27 bp, Artificial Sequence
  Synthetic
aagatgagttctggttgtgcattttta

SEQ ID NO: 910 - DNA, 27 bp, Artificial Sequence
  Synthetic
aagatgagttctggttgtgcattttta

SEQ ID NO: 911 - DNA, 27 bp, Artificial Sequence
  Synthetic
aagatgagttctggttgtgcattttta

SEQ ID NO: 912 - DNA, 27 bp, Artificial Sequence
  Synthetic
aaaatgagttctggttgtgcattttta

SEQ ID NO: 913 - DNA, 27 bp, Artificial Sequence
  Synthetic
aaaatgagttctggttgtgcattttta

SEQ ID NO: 914 - DNA, 27 bp, Artificial Sequence
  Synthetic
aaaatgagtagtggatgcgctttttta

SEQ ID NO: 915 - DNA, 27 bp, Artificial Sequence
  Synthetic
aaaatgagttctggttgtgcttttctt

SEQ ID NO: 916 - DNA, 27 bp, Artificial Sequence
  Synthetic
aaaatgagtagtggatgtgctttctta

SEQ ID NO: 917 - DNA, 27 bp, Artificial Sequence
  Synthetic
aaaatgagttctggatgcgcattttta

SEQ ID NO: 918 - DNA, 27 bp, Artificial Sequence
  Synthetic
aaaatgtctagtggttgcgctttctta

SEQ ID NO: 919 - DNA, 27 bp, Artificial Sequence
  Synthetic
agttggtttaaaaattggccatttttc

SEQ ID NO: 920 - DNA, 27 bp, Artificial Sequence
  Synthetic
agttggtttaaaaattggccatttttc

SEQ ID NO: 921 - DNA, 27 bp, Artificial Sequence
  Synthetic
agttggtttaaaaattggccatttttc

SEQ ID NO: 922 - DNA, 27 bp, Artificial Sequence
  Synthetic
tcttggtttaaaaattggccatttttc

SEQ ID NO: 923 - DNA, 27 bp, Artificial Sequence
  Synthetic
tcttggtttaaaaattggccatttttc

SEQ ID NO: 924 - DNA, 27 bp, Artificial Sequence
  Synthetic
tcttggtttaaaaattggccattcttc

SEQ ID NO: 925 - DNA, 27 bp, Artificial Sequence
  Synthetic
agttggtttaaaaattggccatttttc

SEQ ID NO: 926 - DNA, 27 bp, Artificial Sequence
  Synthetic
tcttggtttaagaattggccatttttt

SEQ ID NO: 927 - DNA, 27 bp, Artificial Sequence
  Synthetic
agttggttcaaaaattggccatttttt

SEQ ID NO: 928 - DNA, 27 bp, Artificial Sequence
  Synthetic
tcttggtttaaaaattggccatttttt

SEQ ID NO: 929 - DNA, 27 bp, Artificial Sequence
  Synthetic
catgtttttggttattcttggtataaa

SEQ ID NO: 930 - DNA, 27 bp, Artificial Sequence
  Synthetic
catgtatttggttattcttggtataaa

SEQ ID NO: 931 - DNA, 27 bp, Artificial Sequence
  Synthetic
cacgtattcggttactcttggtataaa

SEQ ID NO: 932 - DNA, 27 bp, Artificial Sequence
  Synthetic
catgtatttggatatagttggtataaa

SEQ ID NO: 933 - DNA, 27 bp, Artificial Sequence
  Synthetic
tatctttttccagtaatttttagtaaa

SEQ ID NO: 934 - DNA, 27 bp, Artificial Sequence
  Synthetic
tatctttttccagtaatttttagtaaa

SEQ ID NO: 935 - DNA, 27 bp, Artificial Sequence
  Synthetic
tatttatttccagttattttttctaaa

SEQ ID NO: 936 - DNA, 27 bp, Artificial Sequence
  Synthetic
tatttatttccagtaatttttagtaaa

SEQ ID NO: 937 - DNA, 27 bp, Artificial Sequence
  Synthetic
attccacaagttcatacacaagtatta

SEQ ID NO: 938 - DNA, 27 bp, Artificial Sequence
  Synthetic
attccacaagttcatacacaagtactt

SEQ ID NO: 939 - DNA, 27 bp, Artificial Sequence
  Synthetic
atcccacaagttcacacacaagttctt

SEQ ID NO: 940 - DNA, 27 bp, Artificial Sequence
  Synthetic
atcccacaagttcatactcaagtatta

SEQ ID NO: 941 - DNA, 27 bp, Artificial Sequence
  Synthetic
atgccatctttacgtgaagctgcttta

SEQ ID NO: 942 - DNA, 27 bp, Artificial Sequence
  Synthetic
atgccaagtttacgtgaagctgcatta

SEQ ID NO: 943 - DNA, 27 bp, Artificial Sequence
  Synthetic
atgcctagtcttcgtgaagctgcttta

SEQ ID NO: 944 - DNA, 27 bp, Artificial Sequence
  Synthetic
atgccaagtcttagagaggcagcttta

SEQ ID NO: 945 - DNA, 27 bp, Artificial Sequence
  Synthetic
tggccacgtccacgtcgttatgttatg

SEQ ID NO: 946 - DNA, 27 bp, Artificial Sequence
  Synthetic
tggccacgtccacgtcgttatgttatg

SEQ ID NO: 947 - DNA, 27 bp, Artificial Sequence
  Synthetic
tggccaagaccaagacgttacgttatg

SEQ ID NO: 948 - DNA, 27 bp, Artificial Sequence
  Synthetic
tggccacgtccacgtcgttatgtaatg

SEQ ID NO: 949 - DNA, 27 bp, Artificial Sequence
  Synthetic
atctatccaaatgcttctcttcttttc

SEQ ID NO: 950 - DNA, 27 bp, Artificial Sequence
  Synthetic
atttatccaaatgcatctcttcttttt

SEQ ID NO: 951 - DNA, 27 bp, Artificial Sequence
  Synthetic
atttacccaaatgcatctcttcttttc

SEQ ID NO: 952 - DNA, 27 bp, Artificial Sequence
  Synthetic
atttatccaaatgctagtttattattc

SEQ ID NO: 953 - DNA, 27 bp, Artificial Sequence
  Synthetic
cgtcttttagaattttatcttgcagtt

SEQ ID NO: 954 - DNA, 27 bp, Artificial Sequence
  Synthetic
cgtcttttagaattttatttagcagtt

SEQ ID NO: 955 - DNA, 27 bp, Artificial Sequence
  Synthetic
cgtttattagaattctatttagcagta

SEQ ID NO: 956 - DNA, 27 bp, Artificial Sequence
  Synthetic
cgtcttttagaattttatcttgctgtt

SEQ ID NO: 957 - DNA, 27 bp, Artificial Sequence
  Synthetic
ttacttcttggcactattcatgctgtt

SEQ ID NO: 958 - DNA, 27 bp, Artificial Sequence
  Synthetic
ttacttttaggcactattcatgctgtt

SEQ ID NO: 959 - DNA, 27 bp, Artificial Sequence
  Synthetic
cttcttcttggtacaattcatgctgtt

SEQ ID NO: 960 - DNA, 27 bp, Artificial Sequence
  Synthetic
ttattattaggtacaatccatgcagta

SEQ ID NO: 961 - DNA, 27 bp, Artificial Sequence
  Synthetic
aatccacaaccagtttggttatgctta

SEQ ID NO: 962 - DNA, 27 bp, Artificial Sequence
  Synthetic
aatccacaaccagtttggctttgtctt

SEQ ID NO: 963 - DNA, 27 bp, Artificial Sequence
  Synthetic
aatccacagccagtttggttatgcctt

SEQ ID NO: 964 - DNA, 27 bp, Artificial Sequence
  Synthetic
aacccacagccagtttggttatgcctt

SEQ ID NO: 965 - DNA, 27 bp, Artificial Sequence
  Synthetic
aaagttccagaaattgtacattttctt

SEQ ID NO: 966 - DNA, 27 bp, Artificial Sequence
  Synthetic
aaagttccagaaattgtacattttctt

SEQ ID NO: 967 - DNA, 27 bp, Artificial Sequence
  Synthetic
aaagttcccgaaattgtacattttctt

SEQ ID NO: 968 - DNA, 27 bp, Artificial Sequence
  Synthetic
aaagttccagaaatcgttcatttctta

SEQ ID NO: 969 - DNA, 27 bp, Artificial Sequence
  Synthetic
cttcttcttggtacaattcatgcagtg

SEQ ID NO: 970 - DNA, 27 bp, Artificial Sequence
  Synthetic
aaatataaaaaatttccatggtggtta

SEQ ID NO: 971 - DNA, 27 bp, Artificial Sequence
  Synthetic
aatccacaaccagtatggctttgtctt

SEQ ID NO: 972 - DNA, 27 bp, Artificial Sequence
  Synthetic
attccacaagttcatacacaagtactt

SEQ ID NO: 973 - DNA, 27 bp, Artificial Sequence
  Synthetic
aatccacaaccagtttggctttgtctt

SEQ ID NO: 974 - DNA, 27 bp, Artificial Sequence
  Synthetic
catgtatttggttatagttggtataaa

SEQ ID NO: 975 - DNA, 27 bp, Artificial Sequence
  Synthetic
attcttattggtgttttagtaggtgtt

SEQ ID NO: 976 - DNA, 27 bp, Artificial Sequence
  Synthetic
aatccacaaccagtttggttatgttta

SEQ ID NO: 977 - DNA, 27 bp, Artificial Sequence
  Synthetic
aatatgacacatgtactttatccatta

(19) United States
(12) Patent Application Publication
(10) Pub. No.: US 2021/0177955 A1
(43) Pub. Date: Jun. 17, 2021

(54) IMMUNOGENIC HETEROCLITIC PEPTIDES FROM CANCER-ASSOCIATED PROTEINS AND METHODS OF USE THEREOF

(71) Applicant: ADVAXIS, INC., Princeton, NJ (US)

(72) Inventors: Robert PETIT, Newtown, PA (US); Michael F. PRINCIOTTA, Hightstown, NJ (US); Brandon CODER, Trenton, NJ (US); David BALLI, Warrington, PA (US)

(73) Assignee: ADVAXIS, INC., Princeton, NJ (US)

(21) Appl. No.: 16/759,670

(22) PCT Filed: Nov. 8, 2018
(86) PCT No.: PCT/US2018/059849

§ 371 Date: Apr. 27, 2020

Related U.S. Application Data
(60) Provisional application No. 62/583,292, filed on Nov. 8, 2017.
(60) Provisional application No. 62/592,884, filed on Nov. 30, 2017.

(51) Int. Cl. A61K 39/00; C07K 14/47

(52) U.S. Cl. CPC A61K 39/001153; C07K 14/4748; A61K 2039/53; A61K 2039/523; A61K 39/001102

(57) ABSTRACT

Provided herein are tumor-associated antigen peptides comprising heteroclitic mutations and fusion polypeptides comprising such heteroclitic peptide. Also provided are nucleic acids encoding such peptides and fusion polypeptides, recombinant bacteria or Listeria strains comprising such peptides, fusion polypeptides, or nucleic acids, and cell banks comprising such recombinant bacteria or Listeria strains. Also provided herein are methods of generating such peptides, fusion polypeptides, nucleic acids, and recombinant bacteria or Listeria strains. Also provided are immunogenic compositions, pharmaceutical compositions, and vaccines comprising such peptides, fusion polypeptides, nucleic acids, or recombinant bacteria or Listeria strains. Also provided are methods of inducing an anti-tumor-associated-antigen immune response in a subject, methods of inducing an anti-tumor or anti-cancer immune response in a subject, methods of treating a tumor or cancer in a subject, methods of preventing a tumor or cancer in a subject, and methods of protecting a subject against a tumor or cancer using such peptides, recombinant fusion polypeptides, nucleic acids, recombinant bacteria or Listeria strains, immunogenic compositions, pharmaceutical compositions, or vaccines.

### CROSS-REFERENCE TO RELATED APPLICATIONS

This application claims the benefit of U.S. Application No. 62/583,292, filed Nov. 8, 2017, and U.S. Application No. 62/592,884, filed Nov. 30, 2017, each of which is herein incorporated by reference in its entirety for all purposes.

### REFERENCE TO A SEQUENCE LISTING SUBMITTED AS A TEXT FILE VIA EFS WEB

The Sequence Listing written in file 522598SEQLIST.txt is 333 kilobytes, was created on Nov. 3, 2018, and is hereby incorporated by reference.

### BACKGROUND

Tumorigenesis involves acquisition of a set of essential capabilities, including uncontrolled growth, resistance to death, potential to migrate and grow at distant sites, and ability to induce growth of new blood vessels. Underlying these hallmarks is genomic instability, which generates the genetic variation that accelerates their acquisition. Tumor-associated antigens such as cancer testis antigens confer several of these capabilities to cancer cells, suggesting that they are directly implicated in tumorigenesis and making them potential targets for immunotherapy. However, many factors, including T cell tolerance, low affinity of self-antigens for MHCs or TCRs, and the immunosuppressive environment of tumors, can contribute to the minimal expansion of tumor-specific T cells in response to peptide vaccines used to treat cancer patients.

### SUMMARY

Methods and compositions are provided for cancer immunotherapy. In one aspect, provided herein are isolated peptides comprising an immunogenic fragment of a cancer-associated protein, wherein the fragment comprises a heteroclitic mutation. In another aspect, provided are recombinant Listeria strains comprising a nucleic acid comprising a first open reading frame encoding a fusion polypeptide, wherein the fusion polypeptide comprises a PEST-containing peptide fused to one or more immunogenic fragments of a cancer-associated protein, wherein the fragments comprise a heteroclitic mutation. Also provided are such fusion polypeptides and nucleic acids encoding such isolated peptides and fusion polypeptides. Also provided are recombinant bacteria strains comprising such nucleic acids.

In another aspect, provided herein are immunogenic compositions, pharmaceutical compositions, or vaccines comprising such isolated peptides, nucleic acids, fusion polypeptides, recombinant bacteria strains, or recombinant Listeria strains.

In another aspect, provided herein are methods of inducing or enhancing an immune response against a tumor or cancer in a subject, comprising administering to the subject such isolated peptides, nucleic acids, fusion polypeptides, recombinant bacteria strains, or recombinant Listeria strains. Also provided are methods of inducing or enhancing an immune response against a tumor or cancer in a subject, comprising administering to the subject an immunogenic composition, a pharmaceutical composition, or a vaccine comprising such isolated peptides, nucleic acids, fusion polypeptides, recombinant bacteria strains, or recombinant Listeria strains.

In another aspect, provided herein are methods of preventing or treating a tumor or cancer in a subject, comprising administering to the subject such isolated peptides, nucleic acids, fusion polypeptides, recombinant bacteria strains, or recombinant Listeria strains. Also provided are methods of preventing or treating a tumor or cancer in a subject, comprising administering to the subject an immunogenic composition, a pharmaceutical composition, or a vaccine comprising such isolated peptides, nucleic acids, fusion polypeptides, recombinant bacteria strains, or recombinant Listeria strains.

In another aspect, provided herein are cell banks comprising one or more of such recombinant bacteria or recombinant Listeria strains.

### BRIEF DESCRIPTION OF THE FIGURES

FIGS. 1A and 1B show schematics of WT1 minigene constructs. FIG. 1A shows a WT1 minigene construct designed to express a single WT1 chimeric polypeptide antigen.

FIG. 1B shows a WT1 minigene construct designed to express three separate WT1 chimeric polypeptide antigens.

FIGS. 2A and 2B show Western blots of the Lmdda-WT1-tLLO-FLAG-Ub-heteroclitic phenylalanine minigene construct (FIG. 2A) and the Lmdda-WT1-tLLO-P1-P2-P3-FLAG-Ub-heteroclitic tyrosine minigene construct (FIG. 2B). In FIG. 2A, lane 1 is the ladder, lane 2 is the Lmdda-WT1-tLLO-P1-P2-P3-FLAG-Ub-heteroclitic tyrosine minigene construct (68 kDa), and lane 3 is a negative control. In FIG. 2B, lane 1 is the ladder, lane 2 is the negative control, and lane 3 is the WT1-tLLO-FLAG-Ub-heteroclitic phenylalanine minigene construct (construct #1).

FIG. 3 shows colony PCR results for several Lm-minigene constructs expressing heteroclitic mutant WT1 peptides. Mutated residues are bolded and underlined.

FIG. 4 shows an ELISPOT assay in splenocytes stimulated ex vivo with WT1 peptides RMFPNAPYL (SEQ ID NO: 197) and FMFPNAPYL (SEQ ID NO: 160). The splenocytes are from HLA2 transgenic mice immunized with the WT1-F minigene construct. PBS and LmddA274 were used as negative controls.

FIG. 5 shows an ELISPOT assay in splenocytes stimulated ex vivo with WT1 peptides RMFPNAPYL (SEQ ID NO: 197) and YMFPNAPYL (SEQ ID NO: 169). The splenocytes are from HLA2 transgenic mice immunized with the WT1-AH1-Tyr minigene construct. PBS and LmddA274 were used as negative controls.

FIGS. 6A and 6B show IFN-γ spot-forming cells (SFC) per million splenocytes stimulated ex vivo with WT1 peptides RMFPNAPYL (SEQ ID NO: 197; FIG. 6A) and FMFPNAPYL (SEQ ID NO: 160; FIG. 6B). The splenocytes are from HLA2 transgenic mice immunized with the WT1-F minigene construct. PBS and LmddA274 were used as negative controls.

FIGS. 7A and 7B show IFN-γ spot-forming cells (SFC) per million splenocytes stimulated ex vivo with WT1 peptides RMFPNAPYL (SEQ ID NO: 197; FIG. 7A) and YMFPNAPYL (SEQ ID NO: 169; FIG. 7B). The splenocytes are from HLA2 transgenic mice immunized with the WT1-AH1-Tyr minigene construct. PBS and LmddA274 were used as negative controls.

FIG. 8 shows CT26 tumor volume in mice treated with PBS control or Lm AH1_HC.

### DEFINITIONS

The terms “protein,” “polypeptide,” and “peptide,” used interchangeably herein, refer to polymeric forms of amino acids of any length, including coded and non-coded amino acids and chemically or biochemically modified or derivatized amino acids. The terms include polymers that have been modified, such as polypeptides having modified peptide backbones.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The term “fusion protein” refers to a protein comprising two or more peptides linked together by peptide bonds or other chemical bonds. The peptides can be linked together directly by a peptide or other chemical bond. For example, a chimeric molecule can be recombinantly expressed as a single-chain fusion protein. Alternatively, the peptides can be linked together by a “linker” such as one or more amino acids or another suitable linker between the two or more peptides.

The terms “nucleic acid” and “polynucleotide,” used interchangeably herein, refer to polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

“Codon optimization” refers to a process of modifying a nucleic acid sequence for enhanced expression in particular host cells by replacing at least one codon of the native sequence with a codon that is more frequently or most frequently used in the genes of the host cell while maintaining the native amino acid sequence. For example, a polynucleotide encoding a fusion polypeptide can be modified to substitute codons having a higher frequency of usage in a given Listeria cell or any other host cell as compared to the naturally occurring nucleic acid sequence. Codon usage tables are readily available, for example, at the “Codon Usage Database.” The optimal codons utilized by L. monocytogenes for each amino acid are shown US 2007/0207170, herein incorporated by reference in its entirety for all purposes. These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

The term “plasmid” or “vector” includes any known delivery vector including a bacterial delivery vector, a viral vector delivery vector, a peptide immunotherapy delivery vector, a DNA immunotherapy delivery vector, an episomal plasmid, an integrative plasmid, or a phage vector. The term “vector” refers to a construct which is capable of delivering, and, optionally, expressing, one or more fusion polypeptides in a host cell.

The term “episomal plasmid” or “extrachromosomal plasmid” refers to a nucleic acid vector that is physically separate from chromosomal DNA (i.e., episomal or extrachromosomal and does not integrated into a host cell's genome) and replicates independently of chromosomal DNA. A plasmid may be linear or circular, and it may be single-stranded or double-stranded. Episomal plasmids may optionally persist in multiple copies in a host cell's cytoplasm (e.g., Listeria), resulting in amplification of any genes of interest within the episomal plasmid.

The term “genomically integrated” refers to a nucleic acid that has been introduced into a cell such that the nucleotide sequence integrates into the genome of the cell and is capable of being inherited by progeny thereof. Any protocol may be used for the stable incorporation of a nucleic acid into the genome of a cell.

The term “stably maintained” refers to maintenance of a nucleic acid molecule or plasmid in the absence of selection (e.g., antibiotic selection) for at least 10 generations without detectable loss. For example, the period can be at least 15 generations, 20 generations, at least 25 generations, at least 30 generations, at least 40 generations, at least 50 generations, at least 60 generations, at least 80 generations, at least 100 generations, at least 150 generations, at least 200 generations, at least 300 generations, or at least 500 generations. Stably maintained can refer to a nucleic acid molecule or plasmid being maintained stably in cells in vitro (e.g., in culture), being maintained stably in vivo, or both.

An “open reading frame” or “ORF” is a portion of a DNA which contains a sequence of bases that could potentially encode a protein. As an example, an ORF can be located between the start-code sequence (initiation codon) and the stop-codon sequence (termination codon) of a gene.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety.

“Operable linkage” or being “operably linked” refers to the juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” refers to the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) refers to a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence.

The term “wild type” refers to entities having a structure and/or activity as found in a normal (as contrasted with mutant, diseased, altered, or so forth) state or context. Wild type gene and polypeptides often exist in multiple different forms (e.g., alleles).

The term “isolated” with respect to proteins and nucleic acid refers to proteins and nucleic acids that are relatively purified with respect to other bacterial, viral or cellular components that may normally be present in situ, up to and including a substantially pure preparation of the protein and the polynucleotide. The term “isolated” also includes proteins and nucleic acids that have no naturally occurring counterpart, have been chemically synthesized and are thus substantially uncontaminated by other proteins or nucleic acids, or has been separated or purified from most other cellular components with which they are naturally accompanied (e.g., other cellular proteins, polynucleotides, or cellular components).

“Exogenous” or “heterologous” molecules or sequences are molecules or sequences that are not normally expressed in a cell or are not normally present in a cell in that form. Normal presence includes presence with respect to the particular developmental stage and environmental conditions of the cell. An exogenous or heterologous molecule or sequence, for example, can include a mutated version of a corresponding endogenous sequence within the cell or can include a sequence corresponding to an endogenous sequence within the cell but in a different form (i.e., not within a chromosome). An exogenous or heterologous molecule or sequence in a particular cell can also be a molecule or sequence derived from a different species than a reference species of the cell or from a different organism within the same species. For example, in the case of a Listeria strain expressing a heterologous polypeptide, the heterologous polypeptide could be a polypeptide that is not native or endogenous to the Listeria strain, that is not normally expressed by the Listeria strain, from a source other than the Listeria strain, derived from a different organism within the same species.

In contrast, “endogenous” molecules or sequences or “native” molecules or sequences are molecules or sequences that are normally present in that form in a particular cell at a particular developmental stage under particular environmental conditions.

The term “variant” refers to an amino acid or nucleic acid sequence (or an organism or tissue) that is different from the majority of the population but is still sufficiently similar to the common mode to be considered to be one of them (e.g., splice variants).

The term “isoform” refers to a version of a molecule (e.g., a protein) with only slight differences compared to another isoform, or version (e.g., of the same protein). For example, protein isoforms may be produced from different but related genes, they may arise from the same gene by alternative splicing, or they may arise from single nucleotide polymorphisms.

The term “fragment” when referring to a protein means a protein that is shorter or has fewer amino acids than the full length protein. The term “fragment” when referring to a nucleic acid means a nucleic acid that is shorter or has fewer nucleotides than the full length nucleic acid. A fragment can be, for example, an N-terminal fragment (i.e., removal of a portion of the C-terminal end of the protein), a C-terminal fragment (i.e., removal of a portion of the N-terminal end of the protein), or an internal fragment. A fragment can also be, for example, a functional fragment or an immunogenic fragment.

The term “analog” when referring to a protein means a protein that differs from a naturally occurring protein by conservative amino acid differences, by modifications which do not affect amino acid sequence, or by both.

The term “functional” refers to the innate ability of a protein or nucleic acid (or a fragment, isoform, or variant thereof) to exhibit a biological activity or function. Such biological activities or functions can include, for example, the ability to elicit an immune response when administered to a subject. Such biological activities or functions can also include, for example, binding to an interaction partner. In the case of functional fragments, isoforms, or variants, these biological functions may in fact be changed (e.g., with respect to their specificity or selectivity), but with retention of the basic biological function.

The terms “immunogenicity” or “immunogenic” refer to the innate ability of a molecule (e.g., a protein, a nucleic acid, an antigen, or an organism) to elicit an immune response in a subject when administered to the subject. Immunogenicity can be measured, for example, by a greater number of antibodies to the molecule, a greater diversity of antibodies to the molecule, a greater number of T-cells specific for the molecule, a greater cytotoxic or helper T-cell response to the molecule, and the like.

The term “antigen” is used herein to refer to a substance that, when placed in contact with a subject or organism (e.g., when present in or when detected by the subject or organism), results in a detectable immune response from the subject or organism. An antigen may be, for example, a lipid, a protein, a carbohydrate, a nucleic acid, or combinations and variations thereof. For example, an “antigenic peptide” refers to a peptide that leads to the mounting of an immune response in a subject or organism when present in or detected by the subject or organism. For example, such an “antigenic peptide” may encompass proteins that are loaded onto and presented on MHC class I and/or class II molecules on a host cell's surface and can be recognized or detected by an immune cell of the host, thereby leading to the mounting of an immune response against the protein. Such an immune response may also extend to other cells within the host, such as diseased cells (e.g., tumor or cancer cells) that express the same protein.

The term “epitope” refers to a site on an antigen that is recognized by the immune system (e.g., to which an antibody binds). An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996), herein incorporated by reference in its entirety for all purposes.

The term “mutation” refers to the any change of the structure of a gene or a protein. For example, a mutation can result from a deletion, an insertion, a substitution, or a rearrangement of chromosome or a protein. An “insertion” changes the number of nucleotides in a gene or the number of amino acids in a protein by adding one or more additional nucleotides or amino acids. A “deletion” changes the number of nucleotides in a gene or the number of amino acids in a protein by reducing one or more additional nucleotides or amino acids.

A “frameshift” mutation in DNA occurs when the addition or loss of nucleotides changes a gene's reading frame. A reading frame consists of groups of 3 bases that each code for one amino acid. A frameshift mutation shifts the grouping of these bases and changes the code for amino acids. The resulting protein is usually nonfunctional. Insertions and deletions can each be frameshift mutations.

A “missense” mutation or substitution refers to a change in one amino acid of a protein or a point mutation in a single nucleotide resulting in a change in an encoded amino acid. A point mutation in a single nucleotide that results in a change in one amino acid is a “nonsynonymous” substitution in the DNA sequence. Nonsynonymous substitutions can also result in a “nonsense” mutation in which a codon is changed to a premature stop codon that results in truncation of the resulting protein. In contrast, a “synonymous” mutation in a DNA is one that does not alter the amino acid sequence of a protein (due to codon degeneracy).

The term “somatic mutation” includes genetic alterations acquired by a cell other than a germ cell (e.g., sperm or egg). Such mutations can be passed on to progeny of the mutated cell in the course of cell division but are not inheritable. In contrast, a germinal mutation occurs in the germ line and can be passed on to the next generation of offspring.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

Compositions or methods “comprising” or “including” one or more recited elements may include other elements not specifically recited. For example, a composition that “comprises” or “includes” a protein may contain the protein alone or in combination with other ingredients.

Designation of a range of values includes all integers within or defining the range, and all subranges defined by integers within the range.

Unless otherwise apparent from the context, the term “about” encompasses values within a standard margin of error of measurement (e.g., SEM) of a stated value or variations ±0.5%, 1%, 5%, or 10% from a specified value.

The singular forms of the articles “a,” “an,” and “the” include plural references unless the context clearly dictates otherwise. For example, the term “an antigen” or “at least one antigen” can include a plurality of antigens, including mixtures thereof.

Statistically significant means p≤0.05.

### DETAILED DESCRIPTION

### I. Overview

Provided herein are peptides comprising immunogenic fragments of cancer-associated proteins, wherein the fragment comprises a heteroclitic mutation. Some such peptides are recombinant fusion polypeptides comprising one or more immunogenic fragments of cancer-associated proteins, wherein each fragment comprises a heteroclitic mutation (e.g., fused to a PEST-containing peptide). Also provided herein are nucleic acids encoding such peptides; immunogenic compositions, pharmaceutical compositions, or vaccines comprising such peptides or nucleic acids; recombinant bacteria or Listeria strains comprising such peptides or nucleic acids; immunogenic compositions, pharmaceutical compositions, or vaccines comprising such recombinant bacteria or Listeria strains; and methods of generating such peptides, such nucleic acids, and such recombinant bacteria or Listeria strains. Also provided are methods of inducing an anti-tumor-associated-antigen immune response in a subject, methods of inducing an anti-tumor or anti-cancer immune response in a subject, methods of treating a tumor or cancer in a subject, methods of preventing a tumor or cancer in a subject, and methods of protecting a subject against a tumor or cancer using such peptides, nucleic acids, recombinant bacteria or Listeria strains, immunogenic compositions, pharmaceutical compositions, or vaccines.

Design and use of heteroclitic sequences (i.e., sequence-optimized peptides) derived from tumor-associated antigen genes (e.g., from cancer testis antigens (CTAs) or oncofetal antigens (OFAs)) can increase presentation by MHC Class I alleles. Heteroclitic sequences have been shown to be sufficient to prime a T cell response, to overcome central tolerance, and to elicit a successful cross-reactive immune response to the wild-type peptide. OFAs and CTAs are expressed in up to 100% of patients within a cancer indication, but are not expressed in healthy tissue of adults (e.g., normally expressed only in embryonic tissues). Many OFAs/CTAs have primary roles in oncogenesis. Because of OFA/CTAs highly restricted tissue expression in cancer, they are attractive targets for immunotherapy.

Such heteroclitic sequences can be combined such that total patient coverage within a cancer type can approach 100%. Using multiple sequence-optimized, proprietary immunogenic OFA/CTA peptides or tumor-associated antigen peptides (i.e., sequence-optimized to improve immunogenicity) can provide additional targets capable of generating strong T cell responses, making it unnecessary to sequence a patient prior to treatment as it can be assumed that they will express a tumor-associated antigen that we have designed heteroclitic peptides for to cover the most prevalent HLAs (HLA-A0201, HLA-A0301, HLA-A2402, and HLA-B0702).

In some compositions described herein, the heteroclitic peptides are expressed in Listeria monocytogenes (Lm) vectors. The Lm technology has a mechanism of action that incorporates potent innate immune stimulation, delivery of a target peptide directly into the cytosol of dendritic cells and antigen presenting cells, generation of a targeted T cell response, and reduced immune suppression by regulatory T cells and myeloid-derived suppressor cells in the tumor microenvironment. Multiple treatments can be given and/or combined without neutralizing antibodies. The Lm technology can use, for example, live, attenuated, bioengineered Lm bacteria to stimulate the immune system to view tumor cells as potentially bacterial-infected cells and target them for elimination. The technology process can start with a live, attenuated strain of Listeria and can add, for example, multiple copies of a plasmid that encodes a fusion protein sequence including a fragment of, for example, the LLO (listeriolysin O) molecule joined to the antigen of interest. This fusion protein is secreted by the Listeria inside antigen-presenting cells. This results in a stimulation of both the innate and adaptive arms of the immune system that reduces tumor defense mechanisms and makes it easier for the immune system to attack and destroy the cancer cells.

Immunologically, Lm-based vectors are a far superior platform for the generation of CD8+ dominant T cell responses compared to peptide vaccines. First, there is no need to add adjuvants of filgrastim injections. This is because the live attenuated bacteria vectors inherently trigger numerous innate immune activation triggers which include several TLRs, PAMP, and DAMP receptors and have a potent ability to agonize the STING receptor within the cytosol of the antigen-presenting cells. This is a much broader alteration of the immunologic microenvironment that primes the patients' immune system for an adaptive immune response. Second, the Lm vector is infused intravenously. This allows it to reach significantly more antigen-presenting cells than may reside in a finite area of subcutaneous tissue. It also eliminates the requirement for subcutaneous injections, the use of filgrastim, and the risk of delayed type hypersensitivity. It is also likely to generate high T cell titers faster as optimum CD8+ T cell numbers typically peak after 3 treatments, not greater than 10. Third, Lm promotes a predominant CD8+ T cell response with CD4+ cross-reactivity for T cell help. CD8+ T cells are the most effective at killing cancer cells and because Lm vectors present their antigen in the cytoplasm of the APC, those peptides are rapidly shunted to the proteasome for processing, complexed with MHC Class 1 and transported to the APC surface for presentation to predominantly CD8+ T cells. This should bring the advantage of generating more CD8+ T cells that a subcutaneous Montanide presentation of antigen peptides. Fourth, Lm vectors increase the expression of chemokine and chemokine receptors on tumors and surrounding lymph nodes. This facilitates the attraction of activated T cells to the vicinity of solid tumors. Fifth, Lm vectors decrease the relative number and suppressive function of immunosuppressive cells that may protect a tumor from T cell attack, better enabling T cell killing of cancer cells. This reduction of the immunosuppressive ability of regulatory T cells and myeloid derived suppressor cells will better enable T cells generated against these peptides to have better activity in solid tumors. Sixth, Lm vectors do not generate neutralizing antibodies. Because of this, these vectors can be administered repeatedly for extended periods of time without the loss of efficacy from neutralizing antibodies and the development of delayed-type hypersensitivity or acute hypersensitivity which may include anaphylaxis.

Lm vectors act via multiple immunotherapy mechanisms: potent innate immune stimulation via toll-like receptors (TLRs) and pathogen-associated molecular patterns (PAMPs) including the stimulator of interferon genes (STING) receptor, strong CD8⁺ and CD4⁺ T cell responses, epitope spreading, and immune suppression by disabling Tregs and myeloid derived suppressor cells (MDSCs) in the tumor microenvironment. In addition, the unique intracellular life cycle of Listeria avoids neutralizing antibodies, allowing for repeat dosing. Lm is also advantageous because it has synergies with checkpoint inhibitors, costimulatory agonists, and others agents. It also has a large capacity and can be adapted to target many different tumor types. As an example, live, attenuated strains of Lm can be bioengineered to secrete an antigen-adjuvant fusion protein comprising, consisting essentially of, or consisting of a truncated fragment of listeriolysin O (tLLO), which has adjuvant properties, and one or more tumor-associated antigens. Upon infusion into a patient, bioengineered Lm can be phagocytosed by antigen-presenting cells, where the fusion protein is secreted by the Lm, processed, and presented onto major histocompatibility complex (MHC) class I and II molecules. Target peptides presented on the surface of the antigen-presenting cells stimulate tumor-associated-antigen-specific CD4⁺ and CD8⁺ T cells. Activated CD8⁺ T cells can then seek out and kill tumor-associated-antigen-expressing cancer cells and modulate the tumor microenvironment to overcome immune suppression.

Lm vectors have some clinical advantages. Any side effects associated with treatment appear in the hours immediately post-infusion while the patient is still in the clinic, are almost exclusively mild-moderate and respond readily to treatment, and resolve the day of dosing without evidence of delayed onset, cumulative toxicity, or lasting sequalae. Practical advantages include the fact that there is no need to administer multiple agents and switch to alternate dosing sites for subsequent administrations.

From a manufacturing standpoint, there are several advantages. First, there is no need to manufacture the individual peptides to high concentrations and high degrees of purity. The Lm bacteria transcribe the DNA simultaneously on multiple copies of DNA plasmids inside the bacteria and secrete these peptides directly into the cytoplasm of the APC, where they are almost immediately transported to the proteasome for processing. Essentially, the peptides are manufactured by the bacteria right at the point of use for antigen processing. Second, Lm vectors are highly scalable. Once the genetic engineering is complete, the bacteria replicate themselves in broth cultures. The cultures can be scaled up to vastly reduce cost of goods. Third, there is no need to formulate in a complex carrier like Montanide or create an emulsion. Fourth, the bacteria are very stable, some more than 5 years, without worry of peptide degradation or breakdown product contamination that can lead to loss of potency of a peptide formulation.

In some Lm vectors disclosed herein, a minigene construct is used as described in more detail elsewhere herein. Use of the minigene construct approach disclosed herein for the expression of specific MHC class I binding antigenic determinants allows for the highly efficient delivery of short peptide sequences to the antigen presentation pathway of professional antigen presenting cells (pAPC). A specific advantage of the minigene technology is that it bypasses the requirement for proteasome mediated degradation of larger proteins in order to liberate short peptide sequences that can be bound and presented on MHC class I molecules. This results in a much higher efficiency of peptide-MHC class I antigen presentation on the surface of the pAPC and, therefore, a much higher level of antigen expression for the priming of antigen specific T cell responses.

### II. Tumor-Associated Antigen Peptides Comprising Heteroclitic Mutations and Nucleic Acids Encoding Such Peptides

Disclosed herein are peptides comprising immunogenic fragments of cancer-associated proteins, wherein the fragment comprises a heteroclitic mutation.

The term “heteroclitic” refers to a peptide that generates an immune response that recognizes the native peptide from which the heteroclitic peptide was derived (e.g., the peptide not containing the anchor residue mutations). For example, YLMPVNSEV (SEQ ID NO: 130) was generated from YMMPVNSEV (SEQ ID NO: 131) by mutation of residue 2 to methionine. A heteroclitic peptide can generate an immune response that recognizes the native peptide from which the heteroclitic peptide was derived. For example, the immune response against the native peptide generated by vaccination with the heteroclitic peptide can be equal or greater in magnitude than the immune response generated by vaccination with the native peptide. The immune response can be increased, for example, by 2-fold, 3-fold, 5-fold, 7-fold, 10-fold, 15-fold, 20-fold, 30-fold, 50-fold, 100-fold, 150-fold, 200-fold, 300-fold, 500-fold, 1000-fold, or more.

A heteroclitic peptide disclosed herein can bind to one or more human leukocyte antigens (HLA) molecules. HLA molecules, also known as major histocompatibility complex (MHC) molecules, bind peptides and present them to immune cells. The immunogenicity of a peptide can be partially determined by its affinity for HLA molecules. HLA class I molecules interact with CD8 molecules, which are generally present on cytotoxic T lymphocytes (CTL). HLA class II molecules interact with CD4 molecules, which are generally present on helper T lymphocytes. For example, a heteroclitic peptide disclosed herein can bind to an HLA molecule with sufficient affinity to activate a T cell precursor or with sufficient affinity to mediate recognition by a T cell.

A heteroclitic peptide disclosed herein can bind to one or more HLA class II molecules. For example, a heteroclitic peptide can bind to an HLA-DRB molecule, an HLA-DRA molecule, an HLA-DQA1 molecule, an HLA-DQB1 molecule, an HLA-DPA1 molecule, an HLA-DPB 1 molecule, an HLA-DMA molecule, an HLA-DMB molecule, an HLA-DOA molecule, or an HLA-DOB molecule.

A native or heteroclitic peptide disclosed herein can bind to one or more HLA class I molecules. For example, a heteroclitic peptide can bind to an HLA-A molecule, an HLA-B molecule, an HLA-C molecule, an HLA-A0201 molecule, HLA A1, HLA A2, HLA A2.1, HLA A3, HLA A3.2, HLA All, HLA A24, HLA B7, HLA B27, or HLA B8. Similarly, a heteroclitic peptide can bind to a superfamily of HLA class I molecules, such as the A2 superfamily, the A3 superfamily, the A24 superfamily, the B7 superfamily, the B27 superfamily, the B44 superfamily, the C1 superfamily, or the C4 superfamily. In a specific example, the heteroclitic peptide or fragment binds to one or more of the following HLA types: HLA-A*02:01, HLA-A*03:01, HLA-A*24:02, and HLA-B*07:02.

Heteroclitic peptides can comprise a mutation that enhances binding of the peptide to an HLA class II molecule relative to the corresponding native peptide. Alternatively, or additionally, heteroclitic peptides can comprise a mutation that enhances binding of the peptide to an HLA class I molecule relative to the corresponding native peptide. For example, the mutated residue can be an HLA class II motif anchor residue. “Anchor motifs” or “anchor residues” refers, in another embodiment, to one or a set of preferred residues at particular positions in an HLA-binding sequence (e.g., an HLA class II binding sequence or an HLA class I binding sequence).

Various methods are well-known for generating predicted heteroclitic epitopes with the potential to elicit cross-reactive immunogenic responses to a wild-type epitope. For example, to design heteroclitic epitopes with the potential to elicit cross-reactive immunogenic responses to a wild-type epitope, baseline predicted peptide-MHC binding affinity of the wild-type epitopes can be determined using NetMHCpan 3.0 Server (www.cbs.dtu.dk/services/NetMHCpan/). A peptide-MHC binding affinity percent rank of less than or equal to 1.0 is considered a strong binder that is likely to elicit an immune response. Potential heteroclitic epitopes are generated by random substitution of 1 or more amino acids at, but not limited to, positions 1, 2, 3, or the C-terminal position of the wild-type epitope that is predicted to be a strong binder. The peptide-MHC binding affinity of the potential heteroclitic epitopes is then estimated using NetMHCpan 3.0 Server. Heteroclitic epitopes with percentage ranking binding affinities similar to wild-type epitopes and less than or equal to 1.0 percentage rank can be considered potential antigens for future validation.

Other methods for identifying HLA class I and class II residues, and for improving HLA binding by mutating the residues, are well-known. See, e.g., U.S. Pat. Nos. 8,765,687, 7,488,718, 9,233,149, and 7,598,221, each of which is herein incorporated by reference in its entirety for all purposes. For example, methods for predicting MHC class II epitopes are well-known. As one example, the MHC class II epitope can be predicted using TEPITOPE (Meister et al. (1995) Vaccine 13:581-591, herein incorporated by reference in its entirety for all purposes). As another example, the MHC class II epitope can be predicted using EpiMatrix (De Groot et al. (1997) AIDS Res. Hum. Retroviruses 13:529-531, herein incorporated by reference in its entirety for all purposes). As yet another example, the MHC class II epitope can be predicted using the Predict Method (Yu K et al. (2002) Mol. Med. 8:137-148, herein incorporated by reference in its entirety for all purposes). As yet another example, the MHC class II epitope can be predicted using the SYFPEITHI epitope prediction algorithm. SYFPEITHI is a database comprising more than 4500 peptide sequences known to bind class I and class II MHC molecules. SYFPEITHI provides a score based on the presence of certain amino acids in certain positions along the MHC-binding groove. Ideal amino acid anchors are valued at 10 points, unusual anchors are worth 6-8 points, auxiliary anchors are worth 4-6 points, preferred residues are worth 1-4 points; negative amino acid effect on the binding score between −1 and −3. The maximum score for HLA-A*0201 is 36. As yet another example, the MHC class II epitope can be predicted using Rankpep. Rankpep uses position specific scoring matrices (PSSMs) or profiles from sets of aligned peptides known to bind to a given MHC molecule as the predictor of MHC-peptide binding. Rankpep includes information on the score of the peptide and the % optimum or percentile score of the predicted peptide relative to that of a consensus sequence that yields the maximum score, with the selected profile. Rankpep includes a selection of 102 and 80 PSSMs for the prediction of peptide binding to MHC I and MHC II molecules, respectively. Several PSSMs for the prediction of peptide binders of different sizes are usually available for each MHC I molecule. As another example, the MHC class II epitope can be identified using SVMHC (Donnes and Elofsson (2002) BMC Bioinformatics 11; 3:25, herein incorporated by reference in its entirety for all purposes).

Methods for identifying MHC class I epitopes are also well-known. As one example, the MHC class I epitope can be predicted using BIMAS software. A BIMAS score is based on the calculation of the theoretical half-life of the MHC-I/β₂-microglobulin/peptide complex, which is a measure of peptide-binding affinity. The program uses information about HLA-I peptides of 8-10 amino acids in length. The higher the binding affinity of a peptide to the MHC, the higher the likelihood that this peptide represents an epitope. The BIMAS algorithm assumes that each amino acid in the peptide contributes independently to binding to the class I molecule. Dominant anchor residues, which are critical for binding, have coefficients in the tables that are significantly higher than 1. Unfavorable amino acids have positive coefficients that are less than 1. If an amino acid is not known to make either a favorable or unfavorable contribution to binding, then it is assigned the value 1. All the values assigned to the amino acids are multiplied and the resulting running score is multiplied by a constant to yield an estimate of half-time of dissociation. As another example, the MHC class I epitope can be identified using SYFPEITHI. As yet another example, the MHC class I epitope can be identified using SVMHC. As yet another example, the MHC class I epitope can be identified using NetMHC-2.0 (Buus et al. (2003) Tissue Antigens 62:378-384, herein incorporated by reference in its entirety for all purposes).

Different residues in HLA binding motifs can be mutated to enhance MHC binding. In one example, a mutation that enhances MHC binding is in the residue at position 1 of the HLA class I binding motif (e.g., a mutation to tyrosine, glycine, threonine, or phenylalanine). As another example, the mutation can be in position 2 of the HLA class I binding motif (e.g., a mutation to leucine, valine, isoleucine, or methionine). As another example, the mutation can be in position 6 of the HLA class I binding motif (e.g., to valine, cysteine, glutamine, or histidine). As another example, the mutation can be in position 9 of the HLA class I binding motif or in the C-terminal position (e.g., to valine, threonine, isoleucine, leucine, alanine, or cysteine). The mutation can be in a primary anchor residue or in a secondary anchor residue. For example, the HLA class I primary anchor residues can be positions 2 and 9, and the secondary anchor residues can be positions 1 and 8 or positions 1, 3, 6, 7, and 8. In another example, a point mutation can be in a position selected from positions 4, 5, and 8.

Similarly, different residues in HLA class II binding sites can be mutated. For example, an HLA class II motif anchor residue can be modified. For example, the P1 position, the P2 position, the P6 position, or the P9 position can be mutated. Alternatively, the P4 position, the P5 position, the P10 position, the P11 position, the P12 position, or the P13 position can be mutated.

Individual heteroclitic mutations can be selected based on any criteria as discussed in further detail elsewhere herein. For example, individual heteroclitic mutations or heteroclitic peptides can be selected if they are known to generate CD8+ T lymphocyte responses.

After identification of a set of possible heteroclitic mutations, sequences for heteroclitic immunogenic peptides comprising each heteroclitic mutation can be selected. Different size peptides can be used, as disclosed elsewhere herein. For example, heteroclitic mutations or heteroclitic immunogenic peptides can be focused, for example, on MHC Class I epitopes consisting of 9 amino acids.

The sequence of the heteroclitic immunogenic peptide can then be optimized to enhance binding to MHC Class I molecules. To optimize binding to each HLA, the Peptide MHC Binding Motif and Amino Acid Binding Chart can be assessed from the Immune Epitope Database and Analysis Resource (for example: iedb.org/MHCalleleid/143). The preferred amino acids at the anchor positions can be inserted into the heteroclitic antigenic peptide sequence (e.g., NUF2—wild type: YMMPVNSEV (SEQ ID NO: 131); and NUF2—heteroclitic: YLMPVNSEV (SEQ ID NO: 130)).

The binding affinities of sequence-optimized heteroclitic antigenic peptides can then be assessed, for example, using one of the following algorithms: NetMHC4.0 Server; NetMHCpan4.0 Server; and mhcflurry v0.2.0. The heteroclitic antigenic peptides can be considered, for example, if predicting binding affinity to a specific HLA is equivalent or stronger than the corresponding native sequence. Selected sequence-optimized heteroclitic antigenic peptides can then be screened for in vitro binding to specific HLAs using ProImmune's REVEAL assay. For example, heteroclitic antigenic peptides with binding affinity >=45% of the REVEAL assay's positive control peptide can be considered binders.

The binding affinity (e.g., IC50) for a sequence-optimized heteroclitic antigenic peptide can be, for example, less than 1000, 500, 400, 300, 200, 150, 100, 90, 80, 70, 60, 50, 40, 30, 20, 15, 10, 9, 8, 7, 6, 5, 4, 3, 2, or 1 nM. For example, the binding affinity (e.g., IC50) can be between about 0.5-500, 0.5-300, 0.5-200, 0.5-100, 0.5-50, 0.5-40, 0.5-30, 0.5-20, 0.5-10, or 0.5-5 nM.

The RNA expression level of heteroclitic antigenic peptides can also be measured in a specific-indication in The Cancer Genome Atlas (TCGA) RNAseqV2 dataset. The percentage of TCGA samples with normalized RNA expression reads greater than 0 can be calculated. Heteroclitic antigenic peptides with TCGA expression in a majority of samples can be prioritized.

In a specific example, a literature review can be done to survey the genomic landscape of indication-specific tumor-associated antigens to generate a short-list of potential TAAs. A second literature review can be done to determine if short-list TAAs contain known immunogenic peptides that generate CD8+ T lymphocyte response. This approach can focus, for example, primarily on MHC Class I epitopes consisting of 9 amino acids (9mer) from TAAs. This step can, for example, identify potential target peptides in 9mer format that bind to one of four HLAs types (HLA-A*02:01, HLA-A*03:01, HLA-A*24:02, and HLA-B*07:02).

Target peptides can then be sequence optimized to enhance binding to MHC Class I molecules (aka heteroclitic peptide). To optimize binding to each HLA, the Peptide MHC Binding Motif and Amino Acid Binding Chart can be assessed from the Immune Epitope Database and Analysis Resource (for example: iedb.org/MHCalleleid/143). The preferred amino acids at the anchor positions can be inserted into the target peptide sequence (e.g., NUF2—wild type: YMMPVNSEV (SEQ ID NO: 131); and NUF2—heteroclitic: YLMPVNSEV (SEQ ID NO: 130)). The binding affinities of sequence-optimized target peptides and wild-type target peptides can then be assessed, e.g., using one of the following algorithms: NetMHC4.0 Server; NetMHCpan4.0 Server; and mhcflurry v0.2.0. Sequence-optimized target peptides can be considered, for example, if predicting binding affinity to a specific HLA is equivalent or stronger than the wild-type target peptide sequence. Selected sequence-optimized target peptides can then be screened for in vitro binding to specific HLAs using ProImmune's REVEAL assay. For example, target peptides with binding affinity>=45% of the REVEAL assay's positive control peptide can be considered binders. Finally, the RNA expression level of target peptides can be measured in a specific-indication in the TCGA RNAseqV2 dataset. For example, the percentage of TCGA samples with normalized RNA expression reads greater than 0 can be calculated. For example, target peptides with TCGA expression in a majority of samples can be prioritized.

The term “cancer-associated protein” includes proteins having mutations that occur in multiple types of cancer, that occur in multiple subjects having a particular type of cancer, or that are correlated with the occurrence or progression of one or more types of cancer. For example, a cancer-associated protein can be an oncogenic protein (i.e., a protein with activity that can contribute to cancer progression, such as proteins that regulate cell growth), or it can be a tumor-suppressor protein (i.e., a protein that typically acts to alleviate the potential for cancer formation, such as through negative regulation of the cell cycle or by promoting apoptosis.

The term “cancer-associated protein” in the context of heteroclitic peptides refers to proteins whose expression is correlated with the occurrence or progression of one or more types of cancer. Optionally, such proteins includes proteins having mutations that occur in multiple types of cancer, that occur in multiple subjects having a particular type of cancer, or that are correlated with the occurrence or progression of one or more types of cancer. For example, a cancer-associated protein can be an oncogenic protein (i.e., a protein with activity that can contribute to cancer progression, such as proteins that regulate cell growth), or it can be a tumor-suppressor protein (i.e., a protein that typically acts to alleviate the potential for cancer formation, such as through negative regulation of the cell cycle or by promoting apoptosis). Preferably, a cancer-associated protein from which a heteroclitic peptide is derived is a protein that is expressed in a particular type of cancer but is not normally expressed in healthy adult tissue (i.e., a protein with cancer-specific expression, cancer-restricted expression, tumor-specific expression, or tumor-restricted expression). However, a cancer-associated protein does not have to have cancer-specific, cancer-restricted, tumor-specific, or tumor-restricted expression. Examples of proteins that are considered cancer-specific or cancer-restricted are cancer testis antigens or oncofetal antigens. Cancer testis antigens (CTAs) are a large family of tumor-associated antigens expressed in human tumors of different histological origin but not in normal tissue, except for male germ cells. In cancer, these developmental antigens can be re-expressed and can serve as a locus of immune activation. Oncofetal antigens (OFAs) are proteins that are typically present only during fetal development but are found in adults with certain kinds of cancer. The tumor-restricted pattern of expression of CTAs and OFAs make them ideal targets for tumor-specific immunotherapy. Most OFA/CTA proteins play critical roles in oncogenesis.

For example, the cancer-associated protein can be any one of the cancer-associated proteins listed elsewhere herein. For example, the cancer-associated protein can be encoded by one of the following genes: CEACAM5, GAGE1, hTERT, KLHL7, MAGEA3, MAGEA4, MAGEA6, NUF2, NYESO1, PAGE4, PRAME, PSA, PSMA, RNF43, SART3, SSX2, STEAP1, and SURVIVIN.

| | | | SEQ ID |
|---|---|---|---|
| Gene | Protein | UniProt | NO |
| CEACAM5 (CEA) | Carcinoembryonic antigen-related cell adhesion | P06731 | 170 |
| | molecule 5 | | |
| GAGE1 | G antigen 1 | Q13065 | 171 |
| hTERT (TERT, EST2, TCS1, | Telomerase reverse transcriptase | O14746 | 172 |
| TRT) | | | |
| KLHL7 | Kelch-like protein 7 | Q8IXQ5 | 173 |
| MAGEA3 (MAGE3) | Melanoma-associated antigen 3 | P43357 | 174 |
| MAGEA4 (MAGE4) | Melanoma-associated antigen 4 | P43358 | 175 |
| MAGEA6 (MAGE6) | Melanoma-associated antigen 6 | P43360 | 176 |
| NUF2 (CDCA1, NUF2R) | Kinetochore protein Nuf2 | Q9BZD4 | 177 |
| NYESO1 (NY-ESO-1, CTAG1A, | Cancer/testis antigen 1 (Autoimmunogenic | P78358 | 178 |
| CTAG, CTAG1, ESO1, LAGE2, | cancer/testis antigen NY-ESO-1) | | |
| LAGE2A, CTAG1B, LAGE2B) | | | |
| PAGE4 (GAGEC1, JM27) | P antigen family member 4 | O60829 | 179 |
| PRAME (MAPE, OIP4) | Melanoma antigen preferentially expressed in | P78395 | 180 |
| | tumors | | |
| PSA (KLK3, APS) | Prostate-specific antigen | P07288 | 181 |
| PSMA (FOLH1, FOLH, | Glutamate carboxypeptidase 2 (Prostate-specific | Q04609 | 182 |
| NAALAD1, PSM, GIG27) | membrane antigen) | | |
| RNF43 | E3 ubiquitin-protein ligase RNF43 | Q68DV7 | 183 |
| SART3 (KIAA0156, TIP110) | Squamous cell carcinoma antigen recognized by | Q15020 | 184 |
| | T-cells 3 | | |
| SSX2 (SSX2A, SSX2B) | Protein SSX2 | Q16385 | 185 |
| STEAP1 (PRSS24, STEAP) | Metalloreductase STEAP1 | Q9UHE8 | 186 |
| SURVIVIN (BIRC5, API4, IAP4) | Baculoviral IAP repeat-containing protein 5 | O15392 | 187 |
| | (Apoptosis inhibitor survivin) | | |

Each heteroclitic immunogenic peptide can be a fragment of a cancer-associated protein (i.e., a contiguous sequence of amino acids from a cancer-associated protein) comprising a heteroclitic mutation. Each heteroclitic immunogenic peptide can be of any length sufficient to induce an immune response. For example, a heteroclitic immunogenic peptide disclosed herein can be 5-100, 15-50, or 21-27 amino acids in length, or 15-100, 15-95, 15-90, 15-85, 15-80, 15-75, 15-70, 15-65, 15-60, 15-55, 15-50, 15-45, 15-40, 15-35, 15-30, 20-100, 20-95, 20-90, 20-85, 20-80, 20-75, 20-70, 20-65, 20-60, 20-55, 20-50, 20-45, 20-40, 20-35, 20-30, 11-21, 15-21, 21-31, 31-41, 41-51, 51-61, 61-71, 71-81, 81-91, 91-101, 101-121, 121-141, 141-161, 161-181, 181-201, 8-27, 10-30, 10-40, 15-30, 15-40, 15-25, 1-10, 10-20, 20-30, 30-40, 1-100, 5-75, 5-50, 5-40, 5-30, 5-20, 5-15, 5-10, 1-75, 1-50, 1-40, 1-30, 1-20, 1-15, 1-10, 8-11, or 11-16 amino acids in length. For example, a heteroclitic immunogenic peptide can be 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids in length. For example, a heteroclitic immunogenic peptide can be 8-100, 8-50, 8-30, 8-25, 8-22, 8-20, 8-15, 8-14, 8-13, 8-12, 8-11, 7-11, or 8-10 amino acids in length. In one example, a heteroclitic immunogenic peptide can be 9 amino acids in length.

In some cases, a heteroclitic immunogenic peptide can be hydrophilic or can score up to or below a certain hydropathy threshold, which can be predictive of secretability in Listeria monocytogenes or another bacteria of interest. For example, heteroclitic immunogenic peptides can be scored by a Kyte and Doolittle hydropathy index 21 amino acid window, and all scoring above a cutoff (around 1.6) may be excluded as they are unlikely to be secretable by Listeria monocytogenes.

A heteroclitic immunogenic peptide can comprise a single heteroclitic mutation or can comprise two or more heteroclitic mutations (e.g., two heteroclitic mutations). Exemplary heteroclitic mutant peptides consist of, consist essentially of, or comprise a heteroclitic peptide sequence in the following table, which also provides the corresponding wild type (native) peptides. The residues in the wild type peptides that are modified in the corresponding heteroclitic peptides are bolded and underlined.

| Peptide | | |
|---|---|---|
| (GENE_HLA Type) | Heteroclitic Peptide | Native Peptide |
| CEACAM5_A0201 | ILIGVLVGV (SEQ ID NO: 100) | IMIGVLVGV (SEQ ID NO: 101) |
| CEACAM5_A0201 | ILMGVLVGV (SEQ ID NO: 102) | IMIGVLVGV (SEQ ID NO: 103) |
| CEACAM5_A0301 | HVFGYSWYK (SEQ ID NO: 104) | HLFGYSWYK (SEQ ID NO: 105) |
| CEACAM5_A2402 | IYPNASLLF (SEQ ID NO: 106) | IYPNASLLI (SEQ ID NO: 107) |
| CEACAM5_B0702 | IPQVHTQVL (SEQ ID NO: 108) | IPQQHTQVL (SEQ ID NO: 109) |
| GAGE1_A0301 | SLYWPRPR (SEQ ID NO: 110) | STYWPRPR (SEQ ID NO: 111) |
| GAGE1_B0702 | WPRPRRYVM (SEQ ID NO: 112) | WPRPRRYVQ (SEQ ID NO: 113) |
| hTERT_A0201_A2402 | IMAKFLHWL (SEQ ID NO: 114) | ILAKFLHWL (SEQ ID NO: 115) |
| KLHL7_A2402 | VYILGGSQF (SEQ ID NO: 116) | VYILGGSQL (SEQ ID NO: 117) |
| MAGEA3_A0201_A2402 | KVPEIVHFL (SEQ ID NO: 118) | KVAELVHFL (SEQ ID NO: 119) |
| MAGEA3_A0301 | YMFPVIFSK (SEQ ID NO: 120) | YFFPVIFSK (SEQ ID NO: 121) |
| MAGEA3_A2402 | IMPKAGLLF (SEQ ID NO: 122) | IMPKAGLLFI (SEQ ID NO: 123) |
| MAGEA3_B0702 | LPWTMNYPL (SEQ ID NO: 124) | LPTTMNYPL (SEQ ID NO: 125) |
| MAGEA4_B0702 | MPSLREAAL (SEQ ID NO: 126) | YPSLREAAL (SEQ ID NO: 127) |
| MAGEA6_A0301 | YLFPVIFSK (SEQ ID NO: 128) | YFFPVIFSK (SEQ ID NO: 129) |
| NUF2_A0201 | YLMPVNSEV (SEQ ID NO: 130) | YMMPVNSEV (SEQ ID NO: 131) |
| NUF2_A2402 | VWGIRLEHF (SEQ ID NO: 132) | VYGIRLEHF (SEQ ID NO: 133) |
| NYESO1_A0201 | RLLEFYLAV (SEQ ID NO: 134) | RLLEFYLAM (SEQ ID NO: 135) |
| NYESO1_B0702 | APRGPHGGM (SEQ ID NO: 136) | APRGPHGGA (SEQ ID NO: 137) |
| PAGE4_A0201 | MAPDVVAFV (SEQ ID NO: 138) | EAPDVVAFV (SEQ ID NO: 139) |
| PRAME_A0201 | NMTHVLYPL (SEQ ID NO: 140) | NLTHVLYPV (SEQ ID NO: 141) |
| PSA_A0301 | GMAPLILSR (SEQ ID NO: 142) | GAAPLILSR (SEQ ID NO: 143) |
| PSMA_A2402 | TYSVSFFSW (SEQ ID NO: 144) | TYSVSFDSL (SEQ ID NO: 145) |
| RNF43_B0702 | NPQPVWLCL (SEQ ID NO: 146) | NSQPVWLCL (SEQ ID NO: 147) |
| SART3_A0201 | LMQAEAPRL (SEQ ID NO: 148) | LLQAEAPRL (SEQ ID NO: 149) |
| SSX2_A0201 | RLQGISPKV (SEQ ID NO: 150) | RLQGISPKI (SEQ ID NO: 151) |
| STEAP1_A0201 | LLLGTIHAV (SEQ ID NO: 152) | LLLGTIHAL (SEQ ID NO: 153) |
| STEAP1_A2402 | KYKKFPWWL (SEQ ID NO: 154) | KYKKFPHWL (SEQ ID NO: 155) |
| SURVIVIN_A0201 | KMSSGCAFL (SEQ ID NO: 156) | KHSSGCAFL (SEQ ID NO: 157) |
| SURVIVIN_A2402 | SWFKNWPFF (SEQ ID NO: 158) | STFKNWPFL (SEQ ID NO: 159) |

Nucleic acids encoding such heteroclitic peptides are also disclosed. The nucleic acid can be in any form. The nucleic acid can comprise or consist of DNA or RNA, and can be single-stranded or double-stranded. The nucleic acid can be in the form of a plasmid, such as an episomal plasmid, a multicopy episomal plasmid, or an integrative plasmid. Alternatively, the nucleic acid can be in the form of a viral vector, a phage vector, or in a bacterial artificial chromosome. Such nucleic acids can have one open reading frame or can have two or more open reading frames. In one example, such nucleic acids can comprise two or more open reading frames linked by a Shine-Dalgarno ribosome binding site nucleic acid sequence between each open reading frame. For example, a nucleic acid can comprise two to four open reading frames linked by a Shine-Dalgarno ribosome binding site nucleic acid sequence between each open reading frame. Each open reading frame can encode a different peptide. In some nucleic acids, the codon encoding the carboxy terminus of the fusion polypeptide is followed by two stop codons to ensure termination of protein synthesis.

Nucleic acids can be codon optimized. A nucleic acid is codon-optimized if at least one codon in the nucleic acid is replaced with a codon that is more frequently used by a particular organism (e.g., codon optimized for expression in humans or L. monocytogenes) for that amino acid than the codon in the original sequence. Examples of nucleic acids encoding heteroclitic peptides disclosed herein are provided in SEQ ID NOS: 223-977.

### III. Recombinant Fusion Polypeptides

Disclosed herein are recombinant fusion polypeptides comprising a PEST-containing peptide fused to one or more tumor-associated antigen peptides comprising heteroclitic mutations (i.e., fused to one or more immunogenic fragments of cancer-associated proteins, wherein each fragment comprises a heteroclitic mutation) as disclosed elsewhere herein.

Also disclosed herein are recombinant fusion polypeptides comprising one or more tumor-associated antigen peptides comprising heteroclitic mutations (i.e., fused to one or more immunogenic fragments of cancer-associated proteins, wherein each fragment comprises a heteroclitic mutation) as disclosed elsewhere herein, and wherein the fusion polypeptide does not comprise a PEST-containing peptide.

Also provided herein are recombinant fusion polypeptides comprising from N-terminal end to C-terminal end a bacterial secretion sequence, a ubiquitin (Ub) protein, and one or more tumor-associated antigen peptides comprising heteroclitic mutations (i.e., fused to one or more immunogenic fragments of cancer-associated proteins, wherein each fragment comprises a heteroclitic mutation) as disclosed elsewhere herein (i.e., in tandem, such as Ub-peptide1-peptide2). Alternatively, a combination of separate fusion polypeptides can be used in which each antigenic peptide is fused to its own secretion sequence and Ub protein (e.g., Ub1-peptide1; Ub2-peptide2).

Nucleic acids (termed minigene constructs) encoding such recombinant fusion polypeptides are also disclosed. Such minigene nucleic acid constructs can further comprise two or more open reading frames linked by a Shine-Dalgarno ribosome binding site nucleic acid sequence between each open reading frame. For example, a minigene nucleic acid construct can further comprise two to four open reading frames linked by a Shine-Dalgarno ribosome binding site nucleic acid sequence between each open reading frame. Each open reading frame can encode a different polypeptide. In some nucleic acid constructs, the codon encoding the carboxy terminus of the fusion polypeptide is followed by two stop codons to ensure termination of protein synthesis.

The bacterial signal sequence can be a Listerial signal sequence, such as an Hly or an ActA signal sequence, or any other known signal sequence. In other cases, the signal sequence can be an LLO signal sequence. An exemplary LLO signal sequence is set forth in SEQ ID NO: 97. The signal sequence can be bacterial, can be native to a host bacterium (e.g., Listeria monocytogenes, such as a secA1 signal peptide), or can be foreign to a host bacterium. Specific examples of signal peptides include an Usp45 signal peptide from Lactococcus lactis, a Protective Antigen signal peptide from Bacillus anthracis, a secA2 signal peptide such the p60 signal peptide from Listeria monocytogenes, and a Tat signal peptide such as a B. subtilis Tat signal peptide (e.g., PhoD). In specific examples, the secretion signal sequence is from a Listeria protein, such as an ActA₃₀₀ secretion signal or an ActA₁₀₀ secretion signal. An exemplary ActA signal sequence is set forth in SEQ ID NO: 98.

The ubiquitin can be, for example, a full-length protein. An exemplary ubiquitin sequence is set forth in SEQ ID NO: 188. The ubiquitin expressed from the nucleic acid construct provided herein can be cleaved at the carboxy terminus from the rest of the recombinant fusion polypeptide expressed from the nucleic acid construct through the action of hydrolases upon entry to the host cell cytosol. This liberates the amino terminus of the fusion polypeptide, producing a peptide in the host cell cytosol.

Selection of, variations of, and arrangement of antigenic peptides within a fusion polypeptide are discussed in detail elsewhere herein, and tumor-associated antigen peptides comprising heteroclitic mutations are discussed in more detail elsewhere herein.

The recombinant fusion polypeptides can comprise one or more tags. For example, the recombinant fusion polypeptides can comprise one or more peptide tags N-terminal and/or C-terminal to the one or more antigenic peptides. A tag can be fused directly to an antigenic peptide or linked to an antigenic peptide via a linker (examples of which are disclosed elsewhere herein). Examples of tags include the following: FLAG tag; 2×FLAG tag; 3×FLAG tag; His tag, 6×His tag; and SIINFEKL tag. An exemplary SIINFEKL tag is set forth in SEQ ID NO: 16 (encoded by any one of the nucleic acids set forth in SEQ ID NOS: 1-15). An exemplary 3×FLAG tag is set forth in SEQ ID NO: 32 (encoded by any one of the nucleic acids set forth in SEQ ID NOS: 17-31). An exemplary variant 3×FLAG tag is set forth in SEQ ID NO: 99. Two or more tags can be used together, such as a 2×FLAG tag and a SIINFEKL tag, a 3×FLAG tag and a SIINFEKL tag, or a 6×His tag and a SIINFEKL tag. If two or more tags are used, they can be located anywhere within the recombinant fusion polypeptide and in any order. For example, the two tags can be at the C-terminus of the recombinant fusion polypeptide, the two tags can be at the N-terminus of the recombinant fusion polypeptide, the two tags can be located internally within the recombinant fusion polypeptide, one tag can be at the C-terminus and one tag at the N-terminus of the recombinant fusion polypeptide, one tag can be at the C-terminus and one internally within the recombinant fusion polypeptide, or one tag can be at the N-terminus and one internally within the recombinant fusion polypeptide. Other tags include chitin binding protein (CBP), maltose binding protein (MBP), glutathione-S-transferase (GST), thioredoxin (TRX), and poly(NANP). Particular recombinant fusion polypeptides comprise a C-terminal SIINFEKL tag. Such tags can allow for easy detection of the recombinant fusion protein, confirmation of secretion of the recombinant fusion protein, or for following the immunogenicity of the secreted fusion polypeptide by following immune responses to these “tag” sequence peptides. Such immune response can be monitored using a number of reagents including, for example, monoclonal antibodies and DNA or RNA probes specific for these tags.

The recombinant fusion polypeptides disclosed herein can be expressed by recombinant Listeria strains or can be expressed and isolated from other vectors and cell systems used for protein expression and isolation. Recombinant Listeria strains comprising expressing such antigenic peptides can be used, for example in immunogenic compositions comprising such recombinant Listeria and in vaccines comprising the recombinant Listeria strain and an adjuvant. Expression of one or more antigenic peptides as a fusion polypeptides with a nonhemolytic truncated form of LLO, ActA, or a PEST-like sequence in host cell systems in Listeria strains and host cell systems other than Listeria can result in enhanced immunogenicity of the antigenic peptides.

Nucleic acids encoding such recombinant fusion polypeptides are also disclosed. The nucleic acid can be in any form. The nucleic acid can comprise or consist of DNA or RNA, and can be single-stranded or double-stranded. The nucleic acid can be in the form of a plasmid, such as an episomal plasmid, a multicopy episomal plasmid, or an integrative plasmid. Alternatively, the nucleic acid can be in the form of a viral vector, a phage vector, or in a bacterial artificial chromosome. Such nucleic acids can have one open reading frame or can have two or more open reading frames (e.g., an open reading frame encoding the recombinant fusion polypeptide and a second open reading frame encoding a metabolic enzyme). In one example, such nucleic acids can comprise two or more open reading frames linked by a Shine-Dalgarno ribosome binding site nucleic acid sequence between each open reading frame. For example, a nucleic acid can comprise two to four open reading frames linked by a Shine-Dalgarno ribosome binding site nucleic acid sequence between each open reading frame. Each open reading frame can encode a different polypeptide. In some nucleic acids, the codon encoding the carboxy terminus of the fusion polypeptide is followed by two stop codons to ensure termination of protein synthesis.

A. Antigenic Peptides

The recombinant fusion polypeptides disclosed herein comprise one or more tumor-associated antigenic peptides comprising heteroclitic mutations (i.e., immunogenic fragments of cancer-associated proteins, wherein each fragment comprises a heteroclitic mutation) as disclosed elsewhere herein. The fusion polypeptide can include a single antigenic peptide or can includes two or more antigenic peptides. Each antigenic peptide can be of any length sufficient to induce an immune response, and each antigenic peptide can be the same length or the antigenic peptides can have different lengths. Examples of suitable lengths for heteroclitic antigenic peptides are disclosed elsewhere herein.

Each antigenic peptide can also be hydrophilic or can score up to or below a certain hydropathy threshold, which can be predictive of secretability in Listeria monocytogenes or another bacteria of interest. For example, antigenic peptides can be scored by a Kyte and Doolittle hydropathy index 21 amino acid window, and all scoring above a cutoff (around 1.6) can be excluded as they are unlikely to be secretable by Listeria monocytogenes. Likewise, the combination of antigenic peptides or the fusion polypeptide can be hydrophilic or can score up to or below a certain hydropathy threshold, which can be predictive of secretability in Listeria monocytogenes or another bacteria of interest.

The antigenic peptides can be linked together in any manner. For example, the antigenic peptides can be fused directly to each other with no intervening sequence. Alternatively, the antigenic peptides can be linked to each other indirectly via one or more linkers, such as peptide linkers. In some cases, some pairs of adjacent antigenic peptides can be fused directly to each other, and other pairs of antigenic peptides can be linked to each other indirectly via one or more linkers. The same linker can be used between each pair of adjacent antigenic peptides, or any number of different linkers can be used between different pairs of adjacent antigenic peptides. In addition, one linker can be used between a pair of adjacent antigenic peptides, or multiple linkers can be used between a pair of adjacent antigenic peptides.

Any suitable sequence can be used for a peptide linker. As an example, a linker sequence may be, for example, from 1 to about 50 amino acids in length. Some linkers may be hydrophilic. The linkers can serve varying purposes. For example, the linkers can serve to increase bacterial secretion, to facilitate antigen processing, to increase flexibility of the fusion polypeptide, to increase rigidity of the fusion polypeptide, or any other purpose. As a specific example, one or more or all of a flexibility linker, a rigidity linker, and an immunoproteasome processing linker can be used. Examples of such linkers are provided below. In some cases, different amino acid linker sequences are distributed between the antigenic peptides or different nucleic acids encoding the same amino acid linker sequence are distributed between the antigenic peptides (e.g., SEQ ID NOS: 84-94) in order to minimize repeats. This can also serve to reduce secondary structures, thereby allowing efficient transcription, translation, secretion, maintenance, or stabilization of the nucleic acid (e.g., plasmid) encoding the fusion polypeptide within a Lm recombinant vector strain population. Other suitable peptide linker sequences may be chosen, for example, based on one or more of the following factors: (1) their ability to adopt a flexible extended conformation; (2) their inability to adopt a secondary structure that could interact with functional epitopes on the antigenic peptides; and (3) the lack of hydrophobic or charged residues that might react with the functional epitopes. For example, peptide linker sequences may contain Gly, Asn and Ser residues. Other near neutral amino acids, such as Thr and Ala may also be used in the linker sequence. Amino acid sequences which may be usefully employed as linkers include those disclosed in Maratea et al. (1985) Gene 40:39-46; Murphy et al. (1986) Proc Natl Acad Sci USA 83:8258-8262; U.S. Pat. Nos. 4,935,233; and 4,751,180, each of which is herein incorporated by reference in its entirety for all purposes. Specific examples of linkers include those in the following table (each of which can be used by itself as a linker, in a linker comprising repeats of the sequence, or in a linker further comprising one or more of the other sequences in the table), although others can also be envisioned (see, e.g., Reddy Chichili et al. (2013) Protein Science 22:153-167, herein incorporated by reference in its entirety for all purposes). Unless specified, “n” represents an undetermined number of repeats in the listed linker.

| Peptide Linker | Example | SEQ ID NO: | Hypothetical Purpose |
|---|---|---|---|
| (GAS)ₙ | GASGAS | 33 | Flexibility |
| (GSA)ₙ | GSAGSA | 34 | Flexibility |
| (G)ₙ; n = 4-8 | GGGG | 35 | Flexibility |
| (GGGGS)ₙ; n = 1-3 | GGGGS | 36 | Flexibility |
| VGKGGSGG | VGKGGSGG | 37 | Flexibility |
| (PAPAP)ₙ | PAPAP | 38 | Rigidity |
| (EAAAK)ₙ; n = 1-3 | EAAAK | 39 | Rigidity |
| (AYL)ₙ | AYLAYL | 40 | Antigen Processing |
| (LRA)ₙ | LRALRA | 41 | Antigen Processing |
| (RLRA)ₙ | RLRA | 42 | Antigen Processing |
| AAY | AAY | N/A | Immunoproteasome Processing |
| ADLVVG | ADLVVG | 209 | Immunoproteasome Processing |
| ADLIEATAEEVL | ADLIEATAEEVL | 210 | Immunoproteasome Processing |
| GDGSIVSLAKTA | GDGSIVSLAKTA | 211 | Immunoproteasome Processing |
| RDGSVADLAKVA | RDGSVADLAKVA | 212 | Immunoproteasome Processing |
| ADGSVKTLSKVL | ADGSVKTLSKVL | 213 | Immunoproteasome Processing |
| GDGSIVDGSKEL | GDGSIVDGSKEL | 214 | Immunoproteasome Processing |
| GDGSIKTAVKSL | GDGSIKTAVKSL | 215 | Immunoproteasome Processing |
| ADLSVATLAKSL | ADLSVATLAKSL | 216 | Immunoproteasome Processing |
| ADLAVKTLAKVL | ADLAVKTLAKVL | 217 | Immunoproteasome Processing |

The VGKGGSGG linker (SEQ ID NO: 37) can be used, for example, to provide flexibility and to charge balance the fusion protein. The EAAAK linker (SEQ ID NO: 39) is a rigid/stiff linker that can be used to facilitate expression and secretion, for example, if a fusion protein would otherwise fold on itself. The GGGGS linker (SEQ ID NO: 36) is a flexible linker that can be used, for example, to add increased flexibility to a fusion protein to help facilitate expression and secretion. The “i20” linkers (e.g., SEQ ID NOS: 209-217) are immunoproteasome linkers that are designed, for example, to help facilitate cleavage of the fusion protein by the immunoproteasome and increase the frequency of obtaining the exact minimal binding fragment that is desired as with the heteroclitic 9mers designed and disclosed herein. Combinations of GGGGS and EAAAK linkers (SEQ ID NOS: 36 and 39, respectively) can be used, for example, to alternate flexibility and rigidity to help balance the construct for improved expression and secretion and to help facilitate DNA synthesis by providing more unique codons to choose from.

The fusion polypeptide can comprise any number of heteroclitic antigenic peptides. In some cases, the fusion polypeptide comprises any number of heteroclitic antigenic peptides such that the fusion polypeptide is able to be produced and secreted from a recombinant Listeria strain. For example, the fusion polypeptide can comprise at least 3, 4, 5, 6, 7, 8, 9, 10, 11, 12, 13, 14, 15, 16, 17, 18, 19, 20, 21, 22, 23, 24, 25, 26, 27, 28, 29, or 30 heteroclitic antigenic peptides, or 2-50, 2-45, 2-40, 2-35, 2-30, 2-25, 2-20, 2-15, 2-10, 2-5, 5-10, 10-15, 15-20, 20-25, 25-30, 30-35, 35-40, 40-45, or 45-50 heteroclitic antigenic polypeptides. In another example, the fusion polypeptide can include a single heteroclitic antigenic peptide. In another example, the fusion polypeptide can include a number of heteroclitic antigenic peptides ranging from about 1-100, 1-5, 5-10, 10-15, 15-20, 10-20, 20-30, 30-40, 40-50, 50-60, 60-70, 70-80, 80-90, 90-100, 5-15, 5-20, 5-25, 15-20, 15-25, 15-30, 15-35, 20-25, 20-35, 20-45, 30-45, 30-55, 40-55, 40-65, 50-65, 50-75, 60-75, 60-85, 70-85, 70-95, 80-95, 80-105, 95-105, 50-100, 1-100, 5-100, 5-75, 5-50, 5-40, 5-30, 5-20, 5-15, 5-10, 1-100, 1-75, 1-50, 1-40, 1-30, 1-20, 1-15, or 1-10 heteroclitic antigenic peptides. In another example, the fusion polypeptide can include up to about 100, 10, 20, 30, 40, or 50 heteroclitic antigenic peptides. In another example, the fusion polypeptide can comprise about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 heteroclitic antigenic peptides.

In addition, the fusion polypeptide can comprise any number of heteroclitic antigenic peptides from the same cancer-associated protein (i.e., any number of non-contiguous fragments from the same cancer-associated protein). Alternatively, the fusion polypeptide can comprise any number of heteroclitic antigenic peptides from two or more different cancer-associated proteins, such as from 2, 3, 4, 5, 6, 7, 8, 9, or 10 cancer-associated proteins. For example, the fusion polypeptide can comprise heteroclitic mutations from at least 2, 3, 4, 5, 6, 7, 8, 9, 10, 11, 12, 13, 14, 15, 16, 17, 18, 19, or 20 cancer-associated proteins, or 2-5, 5-10, 10-15, or 15-20 cancer-associated proteins. For example, the two or more cancer-associated proteins can be about 2-30, about 2-25, about 2-20, about 2-15, or about 2-10 cancer-associated proteins. For example, the fusion polypeptide can comprise at least 3, 4, 5, 6, 7, 8, 9, 10, 11, 12, 13, 14, 15, 16, 17, 18, 19, 20, 21, 22, 23, 24, 25, 26, 27, 28, 29, or 30 heteroclitic antigenic peptides from the same cancer-associated protein, or 2-50, 2-45, 2-40, 2-35, 2-30, 2-25, 2-20, 2-15, 2-10, 2-5, 5-10, 10-15, 15-20, 20-25, 25-30, 30-35, 35-40, 40-45, or 45-50 heteroclitic antigenic polypeptides from the same cancer-associated protein. Likewise, the fusion polypeptide can comprise at least 3, 4, 5, 6, 7, 8, 9, 10, 11, 12, 13, 14, 15, 16, 17, 18, 19, 20, 21, 22, 23, 24, 25, 26, 27, 28, 29, or 30 heteroclitic antigenic peptides from the same cancer-associated protein, or 2-50, 2-45, 2-40, 2-35, 2-30, 2-25, 2-20, 2-15, 2-10, 2-5, 5-10, 10-15, 15-20, 20-25, 25-30, 30-35, 35-40, 40-45, or 45-50 heteroclitic antigenic polypeptides from two or more different cancer-associated proteins. In addition, the fusion polypeptide can comprise any number of non-contiguous heteroclitic antigenic peptides from the same cancer-associated protein (i.e., any number of non-contiguous fragments from the same cancer-associated protein). For example, the fusion polypeptide can comprise at least 3, 4, 5, 6, 7, 8, 9, 10, 11, 12, 13, 14, 15, 16, 17, 18, 19, 20, 21, 22, 23, 24, 25, 26, 27, 28, 29, or 30 non-contiguous heteroclitic antigenic peptides from the same cancer-associated protein, or 2-50, 2-45, 2-40, 2-35, 2-30, 2-25, 2-20, 2-15, 2-10, 2-5, 5-10, 10-15, 15-20, 20-25, 25-30, 30-35, 35-40, 40-45, or 45-50 non-contiguous heteroclitic antigenic polypeptides from the same cancer-associated protein. In some cases, at least 50%, 55%, 60%, 65%, 70%, 75%, 80%, 85%, 90%, 95%, 96%, 97%, 98%, 99%, or all of the heteroclitic antigenic peptides are non-contiguous heteroclitic antigenic peptides from the same cancer-associated protein, or at least 50%, 55%, 60%, 65%, 70%, 75%, 80%, 85%, 90%, 95%, 96%, 97%, 98%, 99%, or all of the heteroclitic antigenic peptides that are from a single cancer-associated protein are non-contiguous heteroclitic antigenic peptides from that cancer-associated protein.

Each heteroclitic antigenic peptide can comprise a different (i.e., unique) heteroclitic mutation. Alternatively, two or more of the heteroclitic antigenic peptides in the fusion polypeptide can comprise the same heteroclitic mutation. For example, two or more copies of the same heteroclitic antigenic polypeptide can be included in the fusion polypeptide (i.e., the fusion polypeptide comprises two or more copies of the same heteroclitic antigenic peptide). In some fusion polypeptides, at least 50%, 55%, 60%, 65%, 70%, 75%, 80%, 85%, 90%, 95%, 96%, 97%, 98%, or 99% of the heteroclitic antigenic peptides comprise a different (i.e., unique) heteroclitic mutation that is not present in any of the other heteroclitic antigenic peptides.

In some cases, at least two of the heteroclitic antigenic peptides can comprise overlapping fragments of the same cancer-associated protein. For example, two or more of the heteroclitic antigenic peptides can comprise different heteroclitic mutations at the same amino acid residue of the cancer-associated protein.

Some heteroclitic antigenic peptides can comprise at least two different heteroclitic mutations, at least three different heteroclitic mutations, or at least four different heteroclitic mutations.

Any combination of heteroclitic mutations can be included in the fusion polypeptide. For example, heteroclitic antigenic peptides can be included that bind to one or more different HLA types. For example, heteroclitic antigenic peptides can be identified that bind to one or more or all of the following HLA types: HLA-A*02:01, HLA-A*03:01, HLA-A*24:02, and HLA-B*07:02.

Each of the heteroclitic antigenic peptides in the fusion polypeptide can comprise a heteroclitic mutation from the same cancer-associated protein, or the combination of heteroclitic antigenic peptides in the fusion polypeptide can comprise heteroclitic mutations from two or more cancer-associated proteins. For example, the fusion polypeptide can comprise heteroclitic mutations from at least 2, 3, 4, 5, 6, 7, 8, 9, 10, 11, 12, 13, 14, 15, 16, 17, 18, 19, or 20 cancer-associated proteins, or 2-5, 5-10, 10-15, or 15-20 cancer-associated proteins. For example, the two or more cancer-associated proteins can be about 2-30, about 2-25, about 2-20, about 2-15, or about 2-10 cancer-associated proteins. In one example, at least 50%, 55%, 60%, 65%, 70%, 75%, 80%, 85%, 90%, 95%, 96%, 97%, 98%, or 99% of the heteroclitic antigenic peptides comprise a heteroclitic mutation from the same cancer-associated protein. In another example, none of the heteroclitic antigenic peptides comprise a heteroclitic mutation from the same cancer-associated protein.

Exemplary sequences of heteroclitic antigenic peptides are disclosed elsewhere herein. As an example, a heteroclitic antigenic peptide can comprise, consist essentially of, or consist of a sequence at least 85%, 90%, 91%, 92%, 93%, 94%, 95%, 96%, 97%, 98%, 99%, or 100% identical to any of the antigenic peptide sequences disclosed herein.

As one example, the recombinant fusion polypeptide can comprise heteroclitic peptides encoded by 1 or more, 2 or more, 3 or more, 4 or more, 5 or more, 6 or more, or all of the following genes: CEACAM5, MAGEA6, MAGEA4, GAGE1, NYESO1, STEAP1, and RNF43.

The heteroclitic antigenic peptides can bind, for example, one or more or all of HLA-A*02:01, HLA-A*03:01, HLA-A*24:02, and HLA-B*07:02. Such cancer-associated proteins are associated with, for example, non-small cell lung cancer (NSCLC). The heteroclitic antigenic peptides can be in any order. The heteroclitic antigenic peptides can be fused directly together or linked together by linkers, examples of which are disclosed elsewhere herein. In a specific example, one or more or all of the heteroclitic antigenic peptides can be 9-mers (e.g., 9-mers linked together by linkers). Examples of such antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, or all 11 of the heteroclitic antigenic peptides in Table 3 or peptides comprising, for example, 1 or more, 2 or more, 3 or more, 4 or more, 5 or more, 6 or more, 7 or more, 8 or more, 9 or more, 10 or more, or all 11 of the sequences in Table 3.

As another example, the recombinant fusion polypeptide can comprise heteroclitic peptides encoded by 1 or more, 2 or more, 3 or more, 4 or more, 5 or more, 6 or more, 7 or more, 8 or more, or all of the following genes: CEACAM5, MAGEA4, STEAP1, RNF43, SSX2, SART3, PAGE4, PSMA, and PSA. The heteroclitic antigenic peptides can bind, for example, one or more or all of HLA-A*02:01, HLA-A*03:01, HLA-A*24:02, and HLA-B*07:02. Such cancer-associated proteins are associated with, for example, prostate cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, or all 10 of the heteroclitic antigenic peptides in Table 5 or peptides comprising, for example, 1 or more, 2 or more, 3 or more, 4 or more, 5 or more, 6 or more, 7 or more, 8 or more, 9 or more, or all 10 of the sequences in Table 5.

As another example, the recombinant fusion polypeptide can comprise heteroclitic peptides encoded by 1 or more, 2 or more, 3 or more, 4 or more, 5 or more, or all of the following genes: CEACAM5, STEAP1, MAGEA3, PRAME, hTERT, and SURVIVIN. The heteroclitic antigenic peptides can bind, for example, one or more or all of HLA-A*02:01, HLA-A*03:01, HLA-A*24:02, and HLA-B*07:02. Such cancer-associated proteins are associated with, for example, pancreatic cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, 11 or more, or all 12 of the heteroclitic antigenic peptides in Table 7 or peptides comprising, for example, 1 or more, 2 or more, 3 or more, 4 or more, 5 or more, 6 or more, 7 or more, 8 or more, 9 or more, 10 or more, 11 or more, or all 12 of the sequences in Table 7.

As another example, the recombinant fusion polypeptide can comprise heteroclitic peptides encoded by 1 or more, 2 or more, 3 or more, 4 or more, 5 or more, 6 or more, 7 or more, or all of the following genes: CEACAM5, GAGE1, NYESO1, RNF43, NUF2, KLHL7, MAGEA3, and PRAME. The heteroclitic antigenic peptides can bind, for example, one or more or all of HLA-A*02:01, HLA-A*03:01, HLA-A*24:02, and HLA-B*07:02. Such cancer-associated proteins are associated with, for example, bladder cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, 11 or more, 12 or more, 13 or more, or all 14 of the heteroclitic antigenic peptides in Table 9 or peptides comprising, for example, 1 or more, 2 or more, 3 or more, 4 or more, 5 or more, 6 or more, 7 or more, 8 or more, 9 or more, 10 or more, 11 or more, 12 or more, or all 13 of the sequences in Table 9.

As another example, the recombinant fusion polypeptide can comprise heteroclitic peptides encoded by 1 or more, 2 or more, 3 or more, 4 or more, 5 or more, or all of the following genes: CEACAM5, STEAP1, RNF43, MAGEA3, PRAME, and hTERT. The heteroclitic antigenic peptides can bind, for example, one or more or all of HLA-A*02:01, HLA-A*03:01, HLA-A*24:02, and HLA-B*07:02. Such cancer-associated proteins are associated with, for example, breast cancer (e.g., ER+ breast cancer). The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, or all 11 of the heteroclitic antigenic peptides in Table 11 or peptides comprising, for example, 1 or more, 2 or more, 3 or more, 4 or more, 5 or more, 6 or more, 7 or more, 8 or more, 9 or more, 10 or more, or all 11 of the sequences in Table 11.

As another example, the recombinant fusion polypeptide can comprise heteroclitic peptides encoded by 1 or more, 2 or more, 3 or more, 4 or more, 5 or more, 6 or more, 7 or more, ore or all of the following genes: CEACAM5, PRAME, hTERT, STEAP1, RNF43, NUF2, KLHL7, and SART3. The heteroclitic antigenic peptides can bind, for example, one or more or all of HLA-A*02:01, HLA-A*03:01, HLA-A*24:02, and HLA-B*07:02. Such cancer-associated proteins are associated with, for example, uterine cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, 11 or more, 12 or more, 13 or more, or all 14 of the heteroclitic antigenic peptides in Table 13 or peptides comprising, for example, 1 or more, 2 or more, 3 or more, 4 or more, 5 or more, 6 or more, 7 or more, 8 or more, 9 or more, 10 or more, 11 or more, 12 or more, 13 or more, or all 14 of the sequences in Table 13.

As another example, the recombinant fusion polypeptide can comprise heteroclitic peptides encoded by 1 or more, 2 or more, 3 or more, 4 or more, 5 or more, 6 or more, 7 or more, or all of the following genes: CEACAM5, STEAP1, RNF43, SART3, NUF2, KLHL7, PRAME, and hTERT. The heteroclitic antigenic peptides can bind, for example, one or more or all of HLA-A*02:01, HLA-A*03:01, HLA-A*24:02, and HLA-B*07:02. Such cancer-associated proteins are associated with, for example, ovarian cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, 11 or more, 12 or more, 13 or more, or all 14 of the heteroclitic antigenic peptides in Table 15 or peptides comprising, for example, 1 or more, 2 or more, 3 or more, 4 or more, 5 or more, 6 or more, 7 or more, 8 or more, 9 or more, 10 or more, 11 or more, 12 or more, 13 or more, or all 14 of the sequences in Table 15.

As another example, the recombinant fusion polypeptide can comprise heteroclitic peptides encoded by 1 or more, 2 or more, 3 or more, 4 or more, 5 or more, 6 or more, 7 or more, or all of the following genes: CEACAM5, MAGEA6, STEAP1, RNF43, SART3, NUF2, KLHL7, and hTERT. The heteroclitic antigenic peptides can bind, for example, one or more or all of HLA-A*02:01, HLA-A*03:01, HLA-A*24:02, and HLA-B*07:02. Such cancer-associated proteins are associated with, for example, low-grade glioma. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, or all 10 of the heteroclitic antigenic peptides in Table 17 or peptides comprising, for example, 1 or more, 2 or more, 3 or more, 4 or more, 5 or more, 6 or more, 7 or more, 8 or more, 9 or more, or all 10 of the sequences in Table 17.

As another example, the recombinant fusion polypeptide can comprise heteroclitic peptides encoded by 1 or more, 2 or more, 3 or more, 4 or more, 5 or more, 6 or more, 7 or more, or all of the following genes: CEACAM5, MAGEA6, MAGEA4, GAGE1, NYESO1, STEAP1, RNF43, and MAGEA3. The heteroclitic antigenic peptides can bind, for example, one or more or all of HLA-A*02:01, HLA-A*03:01, HLA-A*24:02, and HLA-B*07:02. Such cancer-associated proteins are associated with, for example, colorectal cancer (e.g., MSS colorectal cancer). The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, or all 10 of the heteroclitic antigenic peptides in Table 19 or peptides comprising, for example, 1 or more, 2 or more, 3 or more, 4 or more, 5 or more, 6 or more, 7 or more, 8 or more, 9 or more, or all 10 of the sequences in Table 19.

As another example, the recombinant fusion polypeptide can comprise heteroclitic peptides encoded by 1 or more, 2 or more, 3 or more, 4 or more, 5 or more, or all of the following genes: CEACAM5, MAGEA4, STEAP1, NYESO1, PRAME, and hTERT. The heteroclitic antigenic peptides can bind, for example, one or more or all of HLA-A*02:01, HLA-A*03:01, HLA-A*24:02, and HLA-B*07:02. Such cancer-associated proteins are associated with, for example, head and neck cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, or all 10 of the heteroclitic antigenic peptides in Table 21 or peptides comprising, for example, 1 or more, 2 or more, 3 or more, 4 or more, 5 or more, 6 or more, 7 or more, 8 or more, 9 or more, or all 10 of the sequences in Table 21.

B. PEST-Containing Peptides

The recombinant fusion proteins disclosed herein comprise a PEST-containing peptide. The PEST-containing peptide may at the amino terminal (N-terminal) end of the fusion polypeptide (i.e., N-terminal to the antigenic peptides), may be at the carboxy terminal (C-terminal) end of the fusion polypeptide (i.e., C-terminal to the antigenic peptides), or may be embedded within the antigenic peptides. In some recombinant Listeria strains and methods, a PEST containing peptide is not part of and is separate from the fusion polypeptide. Fusion of an antigenic peptides to a PEST-like sequence, such as an LLO peptide, can enhance the immunogenicity of the antigenic peptides and can increase cell-mediated and antitumor immune responses (i.e., increase cell-mediated and anti-tumor immunity). See, e.g., Singh et al. (2005) J Immunol 175(6):3663-3673, herein incorporated by reference in its entirety for all purposes.

A PEST-containing peptide is one that comprises a PEST sequence or a PEST-like sequence. PEST sequences in eukaryotic proteins have long been identified. For example, proteins containing amino acid sequences that are rich in prolines (P), glutamic acids (E), serines (S) and threonines (T) (PEST), generally, but not always, flanked by clusters containing several positively charged amino acids, have rapid intracellular half-lives (Rogers et al. (1986) Science 234:364-369, herein incorporated by reference in its entirety for all purposes). Further, it has been reported that these sequences target the protein to the ubiquitin-proteosome pathway for degradation (Rechsteiner and Rogers (1996) Trends Biochem. Sci. 21:267-271, herein incorporated by reference in its entirety for all purposes). This pathway is also used by eukaryotic cells to generate immunogenic peptides that bind to MHC class I and it has been hypothesized that PEST sequences are abundant among eukaryotic proteins that give rise to immunogenic peptides (Realini et al. (1994) FEBS Lett. 348:109-113, herein incorporated by reference in its entirety for all purposes). Prokaryotic proteins do not normally contain PEST sequences because they do not have this enzymatic pathway. However, a PEST-like sequence rich in the amino acids proline (P), glutamic acid (E), serine (S) and threonine (T) has been reported at the amino terminus of LLO and has been reported to be essential for L. monocytogenes pathogenicity (Decatur and Portnoy (2000) Science 290:992-995, herein incorporated by reference in its entirety for all purposes). The presence of this PEST-like sequence in LLO targets the protein for destruction by proteolytic machinery of the host cell so that once the LLO has served its function and facilitated the escape of L. monocytogenes from the phagosomal or phagolysosomal vacuole, it is destroyed before it can damage the cells.

Identification of PEST and PEST-like sequences is well known in the art and is described, for example, in Rogers et al. (1986) Science 234(4774):364-378 and in Rechsteiner and Rogers (1996) Trends Biochem. Sci. 21:267-271, each of which is herein incorporated by reference in its entirety for all purposes. A PEST or PEST-like sequence can be identified using the PEST-find program. For example, a PEST-like sequence can be a region rich in proline (P), glutamic acid (E), serine (S), and threonine (T) residues. Optionally, the PEST-like sequence can be flanked by one or more clusters containing several positively charged amino acids. For example, a PEST-like sequence can be defined as a hydrophilic stretch of at least 12 amino acids in length with a high local concentration of proline (P), aspartate (D), glutamate (E), serine (S), and/or threonine (T) residues. In some cases, a PEST-like sequence contains no positively charged amino acids, namely arginine (R), histidine (H), and lysine (K). Some PEST-like sequences can contain one or more internal phosphorylation sites, and phosphorylation at these sites precedes protein degradation.

In one example, the PEST-like sequence fits an algorithm disclosed in Rogers et al. In another example, the PEST-like sequence fits an algorithm disclosed in Rechsteiner and Rogers. PEST-like sequences can also be identified by an initial scan for positively charged amino acids R, H, and K within the specified protein sequence. All amino acids between the positively charged flanks are counted, and only those motifs containing a number of amino acids equal to or higher than the window-size parameter are considered further. Optionally, a PEST-like sequence must contain at least one P, at least one D or E, and at least one S or T.

The quality of a PEST motif can be refined by means of a scoring parameter based on the local enrichment of critical amino acids as well as the motifs hydrophobicity. Enrichment of D, E, P, S, and T is expressed in mass percent (w/w) and corrected for one equivalent of D or E, one1 of P, and one of S or T. Calculation of hydrophobicity can also follow in principle the method of Kyte and Doolittle (1982) J. Mol. Biol. 157:105, herein incorporated by reference in its entirety for all purposes. For simplified calculations, Kyte-Doolittle hydropathy indices, which originally ranged from −4.5 for arginine to +4.5 for isoleucine, are converted to positive integers, using the following linear transformation, which yielded values from 0 for arginine to 90 for isoleucine: Hydropathy index=10*Kyte-Doolittle hydropathy index+45.

A potential PEST motif's hydrophobicity can also be calculated as the sum over the products of mole percent and hydrophobicity index for each amino acid species. The desired PEST score is obtained as combination of local enrichment term and hydrophobicity term as expressed by the following equation: PEST score=0.55*DEPST−0.5*hydrophobicity index.

Thus, a PEST-containing peptide can refer to a peptide having a score of at least +5 using the above algorithm. Alternatively, it can refer to a peptide having a score of at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 32, at least 35, at least 38, at least 40, or at least 45.

Any other available methods or algorithms known in the art can also be used to identify PEST-like sequences. See, e.g., the CaSPredictor (Garay-Malpartida et al. (2005) Bioinformatics 21 Suppl 1:i169-76, herein incorporated by reference in its entirety for all purposes). Another method that can be used is the following: a PEST index is calculated for each stretch of appropriate length (e.g. a 30-35 amino acid stretch) by assigning a value of one to the amino acids Ser, Thr, Pro, Glu, Asp, Asn, or Gln. The coefficient value (CV) for each of the PEST residues is one and the CV for each of the other AA (non-PEST) is zero.

Examples of PEST-like amino acid sequences are those set forth in SEQ ID NOS: 43-51. One example of a PEST-like sequence is KENSISSMAPPASPPASPKTPIEKKHADEIDK (SEQ ID NO: 43). Another example of a PEST-like sequence is KENSISSMAPPASPPASPK (SEQ ID NO: 44). However, any PEST or PEST-like amino acid sequence can be used. PEST sequence peptides are known and are described, for example, in U.S. Pat. Nos. 7,635,479; 7,665,238; and US 2014/0186387, each of which is herein incorporated by reference in its entirety for all purposes.

The PEST-like sequence can be from a Listeria species, such as from Listeria monocytogenes. For example, the Listeria monocytogenes ActA protein contains at least four such sequences (SEQ ID NOS: 45-48), any of which are suitable for use in the compositions and methods disclosed herein. Other similar PEST-like sequences include SEQ ID NOS: 52-54. Streptolysin O proteins from Streptococcus sp. also contain a PEST sequence. For example, Streptococcus pyogenes streptolysin O comprises the PEST sequence KQNTASTETTTTNEQPK (SEQ ID NO: 49) at amino acids 35-51 and Streptococcus equisimilis streptolysin O comprises the PEST-like sequence KQNTANTETTTTNEQPK (SEQ ID NO: 50) at amino acids 38-54. Another example of a PEST-like sequence is from Listeria seeligeri cytolysin, encoded by the lso gene: RSEVTISPAETPESPPATP (e.g., SEQ ID NO: 51).

Alternatively, the PEST-like sequence can be derived from other prokaryotic organisms. Other prokaryotic organisms wherein PEST-like amino acid sequences would be expected include, for example, other Listeria species.

(I) Listeriolysin O (LLO)

One example of a PEST-containing peptide that can be utilized in the compositions and methods disclosed herein is a listeriolysin O (LLO) peptide. An example of an LLO protein is the protein assigned GenBank Accession No. P13128 (SEQ ID NO: 55; nucleic acid sequence is set forth in GenBank Accession No. X15127). SEQ ID NO: 55 is a proprotein including a signal sequence. The first 25 amino acids of the proprotein is the signal sequence and is cleaved from LLO when it is secreted by the bacterium, thereby resulting in the full-length active LLO protein of 504 amino acids without the signal sequence. An LLO peptide disclosed herein can comprise the signal sequence or can comprise a peptide that does not include the signal sequence. Exemplary LLO proteins that can be used comprise, consist essentially of, or consist of the sequence set forth in SEQ ID NO: 55 or homologues, variants, isoforms, analogs, fragments, fragments of homologues, fragments of variants, fragments of analogs, and fragments of isoforms of SEQ ID NO: 55. Any sequence that encodes a fragment of an LLO protein or a homologue, variant, isoform, analog, fragment of a homologue, fragment of a variant, or fragment of an analog of an LLO protein can be used. A homologous LLO protein can have a sequence identity with a reference LLO protein, for example, of greater than 70%, 72%, 75%, 78%, 80%, 82%, 83%, 85%, 87%, 88%, 90%, 92%, 93%, 95%, 96%, 97%, 98%, or 99%.

Another example of an LLO protein is set forth in SEQ ID NO: 56. LLO proteins that can be used can comprise, consist essentially of, or consist of the sequence set forth in SEQ ID NO: 56 or homologues, variants, isoforms, analogs, fragments, fragments of homologues, fragments of variants, fragments of analogs, and fragments of isoforms of SEQ ID NO: 56.

Another example of an LLO protein is an LLO protein from the Listeria monocytogenes 10403S strain, as set forth in GenBank Accession No.: ZP_01942330 or EBA21833, or as encoded by the nucleic acid sequence as set forth in GenBank Accession No.: NZ_AARZ01000015 or AARZ01000015.1. Another example of an LLO protein is an LLO protein from the Listeria monocytogenes 4b F2365 strain (see, e.g., GenBank Accession No.: YP_012823), EGD-e strain (see, e.g., GenBank Accession No.: NP_463733), or any other strain of Listeria monocytogenes. Yet another example of an LLO protein is an LLO protein from Flavobacteriales bacterium HTCC2170 (see, e.g., GenBank Accession No.: ZP_01106747 or EAR01433, or encoded by GenBank Accession No.: NZ_AAOC01000003). LLO proteins that can be used can comprise, consist essentially of, or consist of any of the above LLO proteins or homologues, variants, isoforms, analogs, fragments, fragments of homologues, fragments of variants, fragments of analogs, and fragments of isoforms of the above LLO proteins.

Proteins that are homologous to LLO, or homologues, variants, isoforms, analogs, fragments, fragments of homologues, fragments of variants, fragments of analogs, and fragments of isoforms thereof, can also be used. One such example is alveolysin, which can be found, for example, in Paenibacillus alvei (see, e.g., GenBank Accession No.: P23564 or AAA22224, or encoded by GenBank Accession No.: M62709). Other such homologous proteins are known.

The LLO peptide can be a full-length LLO protein or a truncated LLO protein or LLO fragment. Likewise, the LLO peptide can be one that retains one or more functionalities of a native LLO protein or lacks one or more functionalities of a native LLO protein. For example, the retained LLO functionality can be allowing a bacteria (e.g., Listeria) to escape from a phagosome or phagolysosome, or enhancing the immunogenicity of a peptide to which it is fused. The retained functionality can also be hemolytic function or antigenic function. Alternatively, the LLO peptide can be a non-hemolytic LLO. Other functions of LLO are known, as are methods and assays for evaluating LLO functionality.

An LLO fragment can be a PEST-like sequence or can comprise a PEST-like sequence. LLO fragments can comprise one or more of an internal deletion, a truncation from the C-terminal end, and a truncation from the N-terminal end. In some cases, an LLO fragment can comprise more than one internal deletion. Other LLO peptides can be full-length LLO proteins with one or more mutations.

Some LLO proteins or fragments have reduced hemolytic activity relative to wild type LLO or are non-hemolytic fragments. For example, an LLO protein can be rendered non-hemolytic by deletion or mutation of the activation domain at the carboxy terminus, by deletion or mutation of cysteine 484, or by deletion or mutation at another location.

Other LLO proteins are rendered non-hemolytic by a deletion or mutation of the cholesterol binding domain (CBD) as detailed in U.S. Pat. No. 8,771,702, herein incorporated by reference in its entirety for all purposes. The mutations can comprise, for example, a substitution or a deletion. The entire CBD can be mutated, portions of the CBD can be mutated, or specific residues within the CBD can be mutated. For example, the LLO protein can comprise a mutation of one or more of residues C484, W491, and W492 (e.g., C484, W491, W492, C484 and W491, C484 and W492, W491 and W492, or all three residues) of SEQ ID NO: 55 or corresponding residues when optimally aligned with SEQ ID NO: 55 (e.g., a corresponding cysteine or tryptophan residue). As an example, a mutant LLO protein can be created wherein residues C484, W491, and W492 of LLO are substituted with alanine residues, which will substantially reduce hemolytic activity relative to wild type LLO. The mutant LLO protein with C484A, W491A, and W492A mutations is termed “mutLLO.”

As another example, a mutant LLO protein can be created with an internal deletion comprising the cholesterol-binding domain. The sequence of the cholesterol-binding domain of SEQ ID NO: 55 set forth in SEQ ID NO: 74. For example, the internal deletion can be a 1-11 amino acid deletion, an 11-50 amino acid deletion, or longer. Likewise, the mutated region can be 1-11 amino acids, 11-50 amino acids, or longer (e.g., 1-50, 1-11, 2-11, 3-11, 4-11, 5-11, 6-11, 7-11, 8-11, 9-11, 10-11, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 12-50, 11-15, 11-20, 11-25, 11-30, 11-35, 11-40, 11-50, 11-60, 11-70, 11-80, 11-90, 11-100, 11-150, 15-20, 15-25, 15-30, 15-35, 15-40, 15-50, 15-60, 15-70, 15-80, 15-90, 15-100, 15-150, 20-25, 20-30, 20-35, 20-40, 20-50, 20-60, 20-70, 20-80, 20-90, 20-100, 20-150, 30-35, 30-40, 30-60, 30-70, 30-80, 30-90, 30-100, or 30-150 amino acids). For example, a mutated region consisting of residues 470-500, 470-510, or 480-500 of SEQ ID NO: 55 will result in a deleted sequence comprising the CBD (residues 483-493 of SEQ ID NO: 55). However, the mutated region can also be a fragment of the CBD or can overlap with a portion of the CBD. For example, the mutated region can consist of residues 470-490, 480-488, 485-490, 486-488, 490-500, or 486-510 of SEQ ID NO: 55. For example, a fragment of the CBD (residues 484-492) can be replaced with a heterologous sequence, which will substantially reduce hemolytic activity relative to wild type LLO. For example, the CBD (ECTGLAWEWWR; SEQ ID NO: 74) can be replaced with a CTL epitope from the antigen NY-ESO-1 (ESLLMWITQCR; SEQ ID NO: 75), which contains the HLA-A2 restricted epitope 157-165 from NY-ESO-1. The resulting LLO is termed “ctLLO.”

In some mutated LLO proteins, the mutated region can be replaced by a heterologous sequence. For example, the mutated region can be replaced by an equal number of heterologous amino acids, a smaller number of heterologous amino acids, or a larger number of amino acids (e.g., 1-50, 1-11, 2-11, 3-11, 4-11, 5-11, 6-11, 7-11, 8-11, 9-11, 10-11, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 12-50, 11-15, 11-20, 11-25, 11-30, 11-35, 11-40, 11-50, 11-60, 11-70, 11-80, 11-90, 11-100, 11-150, 15-20, 15-25, 15-30, 15-35, 15-40, 15-50, 15-60, 15-70, 15-80, 15-90, 15-100, 15-150, 20-25, 20-30, 20-35, 20-40, 20-50, 20-60, 20-70, 20-80, 20-90, 20-100, 20-150, 30-35, 30-40, 30-60, 30-70, 30-80, 30-90, 30-100, or 30-150 amino acids). Other mutated LLO proteins have one or more point mutations (e.g., a point mutation of 1 residue, 2 residues, 3 residues, or more). The mutated residues can be contiguous or not contiguous.

In one example embodiment, an LLO peptide may have a deletion in the signal sequence and a mutation or substitution in the CBD.

Some LLO peptides are N-terminal LLO fragments (i.e., LLO proteins with a C-terminal deletion). Some LLO peptides are at least 494, 489, 492, 493, 500, 505, 510, 515, 520, or 525 amino acids in length or 492-528 amino acids in length. For example, the LLO fragment can consist of about the first 440 or 441 amino acids of an LLO protein (e.g., the first 441 amino acids of SEQ ID NO: 55 or 56, or a corresponding fragment of another LLO protein when optimally aligned with SEQ ID NO: 55 or 56). Other N-terminal LLO fragments can consist of the first 420 amino acids of an LLO protein (e.g., the first 420 amino acids of SEQ ID NO: 55 or 56, or a corresponding fragment of another LLO protein when optimally aligned with SEQ ID NO: 55 or 56). Other N-terminal fragments can consist of about amino acids 20-442 of an LLO protein (e.g., amino acids 20-442 of SEQ ID NO: 55 or 56, or a corresponding fragment of another LLO protein when optimally aligned with SEQ ID NO: 55 or 56). Other N-terminal LLO fragments comprise any ALLO without the activation domain comprising cysteine 484, and in particular without cysteine 484. For example, the N-terminal LLO fragment can correspond to the first 425, 400, 375, 350, 325, 300, 275, 250, 225, 200, 175, 150, 125, 100, 75, 50, or 25 amino acids of an LLO protein (e.g., the first 425, 400, 375, 350, 325, 300, 275, 250, 225, 200, 175, 150, 125, 100, 75, 50, or 25 amino acids of SEQ ID NO: 55 or 56, or a corresponding fragment of another LLO protein when optimally aligned with SEQ ID NO: 55 or 56). Preferably, the fragment comprises one or more PEST-like sequences. LLO fragments and truncated LLO proteins can contain residues of a homologous LLO protein that correspond to any one of the above specific amino acid ranges. The residue numbers need not correspond exactly with the residue numbers enumerated above (e.g., if the homologous LLO protein has an insertion or deletion relative to a specific LLO protein disclosed herein). Examples of N-terminal LLO fragments include SEQ ID NOS: 57, 58, and 59. LLO proteins that can be used comprise, consist essentially of, or consist of the sequence set forth in SEQ ID NO: 57, 58, or 59 or homologues, variants, isoforms, analogs, fragments, fragments of homologues, fragments of variants, fragments of analogs, and fragments of isoforms of SEQ ID NO: 57, 58, or 59. In some compositions and methods, the N-terminal LLO fragment set forth in SEQ ID NO: 59 is used. An example of a nucleic acid encoding the N-terminal LLO fragment set forth in SEQ ID NO: 59 is SEQ ID NO: 60.

(2) ActA

Another example of a PEST-containing peptide that can be utilized in the compositions and methods disclosed herein is an ActA peptide. ActA is a surface-associated protein and acts as a scaffold in infected host cells to facilitate the polymerization, assembly, and activation of host actin polymers in order to propel a Listeria monocytogenes through the cytoplasm. Shortly after entry into the mammalian cell cytosol, L. monocytogenes induces the polymerization of host actin filaments and uses the force generated by actin polymerization to move, first intracellularly and then from cell to cell. ActA is responsible for mediating actin nucleation and actin-based motility. The ActA protein provides multiple binding sites for host cytoskeletal components, thereby acting as a scaffold to assemble the cellular actin polymerization machinery. The N-terminus of ActA binds to monomeric actin and acts as a constitutively active nucleation promoting factor by stimulating the intrinsic actin nucleation activity. The actA and hly genes are both members of the 10-kb gene cluster regulated by the transcriptional activator PrfA, and actA is upregulated approximately 226-fold in the mammalian cytosol. Any sequence that encodes an ActA protein or a homologue, variant, isoform, analog, fragment of a homologue, fragment of a variant, or fragment of an analog of an ActA protein can be used. A homologous ActA protein can have a sequence identity with a reference ActA protein, for example, of greater than 70%, 72%, 75%, 78%, 80%, 82%, 83%, 85%, 87%, 88%, 90%, 92%, 93%, 95%, 96%, 97%, 98%, or 99%.

One example of an ActA protein comprises, consists essentially of, or consists of the sequence set forth in SEQ ID NO: 61. Another example of an ActA protein comprises, consists essentially of, or consists of the sequence set forth in SEQ ID NO: 62. The first 29 amino acid of the proprotein corresponding to either of these sequences are the signal sequence and are cleaved from ActA protein when it is secreted by the bacterium. An ActA peptide can comprise the signal sequence (e.g., amino acids 1-29 of SEQ ID NO: 61 or 62), or can comprise a peptide that does not include the signal sequence. Other examples of ActA proteins comprise, consist essentially of, or consist of homologues, variants, isoforms, analogs, fragments, fragments of homologues, fragments of isoforms, or fragments of analogs of SEQ ID NO: 61 or 62.

Another example of an ActA protein is an ActA protein from the Listeria monocytogenes 10403S strain (GenBank Accession No.: DQ054585) the NICPBP 54002 strain (GenBank Accession No.: EU394959), the S3 strain (GenBank Accession No.: EU394960), NCTC 5348 strain (GenBank Accession No.: EU394961), NICPBP 54006 strain (GenBank Accession No.: EU394962), M7 strain (GenBank Accession No.: EU394963), S19 strain (GenBank Accession No.: EU394964), or any other strain of Listeria monocytogenes. LLO proteins that can be used can comprise, consist essentially of, or consist of any of the above LLO proteins or homologues, variants, isoforms, analogs, fragments, fragments of homologues, fragments of variants, fragments of analogs, and fragments of isoforms of the above LLO proteins.

ActA peptides can be full-length ActA proteins or truncated ActA proteins or ActA fragments (e.g., N-terminal ActA fragments in which a C-terminal portion is removed). Preferably, truncated ActA proteins comprise at least one PEST sequence (e.g., more than one PEST sequence). In addition, truncated ActA proteins can optionally comprise an ActA signal peptide. Examples of PEST-like sequences contained in truncated ActA proteins include SEQ ID NOS: 45-48. Some such truncated ActA proteins comprise at least two of the PEST-like sequences set forth in SEQ ID NOS: 45-48 or homologs thereof, at least three of the PEST-like sequences set forth in SEQ ID NOS: 45-48 or homologs thereof, or all four of the PEST-like sequences set forth in SEQ ID NOS: 45-48 or homologs thereof. Examples of truncated ActA proteins include those comprising, consisting essentially of, or consisting of about residues 30-122, about residues 30-229, about residues 30-332, about residues 30-200, or about residues 30-399 of a full length ActA protein sequence (e.g., SEQ ID NO: 62). Other examples of truncated ActA proteins include those comprising, consisting essentially of, or consisting of about the first 50, 100, 150, 200, 233, 250, 300, 390, 400, or 418 residues of a full length ActA protein sequence (e.g., SEQ ID NO: 62). Other examples of truncated ActA proteins include those comprising, consisting essentially of, or consisting of about residues 200-300 or residues 300-400 of a full length ActA protein sequence (e.g., SEQ ID NO: 62). For example, the truncated ActA consists of the first 390 amino acids of the wild type ActA protein as described in U.S. Pat. No. 7,655,238, herein incorporated by reference in its entirety for all purposes. As another example, the truncated ActA can be an ActA-N100 or a modified version thereof (referred to as ActA-N100*) in which a PEST motif has been deleted and containing the nonconservative QDNKR (SEQ ID NO: 73) substitution as described in US 2014/0186387, herein incorporated by references in its entirety for all purposes. Alternatively, truncated ActA proteins can contain residues of a homologous ActA protein that corresponds to one of the above amino acid ranges or the amino acid ranges of any of the ActA peptides disclosed herein. The residue numbers need not correspond exactly with the residue numbers enumerated herein (e.g., if the homologous ActA protein has an insertion or deletion, relative to an ActA protein utilized herein, then the residue numbers can be adjusted accordingly).

Examples of truncated ActA proteins include, for example, proteins comprising, consisting essentially of, or consisting of the sequence set forth in SEQ ID NO: 63, 64, 65, or 66 or homologues, variants, isoforms, analogs, fragments of variants, fragments of isoforms, or fragments of analogs of SEQ ID NO: 63, 64, 65, or 66. SEQ ID NO: 63 referred to as ActA/PEST1 and consists of amino acids 30-122 of the full length ActA sequence set forth in SEQ ID NO: 62. SEQ ID NO: 64 is referred to as ActA/PEST2 or LA229 and consists of amino acids 30-229 of the full length ActA sequence set forth in the full-length ActA sequence set forth in SEQ ID NO: 62. SEQ ID NO: 65 is referred to as ActA/PEST3 and consists of amino acids 30-332 of the full-length ActA sequence set forth in SEQ ID NO: 62. SEQ ID NO: 66 is referred to as ActA/PEST4 and consists of amino acids 30-399 of the full-length ActA sequence set forth in SEQ ID NO: 62. As a specific example, the truncated ActA protein consisting of the sequence set forth in SEQ ID NO: 64 can be used.

Examples of truncated ActA proteins include, for example, proteins comprising, consisting essentially of, or consisting of the sequence set forth in SEQ ID NO: 67, 69, 70, or 72 or homologues, variants, isoforms, analogs, fragments of variants, fragments of isoforms, or fragments of analogs of SEQ ID NO: 67, 69, 70, or 72. As a specific example, the truncated ActA protein consisting of the sequence set forth in SEQ ID NO: 67 (encoded by the nucleic acid set forth in SEQ ID NO: 68) can be used. As another specific example, the truncated ActA protein consisting of the sequence set forth in SEQ ID NO: 70 (encoded by the nucleic acid set forth in SEQ ID NO: 71) can be used. SEQ ID NO: 71 is the first 1170 nucleotides encoding ActA in the Listeria monocytogenes 10403S strain. In some cases, the ActA fragment can be fused to a heterologous signal peptide. For example, SEQ ID NO: 72 sets forth an ActA fragment fused to an Hly signal peptide.

C. Generating Immunotherapy Constructs Encoding Recombinant Fusion Polypeptides

Also provided herein are methods for generating immunotherapy constructs encoding or compositions comprising the recombinant fusion polypeptides disclosed herein. For example, such methods can comprise selecting and designing antigenic peptides to include in the immunotherapy construct (and, for example, testing the hydropathy of the each antigenic peptide, and modifying or deselecting an antigenic peptide if it scores above a selected hydropathy index threshold value), designing one or more fusion polypeptides comprising each of the selected antigenic peptides, and generating a nucleic acid construct encoding the fusion polypeptide.

The antigenic peptides can be screened for hydrophobicity or hydrophilicity. Antigenic peptides can be selected, for example, if they are hydrophilic or if they score up to or below a certain hydropathy threshold, which can be predictive of secretability in a particular bacteria of interest (e.g., Listeria monocytogenes). For example, antigenic peptides can be scored by Kyte and Doolittle hydropathy index with a 21 amino acid window, all scoring above cutoff (around 1.6) are excluded as they are unlikely to be secretable by Listeria monocytogenes. See, e.g., Kyte-Doolittle (1982) J Mol Biol 157(1):105-132; herein incorporated by reference in its entirety for all purposes. Alternatively, an antigenic peptide scoring about a selected cutoff can be altered (e.g., changing the length of the antigenic peptide). Other sliding window sizes that can be used include, for example, 9, 11, 13, 15, 17, 19, 21, 23, 25, 27 or more amino acids. For example, the sliding window size can be 9-11 amino acids, 11-13 amino acids, 13-15 amino acids, 15-17 amino acids, 17-19 amino acids, 19-21 amino acids, 21-23 amino acids, 23-25 amino acids, or 25-27 amino acids. Other cutoffs that can be used include, for example, the following ranges 1.2-1.4, 1.4-1.6, 1.6-1.8, 1.8-2.0, 2.0-2.2 2.2-2.5, 2.5-3.0, 3.0-3.5, 3.5-4.0, or 4.0-4.5, or the cutoff can be 1.4, 1.5, 1.7, 1.8, 1.9, 2.0, 2.1, 2.2, 2.3, 2.3, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5. The cutoff can vary, for example, depending on the genus or species of the bacteria being used to deliver the fusion polypeptide.

Other suitable hydropathy plots or other appropriate scales include, for example, those reported in Rose et al. (1993) Annu Rev Biomol Struct 22:381-415; Biswas et al. (2003) Journal of Chromatography A 1000:637-655; Eisenberg (1984) Ann Rev Biochem 53:595-623; Abraham and Leo (1987) Proteins: Structure, Function and Genetics 2:130-152; Sweet and Eisenberg (1983) Mol Biol 171:479-488; Bull and Breese (1974) Arch Biochem Biophys 161:665-670; Guy (1985) Biophys J 47:61-70; Miyazawa et al. (1985) Macromolecules 18:534-552; Roseman (1988) J Mol Biol 200:513-522; Wolfenden et al. (1981) Biochemistry 20:849-855; Wilson (1981) Biochem J 199:31-41; Cowan and Whittaker (1990) Peptide Research 3:75-80; Aboderin (1971) Int J Biochem 2:537-544; Eisenberg et al. (1984) J Mol Biol 179:125-142; Hopp and Woods (1981) Proc Natl Acad Sci USA 78:3824-3828; Manavalan and Ponnuswamy (1978) Nature 275:673-674; Black and Mould (1991) Anal Biochem 193:72-82; Fauchere and Pliska (1983) Eur J Med Chem 18:369-375; Janin (1979) Nature 277:491-492; Rao and Argos (1986) Biochim Biophys Acta 869:197-214; Tanford (1962) Am Chem Soc 84:4240-4274; Welling et al. (1985) FEBS Lett 188:215-218; Parker et al. (1986) Biochemistry 25:5425-5431; and Cowan and Whittaker (1990) Peptide Research 3:75-80, each of which is herein incorporated by reference in its entirety for all purposes.

Optionally, the antigenic peptides can be scored for their ability to bind to the subject human leukocyte antigen (HLA) type (for example by using the Immune Epitope Database (IED) available at www.iedb.org, which includes netMHCpan, ANN, SMMPMBEC. SMM, CombLib_Sidney2008, PickPocket, and netMHCcons) and ranked by best MHC binding score from each antigenic peptide. Other sources include TEpredict (tepredict.sourceforge.net/help.html) or other available MHC binding measurement scales. Cutoffs may be different for different expression vectors such as Salmonella.

Optionally, the antigenic peptides can be screened for immunosuppressive epitopes (e.g., T-reg epitopes, IL-10-inducing T helper epitopes, and so forth) to deselect antigenic peptides or to avoid immunosuppressive influences.

Optionally, a predicative algorithm for immunogenicity of the epitopes can be used to screen the antigenic peptides. However, these algorithms are at best 20% accurate in predicting which peptide will generate a T cell response. Alternatively, no screening/predictive algorithms are used. Alternatively, the antigenic peptides can be screened for immunogenicity. For example, this can comprise contacting one or more T cells with an antigenic peptide, and analyzing for an immunogenic T cell response, wherein an immunogenic T cell response identifies the peptide as an immunogenic peptide. This can also comprise using an immunogenic assay to measure secretion of at least one of CD25, CD44, or CD69 or to measure secretion of a cytokine selected from the group comprising IFN-γ, TNF-α, IL-1, and IL-2 upon contacting the one or more T cells with the peptide, wherein increased secretion identifies the peptide as comprising one or more T cell epitopes.

The selected antigenic peptides can be arranged into one or more candidate orders for a potential fusion polypeptide. If there are more usable antigenic peptides than can fit into a single plasmid, different antigenic peptides can be assigned priority ranks as needed/desired and/or split up into different fusion polypeptides (e.g., for inclusion in different recombinant Listeria strains). Priority rank can be determined by factors such as relative size, priority of transcription, and/or overall hydrophobicity of the translated polypeptide. The antigenic peptides can be arranged so that they are joined directly together without linkers, or any combination of linkers between any number of pairs of antigenic peptides, as disclosed in more detail elsewhere herein. The number of linear antigenic peptides to be included can be determined based on consideration of the number of constructs needed versus the mutational burden, the efficiency of translation and secretion of multiple epitopes from a single plasmid, and the MOI needed for each bacteria or Lm comprising a plasmid.

The combination of antigenic peptides or the entire fusion polypeptide (i.e., comprising the antigenic peptides and the PEST-containing peptide and any tags) also be scored for hydrophobicity. For example, the entirety of the fused antigenic peptides or the entire fusion polypeptide can be scored for hydropathy by a Kyte and Doolittle hydropathy index with a sliding 21 amino acid window. If any region scores above a cutoff (e.g., around 1.6), the antigenic peptides can be reordered or shuffled within the fusion polypeptide until an acceptable order of antigenic peptides is found (i.e., one in which no region scores above the cutoff). Alternatively, any problematic antigenic peptides can be removed or redesigned to be of a different size. Alternatively or additionally, one or more linkers between antigenic peptides as disclosed elsewhere herein can be added or modified to change the hydrophobicity. As with hydropathy testing for the individual antigenic peptides, other window sizes can be used, or other cutoffs can be used (e.g., depending on the genus or species of the bacteria being used to deliver the fusion polypeptide). In addition, other suitable hydropathy plots or other appropriate scales could be used.

Optionally, the combination of antigenic peptides or the entire fusion polypeptide can be further screened for immunosuppressive epitopes (e.g., T-reg epitopes, IL-10-inducing T helper epitopes, and so forth) to deselect antigenic peptides or to avoid immunosuppressive influences.

A nucleic acid encoding a candidate combination of antigenic peptides or fusion polypeptide can then be designed and optimized. For example, the sequence can be optimized for increased levels of translation, duration of expression, levels of secretion, levels of transcription, and any combination thereof. For example, the increase can be 2-fold to 1000-fold, 2-fold to 500-fold, 2-fold to 100-fold, 2-fold to 50-fold, 2-fold to 20-fold, 2-fold to 10-fold, or 3-fold to 5-fold relative to a control, non-optimized sequence.

For example, the fusion polypeptide or nucleic acid encoding the fusion polypeptide can be optimized for decreased levels of secondary structures possibly formed in the oligonucleotide sequence, or alternatively optimized to prevent attachment of any enzyme that may modify the sequence. Expression in bacterial cells can be hampered, for example, by transcriptional silencing, low mRNA half-life, secondary structure formation, attachment sites of oligonucleotide binding molecules such as repressors and inhibitors, and availability of rare tRNAs pools. The source of many problems in bacterial expressions is found within the original sequence. The optimization of RNAs may include modification of cis acting elements, adaptation of its GC-content, modifying codon bias with respect to non-limiting tRNAs pools of the bacterial cell, and avoiding internal homologous regions. Thus, optimizing a sequence can entail, for example, adjusting regions of very high (>80%) or very low (<30%) GC content. Optimizing a sequence can also entail, for example, avoiding one or more of the following cis-acting sequence motifs: internal TATA-boxes, chi-sites, and ribosomal entry sites; AT-rich or GC-rich sequence stretches; repeat sequences and RNA secondary structures; (cryptic) splice donor and acceptor sites; branch points; or a combination thereof. Optimizing expression can also entail adding sequence elements to flanking regions of a gene and/or elsewhere in the plasmid.

Optimizing a sequence can also entail, for example, adapting the codon usage to the codon bias of host genes (e.g., Listeria monocytogenes genes). For example, the codons below can be used for Listeria monocytogenes.

| | | | | |
|---|---|---|---|---|
| A = GCA | G = GGT | L = TTA | Q = CAA | V = GTT |
| C = TGT | H = CAT | M = ATG | R = CGT | W = TGG |
| D = GAT | I = ATT | N = AAC | S = TCT | Y = TAT |
| E = GAA | K = AAA | P = CCA | T = ACA | STOP = TAA |
| F = TTC | | | | |

A nucleic acid encoding a fusion polypeptide can be generated and introduced into a delivery vehicle such as a bacteria strain or Listeria strain. Other delivery vehicles may be suitable for DNA immunotherapy or peptide immunotherapy, such as a vaccinia virus or virus-like particle. Once a plasmid encoding a fusion polypeptide is generated and introduced into a bacteria strain or Listeria strain, the bacteria or Listeria strain can be cultured and characterized to confirm expression and secretion of the fusion polypeptide comprising the antigenic peptides.

### IV. Recombinant Bacteria or Listeria Strains

Also provided herein are recombinant bacterial strains, such as a Listeria strain, comprising a heteroclitic peptide or recombinant fusion polypeptide disclosed herein or a nucleic acid encoding the heteroclitic peptide or recombinant fusion polypeptide as disclosed elsewhere herein. Preferably, the bacterial strain is a Listeria strain, such as a Listeria monocytogenes (Lm) strain. However, other bacteria strains can also be used, such as a Salmonella, Yersinia, Shigella, or Mycobacterium strain. Lm has a number of inherent advantages as a vaccine vector. The bacterium grows very efficiently in vitro without special requirements, and it lacks LPS, which is a major toxicity factor in gram-negative bacteria, such as Salmonella. Genetically attenuated Lm vectors also offer additional safety as they can be readily eliminated with antibiotics, in case of serious adverse effects, and unlike some viral vectors, no integration of genetic material into the host genome occurs.

The recombinant Listeria strain can be any Listeria strain. Examples of suitable Listeria strains include Listeria seeligeri, Listeria grayi, Listeria ivanovii, Listeria murrayi, Listeria welshimeri, Listeria monocytogenes (Lm), or any other Listeria species known in the art. Preferably, the recombinant listeria strain is a strain of the species Listeria monocytogenes. Examples of Listeria monocytogenes strains include the following: L. monocytogenes 10403S wild type (see, e.g., Bishop and Hinrichs (1987) J Immunol 139:2005-2009; Lauer et al. (2002) J Bact 184:4177-4186); L. monocytogenes DP-L4056, which is phage cured (see, e.g., Lauer et al. (2002) J Bact 184:4177-4186); L. monocytogenes DP-L4027, which is phage cured and has an hly gene deletion (see, e.g., Lauer et al. (2002) J Bact 184:4177-4186; Jones and Portnoy (1994) Infect Immunity 65:5608-5613); L. monocytogenes DP-L4029, which is phage cured and has an actA gene deletion (see, e.g., Lauer et al. (2002) J Bact 184:4177-4186; Skoble et al. (2000) J Cell Biol 150:527-538); L. monocytogenes DP-L4042 (delta PEST) (see, e.g., Brockstedt et al. (2004) Proc Natl Acad Sci. USA 101:13832-13837 and supporting information); L. monocytogenes DP-L4097 (LLO-S44A) (see, e.g., Brockstedt et al. (2004) Proc Natl Acad Sci USA 101:13832-13837 and supporting information); L. monocytogenes DP-L4364 (delta lplA; lipoate protein ligase) (see, e.g., Brockstedt et al. (2004) Proc Natl Acad Sci USA 101:13832-13837 and supporting information); L. monocytogenes DP-L4405 (delta inlA) (see, e.g., Brockstedt et al. (2004) Proc Natl Acad Sci USA 101:13832-13837 and supporting information); L. monocytogenes DP-L4406 (delta inlB) (see, e.g., Brockstedt et al. (2004) Proc Natl Acad Sci USA 101:13832-13837 and supporting information); L. monocytogenes CS-L0001 (delta actA; delta inlB) (see, e.g., Brockstedt et al. (2004) Proc Natl Acad Sci USA 101:13832-13837 and supporting information); L. monocytogenes CS-L0002 (delta actA; delta lplA) (see, e.g., Brockstedt et al. (2004) Proc Natl Acad Sci USA 101:13832-13837 and supporting information); L. monocytogenes CS-L0003 (LLO L461T; delta lplA) (see, e.g., Brockstedt et al. (2004) Proc Natl Acad Sci USA 101:13832-13837 and supporting information); L. monocytogenes DP-L4038 (delta actA; LLO L461T) (see, e.g., Brockstedt et al. (2004) Proc Natl Acad Sci USA 101:13832-13837 and supporting information); L. monocytogenes DP-L4384 (LLO S44A; LLO L461T) (see, e.g., Brockstedt et al. (2004) Proc Natl Acad Sci USA 101:13832-13837 and supporting information); a L. monocytogenes strain with an lplA1 deletion (encoding lipoate protein ligase LplA1) (see, e.g., O'Riordan et al. (2003) Science 302:462-464); L. monocytogenes DP-L4017 (10403S with LLO L461T) (see, e.g., U.S. Pat. No. 7,691,393); L. monocytogenes EGD (see, e.g., GenBank Accession No. AL591824). In another embodiment, the Listeria strain is L. monocytogenes EGD-e (see GenBank Accession No. NC_003210; ATCC Accession No. BAA-679); L. monocytogenes DP-L4029 (actA deletion, optionally in combination with uvrAB deletion (DP-L4029uvrAB) (see, e.g., U.S. Pat. No. 7,691,393); L. monocytogenes actA-linlB—double mutant (see, e.g., ATCC Accession No. PTA-5562); L. monocytogenes lplA mutant or hly mutant (see, e.g., US 2004/0013690); L. monocytogenes dalldat double mutant (see, e.g., US 2005/0048081). Other L. monocytogenes strains includes those that are modified (e.g., by a plasmid and/or by genomic integration) to contain a nucleic acid encoding one of, or any combination of, the following genes: hly (LLO; listeriolysin); iap (p60); inlA; inlB; inlC; dal (alanine racemase); dat (D-amino acid aminotransferase); plcA; plcB; actA; or any nucleic acid that mediates growth, spread, breakdown of a single walled vesicle, breakdown of a double walled vesicle, binding to a host cell, or uptake by a host cell. Each of the above references is herein incorporated by reference in its entirety for all purposes.

The recombinant bacteria or Listeria can have wild-type virulence, can have attenuated virulence, or can be avirulent. For example, a recombinant Listeria of can be sufficiently virulent to escape the phagosome or phagolysosome and enter the cytosol. Such Listeria strains can also be live-attenuated Listeria strains, which comprise at least one attenuating mutation, deletion, or inactivation as disclosed elsewhere herein. Preferably, the recombinant Listeria is an attenuated auxotrophic strain. An auxotrophic strain is one that is unable to synthesize a particular organic compound required for its growth. Examples of such strains are described in U.S. Pat. No. 8,114,414, herein incorporated by reference in its entirety for all purposes.

Preferably, the recombinant Listeria strain lacks antibiotic resistance genes. For example, such recombinant Listeria strains can comprise a plasmid that does not encode an antibiotic resistance gene. However, some recombinant Listeria strains provided herein comprise a plasmid comprising a nucleic acid encoding an antibiotic resistance gene. Antibiotic resistance genes may be used in the conventional selection and cloning processes commonly employed in molecular biology and vaccine preparation. Exemplary antibiotic resistance genes include gene products that confer resistance to ampicillin, penicillin, methicillin, streptomycin, erythromycin, kanamycin, tetracycline, chloramphenicol (CAT), neomycin, hygromycin, and gentamicin.

A. Bacteria or Listeria Strains Comprising Heteroclitic Peptides or Recombinant Fusion Polypeptides or Nucleic Acids Encoding Heteroclitic Peptides or Recombinant Fusion Polypeptides

The recombinant bacterial strains (e.g., Listeria strains) disclosed herein can comprise a heteroclitic peptide or recombinant fusion polypeptide disclosed herein or a nucleic acid encoding the heteroclitic peptide or recombinant fusion polypeptide as disclosed elsewhere herein.

In bacteria or Listeria strains comprising a nucleic acid encoding a heteroclitic peptide or recombinant fusion protein, the nucleic acid can be codon optimized. Examples of optimal codons utilized by L. monocytogenes for each amino acid are shown US 2007/0207170, herein incorporated by reference in its entirety for all purposes. A nucleic acid is codon-optimized if at least one codon in the nucleic acid is replaced with a codon that is more frequently used by L. monocytogenes for that amino acid than the codon in the original sequence.

The nucleic acid can be present in an episomal plasmid within the bacteria or Listeria strain and/or the nucleic acid can be genomically integrated in the bacteria or Listeria strain. Some recombinant bacteria or Listeria strains comprise two separate nucleic acids encoding two heteroclitic peptides or recombinant fusion polypeptides as disclosed herein: one nucleic acid in an episomal plasmid, and one genomically integrated in the bacteria or Listeria strain.

The episomal plasmid can be one that is stably maintained in vitro (in cell culture), in vivo (in a host), or both in vitro and in vivo. If in an episomal plasmid, the open reading frame encoding the heteroclitic peptide or recombinant fusion polypeptide can be operably linked to a promoter/regulatory sequence in the plasmid. If genomically integrated in the bacteria or Listeria strain, the open reading frame encoding the heteroclitic peptide or recombinant fusion polypeptide can be operably linked to an exogenous promoter/regulatory sequence or to an endogenous promoter/regulatory sequence. Examples of promoters/regulatory sequences useful for driving constitutive expression of a gene are well known and include, for example, an hly, hlyA, actA, prfA, and p60 promoters of Listeria, the Streptococcus bac promoter, the Streptomyces griseus sgiA promoter, and the B. thuringiensis phaZ promoter. In some cases, an inserted gene of interest is not interrupted or subjected to regulatory constraints which often occur from integration into genomic DNA, and in some cases, the presence of the inserted heterologous gene does not lead to rearrangement or interruption of the cell's own important regions.

Such recombinant bacteria or Listeria strains can be made by transforming a bacteria or Listeria strain or an attenuated bacteria or Listeria strain described elsewhere herein with a plasmid or vector comprising a nucleic acid encoding the heteroclitic peptide or recombinant fusion polypeptide. The plasmid can be an episomal plasmid that does not integrate into a host chromosome. Alternatively, the plasmid can be an integrative plasmid that integrates into a chromosome of the bacteria or Listeria strain. The plasmids used herein can also be multicopy plasmids. Methods for transforming bacteria are well known, and include calcium-chloride competent cell-based methods, electroporation methods, bacteriophage-mediated transduction, chemical transformation techniques, and physical transformation techniques. See, e.g., de Boer et al. (1989) Cell 56:641-649; Miller et al. (1995) FASEB J. 9:190-199; Sambrook et al. (1989) Molecular Cloning: A Laboratory Manual, Cold Spring Harbor Laboratory, New York; Ausubel et al. (1997) Current Protocols in Molecular Biology, John Wiley & Sons, New York; Gerhardt et al., eds., 1994, Methods for General and Molecular Bacteriology, American Society for Microbiology, Washington, D.C.; and Miller, 1992, A Short Course in Bacterial Genetics, Cold Spring Harbor Laboratory Press, Cold Spring Harbor, N.Y., each of which is herein incorporated by reference in its entirety for all purposes.

Bacteria or Listeria strains with genomically integrated heterologous nucleic acids can be made, for example, by using a site-specific integration vector, whereby the bacteria or Listeria comprising the integrated gene is created using homologous recombination. The integration vector can be any site-specific integration vector that is capable of infecting a bacteria or Listeria strain. Such an integration vector can comprise, for example, a PSA attPP′ site, a gene encoding a PSA integrase, a U153 attPP′ site, a gene encoding a U153 integrase, an A118 attPP′ site, a gene encoding an A118 integrase, or any other known attPP′ site or any other phage integrase.

Such bacteria or Listeria strains comprising an integrated gene can also be created using any other known method for integrating a heterologous nucleic acid into a bacteria or Listeria chromosome. Techniques for homologous recombination are well known, and are described, for example, in Baloglu et al. (2005) Vet Microbiol 109(1-2):11-17); Jiang et al. 2005) Acta Biochim Biophys Sin (Shanghai) 37(1):19-24), and U.S. Pat. No. 6,855,320, each of which is herein incorporated by reference in its entirety for all purposes.

Integration into a bacteria or Listerial chromosome can also be achieved using transposon insertion. Techniques for transposon insertion are well known, and are described, for example, for the construction of DP-L967 by Sun et al. (1990) Infection and Immunity 58: 3770-3778, herein incorporated by reference in its entirety for all purposes. Transposon mutagenesis can achieve stable genomic insertion, but the position in the genome where the heterologous nucleic acids has been inserted is unknown.

Integration into a bacterial or Listerial chromosome can also be achieved using phage integration sites (see, e.g., Lauer et al. (2002) J Bacteriol 184(15):4177-4186, herein incorporated by reference in its entirety for all purposes). For example, an integrase gene and attachment site of a bacteriophage (e.g., U153 or PSA listeriophage) can be used to insert a heterologous gene into the corresponding attachment site, which may be any appropriate site in the genome (e.g. comK or the 3′ end of the arg tRNA gene). Endogenous prophages can be cured from the utilized attachment site prior to integration of the heterologous nucleic acid. Such methods can result, for example, in single-copy integrants. In order to avoid a “phage curing step,” a phage integration system based on PSA phage can be used (see, e.g., Lauer et al. (2002) J Bacteriol 184:4177-4186, herein incorporated by reference in its entirety for all purposes). Maintaining the integrated gene can require, for example, continuous selection by antibiotics. Alternatively, a phage-based chromosomal integration system can be established that does not require selection with antibiotics. Instead, an auxotrophic host strain can be complemented. For example, a phage-based chromosomal integration system for clinical applications can be used, where a host strain that is auxotrophic for essential enzymes, including, for example, D-alanine racemase is used (e.g., Lm dal(−)dat(−)).

Conjugation can also be used to introduce genetic material and/or plasmids into bacteria. Methods for conjugation are well known, and are described, for example, in Nikodinovic et al. (2006) Plasmid 56(3):223-227 and Auchtung et al. (2005) Proc Natl Acad Sci USA 102(35):12554-12559, each of which is herein incorporated by reference in its entirety for all purposes.

In a specific example, a recombinant bacteria or Listeria strain can comprise a nucleic acid encoding a heteroclitic peptide or recombinant fusion polypeptide genomically integrated into the bacteria or Listeria genome as an open reading frame with an endogenous actA sequence (encoding an ActA protein) or an endogenous hly sequence (encoding an LLO protein). For example, the expression and secretion of the heteroclitic peptide or fusion polypeptide can be under the control of the endogenous actA promoter and ActA signal sequence or can be under the control of the endogenous hly promoter and LLO signal sequence. As another example, the nucleic acid encoding a heteroclitic peptide or recombinant fusion polypeptide can replace an actA sequence encoding an ActA protein or an hly sequence encoding an LLO protein.

Selection of recombinant bacteria or Listeria strains can be achieved by any means. For example, antibiotic selection can be used. Antibiotic resistance genes may be used in the conventional selection and cloning processes commonly employed in molecular biology and vaccine preparation. Exemplary antibiotic resistance genes include gene products that confer resistance to ampicillin, penicillin, methicillin, streptomycin, erythromycin, kanamycin, tetracycline, chloramphenicol (CAT), neomycin, hygromycin, and gentamicin. Alternatively, auxotrophic strains can be used, and an exogenous metabolic gene can be used for selection instead of or in addition to an antibiotic resistance gene. As an example, in order to select for auxotrophic bacteria comprising a plasmid encoding a metabolic enzyme or a complementing gene provided herein, transformed auxotrophic bacteria can be grown in a medium that will select for expression of the gene encoding the metabolic enzyme (e.g., amino acid metabolism gene) or the complementing gene. Alternatively, a temperature-sensitive plasmid can be used to select recombinants or any other known means for selecting recombinants.

B. Attenuation of Bacteria or Listeria Strains

The recombinant bacteria strains (e.g., recombinant Listeria strains) disclosed herein can be attenuated. The term “attenuation” encompasses a diminution in the ability of the bacterium to cause disease in a host animal. For example, the pathogenic characteristics of an attenuated Listeria strain may be lessened compared with wild-type Listeria, although the attenuated Listeria is capable of growth and maintenance in culture. Using as an example the intravenous inoculation of BALB/c mice with an attenuated Listeria, the lethal dose at which 50% of inoculated animals survive (LD₅₀) is preferably increased above the LD₅₀ of wild-type Listeria by at least about 10-fold, more preferably by at least about 100-fold, more preferably at least about 1,000 fold, even more preferably at least about 10,000 fold, and most preferably at least about 100,000-fold. An attenuated strain of Listeria is thus one that does not kill an animal to which it is administered, or is one that kills the animal only when the number of bacteria administered is vastly greater than the number of wild-type non-attenuated bacteria which would be required to kill the same animal. An attenuated bacterium should also be construed to mean one which is incapable of replication in the general environment because the nutrient required for its growth is not present therein. Thus, the bacterium is limited to replication in a controlled environment wherein the required nutrient is provided. Attenuated strains are environmentally safe in that they are incapable of uncontrolled replication

(1) Methods of Attenuating Bacteria and Listeria Strains

Attenuation can be accomplished by any known means. For example, such attenuated strains can be deficient in one or more endogenous virulence genes or one or more endogenous metabolic genes. Examples of such genes are disclosed herein, and attenuation can be achieved by inactivation of any one of or any combination of the genes disclosed herein. Inactivation can be achieved, for example, through deletion or through mutation (e.g., an inactivating mutation). The term “mutation” includes any type of mutation or modification to the sequence (nucleic acid or amino acid sequence) and may encompass a deletion, a truncation, an insertion, a substitution, a disruption, or a translocation. For example, a mutation can include a frameshift mutation, a mutation which causes premature termination of a protein, or a mutation of regulatory sequences which affect gene expression. Mutagenesis can be accomplished using recombinant DNA techniques or using traditional mutagenesis technology using mutagenic chemicals or radiation and subsequent selection of mutants. Deletion mutants may be preferred because of the accompanying low probability of reversion. The term “metabolic gene” refers to a gene encoding an enzyme involved in or required for synthesis of a nutrient utilized or required by a host bacteria. For example, the enzyme can be involved in or required for the synthesis of a nutrient required for sustained growth of the host bacteria. The term “virulence” gene includes a gene whose presence or activity in an organism's genome that contributes to the pathogenicity of the organism (e.g., enabling the organism to achieve colonization of a niche in the host (including attachment to cells), immunoevasion (evasion of host's immune response), immunosuppression (inhibition of host's immune response), entry into and exit out of cells, or obtaining nutrition from the host).

A specific example of such an attenuated strain is Listeria monocytogenes (Lm) dal(−) dat(−) (Lmdd). Another example of such an attenuated strain is Lm dal(−)dat(−)AactA (LmddA). See, e.g., US 2011/0142791, herein incorporated by references in its entirety for all purposes. LmddA is based on a Listeria strain which is attenuated due to the deletion of the endogenous virulence gene actA. Such strains can retain a plasmid for antigen expression in vivo and in vitro by complementation of the dal gene. Alternatively, the LmddA can be a dal/dat/actA Listeria having mutations in the endogenous dal, dat, and actA genes. Such mutations can be, for example, a deletion or other inactivating mutation.

Another specific example of an attenuated strain is Lm prfA(−) or a strain having a partial deletion or inactivating mutation in the prfA gene. The PrfA protein controls the expression of a regulon comprising essential virulence genes required by Lm to colonize its vertebrate hosts; hence the prfA mutation strongly impairs PrfA ability to activate expression of PrfA-dependent virulence genes.

Yet another specific example of an attenuated strain is Lm inlB(−)actA(−) in which two genes critical to the bacterium's natural virulence—internalin B and act A—are deleted.

Other examples of attenuated bacteria or Listeria strains include bacteria or Listeria strains deficient in one or more endogenous virulence genes. Examples of such genes include actA, prfA, plcB, plcA, plcA, inlB, inlC, inlJ, and bsh in Listeria. Attenuated Listeria strains can also be the double mutant or triple mutant of any of the above-mentioned strains. Attenuated Listeria strains can comprise a mutation or deletion of each one of the genes, or comprise a mutation or deletion of, for example, up to ten of any of the genes provided herein (e.g., including the actA, prfA, and dal/dat genes). For example, an attenuated Listeria strain can comprise a mutation or deletion of an endogenous internalin C (inlC) gene and/or a mutation or deletion of an endogenous actA gene. Alternatively, an attenuated Listeria strain can comprise a mutation or deletion of an endogenous internalin B (inlB) gene and/or a mutation or deletion of an endogenous actA gene. Alternatively, an attenuated Listeria strain can comprise a mutation or deletion of endogenous inlB, inlC, and actA genes. Translocation of Listeria to adjacent cells is inhibited by the deletion of the endogenous actA gene and/or the endogenous inlC gene or endogenous inlB gene, which are involved in the process, thereby resulting in high levels of attenuation with increased immunogenicity and utility as a strain backbone. An attenuated Listeria strain can also be a double mutant comprising mutations or deletions of both plcA and plcB. In some cases, the strain can be constructed from the EGD Listeria backbone.

A bacteria or Listeria strain can also be an auxotrophic strain having a mutation in a metabolic gene. As one example, the strain can be deficient in one or more endogenous amino acid metabolism genes. For example, the generation of auxotrophic strains of Listeria deficient in D-alanine, for example, may be accomplished in a number of ways that are well known, including deletion mutations, insertion mutations, frameshift mutations, mutations which cause premature termination of a protein, or mutation of regulatory sequences which affect gene expression. Deletion mutants may be preferred because of the accompanying low probability of reversion of the auxotrophic phenotype. As an example, mutants of D-alanine which are generated according to the protocols presented herein may be tested for the ability to grow in the absence of D-alanine in a simple laboratory culture assay. Those mutants which are unable to grow in the absence of this compound can be selected.

Examples of endogenous amino acid metabolism genes include a vitamin synthesis gene, a gene encoding pantothenic acid synthase, a D-glutamic acid synthase gene, a D-alanine amino transferase (dat) gene, a D-alanine racemase (dal) gene, dga, a gene involved in the synthesis of diaminopimelic acid (DAP), a gene involved in the synthesis of Cysteine synthase A (cysK), a vitamin-B12 independent methionine synthase, trpA, trpB, trpE, asnB, gltD, gltB, leuA, argG, and thrC. The Listeria strain can be deficient in two or more such genes (e.g., dat and dal). D-glutamic acid synthesis is controlled in part by the dal gene, which is involved in the conversion of D-glu+pyr to alpha-ketoglutarate+D-ala, and the reverse reaction.

As another example, an attenuated Listeria strain can be deficient in an endogenous synthase gene, such as an amino acid synthesis gene. Examples of such genes include folP, a gene encoding a dihydrouridine synthase family protein, ispD, ispF, a gene encoding a phosphoenolpyruvate synthase, hisF, hisH, filI, a gene encoding a ribosomal large subunit pseudouridine synthase, ispD, a gene encoding a bifunctional GMP synthase/glutamine amidotransferase protein, cobS, cobB, cbiD, a gene encoding a uroporphyrin-III C-methyltransferase/uroporphyrinogen-III synthase, cobQ, uppS, truB, dxs, mvaS, dapA, ispG, folC, a gene encoding a citrate synthase, argJ, a gene encoding a 3-deoxy-7-phosphoheptulonate synthase, a gene encoding an indole-3-glycerol-phosphate synthase, a gene encoding an anthranilate synthase/glutamine amidotransferase component, menB, a gene encoding a menaquinone-specific isochorismate synthase, a gene encoding a phosphoribosylformylglycinamidine synthase I or II, a gene encoding a phosphoribosylaminoimidazole-succinocarboxamide synthase, carB, carA, thyA, mgsA, aroB, hepB, rluB, ilvB, ilvN, alsS, fabF, fabH, a gene encoding a pseudouridine synthase, pyrG, truA, pabB, and an atp synthase gene (e.g., atpC, atpD-2, aptG, atpA-2, and so forth).

Attenuated Listeria strains can be deficient in endogenous phoP, aroA, aroC, aroD, or plcB. As yet another example, an attenuated Listeria strain can be deficient in an endogenous peptide transporter. Examples include genes encoding an ABC transporter/ATP-binding/permease protein, an oligopeptide ABC transporter/oligopeptide-binding protein, an oligopeptide ABC transporter/permease protein, a zinc ABC transporter/zinc-binding protein, a sugar ABC transporter, a phosphate transporter, a ZIP zinc transporter, a drug resistance transporter of the EmrB/QacA family, a sulfate transporter, a proton-dependent oligopeptide transporter, a magnesium transporter, a formate/nitrite transporter, a spermidine/putrescine ABC transporter, a Na/Pi-cotransporter, a sugar phosphate transporter, a glutamine ABC transporter, a major facilitator family transporter, a glycine betaine/L-proline ABC transporter, a molybdenum ABC transporter, a techoic acid ABC transporter, a cobalt ABC transporter, an ammonium transporter, an amino acid ABC transporter, a cell division ABC transporter, a manganese ABC transporter, an iron compound ABC transporter, a maltose/maltodextrin ABC transporter, a drug resistance transporter of the Bcr/CflA family, and a subunit of one of the above proteins.

Other attenuated bacteria and Listeria strains can be deficient in an endogenous metabolic enzyme that metabolizes an amino acid that is used for a bacterial growth process, a replication process, cell wall synthesis, protein synthesis, metabolism of a fatty acid, or for any other growth or replication process. Likewise, an attenuated strain can be deficient in an endogenous metabolic enzyme that can catalyze the formation of an amino acid used in cell wall synthesis, can catalyze the synthesis of an amino acid used in cell wall synthesis, or can be involved in synthesis of an amino acid used in cell wall synthesis. Alternatively, the amino acid can be used in cell wall biogenesis. Alternatively, the metabolic enzyme is a synthetic enzyme for D-glutamic acid, a cell wall component.

Other attenuated Listeria strains can be deficient in metabolic enzymes encoded by a D-glutamic acid synthesis gene, dga, an alr (alanine racemase) gene, or any other enzymes that are involved in alanine synthesis. Yet other examples of metabolic enzymes for which the Listeria strain can be deficient include enzymes encoded by serC (a phosphoserine aminotransferase), asd (aspartate betasemialdehyde dehydrogenase; involved in synthesis of the cell wall constituent diaminopimelic acid), the gene encoding gsaB-glutamate-1-semialdehyde aminotransferase (catalyzes the formation of 5-aminolevulinate from (S)-4-amino-5-oxopentanoate), hemL (catalyzes the formation of 5-aminolevulinate from (S)-4-amino-5-oxopentanoate), aspB (an aspartate aminotransferase that catalyzes the formation of oxalozcetate and L-glutamate from L-aspartate and 2-oxoglutarate), argF-1 (involved in arginine biosynthesis), aroE (involved in amino acid biosynthesis), aroB (involved in 3-dehydroquinate biosynthesis), aroD (involved in amino acid biosynthesis), aroC (involved in amino acid biosynthesis), hisB (involved in histidine biosynthesis), hisD (involved in histidine biosynthesis), hisG (involved in histidine biosynthesis), metX (involved in methionine biosynthesis), proB (involved in proline biosynthesis), argR (involved in arginine biosynthesis), argJ (involved in arginine biosynthesis), thil (involved in thiamine biosynthesis), LMOf2365_1652 (involved in tryptophan biosynthesis), aroA (involved in tryptophan biosynthesis), ilvD (involved in valine and isoleucine biosynthesis), ilvC (involved in valine and isoleucine biosynthesis), leuA (involved in leucine biosynthesis), dapF (involved in lysine biosynthesis), and thrB (involved in threonine biosynthesis) (all GenBank Accession No. NC_002973).

An attenuated Listeria strain can be generated by mutation of other metabolic enzymes, such as a tRNA synthetase. For example, the metabolic enzyme can be encoded by the trpS gene, encoding tryptophanyltRNA synthetase. For example, the host strain bacteria can be Δ(trpS aroA), and both markers can be contained in an integration vector.

Other examples of metabolic enzymes that can be mutated to generate an attenuated Listeria strain include an enzyme encoded by murE (involved in synthesis of diaminopimelic acid; GenBank Accession No: NC_003485), LMOf2365_2494 (involved in teichoic acid biosynthesis), WecE (Lipopolysaccharide biosynthesis protein rffA; GenBank Accession No: AE014075.1), or amiA (an N-acetylmuramoyl-L-alanine amidase). Yet other examples of metabolic enzymes include aspartate aminotransferase, histidinol-phosphate aminotransferase (GenBank Accession No. NP_466347), or the cell wall teichoic acid glycosylation protein GtcA.

Other examples of metabolic enzymes that can be mutated to generate an attenuated Listeria strain include a synthetic enzyme for a peptidoglycan component or precursor. The component can be, for example, UDP-N-acetylmuramylpentapeptide, UDP-N-acetylglucosamine, MurNAc-(pentapeptide)-pyrophosphoryl-undecaprenol, GlcNAc-p-(1,4)-MurNAc-(pentapeptide)-pyrophosphorylundecaprenol, or any other peptidoglycan component or precursor.

Yet other examples of metabolic enzymes that can be mutated to generate an attenuated Listeria strain include metabolic enzymes encoded by murG, murD, murA-1, or murA-2 (all set forth in GenBank Accession No. NC_002973). Alternatively, the metabolic enzyme can be any other synthetic enzyme for a peptidoglycan component or precursor. The metabolic enzyme can also be a trans-glycosylase, a trans-peptidase, a carboxy-peptidase, any other class of metabolic enzyme, or any other metabolic enzyme. For example, the metabolic enzyme can be any other Listeria metabolic enzyme or any other Listeria monocytogenes metabolic enzyme.

Other bacterial strains can be attenuated as described above for Listeria by mutating the corresponding orthologous genes in the other bacterial strains.

(2) Methods of Complementing Attenuated Bacteria and Listeria Strains

The attenuated bacteria or Listeria strains disclosed herein can further comprise a nucleic acid comprising a complementing gene or encoding a metabolic enzyme that complements an attenuating mutation (e.g., complements the auxotrophy of the auxotrophic Listeria strain). For example, a nucleic acid having a first open reading frame encoding a fusion polypeptide as disclosed herein can further comprise a second open reading frame comprising the complementing gene or encoding the complementing metabolic enzyme. Alternatively, a first nucleic acid can encode the fusion polypeptide and a separate second nucleic acid can comprise the complementing gene or encode the complementing metabolic enzyme.

The complementing gene can be extrachromosomal or can be integrated into the bacteria or Listeria genome. For example, the auxotrophic Listeria strain can comprise an episomal plasmid comprising a nucleic acid encoding a metabolic enzyme. Such plasmids will be contained in the Listeria in an episomal or extrachromosomal fashion. Alternatively, the auxotrophic Listeria strain can comprise an integrative plasmid (i.e., integration vector) comprising a nucleic acid encoding a metabolic enzyme. Such integrative plasmids can be used for integration into a Listeria chromosome. Preferably, the episomal plasmid or the integrative plasmid lacks an antibiotic resistance marker.

The metabolic gene can be used for selection instead of or in addition to an antibiotic resistance gene. As an example, in order to select for auxotrophic bacteria comprising a plasmid encoding a metabolic enzyme or a complementing gene provided herein, transformed auxotrophic bacteria can be grown in a medium that will select for expression of the gene encoding the metabolic enzyme (e.g., amino acid metabolism gene) or the complementing gene. For example, a bacteria auxotrophic for D-glutamic acid synthesis can be transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. Similarly, a bacterium auxotrophic for D-alanine synthesis will grow in the absence of D-alanine when transformed and expressing a plasmid comprising a nucleic acid encoding an amino acid metabolism enzyme for D-alanine synthesis. Such methods for making appropriate media comprising or lacking necessary growth factors, supplements, amino acids, vitamins, antibiotics, and the like are well-known and are available commercially.

Once the auxotrophic bacteria comprising the plasmid encoding a metabolic enzyme or a complementing gene provided herein have been selected in appropriate medium, the bacteria can be propagated in the presence of a selective pressure. Such propagation can comprise growing the bacteria in media without the auxotrophic factor. The presence of the plasmid expressing the metabolic enzyme or the complementing gene in the auxotrophic bacteria ensures that the plasmid will replicate along with the bacteria, thus continually selecting for bacteria harboring the plasmid. Production of the bacteria or Listeria strain can be readily scaled up by adjusting the volume of the medium in which the auxotrophic bacteria comprising the plasmid are growing.

In one specific example, the attenuated strain is a strain having a deletion of or an inactivating mutation in dal and dat (e.g., Listeria monocytogenes (Lm) dal(−)dat(−) (Lmdd) or Lm dal(−)dat(−)AactA (LmddA)), and the complementing gene encodes an alanine racemase enzyme (e.g., encoded by dal gene) or a D-amino acid aminotransferase enzyme (e.g., encoded by dat gene). An exemplary alanine racemase protein can have the sequence set forth in SEQ ID NO: 76 (encoded by SEQ ID NO: 78; GenBank Accession No: AF038438) or can be a homologue, variant, isoform, analog, fragment, fragment of a homologue, fragment of a variant, fragment of an analog, or fragment of an isoform of SEQ ID NO: 76. The alanine racemase protein can also be any other Listeria alanine racemase protein. Alternatively, the alanine racemase protein can be any other gram-positive alanine racemase protein or any other alanine racemase protein. An exemplary D-amino acid aminotransferase protein can have the sequence set forth in SEQ ID NO: 77 (encoded by SEQ ID NO: 79; GenBank Accession No: AF038439) or can be a homologue, variant, isoform, analog, fragment, fragment of a homologue, fragment of a variant, fragment of an analog, or fragment of an isoform of SEQ ID NO: 77. The D-amino acid aminotransferase protein can also be any other Listeria D-amino acid aminotransferase protein. Alternatively, the D-amino acid aminotransferase protein can be any other gram-positive D-amino acid aminotransferase protein or any other D-amino acid aminotransferase protein.

In another specific example, the attenuated strain is a strain having a deletion of or an inactivating mutation in prfA (e.g., Lm prfA(−)), and the complementing gene encodes a PrfA protein. For example, the complementing gene can encode a mutant PrfA (D133V) protein that restores partial PrfA function. An example of a wild type PrfA protein is set forth in SEQ ID NO: 80 (encoded by nucleic acid set forth in SEQ ID NO: 81), and an example of a D133V mutant PrfA protein is set forth in SEQ ID NO: 82 (encoded by nucleic acid set forth in SEQ ID NO: 83). The complementing PrfA protein can be a homologue, variant, isoform, analog, fragment, fragment of a homologue, fragment of a variant, fragment of an analog, or fragment of an isoform of SEQ ID NO: 80 or 82. The PrfA protein can also be any other Listeria PrfA protein. Alternatively, the PrfA protein can be any other gram-positive PrfA protein or any other PrfA protein.

In another example, the bacteria strain or Listeria strain can comprise a deletion of or an inactivating mutation in an actA gene, and the complementing gene can comprise an actA gene to complement the mutation and restore function to the Listeria strain.

Other auxotroph strains and complementation systems can also be adopted for the use with the methods and compositions provided herein.

C. Preparation and Storage of Bacteria or Listeria Strains

The recombinant bacteria strain (e.g., Listeria strain) optionally has been passaged through an animal host. Such passaging can maximize efficacy of the Listeria strain as a vaccine vector, can stabilize the immunogenicity of the Listeria strain, can stabilize the virulence of the Listeria strain, can increase the immunogenicity of the Listeria strain, can increase the virulence of the Listeria strain, can remove unstable sub-strains of the Listeria strain, or can reduce the prevalence of unstable sub-strains of the Listeria strain. Methods for passaging a recombinant Listeria strain through an animal host are well known in the art and are described, for example, in US 2006/0233835, herein incorporated by reference in its entirety for all purposes.

The recombinant bacteria strain (e.g., Listeria strain) can be stored in a frozen cell bank or stored in a lyophilized cell bank. Such a cell bank can be, for example, a master cell bank, a working cell bank, or a Good Manufacturing Practice (GMP) cell bank. Examples of “Good Manufacturing Practices” include those defined by 21 CFR 210-211 of the United States Code of Federal Regulations. However, “Good Manufacturing Practices” can also be defined by other standards for production of clinical-grade material or for human consumption, such as standards of a country other than the United States. Such cell banks can be intended for production of clinical-grade material or can conform to regulatory practices for human use.

Recombinant bacteria strains (e.g., Listeria strains) can also be from a batch of vaccine doses, from a frozen stock, or from a lyophilized stock.

Such cell banks, frozen stocks, or batches of vaccine doses can, for example, exhibit viability upon thawing of greater than 90%. The thawing, for example, can follow storage for cryopreservation or frozen storage for 24 hours. Alternatively, the storage can last, for example, for 2 days, 3 days, 4 days, 1 week, 2 weeks, 3 weeks, 1 month, 2 months, 3 months, 5 months, 6 months, 9 months, or 1 year.

The cell bank, frozen stock, or batch of vaccine doses can be cryopreserved, for example, by a method that comprises growing a culture of the bacteria strain (e.g., Listeria strain) in a nutrient media, freezing the culture in a solution comprising glycerol, and storing the Listeria strain at below −20° C. The temperature can be, for example, about −70° C. or between about −70 to about −80° C. Alternatively, the cell bank, frozen stock, or batch of vaccine doses can be cryopreserved by a method that comprises growing a culture of the Listeria strain in a defined medium, freezing the culture in a solution comprising glycerol, and storing the Listeria strain at below −20° C. The temperature can be, for example, about −70° C. or between about −70 to about −80° C. Any defined microbiological medium may be used in this method.

The culture (e.g., the culture of a Listeria vaccine strain that is used to produce a batch of Listeria vaccine doses) can be inoculated, for example, from a cell bank, from a frozen stock, from a starter culture, or from a colony. The culture can be inoculated, for example, at mid-log growth phase, at approximately mid-log growth phase, or at another growth phase.

The solution used for freezing optionally contain another colligative additive or additive with anti-freeze properties in place of glycerol or in addition to glycerol. Examples of such additives include, for example, mannitol, DMSO, sucrose, or any other colligative additive or additive with anti-freeze properties.

The nutrient medium utilized for growing a culture of a bacteria strain (e.g., a Listeria strain) can be any suitable nutrient medium. Examples of suitable media include, for example, LB; TB; a modified, animal-product-free Terrific Broth; or a defined medium.

The step of growing can be performed by any known means of growing bacteria. For example, the step of growing can be performed with a shake flask (such as a baffled shake flask), a batch fermenter, a stirred tank or flask, an airlift fermenter, a fed batch, a continuous cell reactor, an immobilized cell reactor, or any other means of growing bacteria.

Optionally, a constant pH is maintained during growth of the culture (e.g. in a batch fermenter). For example, the pH can be maintained at about 6.0, at about 6.5, at about 7.0, at about 7.5, or about 8.0. Likewise, the pH can be, for example, from about 6.5 to about 7.5, from about 6.0 to about 8.0, from about 6.0 to about 7.0, from about 6.0 to about 7.0, or from about 6.5 to about 7.5.

Optionally, a constant temperature can be maintained during growth of the culture. For example, the temperature can be maintained at about 37° C. or at 37° C. Alternatively, the temperature can be maintained at 25° C., 27° C., 28° C., 30° C., 32° C., 34° C., 35° C., 36° C., 38° C., or 39° C.

Optionally, a constant dissolved oxygen concentration can be maintained during growth of the culture. For example, the dissolved oxygen concentration can be maintained at 20% of saturation, 15% of saturation, 16% of saturation, 18% of saturation, 22% of saturation, 25% of saturation, 30% of saturation, 35% of saturation, 40% of saturation, 45% of saturation, 50% of saturation, 55% of saturation, 60% of saturation, 65% of saturation, 70% of saturation, 75% of saturation, 80% of saturation, 85% of saturation, 90% of saturation, 95% of saturation, 100% of saturation, or near 100% of saturation.

Methods for lyophilization and cryopreservation of recombinant bacteria strains (e.g., Listeria strains are known. For example, a Listeria culture can be flash-frozen in liquid nitrogen, followed by storage at the final freezing temperature. Alternatively, the culture can be frozen in a more gradual manner (e.g., by placing in a vial of the culture in the final storage temperature). The culture can also be frozen by any other known method for freezing a bacterial culture.

The storage temperature of the culture can be, for example, between −20 and −80° C. For example, the temperature can be significantly below −20° C. or not warmer than −70° C. Alternatively, the temperature can be about −70° C., −20° C., −30° C., −40° C., −50° C., −60° C., −80° C., −30 to −70° C., −40 to −70° C., −50 to −70° C., −60 to −70° C., −30 to −80° C., −40 to −80° C., −50 to −80° C., −60 to −80° C., or −70 to −80° C. Alternatively, the temperature can be colder than 70° C. or colder than −80° C.

### V. Immunogenic Compositions, Pharmaceutical Compositions, and Vaccines

Also provided are immunogenic compositions, pharmaceutical compositions, or vaccines comprising a heteroclitic peptide as disclosed herein, a recombinant fusion polypeptide as disclosed herein, a nucleic acid encoding a heteroclitic peptide or recombinant fusion polypeptide as disclosed herein, or a recombinant bacteria or Listeria strain as disclosed herein. An immunogenic composition comprising a Listeria strain can be inherently immunogenic by virtue of its comprising a Listeria strain and/or the composition can also further comprise an adjuvant. Other immunogenic compositions comprise DNA immunotherapy or peptide immunotherapy compositions.

The term “immunogenic composition” refers to any composition containing an antigen that elicits an immune response against the antigen in a subject upon exposure to the composition. The immune response elicited by an immunogenic composition can be to a particular antigen or to a particular epitope on the antigen.

An immunogenic composition can comprise a single heteroclitic peptide or recombinant fusion polypeptide as disclosed herein, nucleic acid encoding a heteroclitic peptide or recombinant fusion polypeptide as disclosed herein, or recombinant bacteria or Listeria strain as disclosed herein, or it can comprise multiple different heteroclitic peptides or recombinant fusion polypeptides as disclosed herein, nucleic acids encoding heteroclitic peptides or recombinant fusion polypeptides as disclosed herein, or recombinant bacteria or Listeria strains as disclosed herein. A first recombinant fusion polypeptide is different from a second recombinant fusion polypeptide, for example, if it includes one antigenic peptide that the second recombinant fusion polypeptide does not. Two recombinant fusion polypeptides can include some of the same antigenic peptides and still be considered different. Such different heteroclitic peptides, recombinant fusion polypeptides, nucleic acids encoding heteroclitic peptides or recombinant fusion polypeptides, or recombinant bacteria or Listeria strains can be administered concomitantly to a subject or sequentially to a subject. Sequential administration can be particularly useful when a drug substance comprising a recombinant Listeria strain (or heteroclitic peptide, recombinant fusion polypeptide, or nucleic acid) disclosed herein is in different dosage forms (e.g., one agent is a tablet or capsule and another agent is a sterile liquid) and/or is administered on different dosing schedules (e.g., one composition from the mixture is administered at least daily and another is administered less frequently, such as once weekly, once every two weeks, or once every three weeks). The multiple heteroclitic peptides, recombinant fusion polypeptides, nucleic acids encoding heteroclitic peptides or recombinant fusion polypeptides, or recombinant bacteria or Listeria strains can each comprise a different set of antigenic peptides. Alternatively, two or more of the heteroclitic peptides, recombinant fusion polypeptides, nucleic acids encoding heteroclitic peptides, recombinant fusion polypeptides, or recombinant bacteria or Listeria strains can comprise the same set of antigenic peptides (e.g., the same set of antigenic peptides in a different order).

The multiple heteroclitic peptides or fragments or the recombinant fusion polypeptide can bind to multiple different HLA types. For example, they can bind to one or more or all of the following HLA types: HLA-A*02:01, HLA-A*03:01, HLA-A*24:02, and HLA-B*07:02.

As one example, an immunogenic composition can comprise heteroclitic peptides (in the form of, e.g., peptides, nucleic acids, or bacterial vectors) encoded by 1 or more, 2 or more, 3 or more, 4 or more, 5 or more, 6 or more, or all of the following genes: CEACAM5, MAGEA6, MAGEA4, GAGE1, NYESO1, STEAP1, and RNF43. The heteroclitic antigenic peptides can bind, for example, one or more or all of HLA-A*02:01, HLA-A*03:01, HLA-A*24:02, and HLA-B*07:02. Such cancer-associated proteins are associated with, for example, non-small cell lung cancer (NSCLC). The heteroclitic antigenic peptides can be in any order. The heteroclitic antigenic peptides can be fused directly together or linked together by linkers, examples of which are disclosed elsewhere herein. In a specific example, one or more or all of the heteroclitic antigenic peptides can be 9-mers (e.g., 9-mers linked together by linkers). Examples of such antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, or all 11 of the heteroclitic antigenic peptides in Table 3 or peptides comprising, for example, 1 or more, 2 or more, 3 or more, 4 or more, 5 or more, 6 or more, 7 or more, 8 or more, 9 or more, 10 or more, or all 11 of the sequences in Table 3.

As another example, an immunogenic composition can comprise heteroclitic peptides (in the form of, e.g., peptides, nucleic acids, or bacterial vectors) encoded by 1 or more, 2 or more, 3 or more, 4 or more, 5 or more, 6 or more, 7 or more, 8 or more, or all of the following genes: CEACAM5, MAGEA4, STEAP1, RNF43, SSX2, SART3, PAGE4, PSMA, and PSA. The heteroclitic antigenic peptides can bind, for example, one or more or all of HLA-A*02:01, HLA-A*03:01, HLA-A*24:02, and HLA-B*07:02. Such cancer-associated proteins are associated with, for example, prostate cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, or all 10 of the heteroclitic antigenic peptides in Table 5 or peptides comprising, for example, 1 or more, 2 or more, 3 or more, 4 or more, 5 or more, 6 or more, 7 or more, 8 or more, 9 or more, or all 10 of the sequences in Table 5.

As another example, an immunogenic composition can comprise heteroclitic peptides (in the form of, e.g., peptides, nucleic acids, or bacterial vectors) encoded by 1 or more, 2 or more, 3 or more, 4 or more, 5 or more, or all of the following genes: CEACAM5, STEAP1, MAGEA3, PRAME, hTERT, and SURVIVIN. The heteroclitic antigenic peptides can bind, for example, one or more or all of HLA-A*02:01, HLA-A*03:01, HLA-A*24:02, and HLA-B*07:02. Such cancer-associated proteins are associated with, for example, pancreatic cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, 11 or more, or all 12 of the heteroclitic antigenic peptides in Table 7 or peptides comprising, for example, 1 or more, 2 or more, 3 or more, 4 or more, 5 or more, 6 or more, 7 or more, 8 or more, 9 or more, 10 or more, 11 or more, or all 12 of the sequences in Table 7.

As another example, an immunogenic composition can comprise heteroclitic peptides (in the form of, e.g., peptides, nucleic acids, or bacterial vectors) encoded by 1 or more, 2 or more, 3 or more, 4 or more, 5 or more, 6 or more, 7 or more, or all of the following genes: CEACAM5, GAGE1, NYESO1, RNF43, NUF2, KLHL7, MAGEA3, and PRAME. The heteroclitic antigenic peptides can bind, for example, one or more or all of HLA-A*02:01, HLA-A*03:01, HLA-A*24:02, and HLA-B*07:02. Such cancer-associated proteins are associated with, for example, bladder cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, 11 or more, 12 or more, 13 or more, or all 14 of the heteroclitic antigenic peptides in Table 9 or peptides comprising, for example, 1 or more, 2 or more, 3 or more, 4 or more, 5 or more, 6 or more, 7 or more, 8 or more, 9 or more, 10 or more, 11 or more, 12 or more, or all 13 of the sequences in Table 9.

As another example, an immunogenic composition can comprise heteroclitic peptides (in the form of, e.g., peptides, nucleic acids, or bacterial vectors) encoded by 1 or more, 2 or more, 3 or more, 4 or more, 5 or more, or all of the following genes: CEACAM5, STEAP1, RNF43, MAGEA3, PRAME, and hTERT. The heteroclitic antigenic peptides can bind, for example, one or more or all of HLA-A*02:01, HLA-A*03:01, HLA-A*24:02, and HLA-B*07:02. Such cancer-associated proteins are associated with, for example, breast cancer (e.g., ER+ breast cancer). The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, or all 11 of the heteroclitic antigenic peptides in Table 11 or peptides comprising, for example, 1 or more, 2 or more, 3 or more, 4 or more, 5 or more, 6 or more, 7 or more, 8 or more, 9 or more, 10 or more, or all 11 of the sequences in Table 11.

As another example, an immunogenic composition can comprise heteroclitic peptides (in the form of, e.g., peptides, nucleic acids, or bacterial vectors) encoded by 1 or more, 2 or more, 3 or more, 4 or more, 5 or more, 6 or more, 7 or more, ore or all of the following genes: CEACAM5, PRAME, hTERT, STEAP1, RNF43, NUF2, KLHL7, and SART3. The heteroclitic antigenic peptides can bind, for example, one or more or all of HLA-A*02:01, HLA-A*03:01, HLA-A*24:02, and HLA-B*07:02. Such cancer-associated proteins are associated with, for example, uterine cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, 11 or more, 12 or more, 13 or more, or all 14 of the heteroclitic antigenic peptides in Table 13 or peptides comprising, for example, 1 or more, 2 or more, 3 or more, 4 or more, 5 or more, 6 or more, 7 or more, 8 or more, 9 or more, 10 or more, 11 or more, 12 or more, 13 or more, or all 14 of the sequences in Table 13.

As another example, an immunogenic composition can comprise heteroclitic peptides (in the form of, e.g., peptides, nucleic acids, or bacterial vectors) encoded by 1 or more, 2 or more, 3 or more, 4 or more, 5 or more, 6 or more, 7 or more, or all of the following genes: CEACAM5, STEAP1, RNF43, SART3, NUF2, KLHL7, PRAME, and hTERT. The heteroclitic antigenic peptides can bind, for example, one or more or all of HLA-A*02:01, HLA-A*03:01, HLA-A*24:02, and HLA-B*07:02. Such cancer-associated proteins are associated with, for example, ovarian cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, 11 or more, 12 or more, 13 or more, or all 14 of the heteroclitic antigenic peptides in Table 15 or peptides comprising, for example, 1 or more, 2 or more, 3 or more, 4 or more, 5 or more, 6 or more, 7 or more, 8 or more, 9 or more, 10 or more, 11 or more, 12 or more, 13 or more, or all 14 of the sequences in Table 15.

As another example, an immunogenic composition can comprise heteroclitic peptides (in the form of, e.g., peptides, nucleic acids, or bacterial vectors) encoded by 1 or more, 2 or more, 3 or more, 4 or more, 5 or more, 6 or more, 7 or more, or all of the following genes: CEACAM5, MAGEA6, STEAP1, RNF43, SART3, NUF2, KLHL7, and hTERT. The heteroclitic antigenic peptides can bind, for example, one or more or all of HLA-A*02:01, HLA-A*03:01, HLA-A*24:02, and HLA-B*07:02. Such cancer-associated proteins are associated with, for example, low-grade glioma. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, or all 10 of the heteroclitic antigenic peptides in Table 17 or peptides comprising, for example, 1 or more, 2 or more, 3 or more, 4 or more, 5 or more, 6 or more, 7 or more, 8 or more, 9 or more, or all 10 of the sequences in Table 17.

As another example, an immunogenic composition can comprise heteroclitic peptides (in the form of, e.g., peptides, nucleic acids, or bacterial vectors) encoded by 1 or more, 2 or more, 3 or more, 4 or more, 5 or more, 6 or more, 7 or more, or all of the following genes: CEACAM5, MAGEA6, MAGEA4, GAGE1, NYESO1, STEAP1, RNF43, and MAGEA3. The heteroclitic antigenic peptides can bind, for example, one or more or all of HLA-A*02:01, HLA-A*03:01, HLA-A*24:02, and HLA-B*07:02. Such cancer-associated proteins are associated with, for example, colorectal cancer (e.g., MSS colorectal cancer). The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, or all 10 of the heteroclitic antigenic peptides in Table 19 or peptides comprising, for example, 1 or more, 2 or more, 3 or more, 4 or more, 5 or more, 6 or more, 7 or more, 8 or more, 9 or more, or all 10 of the sequences in Table 19.

As another example, an immunogenic composition can comprise heteroclitic peptides (in the form of, e.g., peptides, nucleic acids, or bacterial vectors) encoded by 1 or more, 2 or more, 3 or more, 4 or more, 5 or more, or all of the following genes: CEACAM5, MAGEA4, STEAP1, NYESO1, PRAME, and hTERT. The heteroclitic antigenic peptides can bind, for example, one or more or all of HLA-A*02:01, HLA-A*03:01, HLA-A*24:02, and HLA-B*07:02. Such cancer-associated proteins are associated with, for example, head and neck cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, or all 10 of the heteroclitic antigenic peptides in Table 21 or peptides comprising, for example, 1 or more, 2 or more, 3 or more, 4 or more, 5 or more, 6 or more, 7 or more, 8 or more, 9 or more, or all 10 of the sequences in Table 21.

An immunogenic composition can additionally comprise an adjuvant (e.g., two or more adjuvants), a cytokine, a chemokine, or combination thereof. Optionally, an immunogenic composition can additionally comprises antigen presenting cells (APCs), which can be autologous or can be allogeneic to the subject.

The term adjuvant includes compounds or mixtures that enhance the immune response to an antigen. For example, an adjuvant can be a non-specific stimulator of an immune response or substances that allow generation of a depot in a subject which when combined with an immunogenic composition disclosed herein provides for an even more enhanced and/or prolonged immune response. An adjuvant can favor, for example, a predominantly Th1-mediated immune response, a Th1-type immune response, or a Th1-mediated immune response. Likewise, an adjuvant can favor a cell-mediated immune response over an antibody-mediated response. Alternatively, an adjuvant can favor an antibody-mediated response. Some adjuvants can enhance the immune response by slowly releasing the antigen, while other adjuvants can mediate their effects by any of the following mechanisms: increasing cellular infiltration, inflammation, and trafficking to the injection site, particularly for antigen-presenting cells (APC); promoting the activation state of APCs by upregulating costimulatory signals or major histocompatibility complex (MHC) expression; enhancing antigen presentation; or inducing cytokine release for indirect effect.

Examples of adjuvants include saponin QS21, CpG oligonucleotides, unmethylated CpG-containing oligonucleotides, MPL, TLR agonists, TLR4 agonists, TLR9 agonists, Resiquimod®, imiquimod, cytokines or nucleic acids encoding the same, chemokines or nucleic acids encoding same, IL-12 or a nucleic acid encoding the same, IL-6 or a nucleic acid encoding the same, and lipopolysaccharides. Another example of a suitable adjuvant is Montanide ISA 51. Montanide ISA 51 contains a natural metabolizable oil and a refined emulsifier. Other examples of a suitable adjuvant include granulocyte/macrophage colony-stimulating factor (GM-CSF) or a nucleic acid encoding the same and keyhole limpet hemocyanin (KLH) proteins or nucleic acids encoding the same. The GM-CSF can be, for example, a human protein grown in a yeast (S. cerevisiae) vector. GM-CSF promotes clonal expansion and differentiation of hematopoietic progenitor cells, antigen presenting cells (APCs), dendritic cells, and T cells.

Yet another example of a suitable adjuvant is detoxified listeriolysin O (dtLLO) protein. Detoxification can be accomplished by introducing point mutations for three selected amino acids important for binding of LLO to cholesterol and for eventual membrane pore formation. The three targeted amino acids are present in the cholesterol binding domain of LLO (ECTGLAWEWWR; SEQ ID NO: 74) and can be modified in the sequence (EATGLAWEAAR; SEQ ID NO: 96) by point mutations introduced into the DNA sequence by PCR. One example of a dtLLO suitable for use as an adjuvant is encoded by SEQ ID NO: 95. The detoxified, nonhemolytic form of LLO (dtLLO) is an effective adjuvant in tumor immunotherapy and may activate innate and cellular immune responses by acting as a PAMP. A dtLLO encoded by a sequence at least 90%, 95%, 96%, 97%, 98%, or 99% identical to SEQ ID NO: 95 is also suitable for use as an adjuvant.

Yet other examples of adjuvants include growth factors or nucleic acids encoding the same, cell populations, Freund's incomplete adjuvant, aluminum phosphate, aluminum hydroxide, BCG (bacille Calmette-Guerin), alum, interleukins or nucleic acids encoding the same, quill glycosides, monophosphoryl lipid A, liposomes, bacterial mitogens, bacterial toxins, or any other type of known adjuvant (see, e.g., Fundamental Immunology, 5th ed. (August 2003): William E. Paul (Editor); Lippincott Williams & Wilkins Publishers; Chapter 43: Vaccines, GJV Nossal, which is herein incorporated by reference in its entirety for all purposes).

An immunogenic composition can further comprise one or more immunomodulatory molecules. Examples include interferon gamma, a cytokine, a chemokine, and a T cell stimulant.

An immunogenic composition can be in the form of a vaccine or pharmaceutical composition. The terms “vaccine” and “pharmaceutical composition” are interchangeable and refer to an immunogenic composition in a pharmaceutically acceptable carrier for in vivo administration to a subject. A vaccine may be, for example, a peptide vaccine (e.g., comprising a heteroclitic peptide or recombinant fusion polypeptide as disclosed herein), a DNA vaccine (e.g., comprising a nucleic acid encoding a heteroclitic peptide or recombinant fusion polypeptide as disclosed herein), or a vaccine contained within and delivered by a cell (e.g., a recombinant Listeria as disclosed herein). A vaccine may prevent a subject from contracting or developing a disease or condition and/or a vaccine may be therapeutic to a subject having a disease or condition. Methods for preparing peptide vaccines are well known and are described, for example, in EP 1408048, US 2007/0154953, and Ogasawara et al. (1992) Proc. Natl Acad Sci USA 89:8995-8999, each of which is herein incorporated by reference in its entirety for all purposes. Optionally, peptide evolution techniques can be used to create an antigen with higher immunogenicity. Techniques for peptide evolution are well known and are described, for example, in U.S. Pat. No. 6,773,900, herein incorporated by reference in its entirety for all purposes.

A “pharmaceutically acceptable carrier” refers to a vehicle for containing an immunogenic composition that can be introduced into a subject without significant adverse effects and without having deleterious effects on the immunogenic composition. That is, “pharmaceutically acceptable” refers to any formulation which is safe, and provides the appropriate delivery for the desired route of administration of an effective amount of at least one immunogenic composition for use in the methods disclosed herein. Pharmaceutically acceptable carriers or vehicles or excipients are well known. Descriptions of suitable pharmaceutically acceptable carriers, and factors involved in their selection, are found in a variety of readily available sources such as, for example, Remington's Pharmaceutical Sciences, 18th ed., 1990, herein incorporated by reference in its entirety for all purposes. Such carriers can be suitable for any route of administration (e.g., parenteral, enteral (e.g., oral), or topical application). Such pharmaceutical compositions can be buffered, for example, wherein the pH is maintained at a particular desired value, ranging from pH 4.0 to pH 9.0, in accordance with the stability of the immunogenic compositions and route of administration.

Suitable pharmaceutically acceptable carriers include, for example, sterile water, salt solutions such as saline, glucose, buffered solutions such as phosphate buffered solutions or bicarbonate buffered solutions, alcohols, gum arabic, vegetable oils, benzyl alcohols, polyethylene glycols, gelatine, carbohydrates (e.g., lactose, amylose or starch), magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, and the like. Pharmaceutical compositions or vaccines may also include auxiliary agents including, for example, diluents, stabilizers (e.g., sugars and amino acids), preservatives, wetting agents, emulsifiers, pH buffering agents, viscosity enhancing additives, lubricants, salts for influencing osmotic pressure, buffers, vitamins, coloring, flavoring, aromatic substances, and the like which do not deleteriously react with the immunogenic composition.

For liquid formulations, for example, pharmaceutically acceptable carriers may be aqueous or non-aqueous solutions, suspensions, emulsions, or oils. Non-aqueous solvents include, for example, propylene glycol, polyethylene glycol, and injectable organic esters such as ethyl oleate. Aqueous carriers include, for example, water, alcoholic/aqueous solutions, emulsions or suspensions, including saline and buffered media. Examples of oils include those of petroleum, animal, vegetable, or synthetic origin, such as peanut oil, soybean oil, mineral oil, olive oil, sunflower oil, and fish-liver oil. Solid carriers/diluents include, for example, a gum, a starch (e.g., corn starch, pregeletanized starch), a sugar (e.g., lactose, mannitol, sucrose, or dextrose), a cellulosic material (e.g., microcrystalline cellulose), an acrylate (e.g., polymethylacrylate), calcium carbonate, magnesium oxide, talc, or mixtures thereof.

Optionally, sustained or directed release pharmaceutical compositions or vaccines can be formulated. This can be accomplished, for example, through use of liposomes or compositions wherein the active compound is protected with differentially degradable coatings (e.g., by microencapsulation, multiple coatings, and so forth). Such compositions may be formulated for immediate or slow release. It is also possible to freeze-dry the compositions and use the lyophilisates obtained (e.g., for the preparation of products for injection).

An immunogenic composition, pharmaceutical composition, or vaccine disclosed herein may also comprise one or more additional compounds effective in preventing or treating cancer. For example, the additional compound may comprise a compound useful in chemotherapy, such as amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil (5-FU), gemcitabine, gliadelimplants, hydroxycarbamide, idarubicin, ifosfamide, irinotecan, leucovorin, liposomaldoxorubicin, liposomaldaunorubicin, lomustine, melphalan, mercaptopurine, mesna, methotrexate, mitomycin, mitoxantrone, oxaliplatin, paclitaxel (Taxol), pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. The additional compound can also comprise other biologics, including Herceptin® (trastuzumab) against the HER2 antigen, Avastin® (bevacizumab) against VEGF, or antibodies to the EGF receptor, such as Erbitux® (cetuximab), and Vectibix® (panitumumab). The additional compound can also comprise, for example, an additional immunotherapy.

An additional compound can also comprise an immune checkpoint inhibitor antagonist, such as a PD-1 signaling pathway inhibitor, a CD-80/86 and CTLA-4 signaling pathway inhibitor, a T cell membrane protein 3 (TIM3) signaling pathway inhibitor, an adenosine A2a receptor (A2aR) signaling pathway inhibitor, a lymphocyte activation gene 3 (LAG3) signaling pathway inhibitor, a killer immunoglobulin receptor (KIR) signaling pathway inhibitor, a CD40 signaling pathway inhibitor, or any other antigen-presenting cell/T cell signaling pathway inhibitor. Examples of immune checkpoint inhibitor antagonists include an anti-PD-L1/PD-L2 antibody or fragment thereof, an anti-PD-1 antibody or fragment thereof, an anti-CTLA-4 antibody or fragment thereof, or an anti-B7-H4 antibody or fragment thereof. An additional compound can also comprise a T cell stimulator, such as an antibody or functional fragment thereof binding to a T-cell receptor co-stimulatory molecule, an antigen presenting cell receptor binding co-stimulatory molecule, or a member of the TNF receptor superfamily. The T-cell receptor co-stimulatory molecule can comprise, for example, CD28 or ICOS. The antigen presenting cell receptor binding co-stimulatory molecule can comprise, for example, a CD80 receptor, a CD86 receptor, or a CD46 receptor. The TNF receptor superfamily member can comprise, for example, glucocorticoid-induced TNF receptor (GITR), OX40 (CD134 receptor), 4-1BB (CD137 receptor), or TNFR25. See, e.g., WO2016100929, WO2016011362, and WO2016011357, each of which is incorporated by reference in its entirety for all purposes.

### VI. Therapeutic Methods

The heteroclitic peptides, recombinant fusion polypeptides, nucleic acids encoding heteroclitic peptides, nucleic acids encoding recombinant fusion polypeptides, recombinant bacteria or Listeria strains, immunogenic compositions, pharmaceutical compositions, and vaccines disclosed herein can be used in various methods. For example, they can be used in methods of inducing or enhancing an anti-cancer-associated-protein or anti-tumor-associated-antigen immune response in a subject, in methods of inducing or enhancing an anti-tumor or anti-cancer immune response in a subject, in methods of treating a tumor or cancer in a subject, in methods of preventing a tumor or cancer in a subject, or in methods of protecting a subject against a tumor or cancer. They can also be used in methods of increasing the ratio of T effector cells to regulatory T cells (Tregs) in the spleen and tumor of a subject, wherein the T effector cells are targeted to a tumor-associated antigen. They can also be used in methods for increasing tumor-associated-antigen T cells in a subject, increasing survival time of a subject having a tumor or cancer, delaying the onset of cancer in a subject, or reducing tumor or metastasis size in a subject.

A method of inducing or enhancing an anti-tumor-associated-antigen immune response in a subject can comprise, for example, administering to the subject a heteroclitic peptide, a recombinant fusion polypeptide, a nucleic acid encoding a heteroclitic peptide or a recombinant fusion polypeptide, a recombinant bacteria or Listeria strain, an immunogenic composition, a pharmaceutical composition, or a vaccine disclosed herein (e.g., that comprises a heteroclitic peptide or recombinant fusion polypeptide comprising the heteroclitic peptide or a nucleic acid encoding the heteroclitic peptide or recombinant fusion polypeptide). An anti-tumor-associate-antigen immune response can thereby be induced or enhanced in the subject. For example, in the case of a recombinant Listeria strain, the Listeria strain can express the fusion polypeptide, thereby eliciting an immune response in the subject. The immune response can comprise, for example, a T-cell response, such as a CD4+FoxP3− T cell response, a CD8+ T cell response, or a CD4+FoxP3− and CD8+ T cell response. Such methods can also increase the ratio of T effector cells to regulatory T cells (Tregs) in the spleen and tumor microenvironments of the subject, allowing for a more profound anti-tumor response in the subject.

A method of inducing or enhancing an anti-tumor or anti-cancer immune response in a subject can comprise, for example, administering to the subject a heteroclitic peptide, a recombinant fusion polypeptide, a nucleic acid encoding a heteroclitic peptide or a recombinant fusion polypeptide, a recombinant bacteria or Listeria strain, an immunogenic composition, a pharmaceutical composition, or a vaccine disclosed herein. An anti-tumor or anti-cancer immune response can thereby be induced or enhanced in the subject. For example, in the case of a recombinant Listeria strain, the Listeria strain can express the fusion polypeptide, thereby eliciting an anti-tumor or anti-cancer response in the subject.

A method of treating a tumor or cancer in a subject (e.g., wherein the tumor or cancer expresses a particular tumor-associated antigen or cancer-associated protein as disclosed elsewhere herein), can comprise, for example, administering to the subject a heteroclitic peptide, a recombinant fusion polypeptide, a nucleic acid encoding a heteroclitic peptide or recombinant fusion polypeptide, a recombinant bacteria or Listeria strain, an immunogenic composition, a pharmaceutical composition, or a vaccine disclosed herein. The subject can then mount an immune response against the tumor or cancer expressing the tumor-associated antigen, thereby treating the tumor or cancer in the subject.

A method of preventing a tumor or cancer in a subject or protecting a subject against developing a tumor or cancer (e.g., wherein the tumor or cancer is associated with expression of a particular tumor-associated antigen or cancer-associated protein as disclosed elsewhere herein), can comprise, for example, administering to the subject a heteroclitic peptide, a recombinant fusion polypeptide, a nucleic acid encoding a heteroclitic peptide or recombinant fusion polypeptide, a recombinant bacteria or Listeria strain, an immunogenic composition, a pharmaceutical composition, or a vaccine disclosed herein. The subject can then mount an immune response against the tumor-associated antigen, thereby preventing a tumor or cancer or protecting the subject against developing a tumor or cancer.

In some of the above methods, two or more heteroclitic peptides, recombinant fusion polypeptides, nucleic acids encoding heteroclitic peptides or recombinant fusion polypeptides, recombinant bacteria or Listeria strains, immunogenic compositions, pharmaceutical compositions, or vaccines are administered. The multiple heteroclitic peptides, recombinant fusion polypeptides, nucleic acids encoding heteroclitic peptides or recombinant fusion polypeptides, recombinant bacteria or Listeria strains, immunogenic compositions, pharmaceutical compositions, or vaccines can be administered sequentially in any order or combination, or can be administered simultaneously in any combination. As an example, if four different Listeria strains are being administered, they can be administered sequentially, they can be administered simultaneously, or they can be administered in any combination (e.g., administering the first and second strains simultaneously and subsequently administering the third and fourth strains simultaneously). Optionally, in the case of sequential administration, the compositions can be administered during the same immune response, preferably within 0-10 or 3-7 days of each other. The multiple heteroclitic peptides, recombinant fusion polypeptides, nucleic acids encoding heteroclitic peptides or recombinant fusion polypeptides, recombinant bacteria or Listeria strains, immunogenic compositions, pharmaceutical compositions, or vaccines can each comprise a different set of antigenic peptides. Alternatively, two or more can comprise the same set of antigenic peptides (e.g., the same set of antigenic peptides in a different order).

The multiple heteroclitic peptides or fragments or the recombinant fusion polypeptide can bind to multiple different HLA types. For example, they can bind to one or more or all of the following HLA types: HLA-A*02:01, HLA-A*03:01, HLA-A*24:02, and HLA-B*07:02.

As one example, the multiple heteroclitic peptides (in the form of, e.g., peptides, recombinant fusion polypeptides, nucleic acids, or bacterial vectors) can be encoded by 1 or more, 2 or more, 3 or more, 4 or more, 5 or more, 6 or more, or all of the following genes: CEACAM5, MAGEA6, MAGEA4, GAGE1, NYESO1, STEAP1, and RNF43. The heteroclitic antigenic peptides can bind, for example, one or more or all of HLA-A*02:01, HLA-A*03:01, HLA-A*24:02, and HLA-B*07:02. Such cancer-associated proteins are associated with, for example, non-small cell lung cancer (NSCLC). The heteroclitic antigenic peptides can be in any order. The heteroclitic antigenic peptides can be fused directly together or linked together by linkers, examples of which are disclosed elsewhere herein. In a specific example, one or more or all of the heteroclitic antigenic peptides can be 9-mers (e.g., 9-mers linked together by linkers). Examples of such antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, or all 11 of the heteroclitic antigenic peptides in Table 3 or peptides comprising, for example, 1 or more, 2 or more, 3 or more, 4 or more, 5 or more, 6 or more, 7 or more, 8 or more, 9 or more, 10 or more, or all 11 of the sequences in Table 3.

As another example, the multiple heteroclitic peptides (in the form of, e.g., peptides, recombinant fusion polypeptides, nucleic acids, or bacterial vectors) can be encoded by 1 or more, 2 or more, 3 or more, 4 or more, 5 or more, 6 or more, 7 or more, 8 or more, or all of the following genes: CEACAM5, MAGEA4, STEAP1, RNF43, SSX2, SART3, PAGE4, PSMA, and PSA. The heteroclitic antigenic peptides can bind, for example, one or more or all of HLA-A*02:01, HLA-A*03:01, HLA-A*24:02, and HLA-B*07:02. Such cancer-associated proteins are associated with, for example, prostate cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, or all 10 of the heteroclitic antigenic peptides in Table 5 or peptides comprising, for example, 1 or more, 2 or more, 3 or more, 4 or more, 5 or more, 6 or more, 7 or more, 8 or more, 9 or more, or all 10 of the sequences in Table 5.

As another example, the multiple heteroclitic peptides (in the form of, e.g., peptides, recombinant fusion polypeptides, nucleic acids, or bacterial vectors) can be encoded by 1 or more, 2 or more, 3 or more, 4 or more, 5 or more, or all of the following genes: CEACAM5, STEAP1, MAGEA3, PRAME, hTERT, and SURVIVIN. The heteroclitic antigenic peptides can bind, for example, one or more or all of HLA-A*02:01, HLA-A*03:01, HLA-A*24:02, and HLA-B*07:02. Such cancer-associated proteins are associated with, for example, pancreatic cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, 11 or more, or all 12 of the heteroclitic antigenic peptides in Table 7 or peptides comprising, for example, 1 or more, 2 or more, 3 or more, 4 or more, 5 or more, 6 or more, 7 or more, 8 or more, 9 or more, 10 or more, 11 or more, or all 12 of the sequences in Table 7.

As another example, the multiple heteroclitic peptides (in the form of, e.g., peptides, recombinant fusion polypeptides, nucleic acids, or bacterial vectors) can be encoded by 1 or more, 2 or more, 3 or more, 4 or more, 5 or more, 6 or more, 7 or more, or all of the following genes: CEACAM5, GAGE1, NYESO1, RNF43, NUF2, KLHL7, MAGEA3, and PRAME. The heteroclitic antigenic peptides can bind, for example, one or more or all of HLA-A*02:01, HLA-A*03:01, HLA-A*24:02, and HLA-B*07:02. Such cancer-associated proteins are associated with, for example, bladder cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, 11 or more, 12 or more, 13 or more, or all 14 of the heteroclitic antigenic peptides in Table 9 or peptides comprising, for example, 1 or more, 2 or more, 3 or more, 4 or more, 5 or more, 6 or more, 7 or more, 8 or more, 9 or more, 10 or more, 11 or more, 12 or more, or all 13 of the sequences in Table 9.

As another example, the multiple heteroclitic peptides (in the form of, e.g., peptides, recombinant fusion polypeptides, nucleic acids, or bacterial vectors) can be encoded by 1 or more, 2 or more, 3 or more, 4 or more, 5 or more, or all of the following genes: CEACAM5, STEAP1, RNF43, MAGEA3, PRAME, and hTERT. The heteroclitic antigenic peptides can bind, for example, one or more or all of HLA-A*02:01, HLA-A*03:01, HLA-A*24:02, and HLA-B*07:02. Such cancer-associated proteins are associated with, for example, breast cancer (e.g., ER+breast cancer). The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, or all 11 of the heteroclitic antigenic peptides in Table 11 or peptides comprising, for example, 1 or more, 2 or more, 3 or more, 4 or more, 5 or more, 6 or more, 7 or more, 8 or more, 9 or more, 10 or more, or all 11 of the sequences in Table 11.

As another example, the multiple heteroclitic peptides (in the form of, e.g., peptides, recombinant fusion polypeptides, nucleic acids, or bacterial vectors) can be encoded by 1 or more, 2 or more, 3 or more, 4 or more, 5 or more, 6 or more, 7 or more, ore or all of the following genes: CEACAM5, PRAME, hTERT, STEAP1, RNF43, NUF2, KLHL7, and SART3. The heteroclitic antigenic peptides can bind, for example, one or more or all of HLA-A*02:01, HLA-A*03:01, HLA-A*24:02, and HLA-B*07:02. Such cancer-associated proteins are associated with, for example, uterine cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, 11 or more, 12 or more, 13 or more, or all 14 of the heteroclitic antigenic peptides in Table 13 or peptides comprising, for example, 1 or more, 2 or more, 3 or more, 4 or more, 5 or more, 6 or more, 7 or more, 8 or more, 9 or more, 10 or more, 11 or more, 12 or more, 13 or more, or all 14 of the sequences in Table 13.

As another example, the multiple heteroclitic peptides (in the form of, e.g., peptides, recombinant fusion polypeptides, nucleic acids, or bacterial vectors) can be encoded by 1 or more, 2 or more, 3 or more, 4 or more, 5 or more, 6 or more, 7 or more, or all of the following genes: CEACAM5, STEAP1, RNF43, SART3, NUF2, KLHL7, PRAME, and hTERT. The heteroclitic antigenic peptides can bind, for example, one or more or all of HLA-A*02:01, HLA-A*03:01, HLA-A*24:02, and HLA-B*07:02. Such cancer-associated proteins are associated with, for example, ovarian cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, 10 or more, 11 or more, 12 or more, 13 or more, or all 14 of the heteroclitic antigenic peptides in Table 15 or peptides comprising, for example, 1 or more, 2 or more, 3 or more, 4 or more, 5 or more, 6 or more, 7 or more, 8 or more, 9 or more, 10 or more, 11 or more, 12 or more, 13 or more, or all 14 of the sequences in Table 15.

As another example, the multiple heteroclitic peptides (in the form of, e.g., peptides, recombinant fusion polypeptides, nucleic acids, or bacterial vectors) can be encoded by 1 or more, 2 or more, 3 or more, 4 or more, 5 or more, 6 or more, 7 or more, or all of the following genes: CEACAM5, MAGEA6, STEAP1, RNF43, SART3, NUF2, KLHL7, and hTERT. The heteroclitic antigenic peptides can bind, for example, one or more or all of HLA-A*02:01, HLA-A*03:01, HLA-A*24:02, and HLA-B*07:02. Such cancer-associated proteins are associated with, for example, low-grade glioma. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, or all 10 of the heteroclitic antigenic peptides in Table 17 or peptides comprising, for example, 1 or more, 2 or more, 3 or more, 4 or more, 5 or more, 6 or more, 7 or more, 8 or more, 9 or more, or all 10 of the sequences in Table 17.

As another example, the multiple heteroclitic peptides (in the form of, e.g., peptides, recombinant fusion polypeptides, nucleic acids, or bacterial vectors) can be encoded by 1 or more, 2 or more, 3 or more, 4 or more, 5 or more, 6 or more, 7 or more, or all of the following genes: CEACAM5, MAGEA6, MAGEA4, GAGE1, NYESO1, STEAP1, RNF43, and MAGEA3. The heteroclitic antigenic peptides can bind, for example, one or more or all of HLA-A*02:01, HLA-A*03:01, HLA-A*24:02, and HLA-B*07:02. Such cancer-associated proteins are associated with, for example, colorectal cancer (e.g., MSS colorectal cancer). The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, or all 10 of the heteroclitic antigenic peptides in Table 19 or peptides comprising, for example, 1 or more, 2 or more, 3 or more, 4 or more, 5 or more, 6 or more, 7 or more, 8 or more, 9 or more, or all 10 of the sequences in Table 19.

As another example, the multiple heteroclitic peptides (in the form of, e.g., peptides, recombinant fusion polypeptides, nucleic acids, or bacterial vectors) can be encoded by 1 or more, 2 or more, 3 or more, 4 or more, 5 or more, or all of the following genes: CEACAM5, MAGEA4, STEAP1, NYESO1, PRAME, and hTERT. The heteroclitic antigenic peptides can bind, for example, one or more or all of HLA-A*02:01, HLA-A*03:01, HLA-A*24:02, and HLA-B*07:02. Such cancer-associated proteins are associated with, for example, head and neck cancer. The heteroclitic antigenic peptides can be in any order. The heteroclitic antigenic peptides can be fused directly together or linked together by linkers, examples of which are disclosed elsewhere herein. In a specific example, one or more or all of the antigenic peptides can be 9-mers (e.g., 9-mers linked together by linkers). Examples of such heteroclitic antigenic peptides are provided in Example 2. The heteroclitic antigenic peptides can include, for example, 1 or more, 2 or more, 3 or more, 4 or more, 5 or more, 6 or more, 7 or more, 8 or more, 9 or more, or all 10 of the heteroclitic antigenic peptides in Table 21 or peptides comprising, for example, 1 or more, 2 or more, 3 or more, 4 or more, 5 or more, 6 or more, 7 or more, 8 or more, 9 or more, or all 10 of the sequences in Table 21.

Cancer is a physiological condition in mammals that is typically characterized by unregulated cell growth and proliferation. Cancers can be hematopoietic malignancies or solid tumors (i.e., masses of cells that result from excessive cell growth or proliferation, including pre-cancerous legions). Metastatic cancer refers to a cancer that has spread from the place where it first started to another place in the body. Tumors formed by metastatic cancer cells are called a metastatic tumor or a metastasis, which is a term also used to refer to the process by which cancer cells spread to other parts of the body. In general, metastatic cancer has the same name and same type of cancer cells as the original, or primary, cancer. Examples of solid tumors include melanoma, carcinoma, blastoma, and sarcoma. Hematologic malignancies include, for example, leukemia or lymphoid malignancies, such as lymphoma. Exemplary categories of cancers include brain, breast, gastrointestinal, genitourinary, gynecologic, head and neck, heme, skin and thoracic. Brain malignancies include, for example, glioblastoma, high-grade pontine glioma, low-grade glioma, medulloblastoma, neuroblastoma, and pilocytic astrocytoma. Gastrointestinal cancers include, for example, colorectal, gallbladder, hepatocellular, pancreas, PNET, gastric, and esophageal. Genitourinary cancers include, for example, adrenocortical, bladder, kidney chromophobe, renal (clear cell), renal (papillary), rhabdoid cancers, and prostate. Gynecologic cancers include, for example, uterine carcinosarcoma, uterine endometrial, serous ovarian, and cervical. Head and neck cancers include, for example, thyroid, nasopharyngeal, head and neck, and adenoid cystic. Heme cancers include, for example, multiple myeloma, myelodysplasia, mantle-cell lymphoma, acute lymphoblastic leukemia (ALL), non-lymphoma, chronic lymphocytic leukemia (CLL), and acute myeloid leukemia (AML). Skin cancers includes, for example, cutaneous melanoma and squamous cell carcinoma. Thoracic cancers include, for example, squamous lung, small-cell lung, and lung adenocarcinoma.

More particular examples of such cancers include squamous cell cancer or carcinoma (e.g., oral squamous cell carcinoma), myeloma, oral cancer, juvenile nasopharyngeal angiofibroma, neuroendocrine tumors, lung cancer, cancer of the peritoneum, hepatocellular cancer, gastric or stomach cancer including gastrointestinal cancer, pancreatic cancer, glioma, glioblastoma, glial tumors, cervical cancer, ovarian cancer, liver cancer, bladder cancer, hepatoma, hepatocellular carcinoma, breast cancer, triple-negative breast cancer, colon cancer, rectal cancer, colorectal cancer, endometrial cancer or uterine cancer or carcinoma, salivary gland carcinoma, kidney or renal cancer (e.g., renal cell carcinoma), prostate cancer, vulval cancer, thyroid cancer, hepatic carcinoma, anal carcinoma, penile carcinoma, fibrosarcoma, gallbladder cancer, osteosarcoma, mesothelioma, as well as head and neck cancer. A cancer can also be a brain cancer or another type of CNS or intracranial tumor. For example, a subject can have an astrocytic tumor (e.g., astrocytoma, anaplastic astrocytoma, glioblastoma, pilocytic astrocytoma, subependymal giant cell astrocytoma, pleomorphic xanthoastrocytoma), oligodendroglial tumor (e.g., oligodendroglioma, anaplastic oligodendroglioma), ependymal cell tumor (e.g., ependymoma, anaplastic ependymoma, myxopapillary ependymoma, subependymoma), mixed glioma (e.g., mixed oligoastrocytoma, anaplastic oligoastrocytoma), neuroepithelial tumor of uncertain origin (e.g., polar spongioblastoma, astroblastoma, gliomatosis cerebri), tumor of the choroid plexus (e.g., choroid plexus papilloma, choroid plexus carcinoma), neuronal or mixed neuronal-glial tumor (e.g., gangliocytoma, dyplastic gangliocytoma of cerebellum, ganglioglioma, anaplastic ganglioglioma, desmoplastic infantile ganglioma, central neurocytoma, dysembryoplastic neuroepthelial tumor, olfactory neuroblastoma), pineal parenchyma tumor (e.g., pineocytoma, pineoblastoma, mixed pineocytoma/pineoblastoma), or tumor with mixed neuroblastic or glioblastic elements (e.g., medulloepithelioma, medulloblastoma, neuroblastoma, retinoblastoma, ependymoblastoma). Other examples of cancer include low-grade glioma, non-small cell lung cancer (NSCLC), estrogen-receptor-positive (ER+) breast cancer, and DNA mismatch repair deficient cancers or tumors. A cancer is called estrogen-receptor-positive if it has receptors for estrogen. Another example of a cancer is a microsatellite stable (MSS) colorectal cancer.

In a specific example, the cancer is non-small cell lung cancer, prostate cancer, pancreatic cancer, bladder cancer, breast cancer, uterine cancer, ovarian cancer, low-grade glioma, colorectal cancer, or head and neck cancer.

The term “treat” or “treating” refers to both therapeutic treatment and prophylactic or preventative measures, wherein the object is to prevent or lessen the targeted tumor or cancer. Treating may include one or more of directly affecting or curing, suppressing, inhibiting, preventing, reducing the severity of, delaying the onset of, slowing the progression of, stabilizing the progression of, inducing remission of, preventing or delaying the metastasis of, reducing/ameliorating symptoms associated with the tumor or cancer, or a combination thereof. For example, treating may include increasing expected survival time or decreasing tumor or metastasis size. The effect (e.g., suppressing, inhibiting, preventing, reducing the severity of, delaying the onset of, slowing the progression of, stabilizing the progression of, inducing remission of, preventing or delaying the metastasis of, reducing/ameliorating symptoms of, and so forth, can be relative to a control subject not receiving a treatment or receiving a placebo treatment. The term “treat” or “treating” can also refer to increasing percent chance of survival or increasing expected time of survival for a subject with the tumor or cancer (e.g., relative to a control subject not receiving a treatment or receiving a placebo treatment). In one example, “treating” refers to delaying progression, expediting remission, inducing remission, augmenting remission, speeding recovery, increasing efficacy of alternative therapeutics, decreasing resistance to alternative therapeutics, or a combination thereof (e.g., relative to a control subject not receiving a treatment or receiving a placebo treatment). The terms “preventing” or “impeding” can refer, for example to delaying the onset of symptoms, preventing relapse of a tumor or cancer, decreasing the number or frequency of relapse episodes, increasing latency between symptomatic episodes, preventing metastasis of a tumor or cancer, or a combination thereof. The terms “suppressing” or “inhibiting” can refer, for example, to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

The term “subject” refers to a mammal (e.g., a human) in need of therapy for, or susceptible to developing, a tumor or a cancer. The term subject also refers to a mammal (e.g., a human) that receives either prophylactic or therapeutic treatment. The subject may include dogs, cats, pigs, cows, sheep, goats, horses, rats, mice, non-human mammals, and humans. The term “subject” does not necessarily exclude an individual that is healthy in all respects and does not have or show signs of cancer or a tumor.

An individual is at increased risk of developing a tumor or a cancer if the subject has at least one known risk-factor (e.g., genetic, biochemical, family history, and situational exposure) placing individuals with that risk factor at a statistically significant greater risk of developing the tumor or cancer than individuals without the risk factor.

A “symptom” or “sign” refers to objective evidence of a disease as observed by a physician or subjective evidence of a disease, such as altered gait, as perceived by the subject. A symptom or sign may be any manifestation of a disease. Symptoms can be primary or secondary. The term “primary” refers to a symptom that is a direct result of a particular disease or disorder (e.g., a tumor or cancer), while the term “secondary” refers to a symptom that is derived from or consequent to a primary cause. The heteroclitic peptides, recombinant fusion polypeptides, nucleic acids encoding the heteroclitic peptides or recombinant fusion polypeptides, the immunogenic compositions, the pharmaceutical compositions, and the vaccines disclosed herein can treat primary or secondary symptoms or secondary complications.

The heteroclitic peptides, recombinant fusion polypeptides, nucleic acids encoding heteroclitic peptides or recombinant fusion polypeptides, recombinant bacteria or Listeria strains, immunogenic compositions, pharmaceutical compositions, or vaccines are administered in an effective regime, meaning a dosage, route of administration, and frequency of administration that delays the onset, reduces the severity, inhibits further deterioration, and/or ameliorates at least one sign or symptom of the tumor or cancer. Alternatively, the heteroclitic peptides, recombinant fusion polypeptides, nucleic acids encoding heteroclitic peptides or recombinant fusion polypeptides, recombinant bacteria or Listeria strains, immunogenic compositions, pharmaceutical compositions, or vaccines are administered in an effective regime, meaning a dosage, route of administration, and frequency of administration that induces an immune response to a heterologous antigen in the heteroclitic peptide or recombinant fusion polypeptide (or encoded by the nucleic acid), the recombinant bacteria or Listeria strain, the immunogenic composition, the pharmaceutical composition, or the vaccine, or in the case of recombinant bacteria or Listeria strains, that induces an immune response to the bacteria or Listeria strain itself. If a subject is already suffering from the tumor or cancer, the regime can be referred to as a therapeutically effective regime. If the subject is at elevated risk of developing the tumor or cancer relative to the general population but is not yet experiencing symptoms, the regime can be referred to as a prophylactically effective regime. In some instances, therapeutic or prophylactic efficacy can be observed in an individual patient relative to historical controls or past experience in the same patient. In other instances, therapeutic or prophylactic efficacy can be demonstrated in a preclinical or clinical trial in a population of treated patients relative to a control population of untreated patients. For example, a regime can be considered therapeutically or prophylactically effective if an individual treated patient achieves an outcome more favorable than the mean outcome in a control population of comparable patients not treated by methods described herein, or if a more favorable outcome is demonstrated in treated patients versus control patients in a controlled clinical trial (e.g., a phase II, phase II/III or phase III trial) at the p <0.05 or 0.01 or even 0.001 level.

Exemplary dosages for a peptide are, for example, 10, 20, 30, 40, 50, 60, 70, 80, 90, 100, 150, 200, 300, 400, 500, 600, 700, 800, 900, 1000, 1500, 2000, 10-20, 20-40, 30-60, 40-60, 40-80, 50-100, 50-150, 60-80, 80-100, 100-200, 200-300, 300-400, 400-600, 500-800, 600-800, 800-1000, 1000-1500, or 1500-1200m peptide per day. Exemplary dosages for a peptide are, for example, 10, 20, 30, 40, 50, 60, 70, 80, 90, 100, 150, 200, 300, 400, 500, 600, 700, 800, 900, 1000, 1500, 2000, 10-20, 20-40, 30-60, 40-60, 40-80, 50-100, 50-150, 60-80, 80-100, 100-200, 200-300, 300-400, 400-600, 500-800, 600-800, 800-1000, 1000-1500, or 1500-1200 mg peptide per day.

Exemplary dosages for a recombinant Listeria strain are, for example, 1×10⁶-1×10⁷ CFU, 1×10⁷-1×10⁸ CFU, 1×10⁸-3.31×10¹⁰ CFU, 1×10⁹-3.31×10¹⁰ CFU, 5-500×10⁸ CFU, 7-500×10⁸ CFU, 10-500×10⁸ CFU, 20-500×10⁸ CFU, 30-500×10⁸ CFU, 50-500×10⁸ CFU, 70-500×10⁸ CFU, 100-500×10⁸ CFU, 150-500×10⁸ CFU, 5-300×10⁸ CFU, 5-200×10⁸ CFU, 5-15×10⁸ CFU, 5-100×10⁸ CFU, 5-70×10⁸ CFU, 5-50×10⁸ CFU, 5-30×10⁸ CFU, 5-20×10⁸ CFU, 1-30×10⁹ CFU, 1-20×10⁹CFU, 2-30×10⁹ CFU, 1-10×10⁹ CFU, 2-10×10⁹ CFU, 3-10×10⁹ CFU, 2-7×10⁹ CFU, 2-5×10⁹ CFU, and 3-5×10⁹ CFU. Other exemplary dosages for a recombinant Listeria strain are, for example, 1×10⁷ organisms, 1.5×10⁷ organisms, 2×10⁸ organisms, 3×10⁷ organisms, 4×10⁷ organisms, 5×10⁷ organisms, 6×10⁷ organisms, 7×10⁷ organisms, 8×10⁷ organisms, 10×10⁷ organisms, 1.5×10⁸ organisms, 2×10⁸ organisms, 2.5×10⁸ organisms, 3×10⁸ organisms, 3.3×10⁸ organisms, 4×10⁸ organisms, 5×10⁸ organisms, 1×10⁹ organisms, 1.5×10⁹ organisms, 2×10⁹ organisms, 3×10⁹ organisms, 4×10⁹ organisms, 5×10⁹ organisms, 6×10⁹ organisms, 7×10⁹ organisms, 8×10⁹ organisms, 10×10⁹ organisms, 1.5×10¹⁰ organisms, 2×10¹⁰ organisms, 2.5×10¹⁰ organisms, 3×10¹⁰ organisms, 3.3×10¹⁰ organisms, 4×10¹⁰ organisms, and 5×10¹⁰ organisms. The dosage can depend on the condition of the patient and response to prior treatment, if any, whether the treatment is prophylactic or therapeutic, and other factors.

Administration can be by any suitable means. For example, administration can be parenteral, intravenous, oral, subcutaneous, intra-arterial, intracranial, intrathecal, intracerebroventricular, intraperitoneal, topical, intranasal, intramuscular, intra-ocular, intrarectal, conjunctival, transdermal, intradermal, vaginal, rectal, intratumoral, parcanceral, transmucosal, intravascular, intraventricular, inhalation (aerosol), nasal aspiration (spray), sublingual, aerosol, suppository, or a combination thereof. For intranasal administration or application by inhalation, solutions or suspensions of the heteroclitic peptides, recombinant fusion polypeptides, nucleic acids encoding heteroclitic peptides or recombinant fusion polypeptides, recombinant bacteria or Listeria strains, immunogenic compositions, pharmaceutical compositions, or vaccines mixed and aerosolized or nebulized in the presence of the appropriate carrier are suitable. Such an aerosol may comprise any heteroclitic peptide, recombinant fusion polypeptide, nucleic acids encoding a heteroclitic peptide or recombinant fusion polypeptide, recombinant bacteria or Listeria strain, immunogenic composition, pharmaceutical composition, or vaccine described herein. Administration may also be in the form of a suppository (e.g., rectal suppository or urethral suppository), in the form of a pellet for subcutaneous implantation (e.g., providing for controlled release over a period of time), or in the form of a capsule. Administration may also be via injection into a tumor site or into a tumor. Regimens of administration can be readily determined based on factors such as exact nature and type of the tumor or cancer being treated, the severity of the tumor or cancer, the age and general physical condition of the subject, body weight of the subject, response of the individual subject, and the like.

The frequency of administration can depend on the half-life of the heteroclitic peptides, recombinant fusion polypeptides, nucleic acids encoding heteroclitic peptides or recombinant fusion polypeptides, recombinant bacteria or Listeria strains, immunogenic compositions, pharmaceutical compositions, or vaccines in the subject, the condition of the subject, and the route of administration, among other factors. The frequency can be, for example, daily, weekly, monthly, quarterly, or at irregular intervals in response to changes in the subject's condition or progression of the tumor or cancer being treated. The course of treatment can depend on the condition of the subject and other factors. For example, the course of treatment can be several weeks, several months, or several years (e.g., up to 2 years). For example, repeat administrations (doses) may be undertaken immediately following the first course of treatment or after an interval of days, weeks or months to achieve tumor regression or suppression of tumor growth. Assessment may be determined by any known technique, including diagnostic methods such as imaging techniques, analysis of serum tumor markers, biopsy, or the presence, absence, or amelioration of tumor-associated symptoms. As a specific example, the heteroclitic peptides, recombinant fusion polypeptides, nucleic acids encoding heteroclitic peptides or recombinant fusion polypeptides, recombinant bacteria or Listeria strains, immunogenic compositions, pharmaceutical compositions, or vaccines can be administered every 3 weeks for up to 2 years. In one example, a heteroclitic peptide, a recombinant fusion polypeptide, a nucleic acid encoding a heteroclitic peptide or recombinant fusion polypeptide, a recombinant bacteria or Listeria strain, an immunogenic composition, a pharmaceutical composition, or a vaccine disclosed herein is administered in increasing doses in order to increase the T-effector cell to regulatory T cell ratio and generate a more potent anti-tumor immune response. Anti-tumor immune responses can be further strengthened by providing the subject with cytokines including, for example, IFN-γ, TNF-α, and other cytokines known to enhance cellular immune response. See, e.g., U.S. Pat. No. 6,991,785, herein incorporated by reference in its entirety for all purposes.

Some methods may further comprise “boosting” the subject with additional heteroclitic peptides, recombinant fusion polypeptides, nucleic acids encoding heteroclitic peptides or recombinant fusion polypeptides, recombinant bacteria or Listeria strains, immunogenic compositions, pharmaceutical compositions, or vaccines or administering the heteroclitic peptides, recombinant fusion polypeptides, nucleic acids encoding heteroclitic peptides or recombinant fusion polypeptides, recombinant bacteria or Listeria strains, immunogenic compositions, pharmaceutical compositions, or vaccines multiple times. “Boosting” refers to administering an additional dose to a subject. For example, in some methods, 2 boosts (or a total of 3 inoculations) are administered, 3 boosts are administered, 4 boosts are administered, 5 boosts are administered, or 6 or more boosts are administered. The number of dosages administered can depend on, for example, the response of the tumor or cancer to the treatment.

Optionally, the heteroclitic peptide, recombinant fusion polypeptide, nucleic acids encoding a heteroclitic peptide or recombinant fusion polypeptide, recombinant bacteria or Listeria strain, immunogenic composition, pharmaceutical composition, or vaccine used in the booster inoculation is the same as the heteroclitic peptide, recombinant fusion polypeptide, nucleic acid encoding a heteroclitic peptide or recombinant fusion polypeptide, recombinant bacteria or Listeria strain, immunogenic composition, pharmaceutical composition, or vaccine used in the initial “priming” inoculation. Alternatively, the booster heteroclitic peptide, recombinant fusion polypeptide, nucleic acid, recombinant bacteria or Listeria strain, immunogenic composition, pharmaceutical composition, or vaccine is different from the priming heteroclitic peptide, recombinant fusion polypeptide, nucleic acid, recombinant bacteria or Listeria strain, immunogenic composition, pharmaceutical composition, or vaccine. Optionally, the same dosages are used in the priming and boosting inoculations. Alternatively, a larger dosage is used in the booster, or a smaller dosage is used in the booster. The period between priming and boosting inoculations can be experimentally determined. For example, the period between priming and boosting inoculations can be 1 week, 2 weeks, 3 weeks, 4 weeks, 5 weeks, 6-8 weeks, or 8-10 weeks.

Heterologous prime boost strategies have been effective for enhancing immune responses and protection against numerous pathogens. See, e.g., Schneider et al. (1999) Immunol. Rev. 170:29-38; Robinson (2002) Nat. Rev. Immunol. 2:239-250; Gonzalo et al. (2002) Vaccine 20:1226-1231; and Tanghe (2001) Infect. Immun. 69:3041-3047, each of which is herein incorporated by reference in its entirety for all purposes. Providing antigen in different forms in the prime and the boost injections can maximize the immune response to the antigen. DNA vaccine priming followed by boosting with protein in adjuvant or by viral vector delivery of DNA encoding antigen is one effective way of improving antigen-specific antibody and CD4+ T-cell responses or CD8+ T-cell responses. See, e.g., Shiver et al. (2002) Nature 415: 331-335; Gilbert et al. (2002) Vaccine 20:1039-1045; Billaut-Mulot et al. (2000) Vaccine 19:95-102; and Sin et al. (1999) DNA Cell Biol. 18:771-779, each of which is herein incorporated by reference in its entirety for all purposes. As one example, adding CRL1005 poloxamer (12 kDa, 5% POE) to DNA encoding an antigen can enhance T-cell responses when subjects are vaccinated with a DNA prime followed by a boost with an adenoviral vector expressing the antigen. See, e.g., Shiver et al. (2002) Nature 415:331-335, herein incorporated by reference in its entirety for all purposes. As another example, a vector construct encoding an immunogenic portion of an antigen and a protein comprising the immunogenic portion of the antigen can be administered. See, e.g., US 2002/0165172, herein incorporated by reference in its entirety for all purposes. Similarly, an immune response of nucleic acid vaccination can be enhanced by simultaneous administration of (e.g., during the same immune response, preferably within 0-10 or 3-7 days of each other) a polynucleotide and polypeptide of interest. See, e.g., U.S. Pat. No. 6,500,432, herein incorporated by reference in its entirety for all purposes.

The therapeutic methods disclosed herein can also comprise administering one or more additional compounds effective in preventing or treating cancer. For example, an additional compound may comprise a compound useful in chemotherapy, such as amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil (5-FU), gemcitabine, gliadelimplants, hydroxycarbamide, idarubicin, ifosfamide, irinotecan, leucovorin, liposomaldoxorubicin, liposomaldaunorubicin, lomustine, melphalan, mercaptopurine, mesna, methotrexate, mitomycin, mitoxantrone, oxaliplatin, paclitaxel (Taxol), pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Alternatively, an additional compound can also comprise other biologics, including Herceptin® (trastuzumab) against the HER2 antigen, Avastin® (bevacizumab) against VEGF, or antibodies to the EGF receptor, such as Erbitux® (cetuximab), and Vectibix® (panitumumab). Alternatively, an additional compound can comprise other immunotherapies. Alternatively, the additional compound can be an indoleamine 2,3-dioxygenase (IDO) pathway inhibitor, such as 1-methyltryptophan (1MT), 1-methyltryptophan (1MT), Necrostatin-1, Pyridoxal Isonicotinoyl Hydrazone, Ebselen, 5-Methylindole-3-carboxaldehyde, CAY10581, an anti-IDO antibody, or a small molecule IDO inhibitor. IDO inhibition can enhance the efficacy of chemotherapeutic agents. The therapeutic methods disclosed herein can also be combined with radiation, stem cell treatment, surgery, or any other treatment.

Such additional compounds or treatments can precede the administration of a heteroclitic peptide, a recombinant fusion polypeptide, a nucleic acid encoding a heteroclitic peptide or recombinant fusion polypeptide, a recombinant bacteria or Listeria strain, an immunogenic composition, a pharmaceutical composition, or a vaccine disclosed herein, follow the administration of a heteroclitic peptide, a recombinant fusion polypeptide, a nucleic acid encoding a heteroclitic peptide or a recombinant fusion polypeptide, a recombinant bacteria or Listeria strain, an immunogenic composition, a pharmaceutical composition, or a vaccine disclosed herein, or be simultaneous to the administration of a heteroclitic peptide, a recombinant fusion polypeptide, a nucleic acid encoding a heteroclitic peptide or a recombinant fusion polypeptide, a recombinant bacteria or Listeria strain, an immunogenic composition, a pharmaceutical composition, or a vaccine disclosed herein.

Targeted immunomodulatory therapy is focused primarily on the activation of costimulatory receptors, for example by using agonist antibodies that target members of the tumor necrosis factor receptor superfamily, including 4-1BB, OX40, and GITR (glucocorticoid-induced TNF receptor-related). The modulation of GITR has demonstrated potential in both antitumor and vaccine settings. Another target for agonist antibodies are co-stimulatory signal molecules for T cell activation. Targeting costimulatory signal molecules may lead to enhanced activation of T cells and facilitation of a more potent immune response. Co-stimulation may also help prevent inhibitory influences from checkpoint inhibition and increase antigen-specific T cell proliferation.

Listeria-based immunotherapy acts by inducing the de novo generation of tumor antigen-specific T cells that infiltrate and destroy the tumor and by reducing the numbers and activities of immunosuppressive regulatory T cells (Tregs) and myeloid-derived suppressor cells (MDSCs) in the tumor microenvironment. Antibodies (or functional fragments thereof) for T cell co-inhibitory or co-stimulatory receptors (e.g., checkpoint inhibitors CTLA-4, PD-1, TIM-3, LAG3 and co-stimulators CD137, OX40, GITR, and CD40) can have synergy with Listeria-based immunotherapy.

Thus, some methods can comprise further administering a composition comprising an immune checkpoint inhibitor antagonist, such as a PD-1 signaling pathway inhibitor, a CD-80/86 and CTLA-4 signaling pathway inhibitor, a T cell membrane protein 3 (TIM3) signaling pathway inhibitor, an adenosine A2a receptor (A2aR) signaling pathway inhibitor, a lymphocyte activation gene 3 (LAG3) signaling pathway inhibitor, a killer immunoglobulin receptor (KIR) signaling pathway inhibitor, a CD40 signaling pathway inhibitor, or any other antigen-presenting cell/T cell signaling pathway inhibitor. Examples of immune checkpoint inhibitor antagonists include an anti-PD-L1/PD-L2 antibody or fragment thereof, an anti-PD-1 antibody or fragment thereof, an anti-CTLA-4 antibody or fragment thereof, or an anti-B7-H4 antibody or fragment thereof. For example, an anti PD-1 antibody can be administered to a subject at 5-10 mg/kg every 2 weeks, 5-10 mg/kg every 3 weeks, 1-2 mg/kg every 3 weeks, 1-10 mg/kg every week, 1-10 mg/kg every 2 weeks, 1-10 mg/kg every 3 weeks, or 1-10 mg/kg every 4 weeks.

Likewise, some methods can further comprise administering a T cell stimulator, such as an antibody or functional fragment thereof binding to a T-cell receptor co-stimulatory molecule, an antigen presenting cell receptor binding co-stimulatory molecule, or a member of the TNF receptor superfamily. The T-cell receptor co-stimulatory molecule can comprise, for example, CD28 or ICOS. The antigen presenting cell receptor binding co-stimulatory molecule can comprise, for example, a CD80 receptor, a CD86 receptor, or a CD46 receptor. The TNF receptor superfamily member can comprise, for example, glucocorticoid-induced TNF receptor (GITR), OX40 (CD134 receptor), 4-1BB (CD137 receptor), or TNFR25.

For example, some methods can further comprise administering an effective amount of a composition comprising an antibody or functional fragment thereof binding to a T-cell receptor co-stimulatory molecule or an antibody or functional fragment thereof binding to an antigen presenting cell receptor binding a co-stimulatory molecule. The antibody can be, for example, an anti-TNF receptor antibody or antigen-binding fragment thereof (e.g., TNF receptor superfamily member glucocorticoid-induced TNF receptor (GITR), OX40 (CD134 receptor), 4-1BB (CD137 receptor), or TNFR25), an anti-OX40 antibody or antigen-binding fragment thereof, or an anti-GITR antibody or antigen binding fragment thereof. Alternatively, other agonistic molecules can be administered (e.g., GITRL, an active fragment of GITRL, a fusion protein containing GITRL, a fusion protein containing an active fragment of GITRL, an antigen presenting cell (APC)/T cell agonist, CD134 or a ligand or fragment thereof, CD137 or a ligand or fragment thereof, or an inducible T cell costimulatory (ICOS) or a ligand or fragment thereof, or an agonistic small molecule).

In a specific example, some methods can further comprise administering an anti-CTLA-4 antibody or a functional fragment thereof and/or an anti-CD137 antibody or functional fragment thereof. For example, the anti-CTLA-4 antibody or a functional fragment thereof or the anti-CD137 antibody or functional fragment thereof can be administered about 72 hours after the first dose of heteroclitic peptide, recombinant fusion polypeptide, nucleic acids encoding a heteroclitic peptide or recombinant fusion polypeptide, recombinant bacteria or Listeria strain, immunogenic composition, pharmaceutical composition, or vaccine, or about 48 hours after the first dose of heteroclitic peptide, recombinant fusion polypeptide, nucleic acids encoding a heteroclitic peptide or recombinant fusion polypeptide, recombinant bacteria or Listeria strain, immunogenic composition, pharmaceutical composition, or vaccine. The anti-CTLA-4 antibody or a functional fragment thereof or anti-CD137 antibody or functional fragment thereof can be administered at a dose, for example, of about 0.05 mg/kg and about 5 mg/kg. A recombinant Listeria strain or immunogenic composition comprising a recombinant Listeria strain can be administered at a dose, for example, of about 1×10⁹ CFU. Some such methods can further comprise administering an effective amount of an anti-PD-1 antibody or functional fragment thereof.

Methods for assessing efficacy of cancer immunotherapies are well known and are described, for example, in Dzojic et al. (2006) Prostate 66(8):831-838; Naruishi et al. (2006) Cancer Gene Ther. 13(7):658-663, Sehgal et al. (2006) Cancer Cell Int. 6:21), and Heinrich et al. (2007) Cancer Immunol Immunother 56(5):725-730, each of which is herein incorporated by reference in its entirety for all purposes. As one example, for prostate cancer, a prostate cancer model can be to test methods and compositions disclosed herein, such as a TRAMP-C2 mouse model, a 178-2 BMA cell model, a PAIII adenocarcinoma cells model, a PC-3M model, or any other prostate cancer model.

Alternatively or additionally, the immunotherapy can be tested in human subjects, and efficacy can be monitored using known. Such methods can include, for example, directly measuring CD4+ and CD8+ T cell responses, or measuring disease progression (e.g., by determining the number or size of tumor metastases, or monitoring disease symptoms such as cough, chest pain, weight loss, and so forth). Methods for assessing the efficacy of a cancer immunotherapy in human subjects are well known and are described, for example, in Uenaka et al. (2007) Cancer Immun. 7:9 and Thomas-Kaskel et al. (2006) Int J Cancer 119(10):2428-2434, each of which is herein incorporated by reference in its entirety for all purposes.

### VII. Kits

Also provided are kits comprising a reagent utilized in performing a method disclosed herein or kits comprising a composition, tool, or instrument disclosed herein.

For example, such kits can comprise a heteroclitic peptide or recombinant fusion polypeptide disclosed herein, a nucleic acid encoding a heteroclitic peptide or recombinant fusion polypeptide disclosed herein, a recombinant bacteria or Listeria strain disclosed herein, an immunogenic composition disclosed herein, a pharmaceutical composition disclosed herein, or a vaccine disclosed herein. Such kits can additionally comprise an instructional material which describes use of the peptide or recombinant fusion polypeptide, the nucleic acid encoding the peptide or recombinant fusion polypeptide, the recombinant Listeria strain, the immunogenic composition, the pharmaceutical composition, or the vaccine to perform the methods disclosed herein. Such kits can optionally further comprise an applicator. Although model kits are described below, the contents of other useful kits will be apparent in light of the present disclosure.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### Listing of Embodiments

The subject matter disclosed herein includes, but is not limited to, the following embodiments.

1. An isolated peptide comprising an immunogenic fragment of a cancer-associated protein, wherein the fragment comprises a heteroclitic mutation.

2. The isolated peptide of embodiment 1, wherein the heteroclitic mutation is a mutation to a preferred amino acid at an anchor position.

3. The isolated peptide of embodiment 1 or 2, wherein the fragment is between about 7 and about 11 amino acids in length, between about 8 and about 10 amino acids in length, or about 9 amino acids in length.

4. The isolated peptide of any preceding embodiment, wherein the cancer-associated protein is a cancer testis antigen or oncofetal antigen.

5. The isolated peptide of any preceding embodiment, wherein the cancer-associated protein is encoded by one of the following human genes: CEACAM5, GAGE1, TERT, KLHL7, MAGEA3, MAGEA4, MAGEA6, NUF2, NYESO1, PAGE4, PRAME, PSA, PSMA, RNF43, SART3, SSX2, STEAP1, and SURVIVIN.

6. The isolated peptide of embodiment 5, wherein: (a) the cancer-associated protein is encoded by CEACAM5, and the fragment comprises any one of SEQ ID NOS: 100, 102, 104, 106, and 108; (b) the cancer-associated protein is encoded by GAGE1, and the fragment comprises any one of SEQ ID NOS: 110 and 112; (c) the cancer-associated protein is encoded by TERT, and the fragment comprises SEQ ID NO: 114; (d) the cancer-associated protein is encoded by KLHL7, and the fragment comprises SEQ ID NO: 116; (e) the cancer-associated protein is encoded by MAGEA3, and the fragment comprises any one of SEQ ID NOS: 118, 120, 122, and 124; (f) the cancer-associated protein is encoded by MAGEA4, and the fragment comprises SEQ ID NO: 126; (g) the cancer-associated protein is encoded by MAGEA6, and the fragment comprises SEQ ID NO: 128; (h) the cancer-associated protein is encoded by NUF2, and the fragment comprises any one of SEQ ID NOS: 130 and 132; (i) the cancer-associated protein is encoded by NYESO1, and the fragment comprises any one of SEQ ID NOS: 134 and 136; (j) the cancer-associated protein is encoded by PAGE4, and the fragment comprises SEQ ID NO: 138; (k) the cancer-associated protein is encoded by PRAME, and the fragment comprises SEQ ID NO: 140; (1) the cancer-associated protein is encoded by PSA, and the fragment comprises SEQ ID NO: 142; (m) the cancer-associated protein is encoded by PSMA, and the fragment comprises SEQ ID NO: 144; (n) the cancer-associated protein is encoded by RNF43, and the fragment comprises SEQ ID NO: 146; (o) the cancer-associated protein is encoded by SART3, and the fragment comprises SEQ ID NO: 148; (p) the cancer-associated protein is encoded by SSX2, and the fragment comprises SEQ ID NO: 150; (q) the cancer-associated protein is encoded by STEAP1, and the fragment comprises any one of SEQ ID NOS: 152 and 154; or (r) the cancer-associated protein is encoded by SURVIVIN, and the fragment comprises any one of SEQ ID NOS: 156 and 158.

7. The isolated peptide of embodiment 6, wherein: (a) the cancer-associated protein is encoded by CEACAM5, and the fragment consists of any one of SEQ ID NOS: 100, 102, 104, 106, and 108; (b) the cancer-associated protein is encoded by GAGE1, and the fragment consists of any one of SEQ ID NOS: 110 and 112; (c) the cancer-associated protein is encoded by TERT, and the fragment consists of SEQ ID NO: 114; (d) the cancer-associated protein is encoded by KLHL7, and the fragment consists of SEQ ID NO: 116; (e) the cancer-associated protein is encoded by MAGEA3, and the fragment consists of any one of SEQ ID NOS: 118, 120, 122, and 124; (f) the cancer-associated protein is encoded by MAGEA4, and the fragment consists of SEQ ID NO: 126; (g) the cancer-associated protein is encoded by MAGEA6, and the fragment consists of SEQ ID NO: 128; (h) the cancer-associated protein is encoded by NUF2, and the fragment consists of any one of SEQ ID NOS: 130 and 132; (i) the cancer-associated protein is encoded by NYESO1, and the fragment consists of any one of SEQ ID NOS: 134 and 136; (j) the cancer-associated protein is encoded by PAGE4, and the fragment consists of SEQ ID NO: 138; (k) the cancer-associated protein is encoded by PRAME, and the fragment consists of SEQ ID NO: 140; (1) the cancer-associated protein is encoded by PSA, and the fragment consists of SEQ ID NO: 142; (m) the cancer-associated protein is encoded by PSMA, and the fragment consists of SEQ ID NO: 144; (n) the cancer-associated protein is encoded by RNF43, and the fragment consists of SEQ ID NO: 146; (o) the cancer-associated protein is encoded by SART3, and the fragment consists of SEQ ID NO: 148; (p) the cancer-associated protein is encoded by SSX2, and the fragment consists of SEQ ID NO: 150; (q) the cancer-associated protein is encoded by STEAP1, and the fragment consists of any one of SEQ ID NOS: 152 and 154; or (r) the cancer-associated protein is encoded by SURVIVIN, and the fragment consists of any one of SEQ ID NOS: 156 and 158.

8. The isolated peptide of embodiment 7, wherein: (a) the cancer-associated protein is encoded by CEACAM5, and the isolated peptide consists of any one of SEQ ID NOS: 100, 102, 104, 106, and 108; (b) the cancer-associated protein is encoded by GAGE1, and the isolated peptide consists of any one of SEQ ID NOS: 110 and 112; (c) the cancer-associated protein is encoded by TERT, and the isolated peptide consists of SEQ ID NO: 114; (d) the cancer-associated protein is encoded by KLHL7, and the isolated peptide consists of SEQ ID NO: 116; (e) the cancer-associated protein is encoded by MAGEA3, and the isolated peptide consists of any one of SEQ ID NOS: 118, 120, 122, and 124; (f) the cancer-associated protein is encoded by MAGEA4, and the isolated peptide consists of SEQ ID NO: 126; (g) the cancer-associated protein is encoded by MAGEA6, and the isolated peptide consists of SEQ ID NO: 128; (h) the cancer-associated protein is encoded by NUF2, and the isolated peptide consists of any one of SEQ ID NOS: 130 and 132; (i) the cancer-associated protein is encoded by NYESO1, and the isolated peptide consists of any one of SEQ ID NOS: 134 and 136; (j) the cancer-associated protein is encoded by PAGE4, and the isolated peptide consists of SEQ ID NO: 138; (k) the cancer-associated protein is encoded by PRAME, and the isolated peptide consists of SEQ ID NO: 140; (1) the cancer-associated protein is encoded by PSA, and the isolated peptide consists of SEQ ID NO: 142; (m) the cancer-associated protein is encoded by PSMA, and the isolated peptide consists of SEQ ID NO: 144; (n) the cancer-associated protein is encoded by RNF43, and the isolated peptide consists of SEQ ID NO: 146; (o) the cancer-associated protein is encoded by SART3, and the isolated peptide consists of SEQ ID NO: 148; (p) the cancer-associated protein is encoded by SSX2, and the isolated peptide consists of SEQ ID NO: 150; (q) the cancer-associated protein is encoded by STEAP1, and the isolated peptide consists of any one of SEQ ID NOS: 152 and 154; or (r) the cancer-associated protein is encoded by SURVIVIN, and the isolated peptide consists of any one of SEQ ID NOS: 156 and 158.

9. The isolated peptide of any preceding embodiment, wherein the fragment binds to one or more of the following HLA types: HLA-A*02:01, HLA-A*03:01, HLA-A*24:02, and HLA-B*07:02.

10. A nucleic acid encoding the isolated peptide of any preceding embodiment.

11. The nucleic acid of embodiment 10, wherein the nucleic acid is codon optimized for expression in humans.

12. The nucleic acid of embodiment 10, wherein the nucleic acid is codon optimized for expression in Listeria monocytogenes.

13. The nucleic acid of any one of embodiments 10-12, wherein the nucleic acid comprises DNA.

14. The nucleic acid of any one of embodiments 10-12, wherein the nucleic acid comprises RNA.

15. The nucleic acid of any one of embodiments 10-14, wherein the nucleic acid comprises a sequence selected from any one of SEQ ID NOS: 223-977 and degenerate variants thereof that encode the same amino acid sequence.

16. The nucleic acid of embodiment 15, wherein the nucleic acid consists of a sequence selected from any one of SEQ ID NOS: 223-977 and degenerate variants thereof that encode the same amino acid sequence.

17. A pharmaceutical composition comprising: (a) one or more isolated peptides of any one of embodiments 1-9 or one or more nucleic acids of any one of embodiments 10-16; and (b) an adjuvant.

18. The pharmaceutical composition of embodiment 17, wherein the adjuvant comprises a detoxified listeriolysin O (dtLLO), a granulocyte/macrophage colony-stimulating factor (GM-CSF) protein, a nucleotide molecule encoding a GM-CSF protein, saponin QS21, monophosphoryl lipid A, an unmethylated CpG-containing oligonucleotide, or Montanide ISA 51.

19. The pharmaceutical composition of embodiment 17 or 18, wherein the pharmaceutical composition comprises peptides or nucleic acids encoding peptides that bind to each of the following HLA types: HLA-A*02:01, HLA-A*03:01, HLA-A*24:02, and HLA-B *07:02.

20. The pharmaceutical composition of any one of embodiments 17-19, wherein the pharmaceutical composition comprises: (a) two or more of the peptides set forth in Table 3 or nucleic acids encoding two or more of the peptides set forth in Table 3; (b) two or more of the peptides set forth in Table 5 or nucleic acids encoding two or more of the peptides set forth in Table 5; (c) two or more of the peptides set forth in Table 7 or nucleic acids encoding two or more of the peptides set forth in Table 7; (d) two or more of the peptides set forth in Table 9 or nucleic acids encoding two or more of the peptides set forth in Table 9; (e) two or more of the peptides set forth in Table 11 or nucleic acids encoding two or more of the peptides set forth in Table 11; (f) two or more of the peptides set forth in Table 13 or nucleic acids encoding two or more of the peptides set forth in Table 13; (g) two or more of the peptides set forth in Table 15 or nucleic acids encoding two or more of the peptides set forth in Table 15; (h) two or more of the peptides set forth in Table 17 or nucleic acids encoding two or more of the peptides set forth in Table 17; (i) two or more of the peptides set forth in Table 19 or nucleic acids encoding two or more of the peptides set forth in Table 19; or (j) two or more of the peptides set forth in Table 21 or nucleic acids encoding two or more of the peptides set forth in Table 21.

21. The pharmaceutical composition of embodiment 20, wherein the pharmaceutical composition comprises: (a) all of the peptides set forth in Table 3 or nucleic acids encoding all of the peptides set forth in Table 3; (b) all of the peptides set forth in Table 5 or nucleic acids encoding all of the peptides set forth in Table 5; (c) all of the peptides set forth in Table 7 or nucleic acids encoding all of the peptides set forth in Table 7; (d) all of the peptides set forth in Table 9 or nucleic acids encoding all of the peptides set forth in Table 9; (e) all of the peptides set forth in Table 11 or nucleic acids encoding all of the peptides set forth in Table 11; (f) all of the peptides set forth in Table 13 or nucleic acids encoding all of the peptides set forth in Table 13; (g) all of the peptides set forth in Table 15 or nucleic acids encoding all of the peptides set forth in Table 15; (h) all of the peptides set forth in Table 17 or nucleic acids encoding all of the peptides set forth in Table 17; (i) all of the peptides set forth in Table 19 or nucleic acids encoding all of the peptides set forth in Table 19; or (j) all of the peptides set forth in Table 21 or nucleic acids encoding all of the peptides set forth in Table 21.

22. A recombinant bacteria strain comprising a nucleic acid encoding any one of the isolated peptides of embodiments 1-9.

23. A recombinant bacteria strain comprising one or more nucleic acids encoding two or more of the isolated peptides of embodiments 1-9.

24. The recombinant bacteria strain of embodiment 23, wherein the two or more peptides comprise: (a) two or more of the peptides set forth in Table 3 or nucleic acids encoding two or more of the peptides set forth in Table 3; (b) two or more of the peptides set forth in Table 5 or nucleic acids encoding two or more of the peptides set forth in Table 5; (c) two or more of the peptides set forth in Table 7 or nucleic acids encoding two or more of the peptides set forth in Table 7; (d) two or more of the peptides set forth in Table 9 or nucleic acids encoding two or more of the peptides set forth in Table 9; (e) two or more of the peptides set forth in Table 11 or nucleic acids encoding two or more of the peptides set forth in Table 11; (f) two or more of the peptides set forth in Table 13 or nucleic acids encoding two or more of the peptides set forth in Table 13; (g) two or more of the peptides set forth in Table 15 or nucleic acids encoding two or more of the peptides set forth in Table 15; (h) two or more of the peptides set forth in Table 17 or nucleic acids encoding two or more of the peptides set forth in Table 17; (i) two or more of the peptides set forth in Table 19 or nucleic acids encoding two or more of the peptides set forth in Table 19; or (j) two or more of the peptides set forth in Table 21 or nucleic acids encoding two or more of the peptides set forth in Table 21.

25. The recombinant bacteria strain of embodiment 24, wherein the two or more peptides comprise: (a) all of the peptides set forth in Table 3 or nucleic acids encoding all of the peptides set forth in Table 3; (b) all of the peptides set forth in Table 5 or nucleic acids encoding all of the peptides set forth in Table 5; (c) all of the peptides set forth in Table 7 or nucleic acids encoding all of the peptides set forth in Table 7; (d) all of the peptides set forth in Table 9 or nucleic acids encoding all of the peptides set forth in Table 9; (e) all of the peptides set forth in Table 11 or nucleic acids encoding all of the peptides set forth in Table 11; (f) all of the peptides set forth in Table 13 or nucleic acids encoding all of the peptides set forth in Table 13; (g) all of the peptides set forth in Table 15 or nucleic acids encoding all of the peptides set forth in Table 15; (h) all of the peptides set forth in Table 17 or nucleic acids encoding all of the peptides set forth in Table 17; (i) all of the peptides set forth in Table 19 or nucleic acids encoding all of the peptides set forth in Table 19; or (j) all of the peptides set forth in Table 21 or nucleic acids encoding all of the peptides set forth in Table 21.

26. The recombinant bacteria strain of any one of embodiments 23-25, wherein the combination of two or more peptides binds to each of the following HLA types: HLA-A*02:01, HLA-A*03:01, HLA-A*24:02, and HLA-B*07:02.

27. The recombinant bacteria strain of any one of embodiments 22-26, wherein the bacteria strain is a Salmonella, Listeria, Yersinia, Shigella, or Mycobacterium strain.

28. The recombinant bacteria strain of embodiment 27, wherein the bacteria strain is a Listeria strain, optionally wherein the Listeria strain is a Listeria monocytogenes strain.

29. A recombinant Listeria strain comprising a nucleic acid comprising a first open reading frame encoding a fusion polypeptide, wherein the fusion polypeptide comprises a PEST-containing peptide fused to an immunogenic fragment of a cancer-associated protein, wherein the fragment comprises a heteroclitic mutation.

30. The recombinant Listeria strain of embodiment 29, wherein the heteroclitic mutation is a mutation to a preferred amino acid at an anchor position.

31. The recombinant Listeria strain of embodiment 29 or 30, wherein the fragment is between about 7 and about 11 amino acids in length, between about 8 and about 10 amino acids in length, or about 9 amino acids in length.

32. The recombinant Listeria strain of any one of embodiments 29-31, wherein the cancer-associated protein is a cancer testis antigen or oncofetal antigen.

33. The recombinant Listeria strain of any one of embodiments 29-32, wherein the cancer-associated protein is encoded by one of the following human genes: CEACAM5, GAGE1, TERT, KLHL7, MAGEA3, MAGEA4, MAGEA6, NUF2, NYESO1, PAGE4, PRAME, PSA, PSMA, RNF43, SART3, SSX2, STEAP1, and SURVIVIN.

34. The recombinant Listeria strain of embodiment 33, wherein: (a) the cancer-associated protein is encoded by CEACAM5, and the fragment comprises any one of SEQ ID NOS: 100, 102, 104, 106, and 108; (b) the cancer-associated protein is encoded by GAGE1, and the fragment comprises any one of SEQ ID NOS: 110 and 112; (c) the cancer-associated protein is encoded by TERT, and the fragment comprises SEQ ID NO: 114; (d) the cancer-associated protein is encoded by KLHL7, and the fragment comprises SEQ ID NO: 116; (e) the cancer-associated protein is encoded by MAGEA3, and the fragment comprises any one of SEQ ID NOS: 118, 120, 122, and 124; (f) the cancer-associated protein is encoded by MAGEA4, and the fragment comprises SEQ ID NO: 126; (g) the cancer-associated protein is encoded by MAGEA6, and the fragment comprises SEQ ID NO: 128; (h) the cancer-associated protein is encoded by NUF2, and the fragment comprises any one of SEQ ID NOS: 130 and 132; (i) the cancer-associated protein is encoded by NYESO1, and the fragment comprises any one of SEQ ID NOS: 134 and 136; (j) the cancer-associated protein is encoded by PAGE4, and the fragment comprises SEQ ID NO: 138; (k) the cancer-associated protein is encoded by PRAME, and the fragment comprises SEQ ID NO: 140; (1) the cancer-associated protein is encoded by PSA, and the fragment comprises SEQ ID NO: 142; (m) the cancer-associated protein is encoded by PSMA, and the fragment comprises SEQ ID NO: 144; (n) the cancer-associated protein is encoded by RNF43, and the fragment comprises SEQ ID NO: 146; (o) the cancer-associated protein is encoded by SART3, and the fragment comprises SEQ ID NO: 148; (p) the cancer-associated protein is encoded by SSX2, and the fragment comprises SEQ ID NO: 150; (q) the cancer-associated protein is encoded by STEAP1, and the fragment comprises any one of SEQ ID NOS: 152 and 154; or (r) the cancer-associated protein is encoded by SURVIVIN, and the fragment comprises any one of SEQ ID NOS: 156 and 158.

35. The recombinant Listeria strain of embodiment 34, wherein: (a) the cancer-associated protein is encoded by CEACAM5, and the fragment consists of any one of SEQ ID NOS: 100, 102, 104, 106, and 108; (b) the cancer-associated protein is encoded by GAGE1, and the fragment consists of any one of SEQ ID NOS: 110 and 112; (c) the cancer-associated protein is encoded by TERT, and the fragment consists of SEQ ID NO: 114; (d) the cancer-associated protein is encoded by KLHL7, and the fragment consists of SEQ ID NO: 116; (e) the cancer-associated protein is encoded by MAGEA3, and the fragment consists of any one of SEQ ID NOS: 118, 120, 122, and 124; (f) the cancer-associated protein is encoded by MAGEA4, and the fragment consists of SEQ ID NO: 126; (g) the cancer-associated protein is encoded by MAGEA6, and the fragment consists of SEQ ID NO: 128; (h) the cancer-associated protein is encoded by NUF2, and the fragment consists of any one of SEQ ID NOS: 130 and 132; (i) the cancer-associated protein is encoded by NYESO1, and the fragment consists of any one of SEQ ID NOS: 134 and 136; (j) the cancer-associated protein is encoded by PAGE4, and the fragment consists of SEQ ID NO: 138; (k) the cancer-associated protein is encoded by PRAME, and the fragment consists of SEQ ID NO: 140; (1) the cancer-associated protein is encoded by PSA, and the fragment consists of SEQ ID NO: 142; (m) the cancer-associated protein is encoded by PSMA, and the fragment consists of SEQ ID NO: 144; (n) the cancer-associated protein is encoded by RNF43, and the fragment consists of SEQ ID NO: 146; (o) the cancer-associated protein is encoded by SART3, and the fragment consists of SEQ ID NO: 148; (p) the cancer-associated protein is encoded by SSX2, and the fragment consists of SEQ ID NO: 150; (q) the cancer-associated protein is encoded by STEAP1, and the fragment consists of any one of SEQ ID NOS: 152 and 154; or (r) the cancer-associated protein is encoded by SURVIVIN, and the fragment consists of any one of SEQ ID NOS: 156 and 158.

36. The recombinant Listeria strain of any one of embodiments 29-35, wherein the fragment binds to one or more of the following HLA types: HLA-A*02:01, HLA-A*03:01, HLA-A*24:02, and HLA-B*07:02.

37. The recombinant Listeria strain of any one of embodiments 29-36, wherein the PEST-containing peptide comprises a bacterial secretion signal sequence, and the fusion polypeptide further comprises a ubiquitin protein fused to the fragment, wherein the PEST-containing peptide, the ubiquitin, and the carboxy-terminal antigenic peptide are arranged in tandem from the amino-terminal end to the carboxy-terminal end of the fusion polypeptide.

38. The recombinant Listeria strain of any one of embodiments 29-37, wherein the fusion polypeptide comprises the PEST-containing peptide fused to two or more immunogenic fragments of cancer-associated proteins, wherein each of the two or more fragments comprises a heteroclitic mutation.

39. The recombinant Listeria strain of embodiment 38, wherein the two or more immunogenic fragments are fused directly to each other without intervening sequence.

40. The recombinant Listeria strain of embodiment 38, wherein the two or more immunogenic fragments are linked to each other via peptide linkers.

41. The recombinant Listeria strain of embodiment 40, wherein one or more of the linkers set forth in SEQ ID NOS: 209-217 are used to link the two or more immunogenic fragments.

42. The recombinant Listeria strain of any one of embodiments 38-41, wherein the combination of two or more immunogenic fragments in the fusion polypeptide binds to each of the following HLA types: HLA-A*02:01, HLA-A*03:01, HLA-A*24:02, and HLA-B*07:02.

43. The recombinant Listeria strain of any one of embodiments 38-42, wherein the two or more immunogenic fragments comprise: (a) two or more of the peptides set forth in Table 3; (b) two or more of the peptides set forth in Table 5; (c) two or more of the peptides set forth in Table 7; (d) two or more of the peptides set forth in Table 9; (e) two or more of the peptides set forth in Table 11; (f) two or more of the peptides set forth in Table 13; (g) two or more of the peptides set forth in Table 15; (h) two or more of the peptides set forth in Table 17; (i) two or more of the peptides set forth in Table 19; or (j) two or more of the peptides set forth in Table 21.

44. The recombinant Listeria strain of embodiment 43, wherein the two or more immunogenic fragments comprise: (a) all of the peptides set forth in Table 3; (b) all of the peptides set forth in Table 5; (c) all of the peptides set forth in Table 7; (d) all of the peptides set forth in Table 9; (e) all of the peptides set forth in Table 11; (f) all of the peptides set forth in Table 13; (g) all of the peptides set forth in Table 15; (h) all of the peptides set forth in Table 17; (i) all of the peptides set forth in Table 19; or (j) all of the peptides set forth in Table 21.

45. The recombinant Listeria strain of any one of embodiments 29-44, wherein the PEST-containing peptide is on the N-terminal end of the fusion polypeptide.

46. The recombinant Listeria strain of embodiment 45, wherein the PEST-containing peptide is an N-terminal fragment of LLO.

47. The recombinant Listeria strain of embodiment 46, wherein the N-terminal fragment of LLO has the sequence set forth in SEQ ID NO: 59.

48. The recombinant Listeria strain of any one of embodiments 29-47, wherein the nucleic acid is in an episomal plasmid.

49. The recombinant Listeria strain of any one of embodiments 29-48, wherein the nucleic acid does not confer antibiotic resistance upon the recombinant Listeria strain.

50. The recombinant Listeria strain of any one of embodiments 29-49, wherein the recombinant Listeria strain is an attenuated, auxotrophic Listeria strain.

51. The recombinant Listeria strain of embodiment 50, wherein the attenuated, auxotrophic Listeria strain comprises a mutation in one or more endogenous genes that inactivates the one or more endogenous genes.

52. The recombinant Listeria strain of embodiment 51, wherein the one or more endogenous genes comprise actA, dal, and dat.

53. The recombinant Listeria strain of any one of embodiments 29-52, wherein the nucleic acid comprises a second open reading frame encoding a metabolic enzyme.

54. The recombinant Listeria strain of embodiment 53, wherein the metabolic enzyme is an alanine racemase enzyme or a D-amino acid aminotransferase enzyme.

55. The recombinant Listeria strain of any one of embodiments 29-54, wherein the fusion polypeptide is expressed from an hly promoter.

56. The recombinant Listeria strain of any one of embodiments 29-55, wherein the recombinant Listeria strain is a recombinant Listeria monocytogenes strain.

57. The recombinant Listeria strain of any one of embodiments 29-56, wherein the recombinant Listeria strain is an attenuated Listeria monocytogenes strain comprising a deletion of or inactivating mutation in actA, dal, and dat, wherein the nucleic acid is in an episomal plasmid and comprises a second open reading frame encoding an alanine racemase enzyme or a D-amino acid aminotransferase enzyme, and wherein the PEST-containing peptide is an N-terminal fragment of LLO.

58. An immunogenic composition comprising: (a) the recombinant bacteria strain of any one of embodiments 22-28 or the recombinant Listeria strain of any one of embodiments 29-57; and (b) an adjuvant.

59. The immunogenic composition of embodiment 58, wherein the adjuvant comprises a detoxified listeriolysin O (dtLLO), a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide

60. A method of inducing or enhancing an immune response against a tumor or cancer in a subject, comprising administering to the subject the isolated peptide of any one of embodiments 1-9, the nucleic acid of any one of embodiments 10-16, the pharmaceutical composition of any one of embodiments 17-21, the recombinant bacteria strain of any one of embodiments 22-28, the recombinant Listeria strain of any one of embodiments 29-57, or the immunogenic composition of any one of embodiments 58-59.

61. A method of preventing or treating a tumor or cancer in a subject, comprising administering to the subject the isolated peptide of any one of embodiments 1-9, the nucleic acid of any one of embodiments 10-16, the pharmaceutical composition of any one of embodiments 17-21, the recombinant bacteria strain of any one of embodiments 22-28, the recombinant Listeria strain of any one of embodiments 29-57, or the immunogenic composition of any one of embodiments 58-59.

62. The method of embodiment 60 or 61, wherein the cancer is non-small cell lung cancer, prostate cancer, pancreatic cancer, bladder cancer, breast cancer, uterine cancer, ovarian cancer, low-grade glioma, colorectal cancer, or head and neck cancer.

### Brief Description of the Sequences

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

| SEQ ID NO | Type | Description |
|---|---|---|
| 1 | DNA | SIINFEKL Tag v1 |
| 2 | DNA | SIINFEKL Tag v2 |
| 3 | DNA | SIINFEKL Tag v3 |
| 4 | DNA | SIINFEKL Tag v4 |
| 5 | DNA | SIINFEKL Tag v5 |
| 6 | DNA | SIINFEKL Tag v6 |
| 7 | DNA | SIINFEKL Tag v7 |
| 8 | DNA | SIINFEKL Tag v8 |
| 9 | DNA | SIINFEKL Tag v9 |
| 10 | DNA | SIINFEKL Tag v10 |
| 11 | DNA | SIINFEKL Tag v11 |
| 12 | DNA | SIINFEKL Tag v12 |
| 13 | DNA | SIINFEKL Tag v13 |
| 14 | DNA | SIINFEKL Tag v14 |
| 15 | DNA | SIINFEKL Tag v15 |
| 16 | Protein | SIINFEKL Tag |
| 17 | DNA | 3xFLAG Tag v1 |
| 18 | DNA | 3xFLAG Tag v2 |
| 19 | DNA | 3xFLAG Tag v3 |
| 20 | DNA | 3xFLAG Tag v4 |
| 21 | DNA | 3xFLAG Tag v5 |
| 22 | DNA | 3xFLAG Tag v6 |
| 23 | DNA | 3xFLAG Tag v7 |
| 24 | DNA | 3xFLAG Tag v8 |
| 25 | DNA | 3xFLAG Tag v9 |
| 26 | DNA | 3xFLAG Tag v10 |
| 27 | DNA | 3xFLAG Tag v11 |
| 28 | DNA | 3xFLAG Tag v12 |
| 29 | DNA | 3xFLAG Tag v13 |
| 30 | DNA | 3xFLAG Tag v14 |
| 31 | DNA | 3xFLAG Tag v15 |
| 32 | Protein | 3xFLAG Tag |
| 33 | Protein | Peptide Linker v1 |
| 34 | Protein | Peptide Linker v2 |
| 35 | Protein | Peptide Linker v3 |
| 36 | Protein | Peptide Linker v4 |
| 37 | Protein | Peptide Linker v5 |
| 38 | Protein | Peptide Linker v6 |
| 39 | Protein | Peptide Linker v7 |
| 40 | Protein | Peptide Linker v8 |
| 41 | Protein | Peptide Linker v9 |
| 42 | Protein | Peptide Linker v10 |
| 43 | Protein | PEST-Like Sequence v1 |
| 44 | Protein | PEST-Like Sequence v2 |
| 45 | Protein | PEST-Like Sequence v3 |
| 46 | Protein | PEST-Like Sequence v4 |
| 47 | Protein | PEST-Like Sequence v5 |
| 48 | Protein | PEST-Like Sequence v6 |
| 49 | Protein | PEST-Like Sequence v7 |
| 50 | Protein | PEST-Like Sequence v8 |
| 51 | Protein | PEST-Like Sequence v9 |
| 52 | Protein | PEST-Like Sequence v10 |
| 53 | Protein | PEST-Like Sequence v11 |
| 54 | Protein | PEST-Like Sequence v12 |
| 55 | Protein | LLO Protein v1 |
| 56 | Protein | LLO Protein v2 |
| 57 | Protein | N-Terminal Truncated LLO v1 |
| 58 | Protein | N-Terminal Truncated LLO v2 |
| 59 | Protein | N-Terminal Truncated LLO v3 |
| 60 | DNA | Nucleic Acid Encoding N-Terminal Truncated LLO v3 |
| 61 | Protein | ActA Protein v1 |
| 62 | Protein | ActA Protein v2 |
| 63 | Protein | ActA Fragment v1 |
| 64 | Protein | ActA Fragment v2 |
| 65 | Protein | ActA Fragment v3 |
| 66 | Protein | ActA Fragment v4 |
| 67 | Protein | ActA Fragment v5 |
| 68 | DNA | Nucleic Acid Encoding ActA Fragment v5 |
| 69 | Protein | ActA Fragment v6 |
| 70 | Protein | ActA Fragment v7 |
| 71 | DNA | Nucleic Acid Encoding ActA Fragment v7 |
| 72 | Protein | ActA Fragment Fused to Hly Signal Peptide |
| 73 | Protein | ActA Substitution |
| 74 | Protein | Cholesterol-Binding Domain of LLO |
| 75 | Protein | HLA-A2 restricted Epitope from NY-ESO-1 |
| 76 | Protein | Lm Alanine Racemase |
| 77 | Protein | Lm D-Amino Acid Aminotransferase |
| 78 | DNA | Nucleic Acid Encoding Lm Alanine Racemase |
| 79 | DNA | Nucleic Acid Encoding Lm D-Amino Acid Aminotransferase |
| 80 | Protein | Wild Type PrfA |
| 81 | DNA | Nucleic Acid Encoding Wild Type PrfA |
| 82 | Protein | D133V PrfA |
| 83 | DNA | Nucleic Acid Encoding D133V PrfA |
| 84 | DNA | 4X Glycine Linker G1 |
| 85 | DNA | 4X Glycine Linker G2 |
| 86 | DNA | 4X Glycine Linker G3 |
| 87 | DNA | 4X Glycine Linker G4 |
| 88 | DNA | 4X Glycine Linker G5 |
| 89 | DNA | 4X Glycine Linker G6 |
| 90 | DNA | 4X Glycine Linker G7 |
| 91 | DNA | 4X Glycine Linker G8 |
| 92 | DNA | 4X Glycine Linker G9 |
| 93 | DNA | 4X Glycine Linker G10 |
| 94 | DNA | 4X Glycine Linker G11 |
| 95 | Protein | Detoxified Listeriolysin O (dtLLO) |
| 96 | Protein | Modified Cholesterol-Binding Domain of dtLLO |
| 97 | Protein | LLO Signal Sequence |
| 98 | Protein | ActA Signal Sequence |
| 99 | Protein | Variant FLAG Tag |
| 100-159 | Protein | Heteroclitic Peptides and Corresponding Native Peptides |
| 160-169 | Protein | Heteroclitic WT1 Peptides |
| 170 | Protein | Protein Encoded by CEACAM5 |
| 171 | Protein | Protein Encoded by GAGE1 |
| 172 | Protein | Protein Encoded by TERT |
| 173 | Protein | Protein Encoded by KLHL7 |
| 174 | Protein | Protein Encoded by MAGEA3 |
| 175 | Protein | Protein Encoded by MAGEA4 |
| 176 | Protein | Protein Encoded by MAGEA6 |
| 177 | Protein | Protein Encoded by NUF2 |
| 178 | Protein | Protein Encoded by NYESO1 |
| 179 | Protein | Protein Encoded by PAGE4 |
| 180 | Protein | Protein Encoded by PRAME |
| 181 | Protein | Protein Encoded by PSA |
| 182 | Protein | Protein Encoded by PSMA |
| 183 | Protein | Protein Encoded by RNF43 |
| 184 | Protein | Protein Encoded by SART3 |
| 185 | Protein | Protein Encoded by SSX2 |
| 186 | Protein | Protein Encoded by STEAP1 |
| 187 | Protein | Protein Encoded by SURVIVIN |
| 188 | Protein | Ubiquitin |
| 189 | Protein | WT1-FLAG-Ub-heteroclitic phenylalanine minigene construct |
| 190 | Protein | Wild-Type WT1 Peptide v14-WT1-427 long |
| 191 | Protein | Wild-Type WT1 Peptide v15-WT1-331 long |
| 192 | Protein | Heteroclitic WT1 Peptide v1D (WT1-122A1-long) |
| 193 | Protein | Native WT1 Peptide v1B |
| 194 | Protein | WT1-P1-P2-P3-FLAG-Ub-heteroclitic tyrosine minigene construct |
| 195 | DNA | Adv16 f |
| 196 | DNA | Adv295 r |
| 197 | Protein | Wild-Type WT1 Peptide v1 (A1) |
| 198 | Protein | Wild-Type WT1 Peptide v2 |
| 199 | Protein | Wild-Type WT1 Peptide v3 |
| 200 | Protein | Wild-Type WT1 Peptide v5 |
| 201 | Protein | Wild-Type WT1 Peptide v8 |
| 202 | Protein | Wild-Type WT1 Peptide v4 |
| 203 | Protein | Wild-Type WT1 Peptide v7 |
| 204 | Protein | Wild-Type WT1 Peptide v9 |
| 205 | Protein | Wild-Type WT1 Peptide v6 |
| 206 | Protein | Lm-AH1 HC |
| 207 | Protein | AH1 Wild Type |
| 208 | Protein | AH1 Wild Heteroclitic Peptide |
| 209-217 | Protein | Linkers |
| 218 | Protein | NSCLC HC + MG |
| 219 | Protein | NSCLC HC only |
| 220 | DNA | NSCLC HC + MG |
| 221 | DNA | NSCLC HC only |
| 222 | DNA | NSCLC HC only |
| 223-241 | DNA | NSCLC CEACAM5 A0301 Sequences |
| 242-260 | DNA | NSCLC MAGEA6 A0301 Sequences |
| 261-279 | DNA | NSCLC CEACAM5 B0702 Sequences |
| 280-298 | DNA | NSCLC MAGEA4 B0702 Sequences |
| 299-317 | DNA | NSCLC GAGE1 B0702 Sequences |
| 318-336 | DNA | NSCLC CEACAM5 A2402 Sequences |
| 337-355 | DNA | NSCLC NYESO1 A0201 Sequences |
| 356-374 | DNA | NSCLC CEACAM5 A0201 Sequences |
| 375-392 | DNA | Prostate MAGEA4 B0702 Sequences |
| 393-410 | DNA | Prostate STEAP1 A0201 Sequences |
| 411-428 | DNA | Prostate STEAP1 A2402 Sequences |
| 429-446 | DNA | Prostate SSX2 A0201 Sequences |
| 447-464 | DNA | Prostate SART3 A0201 Sequences |
| 465-482 | DNA | Prostate PAGE4 A0201 Sequences |
| 483-500 | DNA | Prostate PSMA A2402 Sequences |
| 501-518 | DNA | Prostate PSA A0301 Sequences |
| 519-536 | DNA | Bladder GAGE1 B0702 Sequences |
| 537-554 | DNA | Bladder NYESO1 A0201 Sequences |
| 555-572 | DNA | Bladder NUF2 A0201 Sequences |
| 573-590 | DNA | Bladder NUF2 A2402 Sequences |
| 591-608 | DNA | Bladder KLHL7 A2402 Sequences |
| 609-626 | DNA | Bladder MAGEA3 A2402 Sequences |
| 627-644 | DNA | Bladder GAGE1 A0301 Sequences |
| 645-662 | DNA | Bladder MAGEA3 A0301 Sequences |
| 663-680 | DNA | Bladder NYESO1 B0702 Sequences |
| 681-698 | DNA | Bladder MAGEA3 B0702 Sequences |
| 699-708 | DNA | Breast CEACAM5 A0301 Sequences |
| 709-718 | DNA | Breast CEACAM5 B0702 Sequences |
| 719-728 | DNA | Breast CEACAM5 A2402 Sequences |
| 729-738 | DNA | Breast CEACAM5 A0201 Sequences |
| 739-748 | DNA | Breast STEAP1 A0201 Sequences |
| 749-758 | DNA | Breast STEAP1 A2402 Sequences |
| 759-768 | DNA | Breast RNFF43 B0702 Sequences |
| 769-778 | DNA | Breast MAGEA3 A2402 Sequences |
| 779-788 | DNA | Breast MAGEA3 A0301 Sequences |
| 789-798 | DNA | Breast PRAME A0201 Sequences |
| 799-808 | DNA | Breast hTERT A0201_A2402 Sequences |
| 809-818 | DNA | Pancreas CEACAM5 A0301 Sequences |
| 819-828 | DNA | Pancreas CEACAM5 B0702 Sequences |
| 829-838 | DNA | Pancreas CEACAM5 A2402 Sequences |
| 839-848 | DNA | Pancreas CEACAM5 A0201 Sequences |
| 849-858 | DNA | Pancreas STEAP1 A0201 Sequences |
| 859-868 | DNA | Pancreas STEAP1 A2402 Sequences |
| 869-878 | DNA | Pancreas MAGEA3 A0301 Sequences |
| 879-888 | DNA | Pancreas PRAME A0201 Sequences |
| 889-898 | DNA | Pancreas hTERT A0201_A2402 Sequences |
| 899-908 | DNA | Pancreas MAGEA3 A0201_A2402 Sequences |
| 909-918 | DNA | Pancreas SURVIVIN A0201 Sequences |
| 919-928 | DNA | Pancreas SURVIVIN A2402 Sequences |
| 929-932 | DNA | Colorectal CEACAM5 A0301 Sequences |
| 933-936 | DNA | Colorectal MAGEA6 A0301 Sequences |
| 937-940 | DNA | Colorectal CEACAM5 B0702 Sequences |
| 941-944 | DNA | Colorectal MAGEA4 B0702 Sequences |
| 945-948 | DNA | Colorectal GAGE1 B0702 Sequences |
| 949-952 | DNA | Colorectal CEACAM5 A2402 Sequences |
| 953-956 | DNA | Colorectal NYESO1 A0201 Sequences |
| 957-960 | DNA | Colorectal STEAP1 A0201 Sequences |
| 961-964 | DNA | Colorectal RNF43 B0702 Sequences |
| 965-968 | DNA | Colorectal MAGEA3 A0201_A2402 Sequences |
| 969 | DNA | NSCLC STEAP1 A0201 Sequence |
| 970 | DNA | NSCLC STEAP1 S2402 Sequence |
| 971 | DNA | NSCLC RNF43 B0702 Sequence |
| 972 | DNA | Prostate CEACAM5 B0702 Sequence |
| 973 | DNA | Prostate RNF43 B0702 Sequence |
| 974 | DNA | Bladder CEACAM5 A0301 Sequence |
| 975 | DNA | Bladder CEACAM5 A0201 Sequence |
| 976 | DNA | Bladder RNF43 B0702 Sequence |
| 977 | DNA | Bladder PRAME A0201 Sequence |

### EXAMPLES

### Example 1. In Silico Methodology for Design of Heteroclitic Peptides

Heteroclitic peptides (i.e., sequence-optimized peptides) derived from cancer-associated proteins were designed to increase presentation by MHC Class I alleles. Heteroclitic peptides were derived by altering peptides expressed by tumor-associated antigen genes, as these represent genes that are expressed in tumor tissue, but have minimal expression in normal, healthy tissue. In particular, the heteroclitic peptides were designed from cancer-associated proteins such as cancer testis antigens or oncofetal antigens (i.e., were designed from tumor-associated antigens). Cancer testis antigens (CTAs) are a large family of tumor-associated antigens expressed in human tumors of different histological origin but not in normal tissue, except for male germ cells. In cancer, these developmental antigens can be re-expressed and can serve as a locus of immune activation. Oncofetal antigens (OFAs) are proteins that are typically present only during fetal development but are found in adults with certain kinds of cancer. The tumor-restricted pattern of expression of CTAs and OFAs make them ideal targets for tumor-specific immunotherapy. The combination of multiple OFA/CTAs can maximize patient coverage. Most OFA/CTA proteins play critical roles in oncogenesis, so targeting them can significantly impair cancer proliferation. Combining multiple OFA/CTAs peptides presents multiple high avidity targets in one treatment that are expressed in potentially all patients with the target disease.

Heteroclitics were designed to the four most prevalent HLAs in North America from genes with up to 100% expression in a cancer type. The HLA types chosen included A0201, A0301, A2402, and B0702, which have frequencies of 47.8%, 20.6%, 20.6%, and 28.7%, respectively in Caucasian in North America, and frequencies of 16.8%, 23.8%, 8.9%, and 16.0% in African Americans in North America. This increases the odds of at least one peptide-MHC combination per patient. Heteroclitic sequences have been shown to be sufficient to prime a T cell response, to overcome central tolerance, and to elicit a successful cross-reactive immune response to the wild-type peptide. Combinations of heteroclitic epitopes can bring total patient coverage within a cancer type to levels approaching 100%. We therefore do not need to sequence a patient prior to treatment as we assume that they will express a tumor-associated antigen that we have designed heteroclitic peptides for to cover the most prevalent HLAs (HLA-A0201, HLA-A0301, HLA-A2402, and HLA-B0702).

A literature review was done to survey the genomic landscape of indication-specific tumor-associated antigens to generate a short-list of potential tumor-associated antigens (TAAs). Heteroclitic peptides to HLA-A0201 that had immunogenicity information from the literature were selected. Heteroclitic peptides to HLA-A2402 were also selected.

A second literature review was done to determine if short-list TAAs contained known immunogenic peptides that generate CD8+ T lymphocyte response. This approach focused primarily on MHC Class I epitopes consisting of 9 amino acids (9mer) from TAAs. This step identified potential tumor-associated antigen peptides (TAAPs) in 9mer format that bind to one of four HLAs types (HLA-A*02:01, HLA-A*03:01, HLA-A*24:02, and HLA-B*07:02).

TAAPs were sequence optimized to enhance binding to MHC Class I molecules (aka heteroclitic peptide). To optimize binding to each HLA, the Peptide MHC Binding Motif and Amino Acid Binding Chart were assessed from the Immune Epitope Database and Analysis Resource (for example: iedb.org/MHCalleleid/143). The preferred amino acids at the anchor positions were inserted into the TAAP sequence (e.g., NUF2—wild type: YMMPVNSEV (SEQ ID NO: 131); and NUF2—heteroclitic: YLMPVNSEV (SEQ ID NO: 130)).

The binding affinities of sequence-optimized TAAPs and wild-type TAAP sequences were then assessed using one of the following algorithms: NetMHC4.0 Server; NetMHCpan4.0 Server; and mhcflurry v0.2.0.

Sequence-optimized TAAPs were considered if predicting binding affinity to a specific HLA was equivalent or stronger than the wild-type TAAP sequence.

Selected sequence-optimized TAAPs were then screened for in vitro binding to specific HLAs using ProImmune's REVEAL assay. TAAPs with binding affinity >=45% of the REVEAL assay's positive control peptide were considered binders.

Finally, the RNA expression level of TAAPs were measured in a specific-indication in TCGA RNAseqV2 dataset. The percentage of TCGA samples with normalized RNA expression reads greater than 0 were calculated. TAAPs with TCGA expression in a majority of samples were prioritized.

Each heteroclitic antigenic peptide can comprise a single heteroclitic mutation or can comprise two or more heteroclitic mutations (e.g., two heteroclitic mutations). Exemplary heteroclitic mutant peptides are provided in the following table along with the corresponding wild type (native) peptides. The residues in the wild type peptides that are modified in the corresponding heteroclitic peptides are bolded and underlined.

TABLE 1
Heteroclitic Antigenic Peptides and Corresponding Native Peptides.

| Peptide | | |
|---|---|---|
| (GENE_HLA Type) | Heteroclitic Peptide | Native Peptide |
| CEACAM5_A0201 | ILIGVLVGV (SEQ ID NO: 100) | IMIGVLVGV (SEQ ID NO: 101) |
| CEACAM5_A0201 | ILMGVLVGV (SEQ ID NO: 102) | IMIGVLVGV (SEQ ID NO: 103) |
| CEACAM5_A0301 | HVFGYSWYK (SEQ ID NO: 104) | HLFGYSWYK (SEQ ID NO: 105) |
| CEACAM5_A2402 | IYPNASLLF (SEQ ID NO: 106) | IYPNASLLI (SEQ ID NO: 107) |
| CEACAM5_B0702 | IPQVHTQVL (SEQ ID NO: 108) | IPQQHTQVL (SEQ ID NO: 109) |
| GAGE1_A0301 | SLYYWPRPR (SEQ ID NO: 110) | STYYWPRPR (SEQ ID NO: 111) |
| GAGE1_B0702 | WPRPRRYVM (SEQ ID NO: 112) | WPRPRRYVQ (SEQ ID NO: 113) |
| hTERT_A0201_A2402 | IMAKFLHWL (SEQ ID NO: 114) | ILAKFLHWL (SEQ ID NO: 115) |
| KLHL7_A2402 | VYILGGSQF (SEQ ID NO: 116) | VYILGGSQL (SEQ ID NO: 117) |
| MAGEA3_A0201_A2402 | KVPEIVHFL (SEQ ID NO: 118) | KVAELVHFL (SEQ ID NO: 119) |
| MAGEA3_A0301 | YMFPVIFSK (SEQ ID NO: 120) | YFFPVIFSK (SEQ ID NO: 121) |
| MAGEA3_A2402 | IMPKAGLLF (SEQ ID NO: 122) | IMPKAGLLFI (SEQ ID NO: 123) |
| MAGEA3_B0702 | LPWTMNYPL (SEQ ID NO: 124) | LPTTMNYPL (SEQ ID NO: 125) |
| MAGEA4_B0702 | MPSLREAAL (SEQ ID NO: 126) | YPSLREAAL (SEQ ID NO: 127) |
| MAGEA6_A0301 | YLFPVIFSK (SEQ ID NO: 128) | YFFPVIFSK (SEQ ID NO: 129) |
| NUF2_A0201 | YLMPVNSEV (SEQ ID NO: 130) | YMMPVNSEV (SEQ ID NO: 131) |
| NUF2_A2402 | VWGIRLEHF (SEQ ID NO: 132) | VYGIRLEHF (SEQ ID NO: 133) |
| NYESO1_A0201 | RLLEFYLAV (SEQ ID NO: 134) | RLLEFYLAM (SEQ ID NO: 135) |
| NYESO1_B0702 | APRGPHGGM (SEQ ID NO: 136) | APRGPHGGA (SEQ ID NO: 137) |
| PAGE4_A0201 | MAPDVVAFV (SEQ ID NO: 138) | EAPDVVAFV (SEQ ID NO: 139) |
| PRAME_A0201 | NMTHVLYPL (SEQ ID NO: 140) | NLTHVLYPV (SEQ ID NO: 141) |
| PSA_A0301 | GMAPLILSR (SEQ ID NO: 142) | GAAPLILSR (SEQ ID NO: 143) |
| PSMA_A2402 | TYSVSFFSW (SEQ ID NO: 144) | TYSVSFDSL (SEQ ID NO: 145) |
| RNF43_B0702 | NPQPVWLCL (SEQ ID NO: 146) | NSQPVWLCL (SEQ ID NO: 147) |
| SART3_A0201 | LMQAEAPRL (SEQ ID NO: 148) | LLQAEAPRL (SEQ ID NO: 149) |
| SSX2_A0201 | RLQGISPKV (SEQ ID NO: 150) | RLQGISPKI (SEQ ID NO: 151) |
| STEAP1_A0201 | LLLGTIHAV (SEQ ID NO: 152) | LLLGTIHAL (SEQ ID NO: 153) |
| STEAP1_A2402 | KYKKFPWWL (SEQ ID NO: 154) | KYKKFPHWL (SEQ ID NO: 155) |
| SURVIVIN_A0201 | KMSSGCAFL (SEQ ID NO: 156) | KHSSGCAFL (SEQ ID NO: 157) |
| SURVIVIN_A2402 | SWFKNWPFF (SEQ ID NO: 158) | STFKNWPFL (SEQ ID NO: 159) |

### Example 2. Design and Binding Affinity of Heteroclitic Peptides

Several cancer types were selected for which to develop heteroclitic immunogenic peptides (sequence-optimized tumor-associated antigen peptides). These included non-small cell lung cancer, prostate cancer, pancreatic cancer, bladder cancer, breast cancer (e.g., ER+breast cancer), uterine cancer, ovarian cancer, low-grade glioma, colorectal cancer (e.g., MSS colorectal cancer), and head and neck cancer. Table 2 provides a summary of tumor-associated genes from which peptides were derived for each type of cancer. The last column indicates the number of tumor-associated antigen (e.g., CTA/OFA) genes in the previous column that were expressed in at least 90% of The Cancer Genome Atlas (TCGA) patients for that indication. For example 3 TAA genes were expressed in over 90% of NSCLC patients. The rest of the TAA genes were expressed in <90% of the population of TCGA NSCLC patients.

TABLE 2
Summary of Tumor-Associate Genes from which Heteroclitic Peptides Derived.

| | | # TAA Genes |
|---|---|---|
| | | Expressed in |
| | Sequence-Optimized Tumor-Associated Antigen (TAA) | >90% of |
| Disease | Peptides (e.g., CTA/OFA Genes) | Patients |
| NSCLC | CEACAM5, MAGE-A6, NY-ESO1, MAGE-A3, MAGE-A4, | 3 |
| | GAGE1 | |
| Prostate | PSA, PSMA, STEAP1, SART3, TARP, PAGE-4, SSX2, | 7 |
| | MAGE-A4 | |
| Breast (ER+) | STEAP1, RNF53, CEACAM5, PRAME, TERT, MAGE-A3 | 4 |
| CRC (MSS) | CEACAM5, MAGE-A6, MAGE-A3, MAGE-A4, NY-ESO1, | 2 |
| | GAGE1 | |
| Head and Neck | CEACAM5, STEAP1, TERT, PRAME, MAGE-A4, NY-ESO1 | 4 |
| Pancreatic | STEAP1, SURVIVN, CEACAM5, PRAME, TERT, MAGE-A3 | 3 |
| Bladder | NUF2, KLHL7, MAGE-A3, NY-ESO1, GAGE1 | 4 |
| Ovarian | STEAP1, RNF43, SART3, KLHL7, NUF2, PRAME, TERT, | 6 |
| | CEACAM5, MAGE-A6 | |
| Glioma | KLHL7, NUF2, RNF43, SART3, STEAP1, TERT, MAGE-A6, | 4 |
| | CEACAM5 | |
| Uterine | STEAP1, RNF43, SART3, KLHL7, NUF2, PRAME, TERT, | 4 |
| | CEACAM5, MAGE-A6 | |

### Non-Small Cell Lung Cancer (NSCLC) Heteroclitic Peptides

A total of 11 peptides with heteroclitic mutations across 7 genes were selected for the NSCLC heteroclitic peptides. For each heteroclitic mutation, a peptide of 9 amino acids in length was designed as described in Example 1 and elsewhere herein. The peptides are shown in Table 3. The heteroclitic mutation in each is as described in Table 1.

TABLE 3
Exemplary NSCLC Heteroclitic 9-Mers.
NSCLC Heteroclitic 9-Mers

| | | | | Representative |
|---|---|---|---|---|
| | | | | Nucleic Acid |
| Gene | HLA Type | Sequence | SEQ ID NO | SEQ ID NOS |
| CEACAM5 | A0301 | HVFGYSWYK | 104 | 223-241 |
| MAGEA6 | A0301 | YLFPVIFSK | 128 | 242-260 |
| CEACAM5 | B0702 | IPQVHTQVL | 108 | 261-279 |
| MAGEA4 | B0702 | MPSLREAAL | 126 | 280-298 |
| GAGE1 | B0702 | WPRPRRYVM | 112 | 299-317 |
| CEACAM5 | A2402 | IYPNASLLF | 106 | 318-336 |
| NYESO1 | A0201 | RLLEFYLAV | 134 | 337-355 |
| CEACAM5 | A0201 | ILIGVLVGV | 100 | 356-374 |
| STEAP1 | A0201 | LLLGTIHAV | 152 | 969 |
| STEAP1 | A2402 | KYKKFPWWL | 154 | 970 |
| RNF43 | B0702 | NPQPVWLCL | 146 | 971 |

The in silico predicted binding affinity and in vitro binding affinity of the heteroclitic 9-mer peptides are provided in Table 4. The in silico predicted binding affinity is based on the NetMHC4.0 algorithm, which predicts peptide binding to MHC class I molecules in terms of 50% inhibitory concentration (IC50) values (nM); a lower number reflects stronger predicted binding affinity. The in vitro binding affinity was determined through a binding assay that determines the ability of each candidate peptide to bind to the indicated MHC class I alleles and stabilize the MHC-peptide complex by comparing the binding to that of a high affinity T cell epitope. Briefly, each peptide is incubated with its specific HLA molecule in an in vitro assay. Binding strength is compared against a known, immunogenic peptide for the same HLA molecule as a positive control with the positive control binding score set to 100%. The sequence-optimized binding score is normalized to the control peptide. That is, each peptide was given a score relative to the positive control peptide, which is a known T cell epitope with very strong binding properties. The score of the heteroclitic test peptide is reported quantitatively as a percentage of the signal generated by the positive control peptide. Peptides with scores greater than or equal to 45% of the positive control are considered binders. Also provided in Table 4 are the percent expression of each gene in patients with NSCLC (The Cancer Genome Atlas (TCGA) database), the HLA allele being tested, and whether the wild-type peptide corresponding to each heteroclitic peptide is known to be immunogenic. For a construct including each of the heteroclitic peptides in Table 4, 100% of NSCLC patients with HLA type A*02:01 express at least one of the TAA genes, 100% of NSCLC patients with HLA type A*03:01 express at least one of the TAA genes, 100% of NSCLC patients with HLA type A*24:02 express at least one of the TAA genes, and 100% of NSCLC patients with HLA type B*07:02 express at least one of the TAA genes.

TABLE 4
Binding Affinities of Heteroclitic 9-Mers to HLA.

| | | | In silico | | |
|---|---|---|---|---|---|
| | | | Predicted | In vitro | |
| | % Expression | | Binding | Binding | Wild-Type Peptide |
| TAA Gene | in TCGA | HLA Allele | Affinity IC50^{#} | Affinity\hat{ } | Immunogenic? |
| CEACAM5 | 100 | A*02:01 | 6.92 | 170.7 | Yes |
| CEACAM5 | 100 | A*24:02 | 6.22 | 77.2 | Yes |
| CEACAM5 | 100 | A*03:01 | 9.69 | 85.4 | Yes |
| CEACAM5 | 100 | B*07:02 | 8.36 | 88.3 | Yes |
| STEAP1 | 100 | A*02:01 | 5.77 | 188.4 | Yes |
| STEAP1 | 100 | A*24:02 | 47.48 | 104.7 | unknown |
| RNF43 | 100 | B*07:02 | 161.95 | 65.4 | Yes |
| MAGE-A6 | 53 | A*03:01 | 12.83 | 103.7 | unknown |
| NY-ESO1 | 50 | A*02:01 | 4.61 | 212.9 | unknown |
| MAGE-A4 | 35 | B*07:02 | 7.67 | 49.5 | unknown |
| GAGE1 | 10 | B*07:02 | 2.58 | 58.5 | unknown |

Additional Heteroclitic 9-Mers

| | | | | | |
|---|---|---|---|---|---|
| MAGE-A3^{&} | 50 | A*02:01 | 50.31 | 168.7 | Yes |
| MAGE-A3^{&} | 50 | A*24:02 | 2966 | 102.4 | unknown |

^{#}NetMHC4.0
\hat{ }%relative to positive control peptide binding
^{&}SEQ ID NO: 118

Constructs were designed to encode a fusion polypeptide comprising tLLO fused to one or more heteroclitic peptides, with the C-terminal heteroclitic peptide following a ubiquitin peptide (i.e., heteroclitic peptides and “minigene”). The tLLO, heteroclitic peptide, and ubiquitin/heteroclitic peptide components of the fusion polypeptides were joined by various linkers selected from those in disclosed elsewhere herein. An exemplary fusion polypeptide insert sequence (i.e., the peptide sequence downstream of the tLLO) is NSCLC HC+MG (SEQ ID NO: 218). An exemplary nucleic acid encoding NSCLC HC+MG is set forth in SEQ ID NO: 220.

Constructs were also designed to encode a fusion polypeptide comprising tLLO fused to one or more heteroclitic peptides without any ubiquitin peptide (i.e., heteroclitic peptides with no “minigene”). The tLLO and heteroclitic peptide components of the fusion polypeptides were joined by various linkers selected from those disclosed elsewhere herein. An exemplary fusion polypeptide insert sequence (i.e., the peptide sequence downstream of the tLLO) is NSCLC HC only (SEQ ID NO: 219). Exemplary nucleic acids encoding NSCLC HC only are set forth in SEQ ID NOS: 221 and 222.

A breakdown of the amino acids positions of the individual components in each construct is provided below.

TABLE 4B
Positions of Components of NSCLC HC + MG Insert.

| | | |
|---|---|---|
| 21-29: CEACAM5_A0301 | 126-134: CEACAM5_A2402 | 239-259: FLAG |
| 42-50: MAGEA6_A0301 | 147-155: NYESOl_A0201 | 260-279: Linker-SIINFEKL |
| 63-71: CEACAM5_B0702 | 168-176: STEAP1_A0201 | 286-360: Ubiquitin |
| 84-92: MAGEA4_B0702 | 189-197: STEAP1_A2402 | 361-369: CEACAMS_ |
| 105-113: GAGE1_B0702 | 210-218: RNF43_B0702 | A0201_MINI |

TABLE 4C
Positions of Components of NSCLC HC Only Insert.

| | | |
|---|---|---|
| 21-29: CEACAM5_A0301 | 126-134: CEACAM5_A2402 | 210-218: RNF43_B0702 |
| 42-50: MAGEA6_A0301 | 147-155: NYESO1_A0201 | 239-259: FLAG |
| 63-71: CEACAM5_B0702 | 168-176: STEAP1_A0201 | 260-279: Linker-SIINFEKL |
| 84-92: MAGEA4_B0702 | 189-197: STEAP1_A2402 | 286-294: CEACAM5_ |
| 105-113: GAGE1_B0702 | | A0201_MINI |

### Prostate Cancer Heteroclitic Peptides

A total of 10 peptides with heteroclitic mutations across 9 genes were selected for the prostate cancer heteroclitic peptides. For each heteroclitic mutation, a peptide of 9 amino acids in length was designed as described in Example 1 and elsewhere herein. The peptides are shown in Table 5. The heteroclitic mutation in each is as described in Table 1.

TABLE 5
Exemplary Prostate Cancer Heteroclitic 9-Mers.
Prostate Cancer Heteroclitic 9-Mers

| | | | | Representative |
|---|---|---|---|---|
| | | | | Nucleic Acid |
| Gene | HLA Type | Sequence | SEQ ID NO | SEQ ID NOS |
| CEACAM5 | B0702 | IPQVHTQVL | 108 | 972 |
| MAGEA4 | B0702 | MPSLREAAL | 126 | 375-392 |
| STEAP1 | A0201 | LLLGTIHAV | 152 | 393-410 |
| STEAP1 | A2402 | KYKKFPWWL | 154 | 411-428 |
| RNF43 | B0702 | NPQPVWLCL | 146 | 973 |
| SSX2 | A0201 | RLQGISPKV | 150 | 429-446 |
| SART3 | A0201 | LMQAEAPRL | 148 | 447-464 |
| PAGE4 | A0201 | MAPDVVAFV | 138 | 465-482 |
| PSMA | A2402 | TYSVSFFSW | 144 | 483-500 |
| PSA | A0301 | GMAPLILSR | 142 | 501-518 |

The in silico predicted binding affinity and in vitro binding affinity of the heteroclitic 9-mer peptides are provided in Table 6. The in silico predicted binding affinity is based on the NetMHC4.0 algorithm, which predicts peptide binding to MHC class I molecules in terms of 50% inhibitory concentration (IC50) values (nM); a lower number reflects stronger predicted binding affinity. The in vitro binding affinity was determined through a binding assay that determines the ability of each candidate peptide to bind to the indicated MHC class I alleles and stabilize the MHC-peptide complex by comparing the binding to that of a high affinity T cell epitope. Briefly, each peptide is incubated with its specific HLA molecule in an in vitro assay. Binding strength is compared against a known, immunogenic peptide for the same HLA molecule as a positive control with the positive control binding score set to 100%. The sequence-optimized binding score is normalized to the control peptide. That is, each peptide was given a score relative to the positive control peptide, which is a known T cell epitope with very strong binding properties. The score of the heteroclitic test peptide is reported quantitatively as a percentage of the signal generated by the positive control peptide. Peptides with scores greater than or equal to 45% of the positive control are considered binders. Also provided in Table 6 are the percent expression of each gene in patients with prostate cancer (The Cancer Genome Atlas database), the HLA allele being tested, and whether the wild-type peptide corresponding to each heteroclitic peptide is known to be immunogenic. For a construct including each of the heteroclitic peptides in Table 6, 100% of prostate cancer patients with HLA type A*02:01 express at least one of the TAA genes, 100% of prostate cancer patients with HLA type A*03:01 express at least one of the TAA genes, 100% of prostate cancer patients with HLA type A*24:02 express at least one of the TAA genes, and 100% of prostate cancer patients with HLA type B*07:02 express at least one of the TAA genes.

TABLE 6
Binding Affinities of Heteroclitic 9-Mers to HLA.

| | | | In silico | | |
|---|---|---|---|---|---|
| | | | Predicted | | |
| | % Expression | | Binding | In vitro Binding | Wild-Type Peptide |
| TAA Gene | in TCGA | HLA Allele | Affinity IC50^{#} | Affinity\hat{ } | Immunogenic? |
| PSA | 100 | A*03:01 | 179.39 | 103.5 | Yes |
| PSMA | 100 | A*24:02 | 20.45 | 96.2 | Yes |
| STEAP1 | 100 | A*02:01 | 5.77 | 188.4 | Yes |
| STEAP1 | 100 | A*24:02 | 47.48 | 104.7 | unknown |
| SART3 | 100 | A*02:01 | 235.57 | 160.0 | Yes |
| RNF43 | 100 | B*07:02 | 161.95 | 65.4 | Yes |
| PAGE4 | 99 | A*02:01 | 39.32 | 126.6 | unknown |
| CEACAM5 | 95 | B*07:02 | 8.36 | 88.3 | Yes |
| SSX2 | 13 | A*02:01 | 31.02 | 179.5 | Yes |
| MAGE-A4 | 6 | B*07:02 | 7.67 | 49.5 | unknown |

^{#}NetMHC4.0
\hat{ }% relative to positive control peptide binding

### Pancreatic Cancer Heteroclitic Peptides

A total of 12 peptides with heteroclitic mutations across 6 genes were selected for the pancreatic cancer heteroclitic peptides. For each heteroclitic mutation, a peptide of 9 amino acids in length was designed as described in Example 1 and elsewhere herein. The peptides are shown in Table 7. The heteroclitic mutation in each is as described in Table 1.

TABLE 7
Exemplary Pancreatic Cancer Heteroclitic 9-Mers.
Pancreatic Cancer Heteroclitic 9-Mers

| | | | SEQ | Representative |
|---|---|---|---|---|
| | | | ID | Nucleic Acid |
| Gene | HLA Type | Sequence | NO | SEQ ID NOS |
| CEACAM5 | A0301 | HVFGYSWYK | 104 | 809-818 |
| CEACAM5 | B0702 | IPQVHTQVL | 108 | 819-828 |
| CEACAM5 | A2402 | IYPNASLLF | 106 | 829-838 |
| CEACAM5 | A0201 | ILIGVLVGV | 100 | 839-848 |
| STEAP1 | A0201 | LLLGTIHAV | 152 | 849-858 |
| STEAP1 | A2402 | KYKKFPWWL | 154 | 859-868 |
| MAGEA3 | A0301 | YMFPVIFSK | 120 | 869-878 |
| PRAME | A0201 | NMTHVLYPL | 140 | 879-888 |
| hTERT | A0201_A2402 | IMAKFLHWL | 114 | 889-898 |
| MAGEA3 | A0201_A2402 | KVPEIVHFL | 118 | 899-908 |
| SURVIVIN | A0201 | KMSSGCAFL | 156 | 909-918 |
| SURVIVIN | A2402 | SWFKNWPFF | 158 | 919-928 |

The in silico predicted binding affinity and in vitro binding affinity of the heteroclitic 9-mer peptides are provided in Table 8. The in silico predicted binding affinity is based on the NetMHC4.0 algorithm, which predicts peptide binding to MHC class I molecules in terms of 50% inhibitory concentration (IC50) values (nM); a lower number reflects stronger predicted binding affinity. The in vitro binding affinity was determined through a binding assay that determines the ability of each candidate peptide to bind to the indicated MHC class I alleles and stabilize the MHC-peptide complex by comparing the binding to that of a high affinity T cell epitope. Briefly, each peptide is incubated with its specific HLA molecule in an in vitro assay. Binding strength is compared against a known, immunogenic peptide for the same HLA molecule as a positive control with the positive control binding score set to 100%. The sequence-optimized binding score is normalized to the control peptide. That is, each peptide was given a score relative to the positive control peptide, which is a known T cell epitope with very strong binding properties. The score of the heteroclitic test peptide is reported quantitatively as a percentage of the signal generated by the positive control peptide. Peptides with scores greater than or equal to 45% of the positive control are considered binders. Also provided in Table 8 are the percent expression of each gene in patients with pancreatic cancer (The Cancer Genome Atlas database), the HLA allele being tested, and whether the wild-type peptide corresponding to each heteroclitic peptide is known to be immunogenic. For a construct including each of the heteroclitic peptides in Table 8, 100% of pancreatic cancer patients with HLA type A*02:01 express at least one of the TAA genes, 98% of pancreatic cancer patients with HLA type A*03:01 express at least one of the TAA genes, 100% of pancreatic cancer patients with HLA type A*24:02 express at least one of the TAA genes, and 98% of pancreatic cancer patients with HLA type B*07:02 express at least one of the TAA genes.

TABLE 8
Binding Affinities of Heteroclitic 9-Mers to HLA.

| | | | In silico | | |
|---|---|---|---|---|---|
| | | | Predicted | | |
| | % Expression | | Binding | In vitro Binding | Wild-Type Peptide |
| TAA Gene | in TCGA | HLA Allele | Affinity IC50^{#} | Affinity\hat{ } | Immunogenic? |
| STEAP1 | 100 | A*02:01 | 5.77 | 188.4 | Yes |
| STEAP1 | 100 | A*24:02 | 47.48 | 104.7 | unknown |
| SURVIVIN | 100 | A*02:01 | 11.66 | 149.0 | Yes |
| SURVIVIN | 100 | A*24:02 | 12.86 | 144.0 | Yes |
| CEACAM5 | 98 | A*02:01 | 6.92 | 170.7 | Yes |
| CEACAM5 | 98 | A*03:01 | 9.69 | 85.4 | Yes |
| CEACAM5 | 98 | B*07:02 | 8.36 | 88.3 | Yes |
| CEACAM5 | 98 | A*24:02 | 6.22 | 77.2 | Yes |
| PRAME | 87 | A*02:01 | 11.72 | 139.4 | Yes |
| TERT | 80 | A*02:01 | 7.04 | 123.3 | Yes |
| TERT | 80 | A*24:02 | 2197.84 | 142.3 | unknown |
| MAGE-A3 | 11 | A*02:01 | 50.31 | 168.7 | Yes |
| MAGE-A3 | 11 | A*24:02 | 2966 | 102.4 | unknown |
| MAGE-A3 | 11 | A*03:01 | 9.40 | 85.4 | unknown |

^{#}NetMHC4.0
\hat{ }% relative to positive control peptide binding

### Bladder Cancer Heteroclitic Peptides

A total of 14 peptides with heteroclitic mutations across 8 genes were selected for the bladder cancer heteroclitic peptides. For each heteroclitic mutation, a peptide of 9 amino acids in length was designed as described in Example 1 and elsewhere herein. The peptides are shown in Table 9. The heteroclitic mutation in each is as described in Table 1.

TABLE 9
Exemplary Bladder Cancer Heteroclitic 9-Mers.
Bladder Cancer Heteroclitic 9-Mers

| | | | SEQ | Representative |
|---|---|---|---|---|
| | | | ID | Nucleic Acid |
| Gene | HLA Type | Sequence | NO | SEQ ID NOS |
| CEACAM5 | A0301 | HVFGYSWYK | 104 | 974 |
| GAGE1 | B0702 | WPRPRRYVM | 112 | 519-536 |
| NYES01 | A0201 | RLLEFYLAV | 134 | 537-554 |
| CEACAM5 | A0201 | ILIGVLVGV | 100 | 975 |
| RNF43 | B0702 | NPQPVWLCL | 146 | 976 |
| NUF2 | A0201 | YLMPVNSEV | 130 | 555-572 |
| NUF2 | A2402 | VWGIRLEHF | 132 | 573-590 |
| KLHL7 | A2402 | VYILGGSQF | 116 | 591-608 |
| MAGEA3 | A2402 | IMPKAGLLF | 112 | 609-626 |
| GAGE1 | A0301 | SLYYWPRPR | 110 | 627-644 |
| MAGEA3 | A0301 | YMFPVIFSK | 120 | 645-662 |
| NYES01 | B0702 | APRGPHGGM | 136 | 663-680 |
| MAGEA3 | B0702 | LPWTMNYPL | 124 | 681-698 |
| PRAME | A0201 | NMTHVLYPL | 140 | 977 |

The in silico predicted binding affinity and in vitro binding affinity of the heteroclitic 9-mer peptides are provided in Table 10. The in silico predicted binding affinity is based on the NetMHC4.0 algorithm, which predicts peptide binding to MHC class I molecules in terms of 50% inhibitory concentration (IC50) values (nM); a lower number reflects stronger predicted binding affinity. The in vitro binding affinity was determined through a binding assay that determines the ability of each candidate peptide to bind to the indicated MHC class I alleles and stabilize the MHC-peptide complex by comparing the binding to that of a high affinity T cell epitope. Briefly, each peptide is incubated with its specific HLA molecule in an in vitro assay. Binding strength is compared against a known, immunogenic peptide for the same HLA molecule as a positive control with the positive control binding score set to 100%. The sequence-optimized binding score is normalized to the control peptide. That is, each peptide was given a score relative to the positive control peptide, which is a known T cell epitope with very strong binding properties. The score of the heteroclitic test peptide is reported quantitatively as a percentage of the signal generated by the positive control peptide. Peptides with scores greater than or equal to 45% of the positive control are considered binders. Also provided in Table 10 are the percent expression of each gene in patients with bladder cancer (The Cancer Genome Atlas database), the HLA allele being tested, and whether the wild-type peptide corresponding to each heteroclitic peptide is known to be immunogenic. For a construct including each of the heteroclitic peptides in Table 10, 100% of bladder cancer patients with HLA type A*02:01 express at least one of the TAA genes, 100% of bladder cancer patients with HLA type A*03:01 express at least one of the TAA genes, 100% of bladder cancer patients with HLA type A*24:02 express at least one of the TAA genes, and 100% of bladder cancer patients with HLA type B*07:02 express at least one of the TAA genes.

TABLE 10
Binding Affinities of Heteroclitic 9-Mers to HLA.

| | | | In silico | | |
|---|---|---|---|---|---|
| | | | Predicted | | |
| | % Expression | | Binding | In vitro Binding | Wild-Type Peptide |
| TAA Gene | in TCGA | HLA Allele | Affinity IC50^{#} | Affinity\hat{ } | Immunogenic? |
| NUF2 | 100 | A*02:01 | 2.79 | 160.0 | Yes |
| NUF2 | 100 | A*24:02 | 149.07 | 88.4 | unknown |
| KLHL7 | 100 | A*24:02 | 60.84 | 97.4 | Yes |
| RNF43 | 99 | B*07:02 | 161.95 | 65.4 | Yes |
| CEACAM5 | 93 | A*02:01 | 6.92 | 170.7 | Yes |
| CEACAM5 | 93 | A*03:01 | 9.69 | 85.4 | Yes |
| PRAME | 77 | A*02:01 | 11.72 | 139.4 | Yes |
| MAGE-A3 | 72 | B*07:02 | 12.52 | 112.2 | unknown |
| MAGE-A3 | 72 | A*24:02 | 28.11 | 92.8 | unknown |
| MAGE-A3 | 72 | A*03:01 | 9.40 | 86.9 | unknown |
| NY-ESO | 58 | A*02:01 | 4.61 | 212.9 | unknown |
| NY-ESO | 58 | B*07:02 | 3.32 | 109.7 | unknown |
| GAGE | 14 | B*07:02 | 2.58 | 58.5 | unknown |
| GAGE | 14 | A*03:01 | 60.49 | 93.1 | unknown |

^{#}NetMHC4.0
\hat{ }% relative to positive control peptide binding

### Breast Cancer Heteroclitic Peptides

A total of 11 peptides with heteroclitic mutations across 6 genes were selected for the breast cancer heteroclitic peptides. For each heteroclitic mutation, a peptide of 9 amino acids in length was designed as described in Example 1 and elsewhere herein. The peptides are shown in Table 11. The heteroclitic mutation in each is as described in Table 1.

TABLE 11
Exemplary Breast Cancer Heteroclitic 9-Mers.
Breast Cancer Heteroclitic 9-Mers

| | | | SEQ | Representative |
|---|---|---|---|---|
| | | | ID | Nucleic Acid |
| Gene | HLA Type | Sequence | NO | SEQ ID NOS |
| CEACAM5 | A0301 | HVFGYSWYK | 104 | 699-708 |
| CEACAM5 | B0702 | IPQVHTQVL | 108 | 709-718 |
| CEACAM5 | A2402 | IYPNASLLF | 106 | 719-728 |
| CEACAM5 | A0201 | ILIGVLVGV | 100 | 729-738 |
| STEAP1 | A0201 | LLLGTIHAV | 152 | 739-748 |
| STEAP1 | A2402 | KYKKFPWWL | 154 | 749-758 |
| RNF43 | B0702 | NPQPVWLCL | 146 | 759-768 |
| MAGEA3 | A2402 | IMPKAGLLF | 122 | 769-778 |
| MAGEA3 | A0301 | YMFPVIFSK | 120 | 779-788 |
| PRAME | A0201 | NMTHVLYPL | 140 | 789-798 |
| hTERT | A0201_A2402 | IMAKFLHWL | 114 | 799-808 |

The in silico predicted binding affinity and in vitro binding affinity of the heteroclitic 9-mer peptides are provided in Table 12. The in silico predicted binding affinity is based on the NetMHC4.0 algorithm, which predicts peptide binding to MHC class I molecules in terms of 50% inhibitory concentration (IC50) values (nM); a lower number reflects stronger predicted binding affinity. The in vitro binding affinity was determined through a binding assay that determines the ability of each candidate peptide to bind to the indicated MHC class I alleles and stabilize the MHC-peptide complex by comparing the binding to that of a high affinity T cell epitope. Briefly, each peptide is incubated with its specific HLA molecule in an in vitro assay. Binding strength is compared against a known, immunogenic peptide for the same HLA molecule as a positive control with the positive control binding score set to 100%. The sequence-optimized binding score is normalized to the control peptide. That is, each peptide was given a score relative to the positive control peptide, which is a known T cell epitope with very strong binding properties. The score of the heteroclitic test peptide is reported quantitatively as a percentage of the signal generated by the positive control peptide. Peptides with scores greater than or equal to 45% of the positive control are considered binders. Also provided in Table 12 are the percent expression of each gene in patients with breast cancer (The Cancer Genome Atlas database), the HLA allele being tested, and whether the wild-type peptide corresponding to each heteroclitic peptide is known to be immunogenic. For a construct including each of the heteroclitic peptides in Table 12, 100% of breast cancer patients with HLA type A*02:01 express at least one of the TAA genes, 95% of breast cancer patients with HLA type A*03:01 express at least one of the TAA genes, 100% of breast cancer patients with HLA type A*24:02 express at least one of the TAA genes, and 100% of breast cancer patients with HLA type B*07:02 express at least one of the TAA genes.

TABLE 12
Binding Affinities of Heteroclitic 9-Mers to HLA.

| | | | In silico | | |
|---|---|---|---|---|---|
| | | | Predicted | | |
| | % Expression | | Binding | In vitro Binding | Wild-Type Peptide |
| TAA Gene | in TCGA | HLA Allele | Affinity IC50^{#} | Affinity\hat{ } | Immunogenic? |
| STEAP1 | 100 | A*02:01 | 5.77 | 188.4 | Yes |
| STEAP1 | 100 | A*24:02 | 47.48 | 104.7 | unknown |
| RNF43 | 100 | B*07:02 | 161.95 | 65.4 | Yes |
| CEACAM5 | 95 | A*02:01 | 6.92 | 170.7 | Yes |
| CEACAM5 | 95 | A*03:01 | 9.69 | 85.4 | Yes |
| CEACAM5 | 95 | A*24:02 | 6.22 | 77.2 | Yes |
| CEACAM5 | 95 | B*07:02 | 8.36 | 88.3 | Yes |
| PRAME | 92 | A*02:01 | 11.72 | 139.4 | Yes |
| TERT | 87 | A*02:01 | 7.04 | 123.3 | Yes |
| TERT | 87 | A*24:02 | 2197.84 | 142.3 | unknown |
| MAGE-A3 | 31 | A*03:01 | 9.40 | 85.4 | unknown |
| MAGE-A3 | 31 | A*24:02 | 28.11 | 92.8 | unknown |

^{#}NetMHC4.0
\hat{ }% relative to positive control peptide binding

### Uterine Cancer Heteroclitic Peptides

A total of 14 peptides with heteroclitic mutations across 8 genes were selected for the uterine cancer heteroclitic peptides. For each heteroclitic mutation, a peptide of 9 amino acids in length was designed as described in Example 1 and elsewhere herein. The peptides are shown in Table 13. The heteroclitic mutation in each is as described in Table 1.

TABLE 13
Exemplary Uterine Cancer Heteroclitic 9-Mers.
Uterine Cancer Heteroclitic 9-Mers

| Gene | HLA Type | Sequence | SEQ ID NO |
|---|---|---|---|
| CEACAM5 | A0201 | ILMGVLVGV | 102 |
| CEACAM5 | A0301 | HVFGYSWYK | 104 |
| CEACAM5 | B0702 | IPQVHTQVL | 108 |
| CEACAM5 | A0201 | ILIGVLVGV | 100 |
| PRAME | A0201 | NMTHVLYPL | 140 |
| hTERT | A0201_A2402 | IMAKFLHWL | 114 |
| STEAP1 | A0201 | LLLGTIHAV | 152 |
| CEACAM5 | A2402 | IYPNASLLF | 106 |
| RNF43 | B0702 | NPQPVWLCL | 146 |
| NUF2 | A0201 | YLMPVNSEV | 130 |
| NUF2 | A2402 | VWGIRLEHF | 132 |
| KLHL7 | A2402 | VYILGGSQF | 116 |
| SART3 | A0201 | LMQAEAPRL | 148 |
| STEAP1 | A2402 | KYKKFPWWL | 154 |

The in silico predicted binding affinity and in vitro binding affinity of the heteroclitic 9-mer peptides are provided in Table 14. The in silico predicted binding affinity is based on the NetMHC4.0 algorithm, which predicts peptide binding to MHC class I molecules in terms of 50% inhibitory concentration (IC50) values (nM); a lower number reflects stronger predicted binding affinity. The in vitro binding affinity was determined through a binding assay that determines the ability of each candidate peptide to bind to the indicated MHC class I alleles and stabilize the MHC-peptide complex by comparing the binding to that of a high affinity T cell epitope. Briefly, each peptide is incubated with its specific HLA molecule in an in vitro assay. Binding strength is compared against a known, immunogenic peptide for the same HLA molecule as a positive control with the positive control binding score set to 100%. The sequence-optimized binding score is normalized to the control peptide. That is, each peptide was given a score relative to the positive control peptide, which is a known T cell epitope with very strong binding properties. The score of the heteroclitic test peptide is reported quantitatively as a percentage of the signal generated by the positive control peptide. Peptides with scores greater than or equal to 45% of the positive control are considered binders. Also provided in Table 14 are the percent expression of each gene in patients with uterine cancer (The Cancer Genome Atlas (TCGA) database), the HLA allele being tested, and whether the wild-type peptide corresponding to each heteroclitic peptide is known to be immunogenic. For a construct including each of the heteroclitic peptides in Table 14, 100% of uterine cancer patients with HLA type A*02:01 express at least one of the TAA genes, 83% of uterine cancer patients with HLA type A*03:01 express at least one of the TAA genes, 100% of uterine cancer patients with HLA type A*24:02 express at least one of the TAA genes, and 100% of uterine cancer patients with HLA type B*07:02 express at least one of the TAA genes.

TABLE 14
Binding Affinities of Heteroclitic 9-Mers to HLA.

| | | | In silico | | |
|---|---|---|---|---|---|
| | | | Predicted | | |
| | % Expression | | Binding | In vitro Binding | Wild-Type Peptide |
| TAA Gene | in TCGA | HLA Allele | Affinity IC50^{#} | Affinity\hat{ } | Immunogenic? |
| CEACAM5¹ | 84 | A*02:01 | 6.92 | 170.7 | Yes |
| CEACAM5² | 84 | A*02:01 | 3.47 | TBD | Yes |
| CEACAM5 | 84 | A*03:01 | 9.69 | 85.4 | Yes |
| CEACAM5 | 84 | B*07:02 | 8.36 | 88.3 | Yes |
| STEAP1 | 100 | A*02:01 | 5.77 | 188.4 | Yes |
| PRAME | 99 | A*02:01 | 11.72 | 139.4 | Yes |
| TERT | 92 | A*02:01 | 7.04 | 123.3 | Yes |
| TERT | 92 | A*24:02 | 2197.84 | 142.3 | unknown |
| STEAP1 | 100 | A*24:02 | 47.48 | 104.7 | unknown |
| CEACAM5 | 84 | A*24:02 | 6.22 | 77.2 | Yes |
| RNF43 | 100 | B*07:02 | 161.95 | 65.4 | Yes |
| NUF2 | 99 | A*02:01 | 2.79 | 160.0 | Yes |
| KLHL7 | 100 | A*24:02 | 60.84 | 97.4 | Yes |
| SART3 | 100 | A*02:01 | 235.57 | 160.0 | Yes |
| NUF2 | 99 | A*24:02 | 149.07 | 88.4 | Yes |

^{#}NetMHC4.0
\hat{ }% relative to positive control peptide binding
¹SEQ ID NO: 100
²SEQ ID NO: 102

### Ovarian Cancer Heteroclitic Peptides

A total of 14 peptides with heteroclitic mutations across 8 genes were selected for the ovarian cancer heteroclitic peptides. For each heteroclitic mutation, a peptide of 9 amino acids in length was designed as described in Example 1 and elsewhere herein. The peptides are shown in Table 15. The heteroclitic mutation in each is as described in Table 1.

TABLE 15
Exemplary Ovarian Cancer Heteroclitic 9-Mers.
Ovarian Cancer Heteroclitic 9-Mers

| Gene | HLA Type | Sequence | SEQ ID NO |
|---|---|---|---|
| CEACAM5 | A0301 | HVFGYSWYK | 104 |
| CEACAM5 | B0702 | IPQVHTQVL | 108 |
| CEACAM5 | A2402 | IYPNASLLF | 106 |
| CEACAM5 | A0201 | ILIGVLVGV | 100 |
| STEAP1 | A0201 | LLLGTIHAV | 152 |
| STEAP1 | A2402 | KYKKFPWWL | 154 |
| RNF43 | B0702 | NPQPVWLCL | 146 |
| SART3 | A0201 | LMQAEAPRL | 148 |
| NUF2 | A0201 | YLMPVNSEV | 130 |
| NUF2 | A2402 | VWGIRLEHF | 132 |
| KLHL7 | A2402 | VYILGGSQF | 116 |
| PRAME | A0201 | NMTHVLYPL | 140 |
| hTERT | A0201_A2402 | IMAKFLHWL | 114 |
| CEACAM5 | A0201 | ILMGVLVGV | 102 |

The in silico predicted binding affinity and in vitro binding affinity of the heteroclitic 9-mer peptides are provided in Table 16. The in silico predicted binding affinity is based on the NetMHC4.0 algorithm, which predicts peptide binding to MHC class I molecules in terms of 50% inhibitory concentration (IC50) values (nM); a lower number reflects stronger predicted binding affinity. The in vitro binding affinity was determined through a binding assay that determines the ability of each candidate peptide to bind to the indicated MHC class I alleles and stabilize the MHC-peptide complex by comparing the binding to that of a high affinity T cell epitope. Briefly, each peptide is incubated with its specific HLA molecule in an in vitro assay. Binding strength is compared against a known, immunogenic peptide for the same HLA molecule as a positive control with the positive control binding score set to 100%. The sequence-optimized binding score is normalized to the control peptide. That is, each peptide was given a score relative to the positive control peptide, which is a known T cell epitope with very strong binding properties. The score of the heteroclitic test peptide is reported quantitatively as a percentage of the signal generated by the positive control peptide. Peptides with scores greater than or equal to 45% of the positive control are considered binders. Also provided in Table 16 are the percent expression of each gene in patients with ovarian cancer (The Cancer Genome Atlas (TCGA) database), the HLA allele being tested, and whether the wild-type peptide corresponding to each heteroclitic peptide is known to be immunogenic. For a construct including each of the heteroclitic peptides in Table 16, 100% of ovarian cancer patients with HLA type A*02:01 express at least one of the TAA genes, 83% of ovarian cancer patients with HLA type A*03:01 express at least one of the TAA genes, 100% of ovarian cancer patients with HLA type A*24:02 express at least one of the TAA genes, and 100% of ovarian cancer patients with HLA type B*07:02 express at least one of the TAA genes.

TABLE 16
Binding Affinities of Heteroclitic 9-Mers to HLA.

| | | | In silico | | |
|---|---|---|---|---|---|
| | | | Predicted | | |
| | % Expression | | Binding | In vitro Binding | Wild-Type Peptide |
| TAA Gene | in TCGA | HLA Allele | Affinity IC50^{#} | Affinity\hat{ } | Immunogenic? |
| CEACAM5¹ | 93 | A*02:01 | 6.92 | 170.7 | Yes |
| CEACAM5² | 93 | A*02:01 | 3.47 | TBD | Yes |
| CEACAM5 | 93 | A*03:01 | 9.69 | 85.4 | Yes |
| CEACAM5 | 93 | B*07:02 | 8.36 | 88.3 | Yes |
| STEAP1 | 100 | A*02:01 | 5.77 | 188.4 | Yes |
| PRAME | 100 | A*02:01 | 11.72 | 139.4 | Yes |
| TERT | 94 | A*02:01 | 7.04 | 123.3 | Yes |
| TERT | 94 | A*24:02 | 2197.84 | 142.3 | unknown |
| STEAP1 | 100 | A*24:02 | 47.48 | 104.7 | unknown |
| CEACAM5 | 93 | A*24:02 | 6.22 | 77.2 | Yes |
| RNF43 | 100 | B*07:02 | 161.95 | 65.4 | Yes |
| NUF2 | 100 | A*02:01 | 2.79 | 160.0 | Yes |
| KLHL7 | 100 | A*24:02 | 60.84 | 97.4 | Yes |
| SART3 | 100 | A*02:01 | 235.57 | 160.0 | Yes |
| NUF2 | 100 | A*24:02 | 149.07 | 88.4 | Yes |

^{#}NetMHC4.0
\hat{ }% relative to positive control peptide binding
¹SEQ ID NO: 100
²SEQ ID NO: 102

### Low-Grade Glioma (LGG) Heteroclitic Peptides

A total of 10 peptides with heteroclitic mutations across 8 genes were selected for the LGG heteroclitic peptides. For each heteroclitic mutation, a peptide of 9 amino acids in length was designed as described in Example 1 and elsewhere herein. The peptides are shown in Table 17. The heteroclitic mutation in each is as described in Table 1.

TABLE 17
Exemplary LGG Heteroclitic 9-Mers.
LGG Heteroclitic 9-Mers

| Gene | HLA Type | Sequence | SEQ ID NO |
|---|---|---|---|
| CEACAM5 | A0301 | HVFGYSWYK | 104 |
| MAGEA6 | A0301 | YLFPVIFSK | 128 |
| STEAP1 | A0201 | LLLGTIHAV | 152 |
| STEAP1 | A2402 | KYKKFPWWL | 154 |
| RNF43 | B0702 | NPQPVWLCL | 146 |
| SART3 | A0201 | LMQAEAPRL | 148 |
| NUF2 | A0201 | YLMPVNSEV | 130 |
| NUF2 | A2402 | VWGIRLEHF | 132 |
| KLHL7 | A2402 | VYILGGSQF | 116 |
| hTERT | A0201_A2402 | IMAKFLHWL | 114 |

The in silico predicted binding affinity and in vitro binding affinity of the heteroclitic 9-mer peptides are provided in Table 18. The in silico predicted binding affinity is based on the NetMHC4.0 algorithm, which predicts peptide binding to MHC class I molecules in terms of 50% inhibitory concentration (IC50) values (nM); a lower number reflects stronger predicted binding affinity. The in vitro binding affinity was determined through a binding assay that determines the ability of each candidate peptide to bind to the indicated MHC class I alleles and stabilize the MHC-peptide complex by comparing the binding to that of a high affinity T cell epitope. Briefly, each peptide is incubated with its specific HLA molecule in an in vitro assay. Binding strength is compared against a known, immunogenic peptide for the same HLA molecule as a positive control with the positive control binding score set to 100%. The sequence-optimized binding score is normalized to the control peptide. That is, each peptide was given a score relative to the positive control peptide, which is a known T cell epitope with very strong binding properties. The score of the heteroclitic test peptide is reported quantitatively as a percentage of the signal generated by the positive control peptide. Peptides with scores greater than or equal to 45% of the positive control are considered binders. Also provided in Table 18 are the percent expression of each gene in patients with low-grade glioma (LGG) (The Cancer Genome Atlas (TCGA) database), the HLA allele being tested, and whether the wild-type peptide corresponding to each heteroclitic peptide is known to be immunogenic. For a construct including each of the heteroclitic peptides in Table 18, 100% of LGG patients with HLA type A*02:01 express at least one of the TAA genes, 43% of LGG patients with HLA type A*03:01 express at least one of the TAA genes, 100% of LGG patients with HLA type A*24:02 express at least one of the TAA genes, and 100% of LGG patients with HLA type B*07:02 express at least one of the TAA genes.

TABLE 18
Binding Affinities of Heteroclitic 9-Mers to HLA.

| | | | In silico | | |
|---|---|---|---|---|---|
| | | | Predicted | | |
| | % Expression | | Binding | In vitro Binding | Wild-Type Peptide |
| TAA Gene | in TCGA | HLA Allele | Affinity IC50^{#} | Affinity\hat{ } | Immunogenic? |
| NUF2 | 100 | A*02:01 | 2.79 | 160.0 | Yes |
| MAGE-A6 | 43 | A*03:01 | 12.83 | 103.7 | unknown |
| CEACAM5 | 27 | A*03:01 | 9.69 | 85.4 | Yes |
| STEAP1 | 99 | A*02:01 | 5.77 | 188.4 | Yes |
| STEAP1 | 99 | A*24:02 | 47.48 | 104.7 | unknown |
| RNF43 | 100 | B*07:02 | 161.95 | 65.4 | Yes |
| hTERT | 100 | A*02:01 | 7.05 | 123.3 | Yes |
| hTERT | 100 | A*24:02 | 2197.85 | 142.3 | unknown |
| NUF2 | 100 | A*24:02 | 149.07 | 88.4 | unknown |
| KLHL7 | 100 | A*24:02 | 60.84 | 97.4 | Yes |
| SART3 | 100 | A*02:01 | 235.57 | 160.0 | Yes |

^{#}NetMHC4.0
\hat{ }% relative to positive control peptide binding

### Colorectal Cancer (CRC) Heteroclitic Peptides

A total of 10 peptides with heteroclitic mutations across 8 genes were selected for the CRC heteroclitic peptides. For each heteroclitic mutation, a peptide of 9 amino acids in length was designed as described in Example 1 and elsewhere herein. The peptides are shown in Table 19. The heteroclitic mutation in each is as described in Table 122.

TABLE 19
Exemplary CRC Heteroclitic 9-Mers.
CRC Heteroclitic 9-Mers

| | | | SEQ | Representative |
|---|---|---|---|---|
| | | | ID | Nucleic Acid |
| Gene | HLA Type | Sequence | NO | SEQ ID NOS |
| CEACAM5 | A0301 | HVFGYSWYK | 104 | 929-932 |
| MAGEA6 | A0301 | YLFPVIFSK | 128 | 933-936 |
| CEACAM5 | B0702 | IPQVHTQVL | 108 | 937-940 |
| MAGEA4 | B0702 | MPSLREAAL | 126 | 941-944 |
| GAGE1 | B0702 | WPRPRRYVM | 112 | 945-948 |
| CEACAM5 | A2402 | IYPNASLLF | 106 | 949-952 |
| NYESO1 | A0201 | RLLEFYLAV | 134 | 953-956 |
| STEAP1 | A0201 | LLLGTIHAV | 152 | 957-960 |
| RNF43 | B0702 | NPQPVWLCL | 146 | 961-964 |
| MAGEA3 | A0201_A2402 | KVPEIVHFL | 118 | 965-968 |

The in silico predicted binding affinity and in vitro binding affinity of the heteroclitic 9-mer peptides are provided in Table 20. The in silico predicted binding affinity is based on the NetMHC4.0 algorithm, which predicts peptide binding to MHC class I molecules in terms of 50% inhibitory concentration (IC50) values (nM); a lower number reflects stronger predicted binding affinity. The in vitro binding affinity was determined through a binding assay that determines the ability of each candidate peptide to bind to the indicated MHC class I alleles and stabilize the MHC-peptide complex by comparing the binding to that of a high affinity T cell epitope. Briefly, each peptide is incubated with its specific HLA molecule in an in vitro assay. Binding strength is compared against a known, immunogenic peptide for the same HLA molecule as a positive control with the positive control binding score set to 100%. The sequence-optimized binding score is normalized to the control peptide. That is, each peptide was given a score relative to the positive control peptide, which is a known T cell epitope with very strong binding properties. The score of the heteroclitic test peptide is reported quantitatively as a percentage of the signal generated by the positive control peptide. Peptides with scores greater than or equal to 45% of the positive control are considered binders. Also provided in Table 20 are the percent expression of each gene in patients with colorectal cancer (The Cancer Genome Atlas database), the HLA allele being tested, and whether the wild-type peptide corresponding to each heteroclitic peptide is known to be immunogenic. For a construct including each of the heteroclitic peptides in Table 20, 100% of colorectal cancer patients with HLA type A*02:01 express at least one of the TAA genes, 98% of colorectal cancer patients with HLA type A*03:01 express at least one of the TAA genes, 100% of colorectal cancer patients with HLA type A*24:02 express at least one of the TAA genes, and 98% of colorectal cancer patients with HLA type B*07:02 express at least one of the TAA genes.

TABLE 20
Binding Affinities of Heteroclitic 9-Mers to HLA.

| | | | In silico | | |
|---|---|---|---|---|---|
| | | | Predicted | | |
| | % Expression | | Binding | In vitro Binding | Wild-Type Peptide |
| TAA Gene | in TCGA | HLA Allele | Affinity IC50^{#} | Affinity\hat{ } | Immunogenic? |
| STEAP1 | 100 | A*02:01 | 5.77 | 188.4 | Yes |
| CEACAM5 | 100 | B*07:02 | 8.36 | 88.3 | Yes |
| CEACAM5 | 100 | A*03:01 | 9.69 | 85.4 | Yes |
| CEACAM5 | 100 | A*24:02 | 6.22 | 77.2 | Yes |
| RNF43 | 100 | B*07:02 | 161.95 | 65.4 | Yes |
| MAGE-A6 | 38 | A*03:01 | 12.83 | 103.7 | unknown |
| MAGE-A3 | 35 | A*02:01 | 50.31 | 168.7 | Yes |
| MAGE-A3 | 35 | A*24:02 | 2966 | 102.4 | unknown |
| MAGE-A4 | 25 | B*07:02 | 7.67 | 49.5 | unknown |
| NY-ESO | 21 | A*02:01 | 4.61 | 212.9 | unknown |
| GAGE | 3 | B*07:02 | 2.58 | 58.5 | unknown |

^{#}NetMHC4.0
\hat{ }% relative to positive control peptide binding

### Head and Neck Cancer Heteroclitic Peptides

A total of 10 peptides with heteroclitic mutations across 6 genes were selected for the head and neck cancer heteroclitic peptides. For each heteroclitic mutation, a peptide of 9 amino acids in length was designed as described in Example 1 and elsewhere herein. The peptides are shown in Table 21. The heteroclitic mutation in each is as described in Table 1.

TABLE 21
Exemplary Head and Neck Cancer Heteroclitic 9-Mers.
Head and Neck Cancer Heteroclitic 9-Mers

| Gene | HLA Type | Sequence | SEQ ID NO |
|---|---|---|---|
| CEACAM5 | A0301 | HVFGYSWYK | 104 |
| CEACAM5 | B0702 | IPQVHTQVL | 108 |
| MAGEA4 | B0702 | MPSLREAAL | 126 |
| CEACAM5 | A2402 | IYPNASLLF | 106 |
| CEACAM5 | A0201 | ILIGVLVGV | 100 |
| STEAP1 | A0201 | LLLGTIHAV | 152 |
| STEAP1 | A2402 | KYKKFPWWL | 154 |
| NYESO1 | B0702 | APRGPHGGM | 136 |
| PRAME | A0201 | NMTHVLYPL | 140 |
| hTERT | A0201_A2402 | IMAKFLHWL | 114 |

The in silico predicted binding affinity and in vitro binding affinity of the heteroclitic 9-mer peptides are provided in Table 22. The in silico predicted binding affinity is based on the NetMHC4.0 algorithm, which predicts peptide binding to MHC class I molecules in terms of 50% inhibitory concentration (IC50) values (nM); a lower number reflects stronger predicted binding affinity. The in vitro binding affinity was determined through a binding assay that determines the ability of each candidate peptide to bind to the indicated MHC class I alleles and stabilize the MHC-peptide complex by comparing the binding to that of a high affinity T cell epitope. Briefly, each peptide is incubated with its specific HLA molecule in an in vitro assay. Binding strength is compared against a known, immunogenic peptide for the same HLA molecule as a positive control with the positive control binding score set to 100%. The sequence-optimized binding score is normalized to the control peptide. That is, each peptide was given a score relative to the positive control peptide, which is a known T cell epitope with very strong binding properties. The score of the heteroclitic test peptide is reported quantitatively as a percentage of the signal generated by the positive control peptide. Peptides with scores greater than or equal to 45% of the positive control are considered binders. Also provided in Table 22 are the percent expression of each gene in patients with head and neck cancer (The Cancer Genome Atlas database), the HLA allele being tested, and whether the wild-type peptide corresponding to each heteroclitic peptide is known to be immunogenic. For a construct including each of the heteroclitic peptides in Table 22, 100% of head and neck cancer patients with HLA type A*02:01 express at least one of the TAA genes, 100% of head and neck cancer patients with HLA type A*03:01 express at least one of the TAA genes, 100% of head and neck cancer patients with HLA type A*24:02 express at least one of the TAA genes, and 100% of head and neck cancer patients with HLA type B*07:02 express at least one of the TAA genes.

TABLE 22
Binding Affinities of Heteroclitic 9-Mers to HLA.

| | | | In silico | | |
|---|---|---|---|---|---|
| | % | | Predicted | | |
| | Expression | | Binding | In vitro Binding | Wild-Type Peptide |
| TAA Gene | in TCGA | HLA Allele | Affinity IC50^{#} | Affinity\hat{ } | Immunogenic? |
| CEACAM5 | 100 | A*02:01 | 6.92 | 170.7 | Yes |
| CEACAM5 | 100 | B*07:02 | 8.36 | 88.3 | Yes |
| CEACAM5 | 100 | A*03:01 | 9.69 | 85.4 | Yes |
| CEACAM5 | 100 | A*24:02 | 6.22 | 77.2 | Yes |
| STEAP1 | 99 | A*02:01 | 5.77 | 188.4 | Yes |
| STEAP1 | 99 | A*24:02 | 47.48 | 104.7 | unknown |
| TERT | 94 | A*02:01 | 7.04 | 123.3 | Yes |
| TERT | 94 | A*24:02 | 2197.84 | 142.3 | unknown |
| PRAME | 91 | A*02:01 | 11.72 | 139.4 | Yes |
| MAGE-A4 | 78 | B*07:02 | 7.67 | 49.5 | unknown |
| NY-ESO1 | 44 | B*07:02 | 3.32 | 109.7 | unknown |

^{#}NetMHC4.0
\hat{ }% relative to positive control peptide binding

### Example 3. Proof of Concept: Efficacy of Lm Heteroclitic WT1 Minigene Fusion Protein Constructs

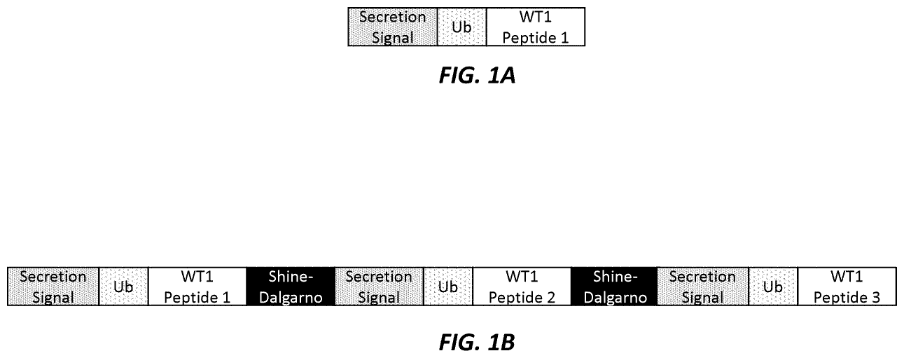

The peptide minigene expression system was used to assess unique heteroclitic minigenes targeting the Wilms tumor protein. This expression system was designed to facilitate cloning of panels of recombinant proteins containing distinct peptide moieties at the carboxy-terminus. This is accomplished by a simple PCR reaction utilizing a sequence encoding one of the Signal Sequence (SS)-Ubiquitin (Ub)-Antigenic Peptide constructs as a template. By using a primer that extends into the carboxy-terminal region of the Ub sequence and introducing codons for the desired peptide sequence at the 3′ end of the primer, a new SS-Ub-Peptide sequence can be generated in a single PCR reaction. The 5′ primer encoding the bacterial promoter and first few nucleotides of the signal sequence (e.g., LLO or ActAmoo secretion signal) can be the same for all constructs. The constructs generated using this strategy are represented schematically in FIGS. 1A and 1B.

One of the advantages of the minigene system is that it will be possible to load cells with multiple peptides using a single Listeria vector construct. Multiple peptides can be introduce into recombinant attenuated Listeria (e.g., Lmdda) using a modification of the single peptide expression system described above. A chimeric protein encoding multiple distinct peptides from sequential SS-Ub-Peptide sequences can be encoded in one insert. See, e.g., FIG. 1B. Shine-Dalgarno ribosome binding sites can be introduced before each SS-Ub-Peptide coding sequence to enable separate translation of each of the peptide constructs. FIG. 1B demonstrates a schematic representation of a construct designed to express three separate peptide antigens from one strain of recombinant Listeria.

To assess the expression of tLLO-WT1-heteroclitic fusion proteins by ADXS Lmdda Listeria constructs, unique heteroclitic minigenes targeting the Wilms Tumor 1 protein were generated in the pAdv134 plasmid and transformed into Lmdda. The pAdv134 tLLO plasmid encodes the N-terminal LLO fragment set forth in SEQ ID NO: 59. The tLLO-WT1 heteroclitic fusion proteins comprise from N-terminal end to C-terminal end: the N-terminal LLO fragment set forth in SEQ ID NO: 59, followed by the FLAG tag set forth in SEQ ID NO: 99, followed by the ubiquitin sequence set forth in SEQ ID NO: 188, followed by a heteroclitic WT1 9-mer listed in Table 23, below.

TABLE 23
Heteroclitic WT1 Peptides.

| | WT1 9-Mer | |
|---|---|---|
| Construct | (Heteroclitic AA | |
| # | Bolded and Underlined) | SEQ ID NO |
| 1 | FMFPNAPYL | 160 |
| 2 | YLGEQQYSV | 161 |
| 3 | YLLPAVPSL | 162 |
| 4 | YLNALLPAV | 163 |
| 5 | ALLLRTPYV | 164 |
| 6 | YLGATLKGV | 165 |
| 7 | KLYFKLSHL | 166 |
| 8 | YMTWNQMNL | 167 |
| 9 | GLRRGIQDV | 168 |
| 10 | YMFPNAPYL | 169 |

The combined WT1-tLLO-FLAG-Ub-heteroclitic phenylalanine construct (construct #1) is set forth in SEQ ID NO: 189 (tLLO=1-441; FLAG=442-462; ubiquitin=463-537; heteroclitic phenylalanine peptide=538-546). One additional construct (Lmdda-WT1-tLLO-P1-P2-P3-FLAG-UB-heteroclitic tyrosine minigene construct) was generated that targets 3 WT1 peptides (P1-P2-P3; SEQ ID NOS: 190 (RSDELVRHHNMHQRNMTKL), 191 (PGCNKRYFKLSHLQMHSRKHTG), and 192 (SGQAYMFPNAPYLPSCLES), respectively). Each ‘P’ peptide is comprised of 19-22 amino acids, sufficient in length to provide additional CD4 T helper epitopes. The three peptides are separated by linkers. The P3 peptide contains a heteroclitic mutation converting SGQARMFPNAPYLPSCLES (SEQ ID NO: 193) to SGQAYMFPNAPYLPSCLES (SEQ ID NO: 192). In addition to the heteroclitic P3 peptide, the Lmdda-WT1-tLLO-P1-P2-P3-FLAG-UB-heteroclitic tyrosine minigene construct contains a ubiquitin-YMFPNAPYL (SEQ ID NO: 169) moiety at the C-terminus. The combined WT1-tLLO-P1-P2-P3-FLAG-UB-heteroclitic tyrosine minigene construct is set forth in SEQ ID NO: 194 (tLLO=1-441; wild-type WT1 peptide v14-WT1-427 long=442-460; wild type WT1 peptide v15-WT1-331 long=466-487; heteroclitic WT1 peptide v1B-WT1-122A1-long=493-511; FLAG=512-532; ubiquitin=533-607; heteroclitic tyrosine peptide=608-616). Each individual Lmdda construct was assayed by Western blot for tLLO-fusion protein expression of the unique heteroclitic WT1 minigene product.

Construct #1 (Lmdda-WT1-tLLO-FLAG-Ub-heteroclitic phenylalanine minigene construct) and the Lmdda-WT1-tLLO-P1-P2-P3-FLAG-UB-heteroclitic tyrosine minigene construct were assayed by Western blot for tLLO-fusion protein expression of the unique heteroclitic WT1 minigene product. Single colonies from plates containing Lm WT1 minigene constructs were used to inoculate an overnight culture in 6 mL of Brain Heart Infusion (BHI) broth in a dry shaking incubator at 37° C. The following day, 1:10 dilution of the original overnight culture were re-suspended in 9 mL of fresh BHI and grown in the dry shaking incubator at 37° C. until reaching an OD₆₀₀=0.6. Cells were pelleted by 2-minute centrifugation at 13000 RPM. Sample supernatant were collected and run on SDS-PAGE. Samples were prepared by diluting 75 μL of sample with 25 μL of 4× LDS Sample Buffer (Cat #161-0747), boiled at 98° C. for 10 minutes, placed on ice, and then centrifuged at max speed for 10 minutes at 4° C. 13 μL of the sample was run on 4-15% precast protein gel (BioRad Cat #4561086). Protein gels were transferred using the Trans-Blot Turbo transfer apparatus (Cat #170-4155) and PVDF Midi transfer packs (Bio-Rad #170-4157). Blots were incubated with anti-FLAG monoclonal Antibody (Sigma F1804) or anti-LLO (Abcam ab200538) as primary and goat anti-mouse IgG-HRP conjugated (sc2005) as a secondary antibody. The blots were then incubated on iBind Flex (Invitrogen cat#1772866), washed, and then developed by Super Signal West Dura Extended Duration Substrate (ThermoFisher #34076); the images were developed on the Amersham Imager 600 (GE).

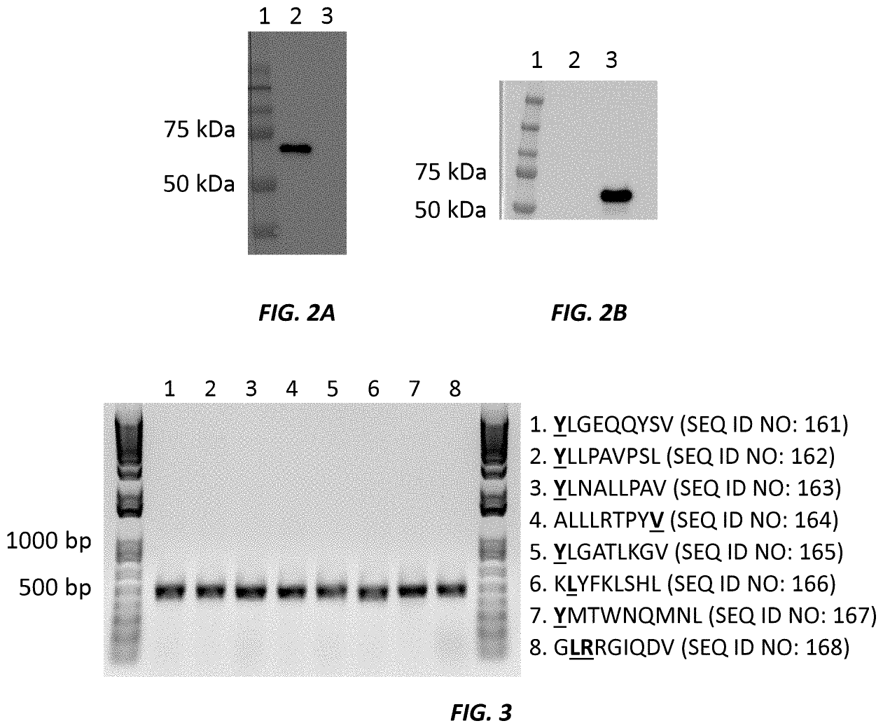

Expression and secretion of the unique tLLO-WT1-heteroclitic minigene fusion proteins was confirmed. Anti-Flag tag antibody Western blots of culture supernatant from construct #1 and the Lmdda-WT1-P1-P2-P3-YMFPNAPYL (SEQ ID NO: 169) Heteroclitic tyrosine+minigene construct are shown in FIGS. 2A and 2B, respectively. We were able to detect a protein band corresponding to the correct size and identity for each individual tLLO-WT1-heteroclitic minigene fusion protein. These data demonstrate the ability for heteroclitic peptides targeting multiple peptide fragments within the WT1 protein to be generated using the pAdv134 plasmid and Lmdda Listeria strain.

For constructs #2-9 in Table 23, each individual Lmdda construct was assayed by colony PCR in order to detect plasmid DNA from each unique tLLO-fusion protein containing heteroclitic WT1 minigenes.

TABLE 24
Materials.

| Material | Vendor | Catalog #/Sequence |
|---|---|---|
| DreamTaq DNA | ThermoFisher | EP0702 |
| Polymerase | | |
| Forward Primer | ThermoFisher | 5′-catcgatcactctgga-3′ |
| (Adv16 f)* | | (SEQ ID NO: 195) |
| Reverse Primer | ThermoFisher | 5′-ctaactccaatgttact |
| (Adv295 r)* | | tg-3′ (SEQ ID NO: 196) |
| 10 mM dNTPs | NEB | N04475 |
| TrackIt 1 kB | ThermoFisher | 10488085 |
| Plus DNA Ladder | | |

### Procedure

The general colony PCR procedure that was used is as follows. Obtained plate with large colonies (generally, plates grown at 37° C. for 24 hours work well for this procedure). Created master mix for PCR as follows.

| Reagent | Volume (μL) |
|---|---|
| PCR water | 16 |
| DreamTaq 10x Buffer | 2 |
| Forward primer | 0.5 |
| Reverse primer | 0.5 |
| 10 mM dNTPs | 0.5 |
| Dream Taq Polymerase | 0.5 |
| | = 20 |

Aliquoted 20 μL of master mix into each PCR tube. Using a pipette tip (10-20 μL volume works best), scooped up a generous volume from one colony. Tapped the pipette tip into the PCR tube several times and swirled around to dislodge the bacteria. Ran the PCR reaction(s) in a thermocycler using the following PCR program.

| Step | Temp (° C.) | Time |
|---|---|---|
| 1 | 94 | 2 minutes |
| 2 | 94 | 30 seconds |
| 3 | 55* | 30 seconds |
| 4 | 72 | 1 minute |
| repeat steps 2-4 an additional 29x | | |
| 5 | 72 | 5 minutes |
| 6 | 4 | ∞ |

Removed PCR tubes from the thermocycler, added 4 μL of 6× loading dye. Ran 10 μL of each PCR reaction on a 1% agarose gel, alongside 10 μL of the 1 kb+DNA ladder. The primers added an additional 163 base pairs to the product. The forward primer bound 70 base pairs upstream of the 3′ end of tLLO (includes the Xhol site). The reverse primer bound 93 base pairs downstream of the stop sites (includes the Xmal site).

Representative colony PCR results showing Lmdda strains containing pAdv134 WT1-heteroclitic plasmids #2-9 from Table 23 are shown in FIG. 3. We were able to detect a DNA band corresponding to the correct size and identity for each individual tLLO-WT1-heteroclitic minigene plasmid. These data demonstrate the ability for heteroclitic peptides targeting multiple peptide fragments within the WT1 protein to be generated using the pAdv134 plasmid and Lmdda Listeria strain, which indicates that such constructs can be used as therapeutic compositions to target WT1 to create or enhance immune responses against WT1 and WT1-expressing cancers and tumors.

To assess the generation of WT1-specific T cell responses in AAD mice using two different WT1 constructs, ELISpots was performed to determine the desired vaccine-induced Ag-specific responses. The AAD mice (B6.Cg-Tg(HLA-A/H2-D)2Enge/J; The Jackson Laboratory—Stock No.: 004191) are transgenic mice that express an interspecies hybrid class I MHC gene, AAD, which contains the alpha-1 and alpha-2 domains of the human HLA-A2.1 gene and the alpha-3 transmembrane and cytoplasmic domains of the mouse H-2D^{d} gene, under the direction of the human HLA-A2.1 promoter. This transgenic strain enables the modeling of human T cell immune responses to HLA-A2 presented antigens, and may be useful in testing of vaccines for infectious diseases or cancer therapy. The immunization schedule is provided in Table 25. The mice that were used were female C57BL/6 mice aged 8-10 weeks.

TABLE 25
Immunization Schedule.

| | | | Dose 1 | Dose 2 | |
|---|---|---|---|---|---|
| Vaccine/ | Titer- | | (IP/200 | (IP/200 | |
| Group | CFU/mL | Mice/Group | μL/mouse) | μL/mouse) | Harvest |
| 1-PBS | N/A | 5 | Day 0 | Day 12 | Day 18 |
| 2-LmddA 274 | ~1 × 10⁹ | 5 | Day 0 | Day 12 | Day 18 |
| 3-WT1Fm-FLAG-Ub-9 | ~1 × 10⁹ | 5 | Day 0 | Day 12 | Day 18 |
| (WT1-F minigene) | | | | | |
| 4-LmddA + pAdv134-WT1m:Ub-9 | ~1 × 10⁹ | 5 | Day 0 | Day 12 | Day 18 |
| (WT1-AH1-Tyr minigene) | | | | | |

Vaccine Preparations. Briefly, each glycerol stock was streaked over required nutrient plate and grown overnight. A single colony was used for growth in an overnight culture of Brain Heart Infusion (BHI) broth under antibiotic selection. Overnight cultures were used at a 1:10 (vol/vol) dilution to inoculate fresh BHI broth. Bacteria were incubated in an orbital shaker for 1-3 hours at 37° C. to mid-log phase, an OD of −0.6-0.7. Mice were infected with 1×10⁹ CFU Lm by i.p. inoculation in PBS.

ELISPOT. On day 18, mice were sacrificed by CO₂ asphyxiation in accordance with IACUC protocols, spleens were harvested, and splenocyte single-cell suspensions were plated on 96-well plates and stimulated with either the wild-type or heteroclitic peptide (Table 26). Similar experiments are done with other wild-type and heteroclitic peptide pairs (Table 27). An ELISPOT assay was used to enumerate antigen specific CD8 T Cells responding to either the wild-type or heteroclitic peptides. The full ELISPOT protocol was as per CTL immunospot (www.immunospot.com/resources/protocols/ELISPOT-protocol.htm).

TABLE 26
Wild-Type and Heteroclitic WT1 Peptides.

| Wild-Type | Negative | |
|---|---|---|
| Peptide | Control | Heteroclitic Peptides |
| RMFPNAPYL | RPMI Empty | FMFPNAPYL (SEQ ID NO: 160) |
| (SEQ ID | Media | YMFPNAPYL (SEQ ID NO: 169) |
| NO: 197) | | |

TABLE 27
Wild-Type and Heteroclitic WT1 Peptides.

| Wild-Type | Heteroclitic |
|---|---|
| SLGEQQYSV | YLGEQQYSV (SEQ ID NO: 161) |
| (SEQ ID NO: 198) | |
| ALLPAVPSL | YLLPAVPSL (SEQ ID NO: 162) |
| (SEQ ID NO: 199) | |
| DLNALLPAV | YLNALLPAV (SEQ ID NO: 163) |
| (SEQ ID NO: 200) | |
| ALLLRTPYS | ALLLRTPYV (SEQ ID NO: 164) |
| (SEQ ID NO: 201) | |
| NLGATLKGV | YLGATLKGV (SEQ ID NO: 165) |
| (SEQ ID NO: 202) | |
| KRYFKLSHL | KLYFKLSHL (SEQ ID NO: 166) |
| (SEQ ID NO: 203) | |
| CMTWNQMNL | YMTWNQMNL (SEQ ID NO: 167) |
| (SEQ ID NO: 204) | |
| GVFRGIQDV | GLRRGIQDV (SEQ ID NO: 168) |
| (SEQ ID NO: 205) | |

A generic ELISPOT protocol is provided below.

DAY 0 (Sterile Conditions). Prepared Capture Solution by diluting the Capture Antibody according to specific protocol. Many cytokines benefit from pre-wetting the PVDF membrane with 70% ethanol for 30 sec and washing with 150 μL of PBS three times before adding 80 μL of the Capture Solution into each well. Incubated plate overnight at 4° C. in a humidified chamber.

DAY 1 (Sterile Conditions). Prepared CTL-Test™ Medium by adding 1% fresh L-glutamine. Prepared antigen/mitogen solutions at 2× final concentration in CTL-Test™ Medium. Decanted plate with coating antibody from Day 0 and washed one time with 150 μL PBS. Plated antigen/mitogen solutions, 100 μL/well. After thawing PBMC or isolating white blood cells with density gradient, adjusted PBMC to desired concentration in CTL-Test™ Medium, e.g., 3 million/mL corresponding to 300,000 cells/well (however, cell numbers can be adjusted according to expected spot counts since 100,000-800,000 cells/well will provide linear results). While processing PBMC and until plating, kept cells at 37° C. in humidified incubator, 5-9% CO₂. Plated PBMC, 100 μL/well using large orifice tips. Once completed, gently tapped the sides of the plate and immediately placed into a 37° C. humidified incubator, 5-9% CO₂. Incubated for 24-72 hours depending on your cytokine. Did not stack plates. Avoided shaking plates by carefully opening and shutting incubator door. Did not touch plates during incubation.

DAY2. Prepared Wash Solutions for the day: PBS, distilled water and Tween-PBS. Prepared Detection Solution by diluting Detection Antibody according to specific protocol. Washed plate two times with PBS and then two times with 0.05% Tween-PBS, 200 μL/well each time. Added 80 μL/well Detection Solution. Incubated at RT, 2h. Prepared Tertiary Solution by diluting the Tertiary Antibody according to specific protocol. Washed plate three times with 0.05% Tween-PBS, 200 μL/well. Added 80 μL/well of Strep-AP Solution. Incubated at RT, 30 min. Prepared Developer Solution according to your specific protocol. Washed plate two times with 0.05% Tween-PBS, and then two times with distilled water, 200 μL/well each time. Add Developer Solution, 80 μL/well. Incubated at RT, 10-20 min. Stopped reaction by gently rinsing membrane with tap water, decanted, and repeated three times. Removed protective underdrain of the plate and rinsed back of plate with tap water. Air dried plate for 2 hours face-down in running hood or on paper towels for 24 hours on bench top. Scanned and counted plate.

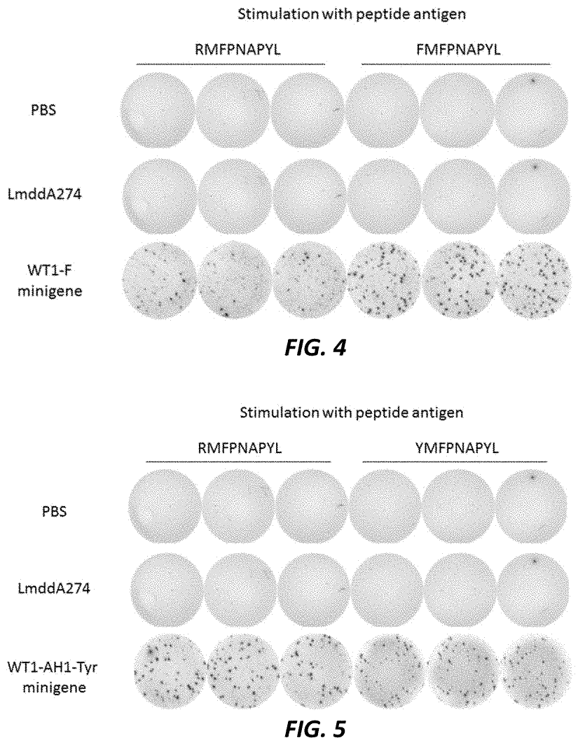

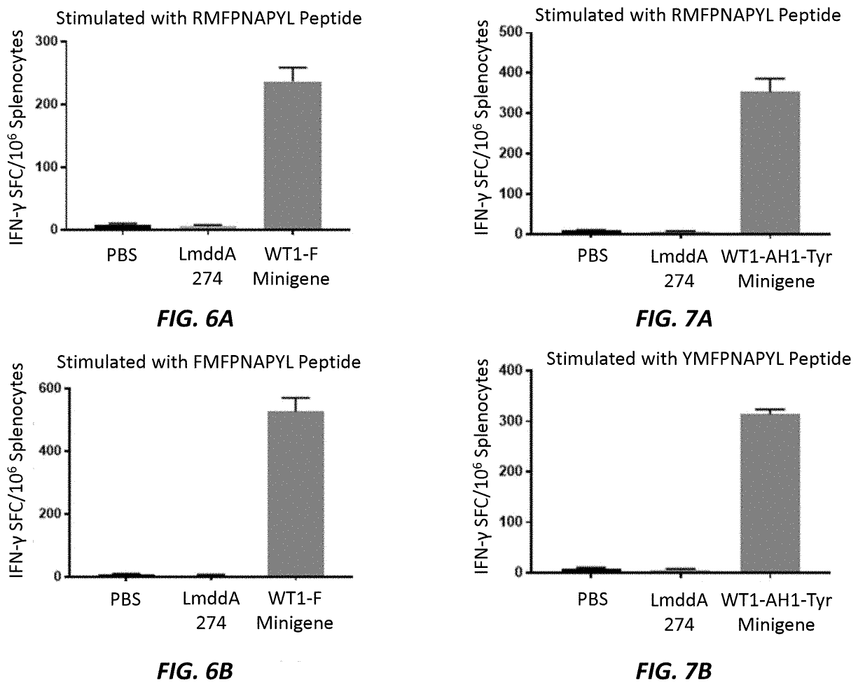

HLA-A2 transgenic B6 mice were vaccinated as described, and splenocytes were stimulated ex vivo with specific WT1 peptides (RMFPNAPYL (SEQ ID NO: 197), FMFPNAPYL (SEQ ID NO 160)) and analyzed by IFNg ELISpot assay. Heteroclitic vaccination (WT1-F minigene: FMFPNAPYL; SEQ ID NO: 160) induced Ag-specific T cell responses in immunized HLA2 transgenic mice. See FIG. 4 and FIG. 6B. In addition, heteroclitic vaccination elicited T cell responses that cross-reacted with the native WT1 tumor antigen (RMFPNAPYL; SEQ ID NO: 197). See FIG. 4 and FIG. 6A. The data demonstrated that vaccination with the WT1-F heteroclitic minigene vaccine can elicit T cells that are cross-reactive with the WT1-native tumor antigen (RMFPNAPYL; SEQ ID NO: 197). Overall, the data demonstrated that the heteroclitic minigene vaccine can elicit T cells that cross-react with the native tumor antigen.

HLA-A2 transgenic B6 mice were vaccinated as described and splenocytes were harvested. The ability of T cells to produce IFNg in response to vaccine-specific YMFPNAPYL peptide (SEQ ID NO: 169) or native WT1 peptide (RMFPNAPYL; SEQ ID NO: 197) was determined by IFNg ELISpot assay. Heteroclitic vaccination (WT1-AH1-Tyr minigene: YMFPNAPYL; SEQ ID NO: 169) induced Ag-specific T cell responses in immunized HLA2 transgenic mice. See FIG. 5 and FIG. 7B. In addition, heteroclitic vaccination elicited T cell responses that cross-react with the native WT1 tumor antigen (RMFPAPYL; SEQ ID NO: 197). See FIG. 5 and FIG. 7A.

### Example 4. Proof of Concept: Therapeutic Efficacy of Heteroclitic Lm-AH1 Constructs in a CT26 Challenge Study

This study examined if Lm AH1-HC heteroclitic minigene vaccine could control or suppress CT26 tumor growth.

### Treatment Schedule

Heteroclitic AH1-HC vaccination began as described in Table 28, followed with two boosts at one-week intervals with the recommended vaccine.

TABLE 28
Treatments Schedule.

| | | | Weekly Dose: | Weekly Dose: | Weekly Dose: |
|---|---|---|---|---|---|
| | CT26 | | Lm 1 × 10⁸ | Lm 1 × 10⁸ | Lm 1 × 10⁸ |
| | Implantation | Titer | (IV/200 uL/ | (IV/200 uL/ | (IV/200 uL/ |
| Group (N = 10) | 3 × 10⁵ cells | CFU/mL | mouse) | mouse) | mouse) |
| Naive | Aug. 7, 2017 | N/A | Aug. 10, 2017 | Aug. 17, 2017 | Aug. 24, 2017 |
| AH1-HC | Aug. 7, 2017 | 6 × 10⁸ | Aug. 10, 2017 | Aug. 17, 2017 | Aug. 24, 2017 |

### Experimental Details

Vaccine Dosing Details. AH1-HC refers to mice primed and boosted with heteroclitic AH1-HC vaccine.

Tumor Cell Line Expansion. CT26 cell line were cultured in RPMI with 10% FBS.

Tumor Inoculation. On Day 0, (14JUN17) CT26 cells will be trypsinized with 0.25% trypsin (1×) and washed twice with media at the appropriate concentration in PBS (3×10⁵ cells/mouse). CT26 cells were implanted subcutaneously in the right flank of each mouse.

Treatment. Vaccine preparation was as follows: (a) thawed 1 vial form −80° C. in 37° C. water bath; (b) spun at 14,000 rpm for 2 min and discarded supernatant; (c) washed 2 times with 1 mL PBS and discarded PBS; and (d) re-suspended to a final concentration of 5×10⁸ CFU/mL. Vaccine dosing began 3-4 days after tumor implantation.

TABLE 29
Construct Sequences.

| Construct | Sequence |
|---|---|
| Lm-AH1 HC | DYKDHDGDYKDHDIDYKDDDKQIFVK |
| | TLTGKTITLEVEPSDTIENVKAKIQ |
| | DKEGIPPDQQRLIFAGKQLEDGRTLSD |
| | YNIQKESTLHLVLRLRGGMPKYAYHML |
| | (SEQ ID NO: 206) |
| | Ubiquitin: 22-96 |
| | Heteroclitic AH1 9mer: 97-105 |
| AH1 Wild Type | SPSYVYHQF (SEQ ID NO: 207) |
| AH1 Heteroclitic | MPKYAYHML (SEQ ID NO: 208) |

### RESULTS AND CONCLUSIONS

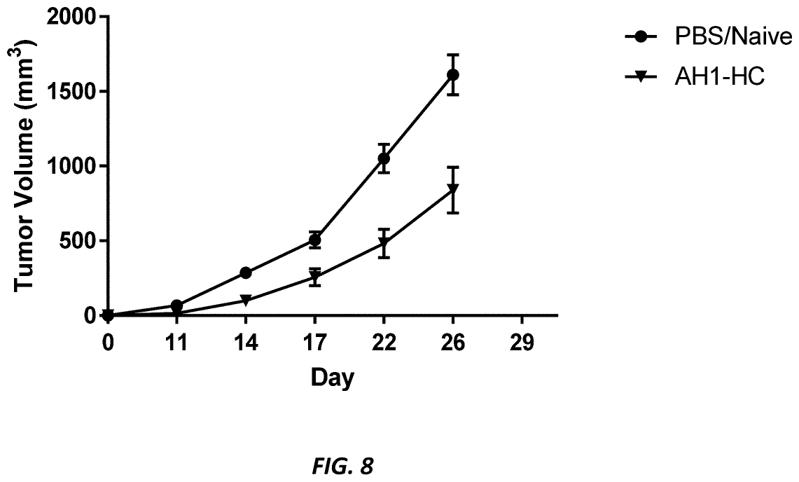

The Lm-AH1 HC construct was able to significantly control tumor growth in the murine CT26 colorectal cancer model. See FIG. 8.

We claim:

1. An isolated peptide comprising an immunogenic fragment of a cancer-associated protein, wherein the fragment comprises a heteroclitic mutation.

2. The isolated peptide of claim 1, wherein the heteroclitic mutation is a mutation to a preferred amino acid at an anchor position.

3. The isolated peptide of claim 1 or 2, wherein the fragment is between about 7 and about 11 amino acids in length, between about 8 and about 10 amino acids in length, or about 9 amino acids in length.

4. The isolated peptide of any preceding claim, wherein the cancer-associated protein is a cancer testis antigen or oncofetal antigen.

5. The isolated peptide of any preceding claim, wherein the cancer-associated protein is encoded by one of the following human genes: CEACAM5, GAGE1, TERT, KLHL7, MAGEA3, MAGEA4, MAGEA6, NUF2, NYESO1, PAGE4, PRAME, PSA, PSMA, RNF43, SART3, SSX2, STEAP1, and SURVIVIN.

6. The isolated peptide of claim 5, wherein:
(a) the cancer-associated protein is encoded by CEACAM5, and the fragment comprises any one of SEQ ID NOS: 100, 102, 104, 106, and 108;
(b) the cancer-associated protein is encoded by GAGE1, and the fragment comprises any one of SEQ ID NOS: 110 and 112;
(c) the cancer-associated protein is encoded by TERT, and the fragment comprises SEQ ID NO: 114;
(d) the cancer-associated protein is encoded by KLHL7, and the fragment comprises SEQ ID NO: 116;
(e) the cancer-associated protein is encoded by MAGEA3, and the fragment comprises any one of SEQ ID NOS: 118, 120, 122, and 124;
(f) the cancer-associated protein is encoded by MAGEA4, and the fragment comprises SEQ ID NO: 126;
(g) the cancer-associated protein is encoded by MAGEA6, and the fragment comprises SEQ ID NO: 128;
(h) the cancer-associated protein is encoded by NUF2, and the fragment comprises any one of SEQ ID NOS: 130 and 132;
(i) the cancer-associated protein is encoded by NYESO1, and the fragment comprises any one of SEQ ID NOS: 134 and 136;
(j) the cancer-associated protein is encoded by PAGE4, and the fragment comprises SEQ ID NO: 138;
(k) the cancer-associated protein is encoded by PRAME, and the fragment comprises SEQ ID NO: 140;
(l) the cancer-associated protein is encoded by PSA, and the fragment comprises SEQ ID NO: 142;
(m) the cancer-associated protein is encoded by PSMA, and the fragment comprises SEQ ID NO: 144;
(n) the cancer-associated protein is encoded by RNF43, and the fragment comprises SEQ ID NO: 146;
(o) the cancer-associated protein is encoded by SART3, and the fragment comprises SEQ ID NO: 148;
(p) the cancer-associated protein is encoded by SSX2, and the fragment comprises SEQ ID NO: 150;
(q) the cancer-associated protein is encoded by STEAP1, and the fragment comprises any one of SEQ ID NOS: 152 and 154; or
(r) the cancer-associated protein is encoded by SURVIVIN, and the fragment comprises any one of SEQ ID NOS: 156 and 158.

7. The isolated peptide of claim 6, wherein:
(a) the cancer-associated protein is encoded by CEACAM5, and the fragment consists of any one of SEQ ID NOS: 100, 102, 104, 106, and 108;
(b) the cancer-associated protein is encoded by GAGE1, and the fragment consists of any one of SEQ ID NOS: 110 and 112;
(c) the cancer-associated protein is encoded by TERT, and the fragment consists of SEQ ID NO: 114;
(d) the cancer-associated protein is encoded by KLHL7, and the fragment consists of SEQ ID NO: 116;
(e) the cancer-associated protein is encoded by MAGEA3, and the fragment consists of any one of SEQ ID NOS: 118, 120, 122, and 124;
(f) the cancer-associated protein is encoded by MAGEA4, and the fragment consists of SEQ ID NO: 126;
(g) the cancer-associated protein is encoded by MAGEA6, and the fragment consists of SEQ ID NO: 128;
(h) the cancer-associated protein is encoded by NUF2, and the fragment consists of any one of SEQ ID NOS: 130 and 132;
(i) the cancer-associated protein is encoded by NYESO1, and the fragment consists of any one of SEQ ID NOS: 134 and 136;
(j) the cancer-associated protein is encoded by PAGE4, and the fragment consists of SEQ ID NO: 138;
(k) the cancer-associated protein is encoded by PRAME, and the fragment consists of SEQ ID NO: 140;
(l) the cancer-associated protein is encoded by PSA, and the fragment consists of SEQ ID NO: 142;
(m) the cancer-associated protein is encoded by PSMA, and the fragment consists of SEQ ID NO: 144;
(n) the cancer-associated protein is encoded by RNF43, and the fragment consists of SEQ ID NO: 146;
(o) the cancer-associated protein is encoded by SART3, and the fragment consists of SEQ ID NO: 148;
(p) the cancer-associated protein is encoded by SSX2, and the fragment consists of SEQ ID NO: 150;
(q) the cancer-associated protein is encoded by STEAP1, and the fragment consists of any one of SEQ ID NOS: 152 and 154; or
(r) the cancer-associated protein is encoded by SURVIVIN, and the fragment consists of any one of SEQ ID NOS: 156 and 158.

8. The isolated peptide of claim 7, wherein:
(a) the cancer-associated protein is encoded by CEACAM5, and the isolated peptide consists of any one of SEQ ID NOS: 100, 102, 104, 106, and 108;
(b) the cancer-associated protein is encoded by GAGE1, and the isolated peptide consists of any one of SEQ ID NOS: 110 and 112;
(c) the cancer-associated protein is encoded by TERT, and the isolated peptide consists of SEQ ID NO: 114;
(d) the cancer-associated protein is encoded by KLHL7, and the isolated peptide consists of SEQ ID NO: 116;
(e) the cancer-associated protein is encoded by MAGEA3, and the isolated peptide consists of any one of SEQ ID NOS: 118, 120, 122, and 124;
(f) the cancer-associated protein is encoded by MAGEA4, and the isolated peptide consists of SEQ ID NO: 126;
(g) the cancer-associated protein is encoded by MAGEA6, and the isolated peptide consists of SEQ ID NO: 128;
(h) the cancer-associated protein is encoded by NUF2, and the isolated peptide consists of any one of SEQ ID NOS: 130 and 132;
(i) the cancer-associated protein is encoded by NYESO1, and the isolated peptide consists of any one of SEQ ID NOS: 134 and 136;
(j) the cancer-associated protein is encoded by PAGE4, and the isolated peptide consists of SEQ ID NO: 138;
(k) the cancer-associated protein is encoded by PRAME, and the isolated peptide consists of SEQ ID NO: 140;
(l) the cancer-associated protein is encoded by PSA, and the isolated peptide consists of SEQ ID NO: 142;
(m) the cancer-associated protein is encoded by PSMA, and the isolated peptide consists of SEQ ID NO: 144;
(n) the cancer-associated protein is encoded by RNF43, and the isolated peptide consists of SEQ ID NO: 146;
(o) the cancer-associated protein is encoded by SART3, and the isolated peptide consists of SEQ ID NO: 148;
(p) the cancer-associated protein is encoded by SSX2, and the isolated peptide consists of SEQ ID NO: 150;
(q) the cancer-associated protein is encoded by STEAP1, and the isolated peptide consists of any one of SEQ ID NOS: 152 and 154; or
(r) the cancer-associated protein is encoded by SURVIVIN, and the isolated peptide consists of any one of SEQ ID NOS: 156 and 158.

9. The isolated peptide of any preceding claim, wherein the fragment binds to one or more of the following HLA types: HLA-A*02:01, HLA-A*03:01, HLA-A*24:02, and HLA-B*07:02.

10. A nucleic acid encoding the isolated peptide of any preceding claim.

11. The nucleic acid of claim 10, wherein the nucleic acid is codon optimized for expression in humans.

12. The nucleic acid of claim 10, wherein the nucleic acid is codon optimized for expression in Listeria monocytogenes.

13. The nucleic acid of any one of claims 10-12, wherein the nucleic acid comprises DNA.

14. The nucleic acid of any one of claims 10-12, wherein the nucleic acid comprises RNA.

15. The nucleic acid of any one of claims 10-14, wherein the nucleic acid comprises a sequence selected from any one of SEQ ID NOS: 223-977 and degenerate variants thereof that encode the same amino acid sequence.

16. The nucleic acid of claim 15, wherein the nucleic acid consists of a sequence selected from any one of SEQ ID NOS: 223-977 and degenerate variants thereof that encode the same amino acid sequence.

17. A pharmaceutical composition comprising:
(a) one or more isolated peptides of any one of claims 1-9 or one or more nucleic acids of any one of claims 10-16; and
(b) an adjuvant.

18. The pharmaceutical composition of claim 17, wherein the adjuvant comprises a detoxified listeriolysin O (dtLLO), a granulocyte/macrophage colony-stimulating factor (GM-CSF) protein, a nucleotide molecule encoding a GM-CSF protein, saponin QS21, monophosphoryl lipid A, an unmethylated CpG-containing oligonucleotide, or Montanide ISA 51.

19. The pharmaceutical composition of claim 17 or 18, wherein the pharmaceutical composition comprises peptides or nucleic acids encoding peptides that bind to each of the following HLA types: HLA-A*02:01, HLA-A*03:01, HLA-A*24:02, and HLA-B *07:02.

20. The pharmaceutical composition of any one of claims 17-19, wherein the pharmaceutical composition comprises:
(a) two or more of the peptides set forth in Table 3 or nucleic acids encoding two or more of the peptides set forth in Table 3;
(b) two or more of the peptides set forth in Table 5 or nucleic acids encoding two or more of the peptides set forth in Table 5;
(c) two or more of the peptides set forth in Table 7 or nucleic acids encoding two or more of the peptides set forth in Table 7;
(d) two or more of the peptides set forth in Table 9 or nucleic acids encoding two or more of the peptides set forth in Table 9;
(e) two or more of the peptides set forth in Table 11 or nucleic acids encoding two or more of the peptides set forth in Table 11;
(f) two or more of the peptides set forth in Table 13 or nucleic acids encoding two or more of the peptides set forth in Table 13;
(g) two or more of the peptides set forth in Table 15 or nucleic acids encoding two or more of the peptides set forth in Table 15;
(h) two or more of the peptides set forth in Table 17 or nucleic acids encoding two or more of the peptides set forth in Table 17;
(i) two or more of the peptides set forth in Table 19 or nucleic acids encoding two or more of the peptides set forth in Table 19; or
(j) two or more of the peptides set forth in Table 21 or nucleic acids encoding two or more of the peptides set forth in Table 21.

21. The pharmaceutical composition of claim 20, wherein the pharmaceutical composition comprises:
(a) all of the peptides set forth in Table 3 or nucleic acids encoding all of the peptides set forth in Table 3;
(b) all of the peptides set forth in Table 5 or nucleic acids encoding all of the peptides set forth in Table 5;
(c) all of the peptides set forth in Table 7 or nucleic acids encoding all of the peptides set forth in Table 7;
(d) all of the peptides set forth in Table 9 or nucleic acids encoding all of the peptides set forth in Table 9;
(e) all of the peptides set forth in Table 11 or nucleic acids encoding all of the peptides set forth in Table 11;
(f) all of the peptides set forth in Table 13 or nucleic acids encoding all of the peptides set forth in Table 13;
(g) all of the peptides set forth in Table 15 or nucleic acids encoding all of the peptides set forth in Table 15;
(h) all of the peptides set forth in Table 17 or nucleic acids encoding all of the peptides set forth in Table 17;
(i) all of the peptides set forth in Table 19 or nucleic acids encoding all of the peptides set forth in Table 19; or
(j) all of the peptides set forth in Table 21 or nucleic acids encoding all of the peptides set forth in Table 21.

22. A recombinant bacteria strain comprising a nucleic acid encoding any one of the isolated peptides of claims 1-9.

23. A recombinant bacteria strain comprising one or more nucleic acids encoding two or more of the isolated peptides of claims 1-9.

24. The recombinant bacteria strain of claim 23, wherein the two or more peptides comprise:
(a) two or more of the peptides set forth in Table 3 or nucleic acids encoding two or more of the peptides set forth in Table 3;
(b) two or more of the peptides set forth in Table 5 or nucleic acids encoding two or more of the peptides set forth in Table 5;
(c) two or more of the peptides set forth in Table 7 or nucleic acids encoding two or more of the peptides set forth in Table 7;
(d) two or more of the peptides set forth in Table 9 or nucleic acids encoding two or more of the peptides set forth in Table 9;
(e) two or more of the peptides set forth in Table 11 or nucleic acids encoding two or more of the peptides set forth in Table 11;
(f) two or more of the peptides set forth in Table 13 or nucleic acids encoding two or more of the peptides set forth in Table 13;
(g) two or more of the peptides set forth in Table 15 or nucleic acids encoding two or more of the peptides set forth in Table 15;
(h) two or more of the peptides set forth in Table 17 or nucleic acids encoding two or more of the peptides set forth in Table 17;
(i) two or more of the peptides set forth in Table 19 or nucleic acids encoding two or more of the peptides set forth in Table 19; or
(j) two or more of the peptides set forth in Table 21 or nucleic acids encoding two or more of the peptides set forth in Table 21.

25. The recombinant bacteria strain of claim 24, wherein the two or more peptides comprise:
(a) all of the peptides set forth in Table 3 or nucleic acids encoding all of the peptides set forth in Table 3;
(b) all of the peptides set forth in Table 5 or nucleic acids encoding all of the peptides set forth in Table 5;
(c) all of the peptides set forth in Table 7 or nucleic acids encoding all of the peptides set forth in Table 7;
(d) all of the peptides set forth in Table 9 or nucleic acids encoding all of the peptides set forth in Table 9;
(e) all of the peptides set forth in Table 11 or nucleic acids encoding all of the peptides set forth in Table 11;
(f) all of the peptides set forth in Table 13 or nucleic acids encoding all of the peptides set forth in Table 13;
(g) all of the peptides set forth in Table 15 or nucleic acids encoding all of the peptides set forth in Table 15;
(h) all of the peptides set forth in Table 17 or nucleic acids encoding all of the peptides set forth in Table 17;
(i) all of the peptides set forth in Table 19 or nucleic acids encoding all of the peptides set forth in Table 19; or
(j) all of the peptides set forth in Table 21 or nucleic acids encoding all of the peptides set forth in Table 21.

26. The recombinant bacteria strain of any one of claims 23-25, wherein the combination of two or more peptides binds to each of the following HLA types: HLA-A*02:01, HLA-A*03:01, HLA-A*24:02, and HLA-B*07:02.

27. The recombinant bacteria strain of any one of claims 22-26, wherein the bacteria strain is a Salmonella, Listeria, Yersinia, Shigella, or Mycobacterium strain.

28. The recombinant bacteria strain of claim 27, wherein the bacteria strain is a Listeria strain, optionally wherein the Listeria strain is a Listeria monocytogenes strain.

29. A recombinant Listeria strain comprising a nucleic acid comprising a first open reading frame encoding a fusion polypeptide, wherein the fusion polypeptide comprises a PEST-containing peptide fused to an immunogenic fragment of a cancer-associated protein, wherein the fragment comprises a heteroclitic mutation.

30. The recombinant Listeria strain of claim 29, wherein the heteroclitic mutation is a mutation to a preferred amino acid at an anchor position.

31. The recombinant Listeria strain of claim 29 or 30, wherein the fragment is between about 7 and about 11 amino acids in length, between about 8 and about 10 amino acids in length, or about 9 amino acids in length.

32. The recombinant Listeria strain of any one of claims 29-31, wherein the cancer-associated protein is a cancer testis antigen or oncofetal antigen.

33. The recombinant Listeria strain of any one of claims 29-32, wherein the cancer-associated protein is encoded by one of the following human genes: CEACAM5, GAGE1, TERT, KLHL7, MAGEA3, MAGEA4, MAGEA6, NUF2, NYESO1, PAGE4, PRAME, PSA, PSMA, RNF43, SART3, SSX2, STEAP1, and SURVIVIN.

34. The recombinant Listeria strain of claim 33, wherein:
(a) the cancer-associated protein is encoded by CEACAM5, and the fragment comprises any one of SEQ ID NOS: 100, 102, 104, 106, and 108;
(b) the cancer-associated protein is encoded by GAGE1, and the fragment comprises any one of SEQ ID NOS: 110 and 112;
(c) the cancer-associated protein is encoded by TERT, and the fragment comprises SEQ ID NO: 114;
(d) the cancer-associated protein is encoded by KLHL7, and the fragment comprises SEQ ID NO: 116;
(e) the cancer-associated protein is encoded by MAGEA3, and the fragment comprises any one of SEQ ID NOS: 118, 120, 122, and 124;
(f) the cancer-associated protein is encoded by MAGEA4, and the fragment comprises SEQ ID NO: 126;
(g) the cancer-associated protein is encoded by MAGEA6, and the fragment comprises SEQ ID NO: 128;
(h) the cancer-associated protein is encoded by NUF2, and the fragment comprises any one of SEQ ID NOS: 130 and 132;
(i) the cancer-associated protein is encoded by NYESO1, and the fragment comprises any one of SEQ ID NOS: 134 and 136;
(j) the cancer-associated protein is encoded by PAGE4, and the fragment comprises SEQ ID NO: 138;
(k) the cancer-associated protein is encoded by PRAME, and the fragment comprises SEQ ID NO: 140;
(l) the cancer-associated protein is encoded by PSA, and the fragment comprises SEQ ID NO: 142;
(m) the cancer-associated protein is encoded by PSMA, and the fragment comprises SEQ ID NO: 144;
(n) the cancer-associated protein is encoded by RNF43, and the fragment comprises SEQ ID NO: 146;
(o) the cancer-associated protein is encoded by SART3, and the fragment comprises SEQ ID NO: 148;
(p) the cancer-associated protein is encoded by SSX2, and the fragment comprises SEQ ID NO: 150;
(q) the cancer-associated protein is encoded by STEAP1, and the fragment comprises any one of SEQ ID NOS: 152 and 154; or
(r) the cancer-associated protein is encoded by SURVIVIN, and the fragment comprises any one of SEQ ID NOS: 156 and 158.

35. The recombinant Listeria strain of claim 34, wherein:
(a) the cancer-associated protein is encoded by CEACAM5, and the fragment consists of any one of SEQ ID NOS: 100, 102, 104, 106, and 108;
(b) the cancer-associated protein is encoded by GAGE1, and the fragment consists of any one of SEQ ID NOS: 110 and 112;
(c) the cancer-associated protein is encoded by TERT, and the fragment consists of SEQ ID NO: 114;
(d) the cancer-associated protein is encoded by KLHL7, and the fragment consists of SEQ ID NO: 116;
(e) the cancer-associated protein is encoded by MAGEA3, and the fragment consists of any one of SEQ ID NOS: 118, 120, 122, and 124;
(f) the cancer-associated protein is encoded by MAGEA4, and the fragment consists of SEQ ID NO: 126;
(g) the cancer-associated protein is encoded by MAGEA6, and the fragment consists of SEQ ID NO: 128;
(h) the cancer-associated protein is encoded by NUF2, and the fragment consists of any one of SEQ ID NOS: 130 and 132;
(i) the cancer-associated protein is encoded by NYESO1, and the fragment consists of any one of SEQ ID NOS: 134 and 136;
(j) the cancer-associated protein is encoded by PAGE4, and the fragment consists of SEQ ID NO: 138;
(k) the cancer-associated protein is encoded by PRAME, and the fragment consists of SEQ ID NO: 140;
(l) the cancer-associated protein is encoded by PSA, and the fragment consists of SEQ ID NO: 142;
(m) the cancer-associated protein is encoded by PSMA, and the fragment consists of SEQ ID NO: 144;
(n) the cancer-associated protein is encoded by RNF43, and the fragment consists of SEQ ID NO: 146;
(o) the cancer-associated protein is encoded by SART3, and the fragment consists of SEQ ID NO: 148;
(p) the cancer-associated protein is encoded by SSX2, and the fragment consists of SEQ ID NO: 150;
(q) the cancer-associated protein is encoded by STEAP1, and the fragment consists of any one of SEQ ID NOS: 152 and 154; or
(r) the cancer-associated protein is encoded by SURVIVIN, and the fragment consists of any one of SEQ ID NOS: 156 and 158.

36. The recombinant Listeria strain of any one of claims 29-35, wherein the fragment binds to one or more of the following HLA types: HLA-A*02:01, HLA-A*03:01, HLA-A*24:02, and HLA-B*07:02.

37. The recombinant Listeria strain of any one of claims 29-36, wherein the PEST-containing peptide comprises a bacterial secretion signal sequence, and the fusion polypeptide further comprises a ubiquitin protein fused to the fragment, wherein the PEST-containing peptide, the ubiquitin, and the carboxy-terminal antigenic peptide are arranged in tandem from the amino-terminal end to the carboxy-terminal end of the fusion polypeptide.

38. The recombinant Listeria strain of any one of claims 29-37, wherein the fusion polypeptide comprises the PEST-containing peptide fused to two or more immunogenic fragments of cancer-associated proteins, wherein each of the two or more fragments comprises a heteroclitic mutation.

39. The recombinant Listeria strain of claim 38, wherein the two or more immunogenic fragments are fused directly to each other without intervening sequence.

40. The recombinant Listeria strain of claim 38, wherein the two or more immunogenic fragments are linked to each other via peptide linkers.

41. The recombinant Listeria strain of claim 40, wherein one or more of the linkers set forth in SEQ ID NOS: 209-217 are used to link the two or more immunogenic fragments.

42. The recombinant Listeria strain of any one of claims 38-41, wherein the combination of two or more immunogenic fragments in the fusion polypeptide binds to each of the following HLA types: HLA-A*02:01, HLA-A*03:01, HLA-A*24:02, and HLA-B*07:02.

43. The recombinant Listeria strain of any one of claims 38-42, wherein the two or more immunogenic fragments comprise:
(a) two or more of the peptides set forth in Table 3;
(b) two or more of the peptides set forth in Table 5;
(c) two or more of the peptides set forth in Table 7;
(d) two or more of the peptides set forth in Table 9;
(e) two or more of the peptides set forth in Table 11;
(f) two or more of the peptides set forth in Table 13;
(g) two or more of the peptides set forth in Table 15;
(h) two or more of the peptides set forth in Table 17;
(i) two or more of the peptides set forth in Table 19; or
(j) two or more of the peptides set forth in Table 21.

44. The recombinant Listeria strain of claim 43, wherein the two or more immunogenic fragments comprise:
(a) all of the peptides set forth in Table 3;
(b) all of the peptides set forth in Table 5;
(c) all of the peptides set forth in Table 7;
(d) all of the peptides set forth in Table 9;
(e) all of the peptides set forth in Table 11;
(f) all of the peptides set forth in Table 13;
(g) all of the peptides set forth in Table 15;
(h) all of the peptides set forth in Table 17;
(i) all of the peptides set forth in Table 19; or
(j) all of the peptides set forth in Table 21.

45. The recombinant Listeria strain of any one of claims 29-44, wherein the PEST-containing peptide is on the N-terminal end of the fusion polypeptide.

46. The recombinant Listeria strain of claim 45, wherein the PEST-containing peptide is an N-terminal fragment of LLO.

47. The recombinant Listeria strain of claim 46, wherein the N-terminal fragment of LLO has the sequence set forth in SEQ ID NO: 59.

48. The recombinant Listeria strain of any one of claims 29-47, wherein the nucleic acid is in an episomal plasmid.

49. The recombinant Listeria strain of any one of claims 29-48, wherein the nucleic acid does not confer antibiotic resistance upon the recombinant Listeria strain.

50. The recombinant Listeria strain of any one of claims 29-49, wherein the recombinant Listeria strain is an attenuated, auxotrophic Listeria strain.

51. The recombinant Listeria strain of claim 50, wherein the attenuated, auxotrophic Listeria strain comprises a mutation in one or more endogenous genes that inactivates the one or more endogenous genes.

52. The recombinant Listeria strain of claim 51, wherein the one or more endogenous genes comprise actA, dal, and dat.

53. The recombinant Listeria strain of any one of claims 29-52, wherein the nucleic acid comprises a second open reading frame encoding a metabolic enzyme.

54. The recombinant Listeria strain of claim 53, wherein the metabolic enzyme is an alanine racemase enzyme or a D-amino acid aminotransferase enzyme.

55. The recombinant Listeria strain of any one of claims 29-54, wherein the fusion polypeptide is expressed from an hly promoter.

56. The recombinant Listeria strain of any one of claims 29-55, wherein the recombinant Listeria strain is a recombinant Listeria monocytogenes strain.

57. The recombinant Listeria strain of any one of claims 29-56, wherein the recombinant Listeria strain is an attenuated Listeria monocytogenes strain comprising a deletion of or inactivating mutation in actA, dal, and dat, wherein the nucleic acid is in an episomal plasmid and comprises a second open reading frame encoding an alanine racemase enzyme or a D-amino acid aminotransferase enzyme, and wherein the PEST-containing peptide is an N-terminal fragment of LLO.

58. An immunogenic composition comprising:
(a) the recombinant bacteria strain of any one of claims 22-28 or the recombinant Listeria strain of any one of claims 29-57; and
(b) an adjuvant.

59. The immunogenic composition of claim 58, wherein the adjuvant comprises a detoxified listeriolysin O (dtLLO), a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide

60. A method of inducing or enhancing an immune response against a tumor or cancer in a subject, comprising administering to the subject the isolated peptide of any one of claims 1-9, the nucleic acid of any one of claims 10-16, the pharmaceutical composition of any one of claims 17-21, the recombinant bacteria strain of any one of claims 22-28, the recombinant Listeria strain of any one of claims 29-57, or the immunogenic composition of any one of claims 58-59.

61. A method of preventing or treating a tumor or cancer in a subject, comprising administering to the subject the isolated peptide of any one of claims 1-9, the nucleic acid of any one of claims 10-16, the pharmaceutical composition of any one of claims 17-21, the recombinant bacteria strain of any one of claims 22-28, the recombinant Listeria strain of any one of claims 29-57, or the immunogenic composition of any one of claims 58-59.

62. The method of claim 60 or 61, wherein the cancer is non-small cell lung cancer, prostate cancer, pancreatic cancer, bladder cancer, breast cancer, uterine cancer, ovarian cancer, low-grade glioma, colorectal cancer, or head and neck cancer.
